(19) United States
(12) Patent Application Publication
(10) Pub. No.: US 2025/0041404 A1
(43) Pub. Date: Feb. 6, 2025

(54) T Cell Modulatory Polypeptides with Conjugation Sites and Methods of Use Thereof

(71) Applicant: Cue Biopharma, Inc., Boston, MA (US)

(72) Inventors: Ronald D. SEIDEL, III, Natick, MA (US); Rodolfo J. CHAPARRO, Cambridge, MA (US); John F. ROSS, Arlington, MA (US); Chee Meng LOW, Boston, MA (US); Anish SURI, Boston, MA (US); Raymond J. MONIZ, Brighton, MA (US); Gurpanna SAGGU, Westwood, MA (US)

(21) Appl. No.: 18/771,965

(22) Filed: Jul. 12, 2024

Related U.S. Application Data
(63) Continuation of application No. PCT/US2023/010767, filed on Jan. 13, 2023.
(60) Provisional application No. 63/299,405, filed on Jan. 13, 2022.

(51) Int. Cl. A61K 39/215; A61K 39/00; A61K 47/64; A61P 31/04; A61P 31/14; C12N 7/00

(52) U.S. Cl. CPC A61K 39/215; A61K 39/00114; A61K 47/646; A61P 31/04; A61P 31/14; C12N 7/00; A61K 2039/605; C12N 2770/20034

(57) ABSTRACT

The present disclosure provides T cell modulatory polypeptide-epitope conjugates (T-Cell-MP-epitope conjugates) comprising a chemical conjugation site at which a coronavirus peptide epitope is covalently attached and at least one immunomodulatory polypeptide sequence. The T-Cell-MP-epitope conjugates are useful for modulating the activity (e.g., increasing proliferation or cytotoxic activity) of T-cells specific to the coronavirus peptide epitope in an epitope selective/specific manner, and accordingly, for treating individuals with a coronavirus infection including long COVID. Alternatively, when the T-Cell-MP-epitope conjugate is redirected to a neoplastic cell by a targeting sequence, the redirected T-Cell-MP-epitope conjugate may be used to treat neoplasms including various cancers.

This application claims the benefit of U.S. Provisional Patent Application No. 63/299,405, filed Jan. 13, 2022.

### I. INCORPORATION OF SEQUENCE LISTING

The sequence listing in ST.26 XML format entitled 2910-30_PCT_ST26.xml, created on Jan. 13, 2023, comprising 2,834,559 bytes, prepared according to 37 CFR 1.822 to 1.824, submitted concurrently with the filing of this application, is incorporated herein by reference in its entirety.

### II. INTRODUCTION

Coronaviruses including MERS-CoV, Bat-SL-CoV, SARS-CoV, and SARS-CoV-2 (COVID-19) have been associated with a number of disease outbreaks having high lethality rates (e.g., Severe Acute Respiratory Syndrome or “SARS” and Middle East Respiratory Syndrome or “MERS”). The ability to induce an adaptive immune response to such viruses involves the engagement of the T cell receptor (TCR), present on the surface of a T-cell, with a small peptide antigen of the virus that is non-covalently presented on the surface of an antigen presenting cell (APC) by a major histocompatibility complex (MHC; also referred to in humans as a human leukocyte antigen (HLA) complex). This engagement represents the adaptive immune system's targeting mechanism and is a requisite molecular interaction for T cell modulation (activation or inhibition) and effector function. Following epitope-specific cell targeting, the targeted T-cells are activated through engagement of costimulatory proteins found on the APC with counterpart costimulatory proteins on the T-cells. Both signals—epitope/TCR binding and engagement of APC costimulatory proteins with T cell costimulatory proteins—are required to drive T cell specificity and activation or inhibition. The TCR is specific for a given epitope; however, the costimulatory protein is not epitope specific and instead is generally expressed on all T-cells or on large T cell subsets.

Cells of the innate immune system, including natural killer (“NK”) cells, may also respond to coronavirus infections including SARS-CoV-2 infections and may be activated in patients with acute coronavirus disease (see e.g., Hammer et al. Cell Reports 38:110503 (2022)).

### III. SUMMARY

The present disclosure provides T cell modulatory polypeptides (a “T-Cell-MP” or multiple “T-Cell-MPs”) that find use in, among other things, methods of in vivo and/or in vitro treatment of various viral infections or diseases (e.g., coronavirus infection or COVID disease) and associated disorders of mammals (e.g., humans) and the preparation of medicaments for such treatments. In one aspect, the T-Cell-MPs described herein comprise a portion of an MHC class I heavy chain (MHC-H) polypeptide, a β2M polypeptide, a chemical conjugation site for covalently attaching an epitope presenting molecule, and at least one immunomodulatory polypeptide (also referred to herein as a “MOD polypeptide” or, simply, a “MOD”). Any one or more of the MODs present in the T-Cell-MP may be wild-type (“wt.”) or a variant that exhibits an altered binding affinity to its cellular binding partner/receptor (e.g., T cell surface), referred to as a Co-MOD.

T-Cell-MPs as initially prepared are unconjugated, in which case they comprise at least one chemical conjugation site at which a molecule comprising a target antigenic determinate (e.g., a peptide, glycopeptide, or non-peptide such as a carbohydrate presenting an epitope from a coronavirus) may be covalently bound to form a T-Cell-MP-epitope conjugate (e.g., a T-Cell-MP-coronavirus epitope conjugate) for presentation of the epitope to a cell bearing a T cell receptor. Unconjugated T-Cell-MPs comprising a chemical conjugation site for linking an epitope are useful for rapidly preparing T-Cell-MP-epitope conjugates that can modulate the activity of T cells specific to the epitope presented and, accordingly, for modulating an immune response involving those T cells in an individual.

The T-Cell-MPs described herein are suitable for production in cell-based expression systems where most, or substantially all (e.g., greater than 75%, 85% or 90%) or all, of the expressed unconjugated T-Cell-MP polypeptide/protein is in a soluble non-aggregated state that is suitably stable at 37° C. for production in tissue culture and use up to at least that temperature. Greater than 85% or 90% of the expressed unconjugated T-Cell-MP polypeptide/protein may be in a soluble non-aggregated state that is suitably stable at 37° C. for production in tissue culture and use at least up to that temperature. The T-Cell-MPs can advantageously be produced as a single polypeptide encoded by a nucleic acid sequence contained in a single vector. The T-Cell-MPs may form higher order structures, such as duplexes (see, e.g., FIG. 1), which may be a homodimer as in FIG. 9, or heterodimeric when formed from two T-Cell-MPs, e.g., as illustrated in FIGS. 10 and 11. Unconjugated T-Cell-MPs can be expressed in favorable yields, e.g., greater than 25, 40, 60, or 80 mg/liter (e.g., about 25 to about 40, about 40 to about 60, or about 60 to about 80 mg/l in CHO cells). Yields can be high especially when a disulfide bond is present between the carboxyl end of the MHC-H chain α₁ helix and the MHC-H chain α₂₋₁ helix (e.g., a Y84C to A139C disulfide bond), and the linker between the MHC-H polypeptide sequence and the β2M polypeptide is of sufficient length (e.g., from about 10 to about 50 aas long). With the disulfide bond present between the α₁ and α₂ helices, unconjugated T-Cell-MP expression levels may exceed 80 mg/l (e.g., from about 80 to about 100, about 100 to about 120, about 120 to about 140, about 140 to about 160, about 160 to about 180, or about 180 to about 200 mg/l).

Once purified, most, substantially all (e.g., greater than 85% or 90% of the T-Cell-MP), or all of the expressed unconjugated T-Cell-MP protein remains in a soluble non-aggregated state even after conjugation to an epitope (e.g., peptide epitopes) and is similarly stable compared to the unconjugated T-Cell-MP.

As indicated above, T-Cell-MP-epitope conjugates may comprise wt. or variant MODs (e.g., IL-2, 4-1BBL, FasL, TGF-β, CD70, CD80, CD86, OX40L, ICOS-L, ICAM, JAG1 or variants thereof). The unconjugated T-Cell-MPs and their epitope conjugates may additionally comprise a targeting sequence that can direct a T-Cell-MP-epitope conjugate to a particular cell or tissue (e.g., a cancer cell or virus infected tissue or cell). The T-Cell-MPs and their epitope conjugates may additionally comprise sites for the conjugation and delivery of payloads such as antiviral agents for co-delivery with a MOD. Payloads include, but are not limited to, bioactive substances and labels, such as a therapeutic (e.g., antiviral agents or immunomodulator molecules) and may be covalently attached to a T-Cell-MP, such as by a crosslinking agent. In view of the foregoing, T-Cell-MP-epitope conjugates may be considered a means by which to deliver MODs and/or payloads (e.g., labels or antivirals) to cells in an epitope-specific manner, optionally with the assistance of a targeting sequence.

The T-Cell-MPs may comprise modifications that assist in the stabilization of the unconjugated T-Cell-MP during intracellular trafficking and/or following secretion by cells expressing the multimeric polypeptide even in the absence of an associated epitope (e.g., a peptide epitope). One such modification is a bond (e.g., disulfide bond) formed between amino acid position 84 at the carboxyl end of the MHC class I α₁ helix (or its flanking amino acid sequences aac1 and aac2) and amino acid position 139 at the amino end of the MHC-class I α₂₋₁ helix (or its flanking amino acid sequences aac3 and aac4). For example, the insertion of cysteine residues at amino acids 84 (Y84C substitution) and 139 (A139C substitution) of an HLA-A heavy chain, or the equivalent positions of other MHC-H polypeptide chains (see, e.g., FIG. 3I), may form a disulfide linkage that stabilize the T-Cell-MP. See, e.g., Z. Hein et al. (2014), Journal of Cell Science 127:2885-2897.

One aspect of the T-Cell-MP molecules described herein is broadly directed to an unconjugated T-Cell-MP, the polypeptide comprising (e.g., from N-terminus to C-terminus):
(i) optionally one or more MOD polypeptide sequences (e.g., two or more MOD polypeptide sequences, such as in tandem, wherein when there are two or more MOD polypeptide sequences they are optionally joined to each other by independently selected L1 linkers);
(ii) an optional L2 linker polypeptide sequence joining the one or more optional MOD polypeptide sequences to a β2M polypeptide sequence;
(iii) the β2M polypeptide sequence;
(iv) an optional L3 linker polypeptide sequence (e.g., from 10-50 aa in length);
(v) a class I MHC-H polypeptide sequence;
(vi) an optional L4 linker polypeptide sequence;
(vii) a scaffold polypeptide sequence (e.g., an immunoglobulin (Ig) Fc sequence);
(viii) an optional L5 linker polypeptide sequence; and
(ix) optionally one or more MOD polypeptide sequences (e.g., two or more MOD polypeptide sequences, such as in tandem, wherein when there are two or more MOD polypeptide sequences they are optionally joined to each other by independently selected L6 linkers);
wherein the unconjugated T cell modulatory polypeptide comprises at least one MOD polypeptide sequence (e.g., the MOD(s) of element (i) and/or (ix); and
wherein at least one of the β2M polypeptide sequence, the L3 linker polypeptide sequence, and/or the MHC-H polypeptide sequence comprises a chemical conjugation site (e.g., provided by protein engineering, such as a cysteine substitution) for epitope conjugation.

It is understood that such unconjugated T-Cell-MPs do not comprise a covalently attached moiety presenting an epitope, such as a peptide presenting an epitope (e.g., peptide, phosphopeptide, or glycopeptide epitope); however, the disclosure includes and provides for T-Cell-MP-epitope conjugates that further comprise a covalently attached peptide presenting an epitope from a coronavirus. The covalently attached peptide presenting a coronavirus epitope can be positioned within the binding cleft of the MHC-H/β2M polypeptide sequences and presented to a TCR, thereby permitting use of the molecules as agents for clinical testing and diagnostics, and as therapeutics, with respect to coronavirus. The T-Cell-MPs and their epitope conjugates described herein represent scalable antigen presenting cell-independent (APC-independent) immunotherapeutics that enable clinically effective levels of coronavirus antigen specific T cell modulation (e.g., inhibition or activation) depending on the MOD(s) present. Moreover, the scaffold portions of T-cell-MPs, which may be Ig Fc domains, permit multivalent presentation of MHC-epitope conjugate and MOD moieties to cognate T cells sufficient for their activation.

The T-Cell-MP-coronavirus epitope conjugates described herein may be employed for the treatment of primary infections by coronaviruses, but also may be employed for the treatment of patients known as “Long Haulers” suffering from prolonged effects of coronavirus infections termed Post Acute Sequelae of COVID-19 (PASC) or “Long COVID”. As defined by the U.S. Dept. of Health & Human Services: “Long COVID is broadly defined as signs, symptoms, and conditions that continue or develop after initial COVID-19 or SARS-CoV-2 infection. The signs, symptoms, and conditions are present four weeks or more after the initial phase of infection; may be multisystemic; and may present with a relapsing-remitting pattern and progression or worsening over time, with the possibility of severe and life-threatening events even months or years after infection. Long COVID is not one condition. It represents many potentially overlapping entities, likely with different biological causes and different sets of risk factors and outcomes.” On the World Wide Web at www.covid.gov/longcovid/definitions.

In addition to β2M, MHC Class I heavy chain, MOD and scaffold sequences, T-Cell-MPs may comprise additional polypeptide sequences, including targeting sequences. Targeting sequences permit T-Cell-MP-epitope conjugates to bind or associate with target cells and cell and tissues, such as cells of benign, precancerous, or malignant neoplasms. T-Cell-MP-epitope conjugate binding to target cells and/or tissues thereby permits T cells specific for the conjugated epitope to act upon (e.g., in granule dependent and/or granule independent manner) and thereby reduce or ablate the target cells and/or tissues. The inclusion of targeting sequences thereby permits T cells specific for conjugated epitopes prevalent in a population (e.g., T cells specific to antigens of utilized for vaccines against coronaviruses or other viruses such as adenovirus) to be recruited and used for the treatment of various disease such as cancers.

### IV. BRIEF DESCRIPTION OF THE DRAWINGS

FIG. 1 depicts preferential activation of an epitope-specific T cell relative to a T cell that is not specific for the epitope presented by an embodiment of a duplex T-Cell-MP-epitope conjugate. That conjugate has an indirect (via a linker) covalent attachment of the epitope to the β2M polypeptides and bears MODs, which can be wt. and/or variant MODs (e.g., having reduced affinity for their receptors (Co-MODs)). The first, epitope-specific T cell is activated due to productive engagement of both the TCRs and Co-MODs. In contrast, the second, T cell that is not specific for the presented epitope is not activated as the epitope cannot engage the TCR, and thus the MODs by themselves do not lead to productive engagement of the T-Cell-MP-epitope conjugate. The location of optional linkers are represented by black lines joining T-Cell-MP elements.

FIGS. 2A-2H provide amino acid sequences of Ig heavy chain polypeptides (including SEQ ID NOs: 1-13).

FIG. 2I provides the sequence of a human Ig J-chain (SEQ ID NO:14).

FIG. 2J provides the sequence of an Ig CH1 domain sequence (SEQ ID NO:15).

FIG. 2K provides sequences of Ig κ and Ig λ chains (SEQ ID NOs: 16-17).

FIGS. 3A, 3B and 3C provide amino acid sequences of human major histocompatibility complex class I heavy chain (MHC-H; also known as human leukocyte antigen (HLA) Class I heavy chain) polypeptides. Signal sequences, aas 1-24, are bolded and underlined. FIG. 3A entry: 3A.1 is the HLA-A heavy chain (HLA-A*01:01:01:01 or A*0101) (NCBI accession NP_001229687.1), SEQ ID NO: 18; entry 3A.2 is HLA-A*1101, SEQ ID NO: 19; entry 3A.3 is HLA-A*2402, SEQ ID NO:20, and entry 3A.4 is HLA-A*3303, SEQ ID NO:21. FIG. 3B provides the sequence for HLA-B*07:02:01 (HLA-B*0702) (NCBI GenBank Accession NP_005505.2), SEQ ID NO:22. FIG. 3C provides the sequence for HLA-C*0701 (GenBank Accession NP_001229971.1; EMBK-EBI Accession HLA00433), SEQ ID NO: 23.

FIG. 3D provides an alignment of all, or substantially all, of the α1, α2, and α3 domains of eleven mature MHC-H polypeptide sequences without all, or substantially all, of their leader, transmembrane and intracellular domain regions. The aligned sequences include human HLA-A*0101, SEQ ID NO:24 (see also SEQ ID NO:18); HLA-B*0702, SEQ ID NO:25; HLA-C, SEQ ID NO:26; HLA-A*0201, SEQ ID NO:27; a mouse H2K protein sequence, SEQ ID NO: 28; three variants of HLA-A (var. 2, var. 2C [having Y84C and A139C substitutions], and var. 2CP), SEQ ID NOs: 29-31; 3 human HLA-A molecules (HLA-A*1101 (HLA-A11), SEQ ID NO:32; HLA-A*2402 (HLA-A24), SEQ ID NO: 33; and HLA-A*3303 (HLA-A33), SEQ ID NO:34). HLA-A*0201 is a variant of HLA-A. The Y84A and A236C variant of HLA-A is marked as HLA-A (var. 2). The seventh HLA-A sequence, marked as HLA-A (var. 2C), shows HLA-A substituted with C residues at positions 84, 139 and 236, and the eighth sequence adds one additional proline to the C-terminus of the preceding sequence. The ninth through the eleventh sequences are from HLA-A11 (HLA-A*1101); HLA-A24 (HLA-A*2402); and HLA-A33 (HLA-A*3303), respectively, which are prevalent in certain Asian populations. Indicated in the alignment are the locations (84 and 139 of the mature proteins) where cysteine residues may be inserted in place of the aa at that position for the formation of a disulfide bond to stabilize the MHC-H-β2M complex in the absence of a bound peptide epitope. Also shown in the alignment is position 236 (of the mature polypeptide), which may be replaced by a cysteine residue that can form an intrachain disulfide bond with β2M (e.g., at aa 12 of the mature polypeptide forming, for example, an HLA-A*0201 A236C to β2M R12C disulfide bond). An arrow appears above each of those locations and the residues are bolded. The boxes flanking residues 84, 139 and 236 show the groups of five aas on either side of those six sets of five residues, denoted aa clusters 1, 2, 3, 4, 5, and 6 (shown in the figure as aac1 through aac6, respectively), that may be replaced by 1 to 5 aas selected independently from (i) any naturally occurring aa or (ii) any naturally occurring aa except proline or glycine.

FIGS. 3E-3G provide alignments of the aa sequences of all, or substantially all, of the α1, α2, and α3 domains of several mature HLA-A, -B, and -C class I heavy chains, respectively. The sequences are provided for a portion of the mature proteins (without all or substantially all of their leader sequences, transmembrane domains or intracellular domains). As described in FIG. 3D, the positions of aa residues 84, 139, and 236 and their flanking residues (aac1 to aac6) that may be replaced by 1 to 5 aas selected independently from (i) any naturally occurring aa or (ii) any naturally occurring aa except proline or glycine are also shown. A consensus sequence is also provided for each group of HLA alleles provided in the figures showing the variable aa positions as “X” residues sequentially numbered and the locations of aas 84, 139 and 236 double underlined.

FIG. 3H provides a consensus sequence for all, or substantially all, of the α1, α2, and α3 domains of each of HLA-E, -F, and -G polypeptides with the variable aa positions indicated as “X” residues sequentially numbered and the locations of aas 84, 139 and 236 double underlined.

FIG. 3I provides an alignment of the consensus aa sequences for HLA-A, -B, -C, -E, -F, and -G, which are given in FIGS. 3E to 3H (SEQ ID NOs: 39, 47, and 57-60). The alignment shows the correspondence of aas between the different sequences. Variable residues in each sequence are listed as “X” with the sequential numbering removed. The permissible aas at each variable residue can be determined by reference to FIGS. 3E-3H. As indicated in FIG. 3D, the locations of aas 84, 139 and 236 with their flanking five-aa clusters that may be replaced by 1 to 5 aas selected independently from (i) any naturally occurring aa or (ii) any naturally occurring aa except proline or glycine are also shown.

FIG. 4 provides a multiple aa sequence alignment of β2M precursors (i.e., including the leader sequence) from Homo sapiens (NP_004039.1; SEQ ID NO:61), Pan troglodytes (NP_001009066.1; SEQ ID NO:62), Macaca mulatta (NP_001040602.1; SEQ ID NO:63), Bos taurus (NP_776318.1; SEQ ID NO:64) and Mus musculus (NP_033865.2; SEQ ID NO:65). Underlined aas 1-20 are the signal peptide (sometime referred to as a leader sequence). The mature β2M sequences starts at aa 21.

FIG. 5 provides six unconjugated T-Cell-MP embodiments (structures) marked as A through F. In each case the T-Cell-MPs comprise: at least one MOD polypeptide sequence; a core structure that comprises the elements, in the N-terminal to C-terminal direction: a β2M polypeptide sequence, a Class I MHC-H polypeptide sequence comprising MHC-H α1, α2, and α3 domain sequences; and a scaffold polypeptide sequence (e.g., an Ig Fc polypeptide sequence). In the embodiments shown the α1 and α2 polypeptide sequences are linked by an intra-peptide bond between cysteines substituted, for example, with Tyr 84 and Ala 139 (Y84C and A139C substitutions). One or more MODs are located at the amino and/or carboxyl side of the core structure. Optional linker polypeptides that are selected independently, denoted as L1 to L6, are indicated by the line segments. The optional linker polypeptides may appear at either the ends of the T-Cell-MP polypeptide or joining the indicated polypeptide sequences. While the chemical conjugation site for coupling the epitope (epitope presenting molecule) can be located at any suitable location on the T-Cell-MP, potential locations in the β2M polypeptide sequence and the MHC-H polypeptide sequence for the chemical conjugation sites are indicated by asterisks. Although not shown, chemical conjugation sites may also be located in the L3 linker joining the β2M polypeptide sequence and MHC-H polypeptide sequence.

FIG. 6 provides six embodiments of unconjugated T-Cell-MPs, marked as A through F, that parallel the embodiments in FIG. 5. In the embodiments shown, the chemical conjugation site for coupling an epitope (marked by an asterisk “*”) is indicated as being present in the β2M polypeptide sequence (e.g., comprising an E44C substitution) and the scaffold is an Ig Fc region, which may be interspecific, thereby permitting two different unconjugated T-Cell-MPs to specifically combine to form a heteroduplex. The conjugation site may be in the MHC-H chain (not shown).

FIG. 7 provides examples of unconjugated T-Cell-MPs having different MOD substitutions (e.g., tandem IL-2 MODs in structure A). The chemical conjugation site for coupling an epitope (marked by an asterisk “*”) is indicated as being present in the β2M polypeptide sequence (e.g., an E44C substitution); however, the chemical conjugation site could be in the MHC-H polypeptide (the α1, α2, and α3 sequence), or in the linker joining the β2M and MHC polypeptides (not shown). The Fc scaffold may be replaced by any other scaffold sequence such as an interspecific Fc polypeptide sequence that can form a heterodimer with its counterpart sequence, and the specific linkers listed are only exemplary and may be replaced by other linker polypeptide sequences.

FIG. 8 shows some schematics of epitopes having a maleimide group appended for conjugation to a free nucleophile (e.g., cysteine) present in a T-Cell-MP to form an epitope conjugate. In “a” the maleimide group is attached by an optional linker (e.g., a peptide linker sequence) to the epitope. In “b”-“e,” the linker is a glycine serine polypeptide GGGGS (SEQ ID NO:130) repeated n times, where n is 1-5 when present, and n is 0 when the linker is absent. In “c”-“e” the attachment of a maleimide group is through a lysine (K) on the end of a GGGGS (SEQ ID NO: 130) containing linker, such as through the epsilon amino group of the lysine. In “d” and “e” the maleimide group is linked to the peptide through an alkyl amide formed with the epsilon amino group of a lysine (K) residue, where m is 1-7.

FIG. 9 depicts the formation of a conjugated T-Cell-MP homoduplex from an unconjugated T-Cell-MP having a scaffold (in this case an Ig Fc scaffold) shown at (A). The conjugated T-Cell-MP polypeptide from (A) forms a homoduplex as shown in (B) via interactions between the scaffold sequences. The unconjugated homoduplex may be isolated from cells stably or transiently expressing the T-Cell-MP protein. The unconjugated homoduplex, generally in a purified form, is subjected to chemical conjugation by coupling an epitope to the conjugation sites, which is exemplified by the reaction between a cysteine in the β2M polypeptide sequence (e.g., comprising an E44C substitution) and a maleimide labeled peptide to yield the T-Cell-MP-epitope conjugate shown in (C). Excess reactive peptide can be removed or substoichiometric amounts of the reactive epitope (relative to the amount of conjugation sites) can be utilized to produce the conjugated T-Cell-MP homoduplex. The constructs are not limited to the linker sequences shown, which are exemplary of the linkers that may be employed.

FIG. 10 depicts the formation of a conjugated T-Cell-MP heteroduplex from unconjugated T-Cell-MPs having scaffolds that selectively form heteroduplexes (in this case interspecific knob-in-hole Ig Fc scaffolds) shown at (A). The conjugated T-Cell-MP polypeptides form a heteroduplex as shown in (B) via interactions between the interspecific scaffold sequences. The unconjugated heteroduplex may be isolated from cells stably or transiently expressing the protein. The unconjugated heteroduplex, generally in a purified form, is subjected to chemical conjugation by coupling an epitope to the conjugation sites, which is exemplified by the reaction between a cysteine in the β2M polypeptide sequence (e.g., an E44C substitution) and a maleimide labeled peptide to yield the T-Cell-MP-epitope conjugate shown in (C). Excess reactive peptide can be removed or substoichiometric amounts of the reactive epitope (relative to the amount of conjugation sites) can be utilized to produce the conjugated T-Cell-MP heteroduplex, which as shown may comprise different MODs on each of the T-Cell-MP polypeptides. The constructs are not limited to the linker sequences shown, which are exemplary of the linkers that may be employed.

FIG. 11 shows three heterodimeric T-Cell-MP-epitope conjugate duplexes. Each has a scaffold comprising an interspecific Ig Fc polypeptide pair; however, the scaffold polypeptides may be replaced by any other interspecific polypeptide pair. The constructs are not limited to the linker sequences shown, which are exemplary of the linkers that may be employed.

FIG. 12 shows comparative results for the expression of a series of molecules including T-Cell-MPs in cultured CHO cells, described in Example 1, with the molecules (constructs) having varied substitutions in the L3 linker and at other locations. The overall structure of the molecules is provided at A, B, and C. The titer (amount of protein) of the molecules and fraction of the molecules that are unaggregated (e.g., existing as soluble duplexes) are provided in histograms D and E respectively.

FIG. 13 shows the production and stability in culture of an unconjugated T-Cell-MP (construct 3861, which has an L3 linker consisting of a Gly₄Ser repeated three times) at 2, 4, and 6 million cells per ml at both 32 and 28° over several days (A and B). The chromatograms show protein A purified material from a culture before (C) and after (D) further purification by size exclusion chromatography. The coomassie blue gel (E) shows that materials run against molecular weight standards (Mw) at 103128 Daltons for reduced (R) and 206213 Daltons for non-reduced samples. See Example 2 for details.

FIG. 14 at A demonstrates the specificity of the T-Cell-MP-epitope conjugates for T cells specific to the conjugated epitope. At B, FIG. 14 shows an electrophoresis gel of non-reduced and reduced samples of epitope conjugates. See Example 3 for details.

FIG. 15 and FIG. 16 show the response of CD8+ T cells present in Leukopak samples from CMV and MART-1 response donors to T-Cell-MP-epitope conjugates and control treatments as described in Example 4.

FIG. 17 shows the effect of L3 linker length on the CHO cell expression of two series of unconjugated T-Cell-MPs, providing the titer in culture media by Octet analysis at A, and the fraction of unaggregated (duplex) molecules present in the samples at B following purification on protein A magnetic beads.

FIG. 18 depicts methods of directing T cells (e.g., CD 8+ cytotoxic T-cells) specific for the epitope (unfilled triangles) presented by a T-Cell-MP to a target cell (e.g., a cancer cell labeled “Cancer Cell”) using one or two a targeting sequences (e.g., cancer targeting polypeptide or “CTP” as shown) and directing the T cell's cytotoxic response to the target cell. The target cell may also be a cell of a benign neoplasm, precancerous neoplasm, or a coronavirus infected cell, in which case the targeting sequence(s) would be directed to one or two distinct antigens present on those cells. In FIG. 18 each targeting peptide (CTP) is located at the C-terminus of the T-Cell-MP.

FIG. 19 provides the amino acid sequences of certain constructs discussed in this disclosure. Linker sequences (e.g., AAAGG SEQ ID NO: 132 and GGGGS SEQ ID NO: 130) may be bolded, italicized, and underlined to permit their identification. The indicated single amino acid substitutions in the MHC class I heavy chain are shown in bold with underlining. Human IL2 sequences are indicated by hIL2, beta-2-microglobin sequences are indicated by β2M, and HLA-A*02:01 sequences are indicated by HLA-A02, with each bearing the indicated aa substitutions.

FIG. 20A to FIG. 20J provide aa sequences of COVID-19 (SARS-CoV-2) proteins/polypeptides, including some derived from the polyprotein of open reading frame 1ab (ORF 1ab) set forth in NCBI Reference Sequence: NC_045512.2. FIG. 20J shows the surface glycoprotein (spike protein) sequences from the Wuhan-Hu-1 strain at (A) (see also Ref. Seq. YP_009724389.1), a delta strain at (B), and an omicron strain at (C). All changes in bold and larger font size with insertions underlined. The numbering of the delta and omicron strain spike protein in FIG. 20J at (B) and (C) have been maintained relative to the Wuhan strain in (A) for ease of reference even though insertions and deletions may have altered the actual positions.

FIG. 21 provides a table of predicted coronavirus epitopes (SARS-CoV and SARS-CoV-2), indicating for each their respective HLA restriction(s); adapted from Grifoni et al., Cell Host & Microbe 27:1-10 (2020); available on the world wide web (www) at doi.org/10.1016/j.chom.2020.03.002. Abbreviations: surface glycoprotein, S; membrane glycoprotein, MG; nucleocapsid phosphoprotein, NCPP; open reading frame, ORF; protein, Pro.

FIG. 22 provides a table of epitopes from SARS-CoV-2 proteins (S, surface glycoprotein; M, membrane protein; N, nucleocapsid phosphoprotein, ORF 1ab polyprotein) indicating for each their respective HLA restriction(s); adapted from Grifoni et al., Cell Host & Microbe 29(4):1076-1092 (2021); available on the www at doi.org/10.1016/j.chom.2021.05.010.

FIG. 23 illustrates the ability of a T-cell-MP-coronavirus epitope conjugate to promote the coronavirus epitope-specific proliferation of T cells within a pool of PBMC from individuals vaccinated with Moderna (MRRNA), Pfizer (PFE), or Johnson & Johnson (JNJ) vaccines.

### V. DEFINITIONS

The term T-Cell-MP is generic to, and includes, both unconjugated T-Cell-MPs and T-Cell-MP-epitope conjugates. The term “unconjugated T-Cell-MP (or “MPs” when plural) refers to T-Cell-MPs that have not been conjugated (covalently linked) to an epitope and/or payload (e.g., a non-epitope molecule such as a label), and therefore comprise at least one chemical conjugation site. Unconjugated T-Cell-MP polypeptides also do not comprise a fused peptide epitope that can be positioned within the MHC-H binding cleft and in conjunction with the β2M polypeptide sequence be presented to a TCR. The terms “T-Cell-MP-epitope conjugate” (or “conjugates” when plural) refers to T-Cell-MPs that have been conjugated (covalently linked) to an epitope at a chemical conjugation site that permits the covalently linked epitope to be present in the MHC binding cleft and presented to a TCR with specificity for the epitope expressed on a T Cell (an epitope-specific T cell). The term “T-Cell-MP-epitope conjugate(s)” includes higher order complexes such as duplexes unless stated otherwise. Where the T-Cell-MP-epitope conjugate comprises a conjugated coronavirus epitope it may be denoted as a “T-Cell-MP-coronavirus epitope conjugate”, which includes its higher order complexes such as duplexes. Throughout the disclosure where higher order complexes such as duplexes are called out in addition to, for example, a T-Cell-MP-coronavirus epitope conjugate, it is done for antecedent basis and/or emphasis.

T-Cell-MPs, unconjugated T-Cell-MPs, and T-Cell-MP-epitope conjugates may comprise one or more independently selected MODs or may be MOD-less. In those instances where this disclosure specifically refers to a T-Cell-MP that does not contain a MOD, terms such as “MOD-less T-Cell-MP” or a “T-Cell-MP without a MOD” and the like are employed. The term “T-Cell-MP” also includes unconjugated T-Cell-MPs and T-Cell-MP-epitope conjugates that comprise either one or more independently selected targeting sequences (discussed below).

“T-Cell-MP-payload conjugate” and “T-Cell-MP-payload conjugates” refer to T-Cell-MPs that have been conjugated (covalently linked) to one or more independently selected payloads. A T-Cell-MP can conjugated to both an epitope and payload to form a T-Cell-MP-epitope and payload conjugate.

The terms “polynucleotide” and “nucleic acid,” used interchangeably herein, refer to a polymeric form of nucleotides of any length, either ribonucleotides or deoxyribonucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” and “protein” are used interchangeably herein, and refer to a polymeric form of amino acids, which unless stated otherwise are the naturally occurring proteinogenic L-amino acids that are incorporated biosynthetically into proteins during translation in a mammalian cell. Furthermore, as used herein, a “polypeptide” and “protein” include modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References to a specific residue or residue number in a known polypeptide, e.g., position 72 or 75 of MHC polypeptide, are understood to refer to the amino acid at that position in the wild-type polypeptide (i.e. I72 or K75). To the extent that the sequence of the wild-type polypeptide is altered, either by addition or deletion of one or more amino acids, the specific residue or residue number will refer to the same specific amino acid in the altered polypeptide (e.g., in the addition of one amino acid at the N-terminus of a peptide reference as position I72, will be understood to indicate the amino acid, Ile, that is now position 73). Substitution of an amino acid at a specific position is denoted by an abbreviation comprising, in order, the original amino acid, the position number, and the substituted amino acid, e.g., substituting the Ile at position 72 with a cysteine is denoted as I72C.

A nucleic acid or polypeptide has a certain percent “sequence identity” to another nucleic acid or polypeptide, meaning that, when aligned, that percentage of bases or amino acids are the same, and in the same relative position, when comparing the two sequences. Unless stated otherwise, to determine sequence identity the sequences are aligned using the computer program BLAST (BLAST+2.10.0 using default parameters), which is available over the World Wide Web at sites including blast.ncbi.nlm.nih.gov/Blast.cgi for BLAST+2.10.0. Unless stated otherwise, for determining positions of corresponding amino acids, sequence comparisons are conducted using Clustal Omega Version 1.2.2 (using default parameters) available at on the World Wide Web at www.ebi.ac.uk/Tools/msa/clustalo/.

As used herein amino acid (“aa” singular or “aas” plural) means the naturally occurring proteinogenic amino acids incorporated into polypeptides and proteins in mammalian cell translation. Unless stated otherwise, these are: L (Leu, leucine), A (Ala, alanine), G (Gly, glycine), S (Ser, serine), V (Val, valine), F (Phe, phenylalanine), Y (Tyr, tyrosine), H (His, histidine), R (Arg, arginine), N (Asn, asparagine), E (Glu, glutamic acid), D (Asp, aspartic acid), C (Cys, cysteine), Q (Gln, glutamine), I (Ile, isoleucine), M (Met, methionine), P (Pro, proline), T (Thr, threonine), K (Lys, lysine), and W (Trp, tryptophan). Amino acid also includes the amino acids, hydroxyproline and selenocysteine, which appear in some proteins found in mammalian cells; however, unless their presence is expressly indicated they are not understood to be included.

The term “conservative amino acid substitution” refers to the interchangeability in proteins of aa residues having similar side chains. For example, a group of aas having aliphatic side chains consists of glycine, alanine, valine, leucine, and isoleucine; a group of aas having aliphatic-hydroxyl side chains consists of serine and threonine; a group of aas having amide containing side chains consists of asparagine and glutamine; a group of aas having aromatic side chains consists of phenylalanine, tyrosine, and tryptophan; a group of aas having basic side chains consists of lysine, arginine, and histidine; a group of aas having acidic side chains consists of glutamate and aspartate; and a group of aas having sulfur containing side chains consists of cysteine and methionine. Exemplary conservative aa substitution groups are: valine-leucine-isoleucine, phenylalanine-tyrosine, lysine-arginine, alanine-valine-glycine, and asparagine-glutamine.

As used herein the term “in vivo” refers to any process or procedure occurring inside of the body, e.g., of a patient.

As used herein, “in vitro” refers to any process or procedure occurring outside of the body.

The term “binding” (or “bound”) refers generically to a direct association between molecules and/or atoms, due to, for example, covalent, electrostatic, hydrophobic, ionic and/or hydrogen-bond interactions, including interactions such as salt bridges and water bridges.

The term “binding” (or “bound”) as used with reference to a T-Cell-MP binding to a polypeptide (e.g., a T cell receptor on a T cell) refers to a non-covalent interaction between two molecules. A non-covalent interaction refers to a direct association between two molecules, due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Covalent bonding” or “covalent binding” as used herein refers to the formation of one or more covalent chemical bonds between two different molecules.

“Affinity” as used herein generally refers to the strength of non-covalent binding, increased binding affinity being correlated with a lower K_{D}. As used herein, the term “affinity” may be described by the dissociation constant (K_{D}) for the reversible binding of two agents (e.g., an antibody and an antigen). Affinity can be at least 1-fold greater to at least 1,000-fold greater (e.g., at least 2-fold to at least 5-fold greater, at least 3-fold to at least 6-fold greater, at least 4-fold to at least 8-fold greater, at least 5-fold to at least 10-fold greater, at least 6-fold to at least 15-fold greater, at least 7-fold to at least 20-fold greater, at least 8-fold to at least 30-fold greater, at least 9-fold to at least 35-fold greater, at least 10-fold to at least 40-fold greater, at least 20-fold to at least 60-fold greater, at least 40-fold to at least 80-fold greater, at least 60-fold to at least 180-fold greater, at least 80-fold to at least 240-fold greater, at least 100-fold to at least 1,000-fold greater, or at least 1,000-fold greater) than the affinity of an antibody or receptor for an unrelated aa sequence (e.g., ligand). Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution.

The term “immunological synapse” or “immune synapse” as used herein generally refers to the natural interface between two interacting immune cells of an adaptive immune response including, e.g., the interface between an antigen-presenting cell (APC) or target cell and an effector cell, e.g., a lymphocyte, an effector T cell, a natural killer cell, and the like. An immunological synapse between an APC and a T cell is generally initiated by the interaction of a T cell antigen receptor and MHC molecules, e.g., as described in Bromley et al., Ann. Rev. Immunol., 19:375-96 (2001); the disclosure of which is incorporated herein by reference in its entirety.

“T cell” includes all types of immune cells expressing CD3, including T-helper cells (CD4⁺ cells), cytotoxic T cells (CD8⁺ cells), regulatory T cells (T reg), and NK-T cells.

The term “immunomodulatory polypeptide” (also referred to as a “costimulatory polypeptide” or, as noted above, a “MOD”) as used herein includes a polypeptide or portion thereof (e.g., an ectodomain) on an APC (e.g., a dendritic cell, a B cell, and the like), or otherwise available to interact with the T cell, that specifically binds a cognate co-immunomodulatory polypeptide (“Co-MOD”) present on a T cell, thereby providing a signal. The signal provided by the MOD engaging its Co-MOD, in addition to the primary signal provided by, for instance, binding of a TCR/CD3 complex with a MHC polypeptide loaded with a peptide epitope, mediates (e.g., directs) a T cell response. The responses include, but are not limited to, proliferation, activation, differentiation, and the like. A MOD can include, but is not limited to, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, lymphotoxin beta receptor, 3/TR6, ILT3, ILT4, HVEM, an agonist or antibody that binds Toll-Like Receptor (TLR), and a ligand that specifically binds with B7-H3. A MOD also encompasses, inter alia, an antibody or antibody fragment that specifically binds with and activates a Co-MOD molecule present on a T cell such as, but not limited, to antibodies against the receptors for any of IL-2, CD27, CD28, 4-1BB, OX40, CD30, CD40, PD-1, ICOS, lymphocyte function-associated antigen-1 (LFA-1), CD2, LIGHT (also known as tumor necrosis factor superfamily member 14 (TNFSF14)), NKG2C, B7-DC, B7-H2, B7-H3, and CD83.

An immunomodulatory domain of a T-Cell-MP is a polypeptide of the T-Cell-MP or part thereof that acts as a MOD.

“Recombinant” as used herein means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. The terms “recombinant expression vector” or “DNA construct,” used interchangeably herein, refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

The terms “treatment,” “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease and, in some cases, after the symptomatic stage of the disease.

The terms “individual,” “subject,” “host,” and “patient” are used interchangeably herein and refer to any mammalian subject for whom diagnosis, treatment, or therapy is desired. Mammals include humans and non-human primates, and in addition include rodents (e.g., rats; mice), lagomorphs (e.g., rabbits), ungulates (e.g., cows, sheep, pigs, horses, goats, and the like), felines, canines, etc.

Unless indicated otherwise, the term “substantially” is intended to encompass both “wholly” and “largely but not wholly”. For example, an Ig Fc that “substantially does not induce cell lysis” means an Ig Fc that induces no cell lysis at all or that largely but not wholly induces no cell lysis.

As used herein, the term “about” used in connection with an amount indicates that the amount can vary by 10%. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

Where a range of values is provided, it is understood that each intervening value between the upper and lower limit of that range to a tenth of the lower limit of the range is encompassed within the disclosure along with any other stated or intervening value in the range. Upper and lower limits may independently be included in smaller ranges that are also encompassed within the disclosure subject to any specifically excluded limit in the stated range. Where the stated range has a value (e.g., an upper or lower limit), ranges excluding those values are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that, as used herein and in the appended claims, the singular forms “a,” “an,” and “the” include plural referents unless the context clearly dictates otherwise. Thus, for example, reference to “a T reg” includes a plurality of such T regs and reference to “the MHC Class I heavy chain” includes reference to one or more MHC Class I heavy chains and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publications by virtue of prior invention. Further, the dates of publication provided may be different from the actual publication dates which may need to be independently confirmed.

Before the present invention is further described, it is to be understood that this invention is not limited to the particular embodiments described, as such may, of course, vary. It is also to be understood that the terminology used herein is for the purpose of describing particular embodiments only, and is not intended to limit the scope of the invention

### VI. DETAILED DESCRIPTION

T Cell Modulatory Polypeptides (T-Cell-MPs) with Chemical Conjugation Sites for Epitope Binding

The present disclosure includes and provides for T-Cell-MPs (both unconjugated T-Cell-MPs having a chemical conjugation site suitable for attaching an epitope and T-Cell-MP-epitope conjugates to which an epitope has been conjugated). Such T-Cell-MPs are useful for modulating the activity of T cells to, for example, modulate an immune response in vitro, or in vivo, and accordingly to effect therapeutic treatments. The present disclosure specifically provides methods of T-Cell-MP-epitope conjugate preparation and the use of the conjugate in modulating an immune response to a coronavirus in an individual that may be a human or non-human test subject or patient. The human or non-human test subject or patient may be suffering from coronavirus infection or COVID disease or may be positive for coronavirus infection. In addition to the other elements present (e.g., MHC-H, β2M, scaffold etc.), the T-Cell-MPs may comprise one or more independently selected wt. and/or variant MOD polypeptides that exhibit reduced binding affinity to their Co-MODs and one or more payloads.

Included in this disclosure are T-Cell-MPs that are homodimeric, comprising identical first and second T-Cell-MP polypeptides. Also included in this disclosure are T-Cell-MPs that are heterodimeric, comprising a first and a second T-Cell-MP polypeptide, wherein at least one of those polypeptides comprises a chemical conjugation site for the attachment of an epitope. Optionally at least one of the heterodimers may comprise a payload such as an antiviral agent and/or a targeting sequence. Included in this disclosure are T-Cell-MPs which have been chemically conjugated to an epitope to form a T-Cell-MP-epitope conjugate and which optionally comprise a targeting sequence and/or a payload.

Depending on the type of MOD(s) present in a T-Cell-MP-epitope conjugate, a T cell bearing a TCR specific to the epitope presented by the T-Cell-MP-epitope conjugate can respond by undergoing activation including, for example, clonal expansion (e.g., when activating MODs such as wt. and/or variants of IL-2, 4-1BBL and/or CD80 that are incorporated into the T-Cell-MP). Activating MODs present in a T-Cell-MP-epitope conjugate may also increase, for example, granule dependent responses by T cells bearing a TCR specific to the epitope presented by the T-Cell-MP-epitope conjugate. Alternatively, the T cell may undergo inhibition that down regulates T cell activity when inhibitory MODs such as wt. and/or variants of FASL and/or PD-L1 are incorporated into the T-Cell-MPs. The incorporation of combinations of MODs such as wt. and/or variants of IL-2 and CD80 or IL2 and PD-L1 into T-Cell-MPs (e.g., T-Cell-MP-epitope conjugates) may lead to synergistic effects where the T cell response more than exceeds the sum of the responses of T cells to otherwise identical T-Cell-MPs lacking one of the MODs. Because MODs are not specific to any epitope, activation or inhibition of T cells can be biased toward epitope-specific interactions by incorporating variant MODs (e.g., MODs having reduced affinity for their Co-MOD) into the T-Cell-MPs such that the binding of a T-Cell-MP to a T cell is strongly affected by, or even dominated by, the MHC-epitope-TCR interaction.

A T-Cell-MP-epitope conjugate bearing MODs may be considered to function as a surrogate APC and, by interacting with a T-Cell, mimic the presentation of epitope in an adaptive immune response. The T-Cell-MP-epitope conjugate does so by engaging and presenting to a TCR present on the surface of a T cell with a covalently bound epitope (e.g., a peptide presenting an epitope). This engagement provides the T-Cell-MP-epitope conjugate with the ability to achieve epitope-specific cell targeting. In embodiments described herein, T-Cell-MP-epitope conjugates also possess at least one MOD that engages a counterpart costimulatory protein (Co-MOD) on the T cell. Both signals-epitope/MHC binding to a TCR and MOD binding to a Co-MOD-then drive both the desired T cell specificity and either inhibition/apoptosis or activation/proliferation.

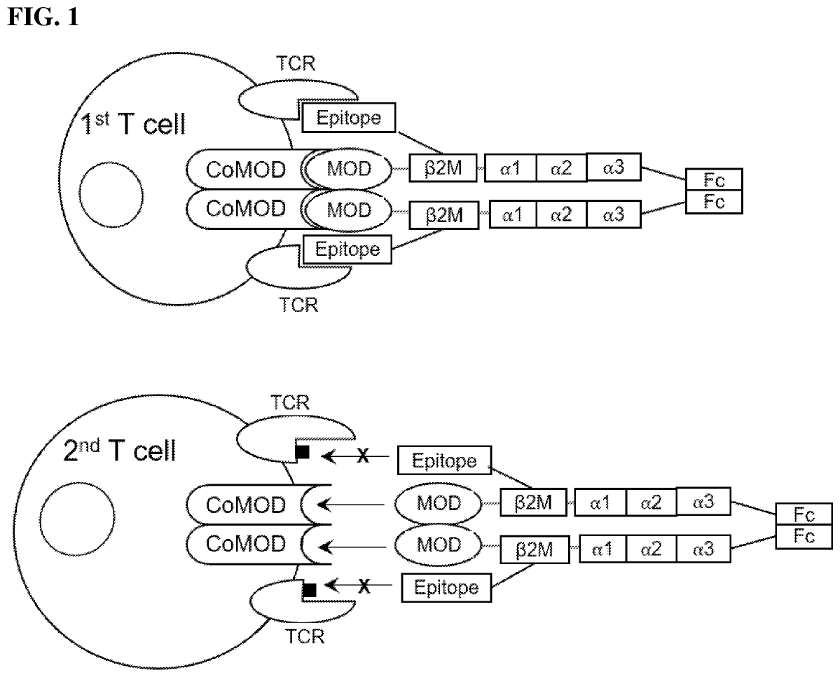

Unconjugated T-Cell-MPs, which have chemical conjugation sites, find use as a platform into which different epitopes may be introduced, either alone or in combination with one or more additional payloads added to the T-Cell-MP, in order to prepare materials for therapeutic, diagnostic and research applications. Because T-Cell-MPs, including duplexes comprised of homodimers, and higher order homomeric complexes require only a single polypeptide sequence, they can advantageously be introduced and expressed by cells using a single vector with a single expression cassette. Similarly, heterodimeric duplex T-Cell-MPs can be introduced into cells using a single vector with two separate expression cassettes or a bicistronic expression cassette (e.g. with the proteins separated by a 2A protein sequence or internal ribosome entry sequence (IRES)), or by using two vectors each bearing a cassette coding one heterodimeric subunit. Where duplex or higher order T-Cell-MPs contain interspecific scaffold sequences, the different T-Cell-MPs may bear different MODs permitting the duplex or higher order structure to contain different MODs, or MODs at different locations on each polypeptide of the heterodimer. The modular nature of T-Cell-MPs enables the rapid preparation and testing of diagnostic and therapeutic candidates for coronavirus treatments by coupling an epitope containing molecule (e.g., a peptide) into prepared T-Cell-MP polypeptides that can then be tested for activation or inhibition of T cells bearing TCRs specific to the coronavirus epitope. The ability to construct unconjugated T-Cell-MPs, and in particular heterodimer T-Cell-MP duplexes with different MODs, permits rapid assembly and assessment of different combinations of MODs with one or more epitope relevant to coronavirus disease or infection. Further to the foregoing, the ability to rapidly attach and access the effectiveness of various payloads, such as antiviral agents, and/or targeting sequences, to the T-Cell-MP facilitates preparation of T-Cell-MPs both for screening and as therapeutics for coronavirus. In some instances, a chemical conjugation site of a T-Cell-MP other than the conjugation site utilized for the attachment of the epitope may be utilized to attach a payload such as an antiviral agent to the T-Cell-MP, or a targeting sequence (e.g. a polypeptide comprising a targeting sequence) such as a cancer-targeting sequence.

Where one or more activating wt. MOD or variant MOD polypeptide sequences are incorporated into a T-Cell-MP-epitope conjugate, contacting the T cells with a TCR specific to the epitope with at least one concentration of the T-Cell-MP-epitope conjugate can result in T cell activation. T cell activation may result in one or more of the following: an increase in the activity of ZAP-70 protein kinase activity, induction in the proliferation of the T-cell(s), granule-dependent effector actions (e.g., the release of granzymes, perforin, and/or granulysin from cytotoxic T-cells), and/or release of T cell cytokines (e.g., interferon γ from CD8+ cells). Where the MOD polypeptide sequence(s) induces T cell proliferation, the T-Cell-MP-epitope conjugate may induce at least a twofold (e.g., at least a 2, 3, 4, 5, 10, 20, 30, 50, 75, or 100 fold) difference in the activation of T cells having a TCR specific to the epitope as compared to T cells contacted with the same concentration of the T-Cell-MP-epitope conjugate that do not have a TCR specific to the epitope (see FIG. 1). Activation of T-cells may be measured by, for example, ZAP-70 activity or T cell proliferation (see, e.g., Wang, et al., Cold Spring Harbor perspectives in biology 2.5 (2010): a002279), or cytokine release. Where one or more wt. or variant MOD polypeptide sequences that inhibit T cell activation are incorporated into a T-Cell-MP-epitope conjugate, contacting the T cells having a TCR specific to the epitope with at least one concentration of the T-Cell-MP-epitope conjugate may result in one or more of the following: prevention or inhibition of the T cell's activation, reduction in the response of activated T cells, and/or down regulation of the epitope-specific T-Cell. In some cases, inhibitory MODs present in a T-Cell-MP-epitope conjugate may result in apoptosis of T cell(s) with a TCR specific to the epitope. The effects of inhibitory MOD sequences may be measured by, for example, one or more of their: effect on T cell proliferation, ZAP-70 activity, reduction in granule-dependent effector actions, and/or cell death.

Where variant MODs that inhibit T cell activation and a peptide presenting a coronavirus epitope are incorporated into a T-Cell-MP, contacting the T-cells with at least one concentration of the T-Cell-MP-coronavirus epitope conjugate prevents activation of T-cells in an epitope-specific manner as measured by, for example, T cell proliferation (inhibition of proliferation) or the inhibition of granule dependent of independent responses. Such molecules may be used, for example, where a patient is experiencing a cytokine storm in response to a coronavirus infection.

The specificity of T-Cell-MP-epitope conjugates depends on the relative contributions of the epitope and the MODs to the binding. Where the affinity of the MOD(s) for the Co-MOD(s) is relatively high such that the MOD(s) dominate the T-Cell-MPs in the binding interactions, the specificity of the T-Cell-MP-epitope conjugates will be reduced relative to T-Cell-MP complexes where the epitope dominates the binding interactions by contributing more to the overall binding energy than the MODs. The greater the contribution of binding energy between an epitope and a TCR specific to the epitope, the greater the specificity of the T-Cell-MP will be for the T cell bearing that type of TCR. Where an epitope MHC complex has strong affinity for its TCR, the use of wt. MODs that have relatively low affinity and/or variant MODs with reduced affinity for their Co-MODs will favor epitope selective interactions of the T-Cell-MP-epitope conjugates with specific T cells, and also facilitate selective delivery of any payload that may be conjugated to the T-Cell-MP-epitope conjugate to the T cell and/or locations where the T cell is located.

Accordingly, the present disclosure provides T-Cell-MP-epitope conjugates presenting coronavirus epitopes that are useful for modulating the activity of T-cells in an epitope-specific manner and, accordingly, for modulating an immune response to coronaviruses (e.g., SARS-CoV or SARS-CoV-2) in an individual. Such T-Cell-MPs may comprise one or more MODs that are either wt. and/or variants (e.g., variants that exhibit reduced binding affinity to a Co-MOD).

### A. Unconjugated T-Cell-MPs and T-Cell-MP-Epitope Conjugates

### 1 The Structure and Composition of Unconjugated T-Cell-MPs and T-Cell-MP-Epitope Conjugate Components

The unconjugated T-Cell-MPs described herein comprise a chemical conjugation site for coupling an epitope directly, or indirectly through a linker. The chemical conjugation site can be situated at any location on the T-Cell-MP. One aspect of the disclosure is directed to T-Cell-MPs that comprise a chemical conjugation site for the attachment of a peptide epitope within the scaffold (e.g., Ig Fc), β2M, or MHC-H polypeptide sequences, or within the linker (L3) joining the β2M and MHC-H polypeptide sequences, and higher order complexes of those T-Cell-MPs. Another aspect of the disclosure is directed to T-Cell-MPs that comprise a chemical conjugation site for the attachment of a peptide epitope within the β2M, or MHC-H polypeptide sequences, or within the linker (L3) joining the β2M and MHC-H polypeptide sequences, and higher order complexes of those T-Cell-MPs. A chemical conjugation site for coupling an epitope directly, or indirectly through a linker, can be situated in the β2M polypeptide sequence. A chemical conjugation site for coupling an epitope directly, or indirectly through a linker, can be situated in the MHC-H polypeptide sequence. A chemical conjugation site for coupling an epitope directly, or indirectly through a linker, can be situated in the linker (L3) joining the β2M polypeptide sequence and MHC-H polypeptide sequence. A chemical conjugation site for coupling an epitope directly, or indirectly through a linker, can be situated within the scaffold (e.g., Ig Fc). Where a chemical conjugation site for coupling an epitope to an unconjugated T-Cell-MP appears in a scaffold (e.g., an Ig Fc), β2M, or MHC-H polypeptide sequence, the chemical conjugation site may be limited to an amino acid or sequence of amino acids not naturally appearing in any of those sequences, and may involve one or more amino acids introduced into one of those sequences (e.g., one or more aas introduced into an aa sequence position at which the one or more aas do not appear in the naturally occurring sequence). In addition, while it is possible to utilize the N-terminal amino group or C-terminal carboxyl group of a T-Cell-MP polypeptide as a chemical conjugation site for epitope attachment, those sites may be excluded as conjugation sites from any of the T-Cell-MPs or their higher order complexes described herein. Indeed, the chemical conjugation site of a T-Cell-MP may be excluded from the N-terminal 10 or 20 aas and/or the C-terminal 10 or 20 aas.

T-Cell-MPs may form higher order complexes (e.g., duplexes, triplexes, etc.). The higher order complexes may be duplexes that are homomeric (e.g., homodimers or homoduplexes) or heteromeric (e.g., heterodimers or heteroduplexes). Pairs of interspecific sequences may be employed as scaffold sequences where the complexes are intended to be heterodimeric as they permit two different T-Cell-MPs to form a specific heteroduplex, as opposed to a mixture of homoduplexes and heteroduplexes that can form if two T-Cell-MPs not having a pair of interspecific binding sequences are mixed (e.g., co-expressed).

A first group of T-Cell-MP molecules described herein is broadly directed to T-Cell-MPs that may form a duplex that associates through interactions in their scaffold sequences. Such T-Cell-MPs may have at least a first T-Cell-MP polypeptide sequence (e.g., duplexed as a homodimer), or non-identical first and second T-Cell-MP polypeptide sequences (e.g., duplexed as a heterodimer), with one or both of the T-Cell-MPs comprising (e.g., from N-terminus to C-terminus):
(i) optionally one or more MOD polypeptide sequences (e.g., two or more MOD polypeptide sequences, such as in tandem, wherein when there are two or more MOD polypeptide sequences they are optionally joined to each other by independently selected L1 linkers);
(ii) an optional L2 linker polypeptide sequence joining the one or more MOD polypeptide sequences to a β2M polypeptide sequence;
(iii) the β2M polypeptide sequence;
(iv) an optional L3 linker polypeptide sequence (e.g., from 10-50 aa in length);
(v) a class I MHC-H polypeptide sequence;
(vi) an optional L4 linker polypeptide sequence;
(vii) a scaffold polypeptide sequence (e.g., an Ig Fc sequence);
(viii) an optional L5 linker polypeptide sequence; and
(ix) optionally one or more MOD polypeptide sequence (e.g., two or more MOD polypeptide sequences, such as in tandem, wherein when there are two or more MOD polypeptide sequences they are optionally joined to each other by independently selected L6 linkers);
wherein the unconjugated T-Cell-MP comprises at least one MOD polypeptide sequence (e.g., the MOD(s) of element (i) and/or (ix)); and
wherein at least one of the β2M polypeptide sequence, the L3 linker polypeptide sequence, and/or the MHC-H polypeptide sequence comprises at least one chemical conjugation site.

A second group of unconjugated T-Cell-MPs described herein may form a duplex between a first T-Cell-MP and a second T-Cell-MP that associate through interactions in their scaffold sequences. Such unconjugated duplex T-Cell-MPs may have an identical first and second T-Cell-MP polypeptide sequence duplexed as a homodimer, or non-identical first and second T-Cell-MP polypeptide sequences duplexed as a heterodimer, with one or both of the T-Cell-MPs comprising from N-terminus to C-terminus:
(i) optionally one or more MOD polypeptide sequences (e.g., two or more MOD polypeptide sequences, such as in tandem, wherein when there are two or more MOD polypeptide sequences they are optionally joined to each other by independently selected L1 linkers);
(ii) an optional L2 linker polypeptide sequence joining the one or more optional MOD polypeptide sequences to a β2M polypeptide sequence;
(iii) the β2M polypeptide sequence;
(iv) an optional L3 linker polypeptide sequence (e.g., from 10-50 aa in length);
(v) a class I MHC-H polypeptide sequence;
(vi) an optional L4 linker polypeptide sequence;
(vii) a scaffold polypeptide sequence (e.g., an Ig Fc sequence);
(viii) an optional L5 linker polypeptide sequence; and
(ix) optionally one or more MOD polypeptide sequence (e.g., two or more MOD polypeptide sequences, such as in tandem, wherein when there are two or more MOD polypeptide sequences they are optionally joined to each other by independently selected L6 linkers);
wherein the unconjugated T cell modulatory polypeptide comprises at least one MOD polypeptide sequence (e.g., the MOD(s) of element (i) and/or (ix)); and
wherein at least one of the β2M polypeptide sequence, the L3 linker polypeptide sequence, and/or the MHC-H polypeptide sequence comprises at least one chemical conjugation site, e.g., for epitope conjugation and/or payload conjugation.

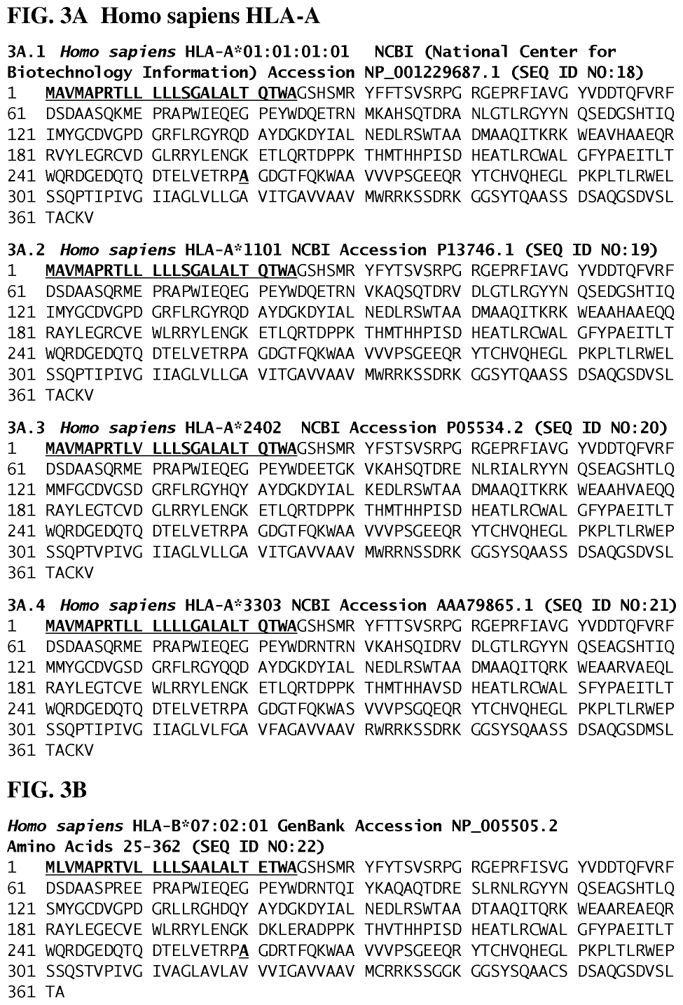

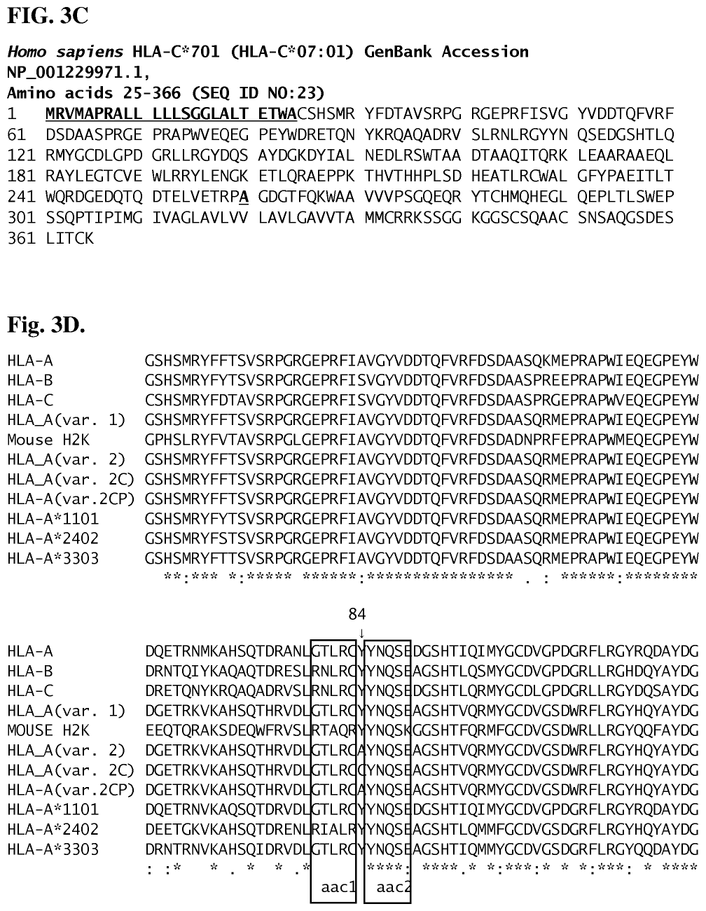

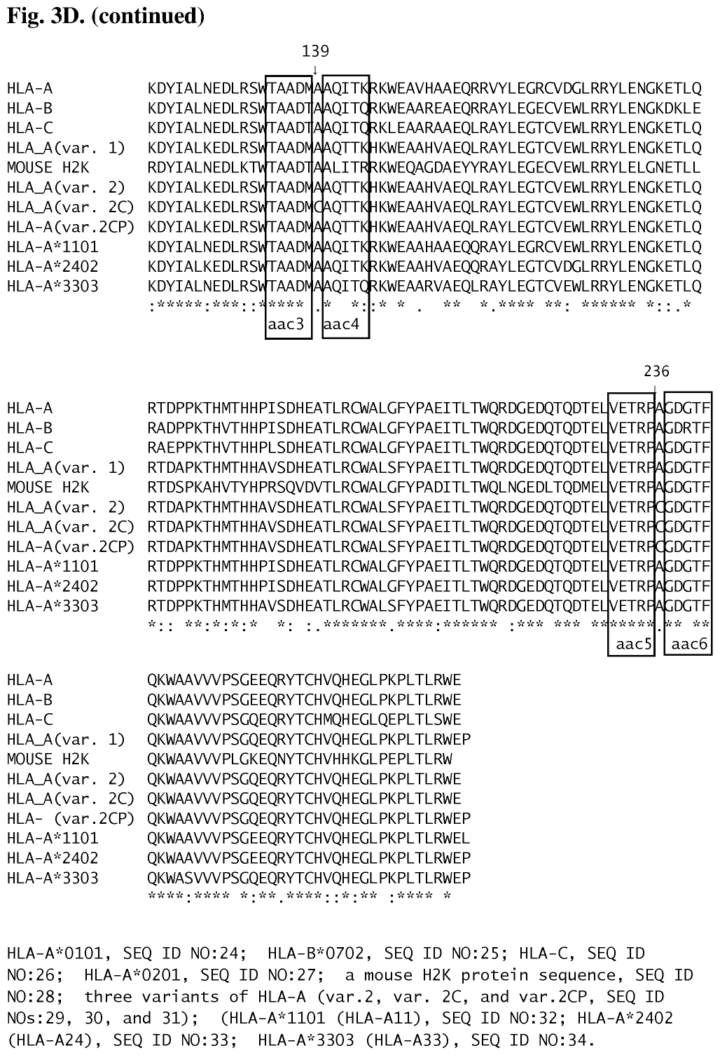

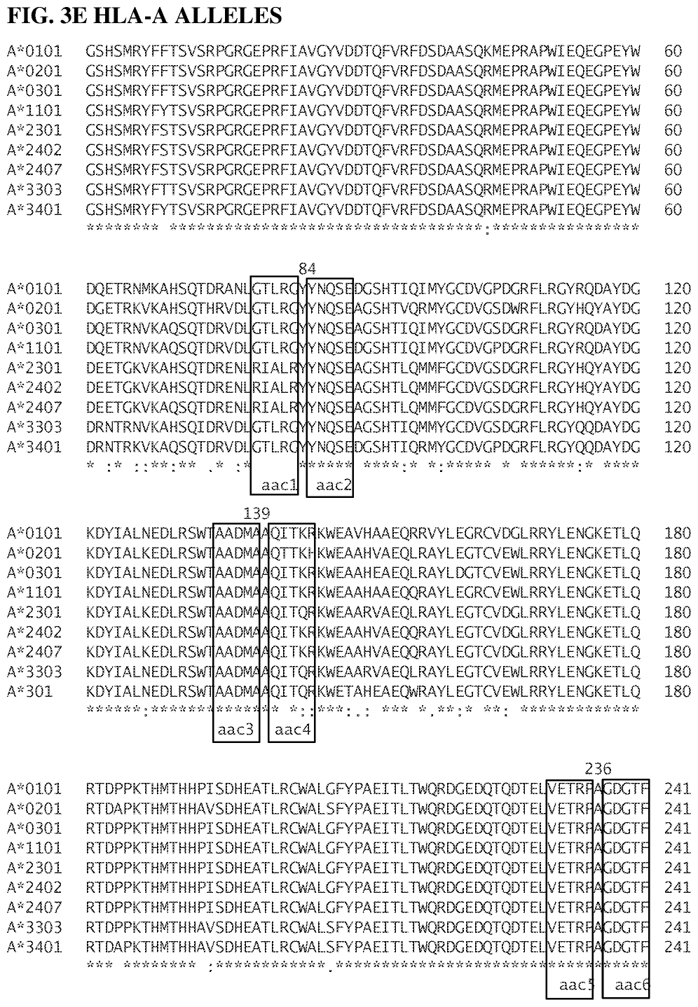

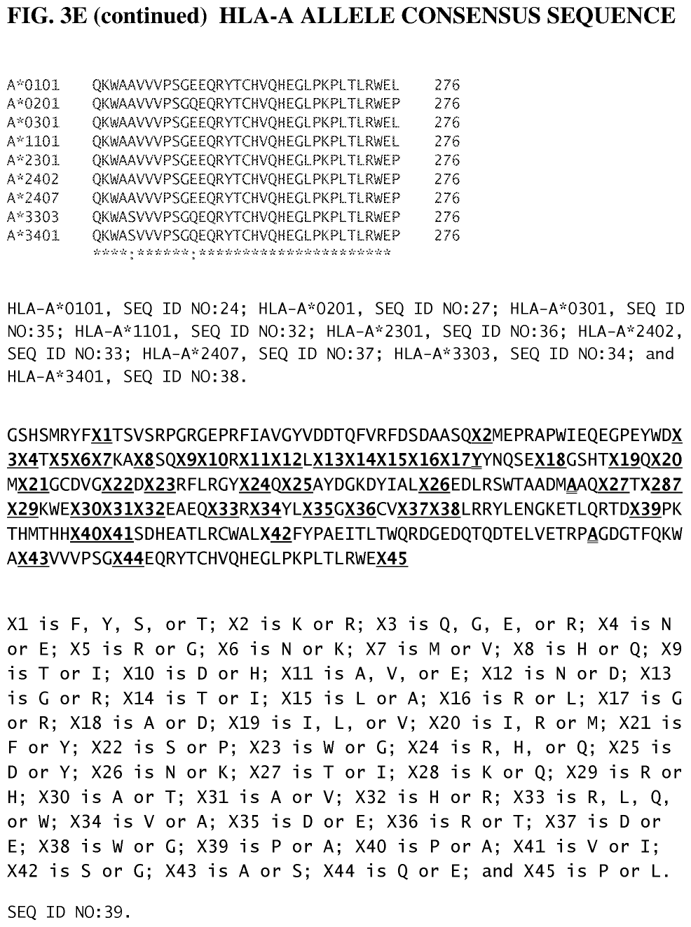

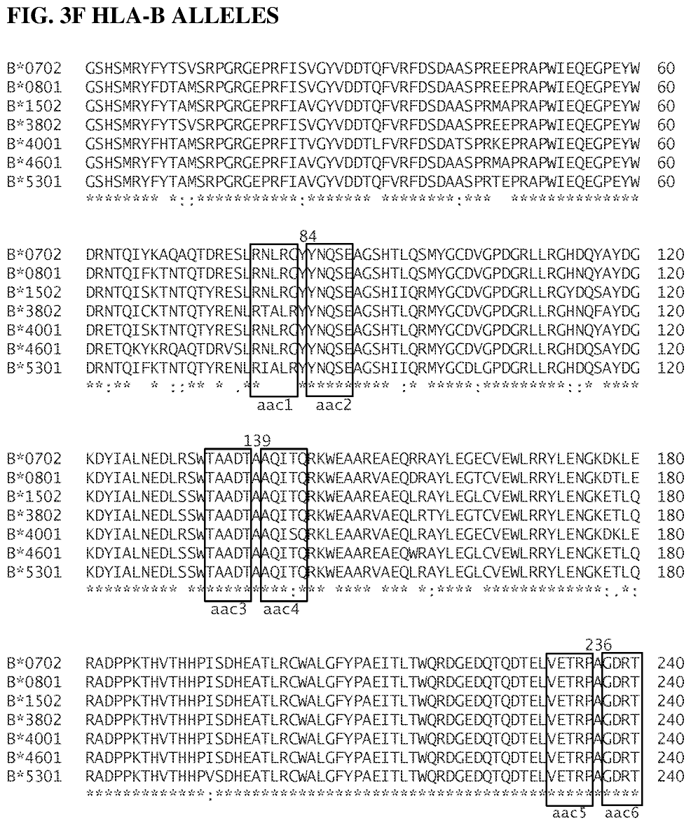

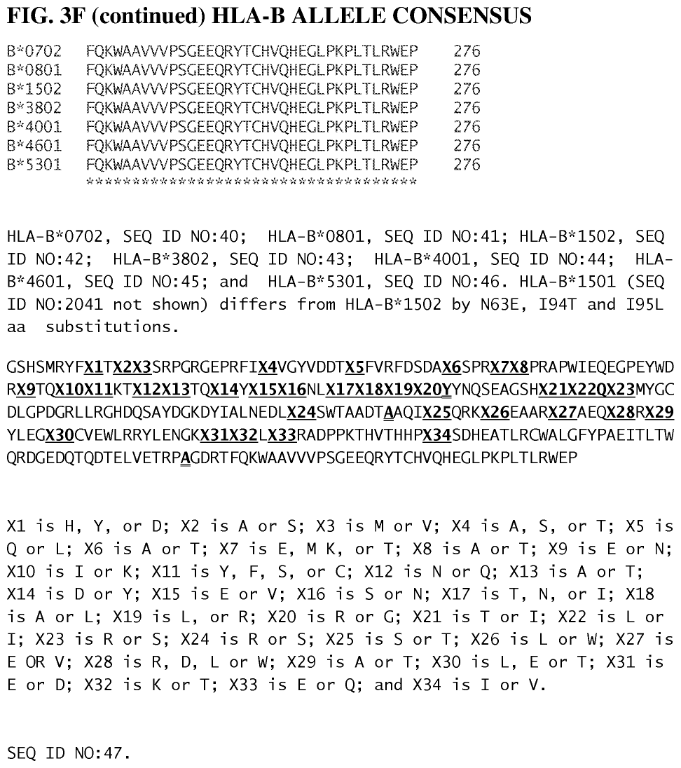

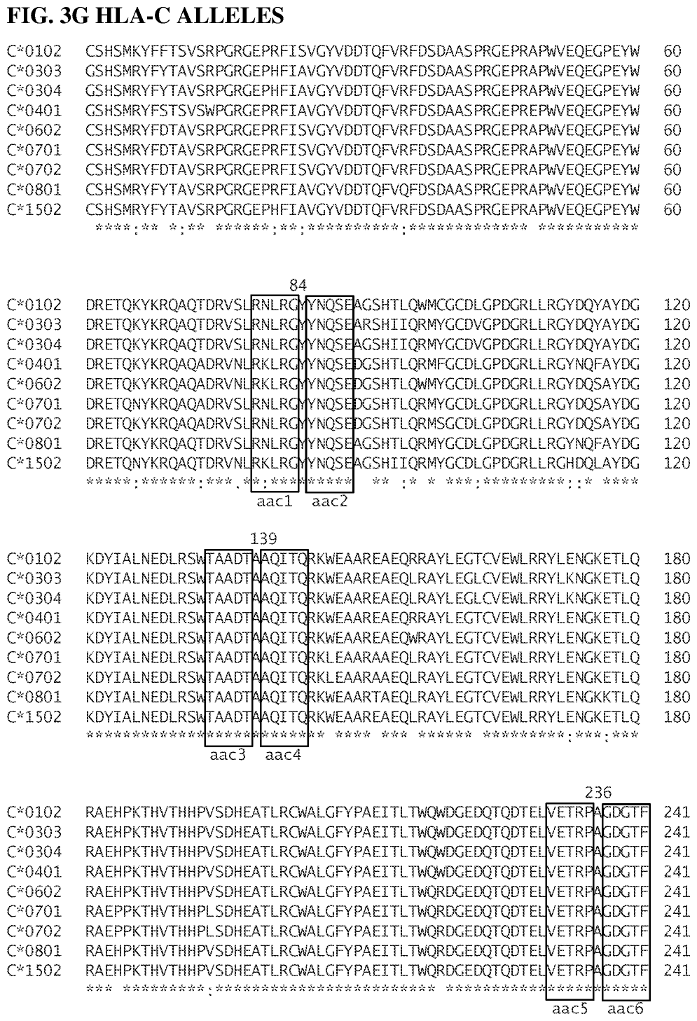

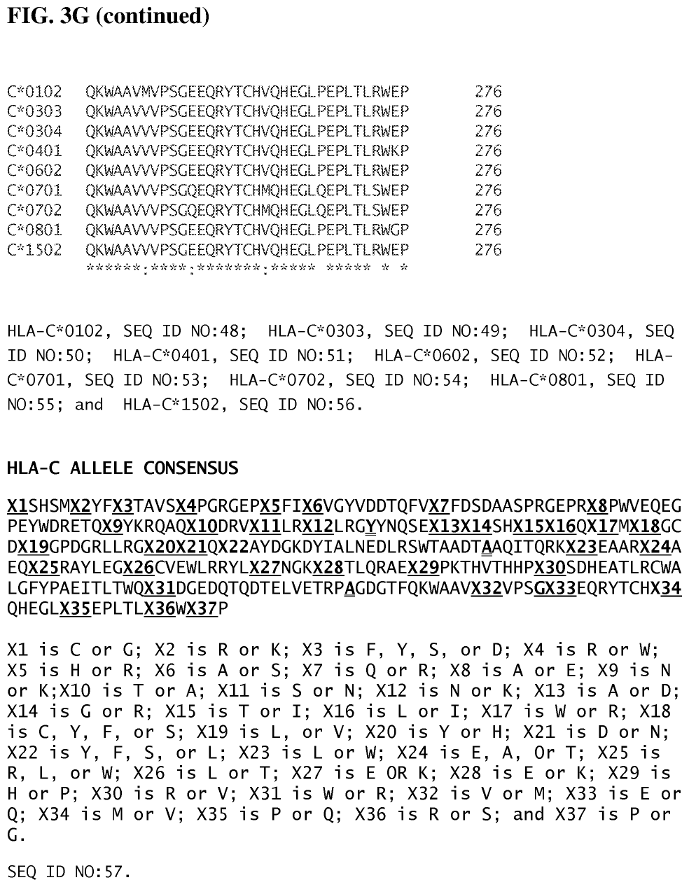

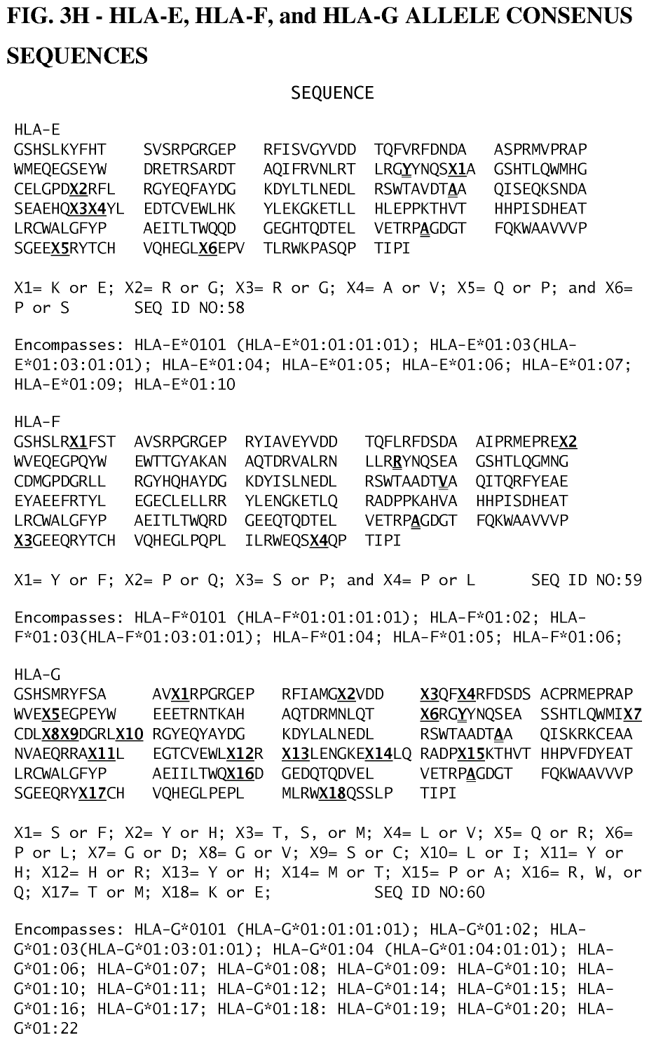

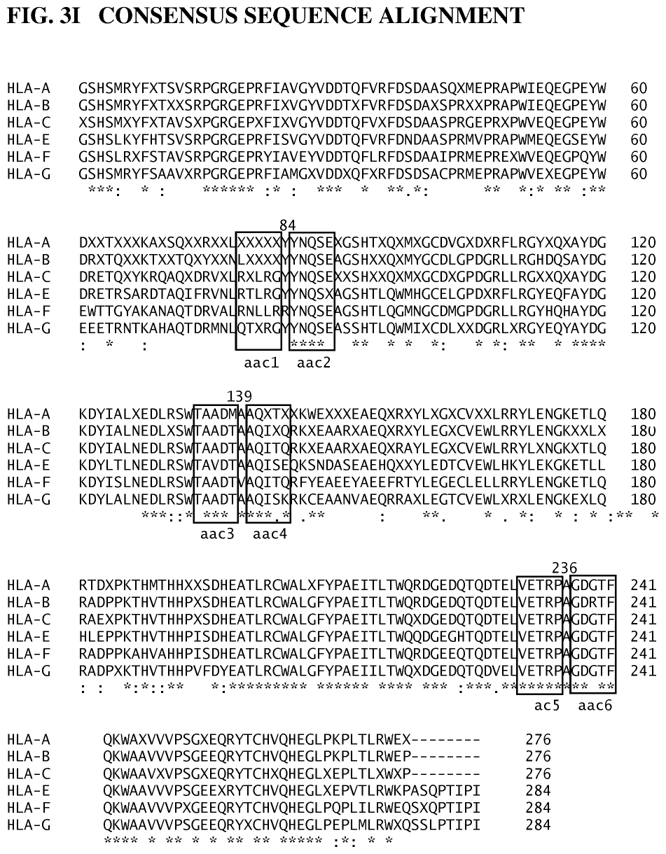

A third group of unconjugated T-Cell-MPs described herein appears as a duplex between a first T-Cell-MP and a second T-Cell-MP that associate through interactions in their scaffold sequences. Such unconjugated duplex T-Cell-MPs may have an identical first and second T-Cell-MP polypeptide sequence duplexed as a homodimer, or non-identical first and second T-Cell-MP polypeptide sequences duplexed as a heterodimer, with one or both of the T-Cell-MPs comprising from N-terminus to C-terminus:
(i) optionally one or more MOD polypeptide sequences (e.g., two or more MOD polypeptide sequences, such as in tandem, wherein when there are two or more MOD polypeptide sequences they are optionally joined to each other by independently selected L1 linkers);
(ii) an optional L2 polypeptide sequence joining the one or more optional MOD polypeptide sequences to a β2M polypeptide sequence;
(iii) the β2M polypeptide sequence;
(iv) an L3 linker polypeptide sequence comprising from 10 to 50 amino acids;
(v) a class I MHC-H polypeptide sequence comprising cysteines substituted at positions 84 and 139 (see FIGS. 3E-3H, e.g., Y84C and A139C substitutions) and forming a disulfide bond;
(vi) an L4 linker polypeptide sequence;
(vii) an interspecific or non-interspecific Ig Fc scaffold sequence;
(viii) an L5 linker polypeptide sequence; and
(ix) optionally one or more MOD polypeptide sequence (e.g., two or more MOD polypeptide sequences, such as in tandem, wherein when there are two or more MOD polypeptide sequences they are optionally joined to each other by independently selected L6 linkers);
wherein at least one of the β2M polypeptide sequence, the L3 linker polypeptide sequence, and/or the MHC-H polypeptide sequence comprises at least one chemical conjugation site, e.g., for epitope conjugation and/or payload conjugation; wherein at least one of the β2M polypeptide sequence, the L3 linker polypeptide sequence, or the MHC-H polypeptide sequence comprises a chemical conjugation site that does not appear in a wt. sequence; and
wherein the first and second T-Cell-MPs are optionally covalently linked through at least one disulfide bond between their Ig Fc scaffold sequences. The chemical conjugation site should be suitable for epitope conjugation in that it does not interfere with the interactions of the T-Cell-MP with a TCR and is preferably solvent accessible permitting its conjugation to the epitope.

The chemical conjugation sites for epitope conjugation to T-Cell-MPs, including those of the above-mentioned first, second, and third groups of unconjugated T-Cell-MPs, permit the covalent attachment of a coronavirus (e.g., SARS-CoV or SARS-CoV-2) epitope presenting molecule (e.g., a peptide epitope) to the T-Cell-MP such that it can be bound (located in the binding cleft) by the MHC-H polypeptide and presented to a TCR. The chemical conjugation sites of an unconjugated T-Cell-MP may be one that does not appear in a wt. sequence (e.g., they are created using the techniques of protein engineering based in biochemistry and/or molecular biology). The chemical conjugation site should also be suitable for epitope conjugation in that it does not interfere with the interactions of the T-Cell-MP with a TCR, and is preferably solvent accessible, permitting its conjugation to the epitope.

It is understood that the unconjugated T-Cell-MPs do not comprise a peptide epitope (either covalently attached to, or as a fusion with, the T-Cell-MP polypeptide) that can be located in the binding cleft of the MHC-H/β2M polypeptide sequences and presented to a TCR. The disclosure does, however, include and provide for T-Cell-MP-epitope conjugates further comprising a molecule presenting an epitope that is directly or indirectly (e.g., through a peptide or non-peptide linker) covalently attached to the T-Cell-MP at a chemical conjugation site; where the epitope can also be associated with (located in or positioned in) the binding cleft of the T-Cell-MP MHC-H polypeptide sequence and functionally presented to a T cell bearing a TCR specific for the epitope, leading to TCR mediated activation or inhibition of the T cell.

The disclosure also provides T-Cell-MPs in which the epitope present in a T-Cell-MP-epitope conjugate may bind to a TCR (e.g., on a T cell) with an affinity of at least 100 micro molar (μM) (e.g., at least 10 μM, at least 1 μM, at least 100 nM, at least 10 nM, or at least 1 nM).

A T-Cell-MP-epitope conjugate may bind to a first T cell with an affinity that is higher than the affinity with which the T-Cell-MP-epitope conjugate binds to a second T cell; where the first T cell expresses on its surface a Co-MOD and a TCR that binds the epitope, and where the second T cell expresses on its surface the same Co-MOD present on the first T cell, but does not express on its surface a TCR that binds the epitope (e.g., as tightly as the TCR of the first cell if it binds at all). See FIG. 1. The increased affinity may be measured in binding assays or inferred from the concentration of the T-Cell-MP-epitope conjugate required to stimulate the first as compared to the second T cell. The increased affinity for epitope-specific T cells (i.e., T cells that express a TCR that recognizes and binds the epitope presented by an MHC molecule) permits the use of the T-Cell-MP-epitope conjugates as agents for clinical testing, diagnostics, and as therapeutics capable of directing epitope-specific T cell actions.

MODs present in T cell-MPs are independently selected wt. MODs and/or variant MODs. Where the T cell-MP forms a heteromeric complex, such as through the use of interspecific scaffold polypeptide sequences, the MODs presented in at least one (e.g., at least two) of the T-Cell-MPs of the heteromer may be selected independently from the other T-Cell-MPs of the heteromeric complex. Accordingly, a heterodimeric duplex T-Cell-MP may have independently selected MODs that are different in the first and second T-Cell-MPs of the duplex. MODs in one aspect are selected to be one or more activating wt. MODs and/or variant MODs capable of stimulating epitope-specific T cell activation/proliferation (e.g., wt. and/or variant IL-2, 4-1BBL and/or CD80). In another embodiment, the MODs are one or more inhibitory wt. MODs and/or variant MODs capable of inhibiting T cell activation/proliferation (e.g., FAS-L and/or PD-L1). When used in conjunction with a T-Cell-MP bearing a suitable epitope, such activating or inhibitory MODs are capable of epitope-specific T cell action, particularly where the MODs are variant MODs and the MHC-epitope-TCR interaction is sufficiently strong to dominate the interaction of the T-Cell-MP with the T cells.

### 2 Chemical Conjugation Sites of Unconjugated T-Cell-MPs

The term “chemical conjugation site” means any suitable site of a T-Cell-MP that permits the selective formation of a direct or indirect (through an intervening linker or spacer) covalent linkage between the T-Cell-MP and an epitope- or payload-containing molecule. Chemical conjugation sites of unconjugated T-Cell-MPs may be (i) active, i.e., capable of forming a direct or indirect (through an intervening linker or spacer) covalent linkage between the T-Cell-MP and an epitope or payload without an additional chemical reaction or transformation of the chemical conjugation site (e.g., a solvent-accessible cysteine sulfhydryl), or (ii) nascent, i.e., requiring a further chemical reaction or enzymatic transformation of the chemical conjugation site to become an active chemical conjugation site (e.g., a sulfatase sequence not yet activated by an fGly enzyme). Selective formation of a direct or indirect linkage between a T-Cell-MP and an epitope may occur at a side chain functional group of an amino acid in a T-Cell-MP (e.g., linkage to an aa in the β2M polypeptide sequence).

The term “selective formation” means that when an epitope- or payload-containing molecule bearing a moiety that is reactive with an active chemical conjugation site of a T-Cell-MP, the epitope- or payload-containing molecule will be covalently bound to the chemical conjugation site in an amount higher than to any other site in the T-Cell-MP.

Chemical conjugation sites may be introduced into a T-Cell-MP using protein engineering techniques (e.g., by use of an appropriate nucleic acid sequence) to achieve a T-Cell-MP having a desired aa sequence. Chemical conjugation sites can be individual aas (e.g., a cysteine or lysine) or aa sequences (e.g., sulfatase, sortase or transglutaminase sequences) in a protein or polypeptide sequence of the T-Cell-MP.

Where the protein or polypeptide sequence of the T-Cell-MP is derived from a naturally occurring protein (e.g., the β2M, MHC-H or an IgG scaffold), the chemical conjugation site may be a site not appearing in the naturally occurring sequence, such as a site resulting from amino acid substitutions (e.g., cysteine substitutions), insertions, and or deletions. The chemical conjugation site may also be a sequence, or part of a sequence, that is not derived from a naturally occurring protein, such as a linker sequence (e.g., the L3 linker of a T-Cell-MP connecting the β2M and MHC-H polypeptide sequences of a T-Cell-MP).

In some embodiments, there is only one chemical conjugation site (e.g., one chemical conjugation site added by protein engineering) in each unconjugated T-Cell-MP polypeptide that permits an epitope to be covalently attached such that it can be located in the MHC polypeptide binding cleft and presented to a TCR. Each individual unconjugated T-Cell-MP may comprise more than one chemical conjugation site each selected individually to be either the same or different types of chemical conjugation sites, thereby permitting the same or different molecules (e.g., an epitope and one or more payloads) to be selectively conjugated to each of the chemical conjugation sites. Accordingly, each individual or duplexed unconjugated T-Cell-MP may comprise one or more chemical conjugations sites that are selected to be either the same or different types of chemical conjugation sites, thereby permitting the same or different molecules to be selectively conjugated to each of the chemical conjugation sites. The chemical conjugations sites (e.g., for the conjugation of epitope) generally will be the same (e.g., of the same type) so that epitope presenting molecules can be covalently attached to all of the desired sites in, for example, a duplex unconjugated T-Cell-MP, using a single reaction. T-Cell-MPs may contain chemical conjugation sites in addition to those for the conjugation to an epitope, including conjugation sites for the incorporation of, for example, targeting sequences (e.g., polypeptides comprising a targeting sequence), and/or payloads such as labels.

Chemical conjugation sites used to incorporate molecules other than epitope presenting molecules will, in most instances, be of a different type (e.g., utilize different chemical reactions) and in different locations than the sites used to incorporate epitopes, thereby permitting different molecules to be selectively conjugated to each of the polypeptides. Where a T-Cell-MP is to comprise a targeting sequence and/or one or more payload molecules, the unconjugated T-Cell-MP may comprise more than one copy of a chemical conjugation site (e.g., chemical conjugation sites added by protein engineering) to permit attachment to multiple molecules of targeting sequence (e.g., as a polypeptides comprising a targeting sequence), and/or payload.

Chemical conjugation sites that may be incorporated into unconjugated T cell-MP polypeptides, include, but are not limited to:
a) peptide sequences that act as enzyme modification sequences (e.g., sulfatase, sortase, and/or transglutaminase sequences);
b) non-natural aas and/or selenocysteines;
c) chemical conjugation sites comprising individual amino acids;
d) carbohydrate or oligosaccharide moieties; and
e) IgG nucleotide binding sites.
   a. Sulfatase Motifs

In those embodiments where enzymatic modification is chosen as the means of chemical conjugation, the chemical conjugation site(s) may comprise a sulfatase motif. Sulfatase motifs are usually 5 or 6 aas in length, and are described, for example, in U.S. Pat. No. 9,540,438 and U.S. Pat. Pub. No. 2017/0166639 A1, which are incorporated by reference. Insertion of the motif results in the formation of a protein or polypeptide that is sometimes referred to as aldehyde tagged or having an aldehyde tag. The motif may be acted on by formylglycine generating enzyme(s) (“FGE” or “FGEs”) to convert a cysteine or serine in the motif to a formylglycine residue (“fGly” although sometimes denoted “FGly”), which is an aldehyde containing aa, sometimes referred to as oxoalanine, that may be utilized for selective (e.g., site specific) chemical conjugation reactions. Accordingly, as used herein, “aldehyde tag” or “aldehyde tagged” polypeptides refer to an aa sequence comprising an unconverted sulfatase motif, as well as to an aa sequence comprising a sulfatase motif in which the cysteine or the serine residue of the motif has been converted to fGly by action of an FGE. Where the term sulfatase motif is utilized in the context of an aa sequence, both the nascent chemical conjugation sequence (e.g., a polypeptide containing the unconverted motif) as well as its fGly containing the active chemical conjugation site counterpart are disclosed. Once present in a polypeptide (e.g., of a T-Cell-MP), a fGly residue may be reacted with molecules (e.g., peptide epitopes with or without an intervening linker) comprising a variety of reactive groups including, but not limited to, thiosemicarbazide, aminooxy, hydrazide, and hydrazino groups to form a conjugate (e.g., a T-Cell-MP-epitope conjugate) having a covalent bond between the peptide and the molecule via the fGly residue. Sulfatase motifs may be used to incorporate not only epitopes (e.g., epitope presenting peptides), but also targeting sequences (e.g., polypeptides comprising a targeting sequence), and/or payloads (e.g., in the formation of conjugates with drugs and diagnostic molecules).

In embodiments, the sulfatase motif is at least 5 or 6 aa residues, but can be, for example, from 5 to 16 (e.g., 6-16, 5-14, 6-14, 5-12, 6-12, 5-10, 6-10, 5-8, or 6-8) aas in length. The sulfatase motif may be limited to a length less than 16, 14, 12, 10, or 8 aa residues.

In an embodiment, the sulfatase motif comprises the sequence of Formula (I): X1Z1X2Z2X3Z3 (SEQ ID NO: 66), where
Z1 is cysteine or serine;
Z2 is either a proline or alanine residue (which can also be represented by “P/A”);
Z3 is a basic aa (arginine, lysine, or histidine, usually lysine), or an aliphatic aa (alanine, glycine, leucine, valine, isoleucine, or proline, usually A, G, L, V, or I);
X1 is present or absent and, when present, can be any aa, though usually an aliphatic aa, a sulfur-containing aa, or a polar uncharged aa (e.g., other than an aromatic aa or a charged aa), usually L, M, V, S or T, more usually L, M, S or V, with the proviso that, when the sulfatase motif is at the N-terminus of the target polypeptide, X1 is present; and
X2 and X3 independently can be any aa, though usually an aliphatic aa, a polar, uncharged aa, or a sulfur containing aa (e.g., other than an aromatic aa or a charged aa), usually S, T, A, V, G or C, more usually S, T, A, V or G.

As indicated above, a sulfatase motif of an aldehyde tag is at least 5 or 6 aa residues, but can be, for example, from 5 to 16 aas in length. The motif can contain additional residues at one or both of the N- and C-termini, such that the aldehyde tag includes both a sulfatase motif and an “auxiliary motif.” In an embodiment, the sulfatase motif includes a C-terminal auxiliary motif (i.e., following the Z3 position of the motif).

A variety of FGEs may be employed for the conversion (oxidation) of cysteine or serine in a sulfatase motif to fGly. As used herein, the term formylglycine generating enzyme, or FGE, refers to fGly-generating enzymes that catalyze the conversion of a cysteine or serine of a sulfatase motif to fGly. As discussed in U.S. Pat. No. 9,540,438, the literature often uses the term formylglycine-generating enzymes for those enzymes that convert a cysteine of the motif to fGly, whereas enzymes that convert a serine in a sulfatase motif to fGly are referred to as Ats-B-like.

Sulfatase motifs of Formula (I) amenable to conversion by a prokaryotic FGE often contain a cysteine or serine at Z1 and a proline at Z2 that may be modified either by the “SUMP I-type” FGE or the “Ats-B-like” FGE, respectively. Prokaryotic FGE enzymes that may be employed include the enzymes from Clostridium perfringens (a cysteine type enzyme), Klebsiella pneumoniae (a Serine-type enzyme) or the FGE of Mycobacterium tuberculosis. Where peptides containing a sulfatase motif are being prepared for conversion into fGly-containing peptides by a eukaryotic FGE, for example by expression and conversion of the peptide in a eukaryotic cell or cell-free system using a eukaryotic FGE, sulfatase motifs amenable to conversion by a eukaryotic FGE may advantageously be employed.

Host cells for production of polypeptides with unconverted sulfatase motifs, or where the cell expresses a suitable FGE for converting fGly-containing polypeptide sequences, include those of a prokaryotic and eukaryotic organism. Non-limiting examples include Escherichia coli strains, Bacillus spp. (e.g., B. subtilis, and the like), yeast or fungi (e.g., S. cerevisiae, Pichia spp., and the like). Examples of other host cells, including those derived from a higher organism such as insects and vertebrates, particularly mammals, include, but are not limited to, Hela cells (e.g., American Type Culture Collection (ATCC) No. CCL-2), CHO cells (e.g., ATCC Nos. CRL9618 and CRL9096), CHO DG44 cells, CHO-KI cells (ATCC CCL-61), 293 cells (e.g., ATCC No. CRL-1573), Vero cells, NIH 3T3 cells (e.g., ATCC No. CRL-1658), Hnh-7 cells, BHK cells (e.g., ATCC No. CCLIO), PC12 cells (ATCC No. CRL1721), COS cells, COS-7 cells (ATCC No. CRL 1651), RAT1 cells, mouse L cells (ATCC No. CCLI.3), human embryonic kidney (HEK) cells (ATCC No. CRL 1573), HLHepG2 cells, and the like.

Sulfatase motifs may be incorporated into any desired location of a T-Cell-MP (for preparation of a T-Cell-MP-epitope conjugate). In an embodiment they may be excluded from the amino or carboxyl terminal 10 or 20 amino acids. In an embodiment, a sulfatase motif may be added in (e.g., at or near the terminus) of any T-Cell-MP element, including the MHC-H or β2M polypeptide sequences or any linker sequence joining them (the L3 linker). Sulfatase motifs may also be added to the scaffold polypeptide (e.g., the Ig Fc) or any of the linkers present in the T-Cell-MP (e.g., L1 to L6).

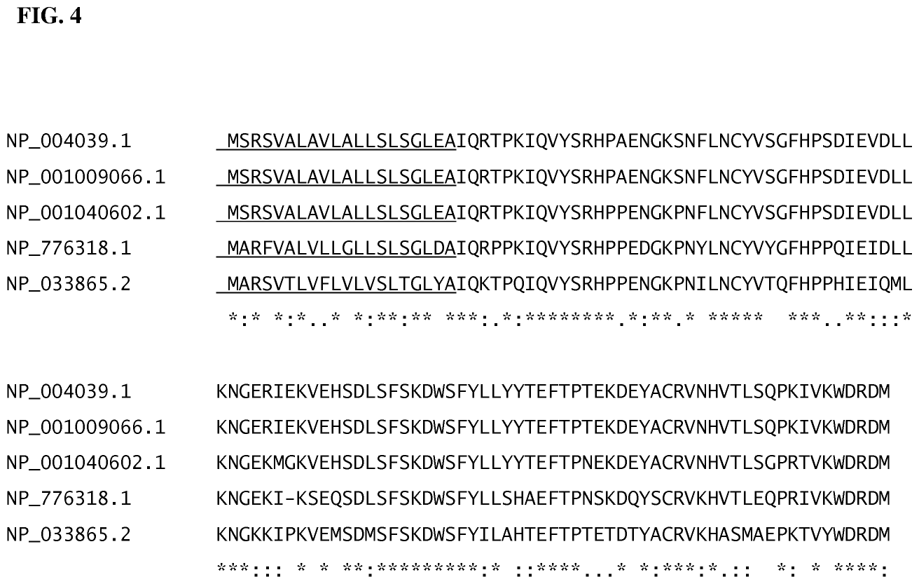

A sulfatase motif may be incorporated into, or attached to (e.g., via a peptide linker), a β2M polypeptide in a T-Cell-MP with a sequence having at least 85% (e.g., at least 90%, 95%, 98% or 99%, or even 100%) aa sequence identity to at least 50 (e.g., at least 60, 70, 80, 90, 96, 97, or 98 or all) contiguous aas of a mature β2M polypeptide sequence shown in FIG. 4 (e.g., the sequences shown in FIG. 4 starting at aa 21 and ending at their C-terminus). The mature human β2M polypeptide sequence in FIG. 4 may be selected for incorporation of the sulfatase motif. Sequence identity to the β2M polypeptides is determined relative to the corresponding portion of a β2M polypeptide in FIG. 4 without consideration of the added sulfatase motif or any linker or other sequences present.

In an embodiment, a sulfatase motif may be incorporated into a β2M polypeptide sequence having 1 to 15 (e.g., 1, 2, 3, 4, 5, 6, 7, 8, 9, 10, 11, 12, 13, 14, or 15) aa deletions, insertions and/or changes compared with a sequence shown in FIG. 4 (either an entire sequence shown in FIG. 4, or the sequence of a mature polypeptide starting at aa 21 and ending at its C-terminus). Changes are assessed without consideration of the aas of the sulfatase motif and any linker sequences present. In one such embodiment a sulfatase motif may be placed and/or be inserted within aas 1-15, 15-35, 35-55, 40-50, or 50-70 of a mature β2M sequence, such as those shown in FIG. 4. In one embodiment, sulfatase motifs may be located between aas 35-55 (e.g., between aas 40 to 50) of the human mature β2M polypeptide sequence of FIG. 4 and may have 0 to 15 aa substitutions compared with a sequence shown in FIG. 4 (either an entire sequence shown in FIG. 4, or the sequence of a mature polypeptides starting at aa 21 and ending at its C-terminus).

A sulfatase motif may be incorporated into, or attached to (e.g., via a peptide linker), a MHC Class I heavy chain polypeptide sequence having at least 85% (e.g., at least 90%, 95%, 98% or 99%, or even 100%) aa sequence identity to at least 150, 175, 200, or 225 contiguous aas of a MHC-H sequence shown in FIGS. 3A to 3I before the addition of the sulfatase motif.

In an embodiment, the added sulfatase motif is attached to the N- or C-terminus of a T-Cell-MP or, if present, attached to or within a linker located at the N- or C-terminus of the T-Cell-MP.

U.S. Pat. No. 9,540,438 discusses the incorporation of sulfatase motifs into the various Ig sequences, including Fc region polypeptides, and is herein incorporated by reference for its teachings on sulfatase motifs and modification of Fc polypeptides and other polypeptides. That patent is also incorporated by reference for its guidance on FGE enzymes, and their use in forming fGly residues, as well as the chemistry related to the coupling of molecules such as epitopes and payloads to fGly residues.

The incorporation of a sulfatase motif may be accomplished by incorporating a nucleic acid sequence encoding the motif at the desired location in a nucleic acid encoding a T-Cell-MP. As discussed below, the nucleic acid sequence may be placed under the control of a transcriptional regulatory sequence(s) (a promoter) and provided with regulatory elements that direct its expression. The expressed protein may be treated with one or more FGEs after expression and partial or complete purification. Alternatively, expression of the nucleic acid in cells that express a FGE that recognizes the sulfatase motif results in the conversion of the cysteine or serine of the motif to fGly.

In view of the foregoing, this disclosure provides for T-Cell-MPs comprising one or more fGly residues incorporated into a T-Cell-MP polypeptide chain as discussed above. The fGly residues may, for example, be in the context of the sequence X1(fGly)X2Z2X3Z3, where: fGly is the formylglycine residue; and Z2, Z3, X1, X2 and X3 are as defined in Formula (I) above. Epitopes and/or payloads may be conjugated either directly or indirectly to the reactive formyl glycine of the sulfatase motif directly or through a peptide or chemical linker. After chemical conjugation the T-Cell-MPs comprise one or more fGly′ residues incorporated in the context of the sequence X1(fGly′)X2Z2X3Z3, where the fGly′ residue is formylglycine that has undergone a chemical reaction and now has a covalently attached epitope or payload.

A number of chemistries and commercially available reagents can be utilized to conjugate a molecule (e.g., an epitope or payload) to an fGly residue, including, but not limited to, the use of thiosemicarbazide, aminooxy, hydrazide, or hydrazino derivatives of the molecules to be coupled at an fGly-containing chemical conjugation site. For example, epitopes (e.g., peptide epitopes) and/or payloads bearing thiosemicarbazide, aminooxy, hydrazide, hydrazino or hydrazinyl functional groups (e.g., attached directly to an aa of a peptide or via a linker such as a PEG) can be reacted with fGly-containing T-Cell-MP polypeptides to form a covalently linked epitope. Similarly, targeting sequences (e.g., polypeptides comprising a targeting sequence), and/or payloads such as drugs and therapeutics can be incorporated using, for example, biotin hydrazide as a linking agent. For example, an epitope (e.g., an epitope presenting peptide, phosphopeptide, lipopeptide, or glycopeptide) such as a peptide epitope having a length from about 4 aa to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) and/or one or more payloads or targeting sequences may be conjugated to a polypeptide comprising fGly sites.

The disclosure provides for methods of preparing conjugated T-Cell-MPs including T-Cell-MP-epitope conjugates and/or T-Cell-MP-payload conjugates comprising:
a) incorporating a nucleotide sequence encoding a sulfatase motif including a serine or cysteine (e.g., a sulfatase motif of Formula (I) or (II) such as X1CX2PX3Z3 (SEQ ID NO:67); CX1PX2Z3 (SEQ ID NO:68) discussed above) into a nucleic acid encoding an unconjugated T-Cell-MP;
b) expressing the sulfatase motif-containing unconjugated T-Cell-MP polypeptide in a cell that
   i) expresses a FGE and converts the serine or cysteine of the sulfatase motif to a fGly and partially or completely purifying the fGly-containing unconjugated T-Cell-MP, or
   ii) does not express a FGE that converts a serine or cysteine of the sulfatase motif to a fGly, and purifying or partially purifying the T-Cell-MP containing the sulfatase motif and contacting the purified or partially purified T-Cell-MP with a FGE that converts the serine or cysteine of the sulfatase motif into a fGly residue; and
c) contacting the fGly-containing polypeptides with an epitope and/or payload that has been functionalized with a group that forms a covalent bond between the aldehyde of the fGly and the epitope and/or payload; thereby forming a T-Cell-MP-epitope conjugate and/or T-Cell-MP payload conjugate.

In such methods the epitope (epitope containing molecule) and/or payload may be functionalized by any suitable function group that reacts selectively with an aldehyde group. Such groups may, for example, be selected from the group consisting of thiosemicarbazide, aminooxy, hydrazide, and hydrazino. In an embodiment a sulfatase motif is incorporated into a second T-Cell-MP polypeptide comprising a β2M aa sequence with at least 85% (e.g., at least 90%, 95%, 98% or 99%, or even 100%) sequence identity to at least 60, 70, 80 or 90 contiguous aas of a β2M sequence shown in FIG. 4 (e.g., a mature β2M polypeptide with identity calculated without including or before the addition of the sulfatase motif sequence).

In an embodiment of the method of preparing a T-Cell-MP-epitope conjugate and/or T-Cell-MP payload conjugate, a sulfatase motif is incorporated into a polypeptide comprising a sequence having at least 85% (e.g., at least 90%, 95%, 98% or 99%, or even 100%) aa sequence identity to at least 150, 175, 200, or 225 contiguous aas of a sequence shown in FIGS. 3A-3I, with sequence identity calculated without including the addition of the sulfatase motif sequence).

b. Sortase A Enzyme Sites

Epitopes (e.g., peptides comprising the sequence of an epitope) and payloads may be attached at the N- and/or C-termini T-Cell-MP by incorporating sites for Sortase A conjugation at those locations.

Sortase A recognizes a C-terminal pentapeptide sequence LP (X5) TG/A (SEQ ID NO:69, with X5 being any single amino acid, and G/A being a glycine or alanine), and creates an amide bond between the threonine within the sequence and glycine or alanine in the N-terminus of the conjugation partner.

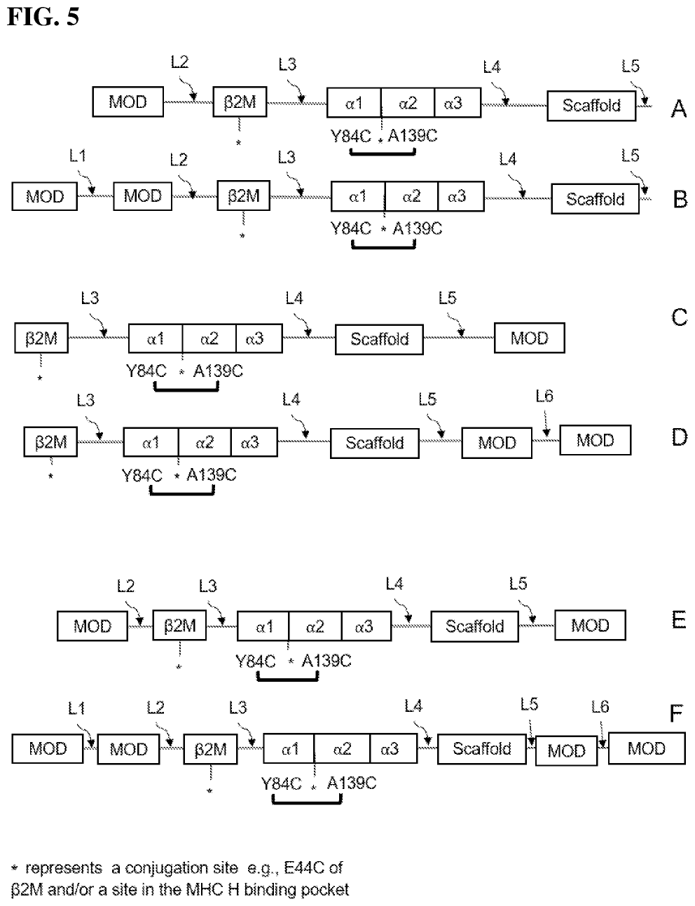

For attachment of epitopes or payloads to the C-terminal portion of a T-Cell-MP polypeptide a LP (X5) TG/A is provided in the carboxy terminal portion of the desired polypeptide(s), such as in an exposed L5 linker (see FIG. 5, structure A). An exposed stretch of glycines or alanines (e.g., (G)₃₋₅ (SEQ ID NOs: 70 and 71 when using Sortase A from Staphylococcus aureus or alanines (A)₃₋₅, SEQ ID NOs: 72 and 73 when using Sortase A from Streptococcus pyogenes) is provided at the N-terminus of a peptide that comprises an epitope (e.g., in a linker attached to the epitope), a peptide payload (or a linker attached thereto), or a peptide covalently attached to a non-peptide epitope or payload.

For attachment of epitopes or payloads to the amino terminus of a T-Cell-MP polypeptide, an aa sequence comprising an exposed stretch of glycines (e.g., (G)_{2, 3, 4, or 5}) or alanines (e.g., (A)_{2, 3, 4, or 5}) is provided at the N-terminus, and a LP (X5) TG/A is provided in the carboxy terminal portion of a peptide that comprises an epitope (or a linker attached thereto), a peptide payload (or a linker attached thereto), or a peptide covalently attached to a non-peptide epitope or payload.

Combining Sortase A with the amino and carboxy modified peptides described above results in a cleavage between the Thr and Gly/Ala residues in the LP (X5) TG/A sequence and formation of a covalently coupled complex of the form: carboxy-modified polypeptide-LP (X5) T*G/A-amino-modified polypeptide, where the “*” represents the bond formed between the threonine of the LP (X5) TG/A motif and the glycine or alanine of the N-terminal modified peptide.

In place of LP (X5) TG/A, a LPETGG (SEQ ID NO:74) peptide may be used for S. aureus Sortase A coupling, or a LPETAA (SEQ ID NO:75) peptide may be used for S. pyogenes Sortase A coupling. The conjugation reaction still occurs between the threonine and the amino terminal oligoglycine or oligoalanine peptide to yield a carboxy-modified polypeptide-LP (X5) T*G/A-amino-modified polypeptide, where the “*” represents the bond formed between the threonine and the glycine or alanine of the N-terminal modified peptide.

c. Transglutaminase Enzyme Sites

Transglutaminases (mTGs) catalyze the formation of a covalent bond between the amide group on the side chain of a glutamine residue and a primary amine donor (e.g., a primary alkyl amine, such as is found on the side chain of a lysine residue in a polypeptide). Transglutaminases may be employed to conjugate epitopes and payloads to T-Cell-MPs, either directly through a free amine, or indirectly via a linker comprising a free amine. As such, glutamine residues added to a T-Cell-MP in the context of a transglutaminase site may be considered as chemical conjugation sites when they can be accessed by enzymes such as Streptoverticillium mobaraense transglutaminase. That enzyme (EC 2.3.2.13) is a stable, calcium-independent enzyme catalyzing the γ-acyl transfer of glutamine to the ε-amino group of lysine. Glutamine residues appearing in a sequence are, however, not always accessible for enzymatic modification. The limited accessibility can be advantageous as it limits the number of locations where modification may occur. For example, bacterial mTGs are generally unable to modify glutamine residues in native IgG1s; however, Schibli and co-workers (Jeger, S., et al. (2010) Angew Chem (Int Engl). 49:99957 and Dennler P, et al. (2014) Bioconjug Chem. 25 (3): 569-78) found that deglycosylating IgG1s at N297 rendered glutamine residue Q295 accessible and permitted enzymatic ligation to create an antibody drug conjugate. Further, by producing a N297 to Q297 IgG1 mutant, they introduced two sites for enzymatic labeling by transglutaminase. Modification at N297 also offers the potential to reduce the interaction of the IgG Fc reaction with complement C1q protein.

Where a T-Cell-MP does not contain a glutamine that may be employed as a chemical conjugation site (e.g., it is not accessible to a transglutaminase or not placed in the desired location), a glutamine residue may be added to a sequence to form a transglutaminase site, or a sequence comprising a transglutaminase accessible glutamine (sometimes referred to as a “glutamine tag” or a “Q-tag”), may be incorporated through protein engineering into the polypeptide. The added glutamine or Q-tag may act as a chemical conjugation site for epitopes or payloads. US Pat. Pub. No. 2017/0043033 A1 describes the incorporation of glutamine residues and Q-tags and the use of transglutaminase for modifying polypeptides and is incorporated herein for those teachings.

Incorporation of glutamine residues and Q-tags may be accomplished chemically where the peptide is synthesized, or by modifying a nucleic acid that encodes the polypeptide and expressing the modified nucleic acid in a cell or cell-free system. In embodiments, the glutamine-containing Q-tag comprises an aa sequence selected from the group consisting of LQG, LLQGG (SEQ ID NO:76), LLQG (SEQ ID NO:77), LSLSQG (SEQ ID NO:78), and LLQLQG (SEQ ID NO:79) (numerous others are available).

Glutamine residues and Q-tags may be incorporated into any desired location of a T-Cell-MP. In an embodiment, a glutamine residue or Q-tag may be added in (e.g., at or near the terminus of) any T-Cell-MP element, including the MHC-H or β2M polypeptide sequences or any linker sequence joining them (the L3 linker). Glutamine residues and Q-tags may also be added to the scaffold polypeptide (e.g., the Ig Fc) or any of the linkers present in the T-Cell-MP (e.g., L1 to L6).

A glutamine residue or Q-tag may be incorporated into, or attached to (e.g., via a peptide linker), a β2M polypeptide in a T-Cell-MP with a sequence having at least 85% (e.g., at least 90%, 95%, 98% or 99%, or even 100%) aa sequence identity to at least 50 (e.g., at least 60, 70, 80, 90, 96, 97, or 98 or all) contiguous aas of a mature β2M polypeptide sequence shown in FIG. 4 (e.g., the sequences shown in FIG. 4 starting at aa 21 and ending at their C-terminus). The mature human β2M polypeptide sequence in FIG. 4 may be selected for incorporation of the glutamine residue or Q-tag. Sequence identity to the β2M polypeptides is determined relative to the corresponding portion of a β2M polypeptide in FIG. 4 without consideration of the added glutamine residue, Q-tag, or any linker or other sequences present.

In an embodiment, a glutamine residue or Q-tag may be incorporated into a β2M polypeptide sequence having 1 to 15 (e.g., 1, 2, 3, 4, 5, 6, 7, 8, 9, 10, 11, 12, 13, 14, or 15) aa deletions, insertions and/or changes compared with a sequence shown in FIG. 4 (either an entire sequence shown in FIG. 4, or the sequence of a mature polypeptide starting at aa 21 and ending at its C-terminus). Changes are assessed without consideration of the aas of the glutamine residue, Q-tag and any linker sequences present. In one such embodiment a glutamine residue or Q-tag may be placed and/or be inserted within aas 1-15, 15-35, 35-55, 40-50, or 50-70 of a mature β2M sequence, such as those shown in FIG. 4. In one embodiment, a glutamine residue or Q-tag may be located between aas 35-55 (e.g., 40 to 50) of the human mature β2M polypeptide sequence of FIG. 4, and may have 0 to 15 aa substitutions.

A glutamine residue or Q-tag may be incorporated into, or attached to (e.g., via a peptide linker), a MHC Class I heavy chain polypeptide sequence having at least 85% (e.g., at least 90%, 95%, 98% or 99%, or even 100%) aa sequence identity to at least 150, 175, 200, or 225 contiguous aas of a MHC-H sequence shown in FIGS. 3A to 3I before the addition of the glutamine residue or Q-tag.

In an embodiment, the added glutamine residue or Q-tag is attached to the N- or C-terminus of a T-Cell-MP or, if present, attached to or within a linker located at the N- or C-terminus of the T-Cell-MP.

Payloads and epitopes that contain, or have been modified to contain, a primary amine group may be used as the amine donor in a transglutaminase-catalyzed reaction forming a covalent bond between a glutamine residue (e.g., a glutamine residue in a Q-tag) and the epitope or payload.

Where an epitope or payload does not comprise a suitable primary amine to permit it to act as the amine donor, the epitope or payload may be chemically modified to incorporate an amine group (e.g., modified to incorporate a primary amine by linkage to a lysine, aminocaproic acid, cadaverine etc.). Where an epitope or payload comprises a peptide and requires a primary amine to act as the amine donor, a lysine or another primary amine that a transglutaminase can act on may be incorporated into the peptide. Other amine containing compounds that may provide a primary amine group and that may be incorporated into, or at the end of, an alpha amino acid chain include, but are not limited to, homolysine, 2,7-diaminoheptanoic acid, and aminoheptanoic acid. Alternatively, the epitope or payload may be attached to a peptide or non-peptide linker that comprises a suitable amine group. Examples of suitable non-peptide linkers include an alkyl linker and a PEG (polyethylene glycol) linker.

Transglutaminase can be obtained from a variety of sources, including enzymes from: mammalian liver (e.g., guinea pig liver); fungi (e.g., Oomycetes, Actinomycetes, Saccharomyces, Candida, Cryptococcus, Monascus, or Rhizopus transglutaminases); myxomycetes (e.g., Physarum polycephalum transglutaminase); and/or bacteria including a variety of Streptoverticillium, Streptomyces, Actinomadura sp., Bacillus, and the like.

Q-tags may be created by inserting a glutamine or by modifying the aa sequence around a glutamine residue appearing in an Ig Fc, β2M, and/or MHC-H chain sequence appearing in a T-Cell-MP and used as a chemical conjugation site for addition of an epitope or payload. Similarly, Q-tags may be incorporated into the Ig Fc region as chemical conjugation sites that may be used for the conjugation of, for example, epitopes and/or payloads either directly or indirectly through a peptide or chemical linker bearing a primary amine.

d. Selenocysteine and Non-Natural Amino Acids as Chemical Conjugation Sites

One strategy for providing site-specific chemical conjugation sites into a T-Cell-MP polypeptide employs the insertion of aas with reactivity distinct from the naturally occurring proteinogenic L-amino acids present in the polypeptide. Such aas include, but are not limited to, selenocysteine (Sec), and the non-natural aas: acetylphenylalanine (p-acetyl-L-phenylalanine, pAcPhe); parazido phenylalanine (4-Azido-L-phenylalanine); and propynyl-tyrosine (2-Amino-3-(4-(prop-2-yn-1-yloxy)phenyl) propanoic acid). Thanos et al. in US Pat. Publication No. 20140051836 A1 discuss some other non-natural aas including O-methyl-L-tyrosine, O-4-allyl-L-tyrosine, tri-O-acetyl-GlcNAcβ-serine, isopropyl-L-phenylalanine, p-benzoyl-L-phenylalanine, L-phosphoserine, and p-propargyloxy-phenylalanine (e.g., 4-propargyloxy-L-phenylalanine). Other non-natural aas include reactive groups such as, for example, amino, carboxy, acetyl, hydrazino, hydrazido, semicarbazido, sulfanyl, azido and alkynyl. See, e.g., US Pat. Publication No. 20140046030 A1.

In addition to directly synthesizing polypeptides in the laboratory, two methods utilizing stop codons have been developed to incorporate non-natural aas into proteins and polypeptides utilizing transcription-translation systems. The first incorporates selenocysteine (Sec) by pairing the opal stop codon, UGA, with a Sec insertion sequence. The second incorporates non-natural aas into a polypeptide generally through the use of amber, ochre, or opal stop codons. The use of other types of codons such as a unique codon, a rare codon, an unnatural codon, a five-base codon, and a four-base codon, and the use of nonsense and frameshift suppression have also been reported. See, e.g., US Pat. Publication No. 20140046030 A1 and Rodriguez et al., PNAS 103(23)8650-8655 (2006). By way of example, the non-natural amino acid acetylphenylalanine may be incorporated at an amber codon using a tRNA/aminoacyl tRNA synthetase pair in an in vivo or cell-free transcription-translation system.

Incorporation of both selenocysteine and non-natural aas requires engineering the necessary stop codon(s) into the nucleic acid coding sequence of the T-Cell-MP polypeptide at the desired location(s), after which the coding sequence is used to express the T-Cell-MP in an in vivo or cell-free transcription-translation system.

In vivo systems generally rely on engineered cell-lines to incorporate non-natural aas that act as bio-orthogonal chemical conjugation sites into polypeptides and proteins. See, e.g., International Published Application No. 2002/085923 entitled “In vivo incorporation of unnatural amino acids.” In vivo non-natural aa incorporation relies on a tRNA and an aminoacyl tRNA synthetase pair that is orthogonal to all the endogenous tRNAs and synthetases in the host cell. The non-natural aa of choice is supplemented to the media during cell culture or fermentation, making cell-permeability and stability important considerations.

Various cell-free synthesis systems provided with the charged tRNA may also be utilized to incorporate non-natural aas. Such systems include those described in US Pat. Publication No. 20160115487A1; Gubens et al., RNA. 2010 August; 16 (8): 1660-1672; Kim, D. M. and Swartz, J. R., Biotechnol. Bioeng. 66:180-8 (1999); Kim, D. M. and Swartz, J. R. Biotechnol. Prog. 16:385-90 (2000); Kim, D. M. and Swartz, J. R. Biotechnol. Bioeng. 74:309-16 (2001); Swartz et al, Methods Mol. Biol. 267:169-82 (2004); Kim, D. M. and Swartz, J. R. Biotechnol. Bioeng. 85:122-29 (2004); Jewett, M. C. and Swartz, J. R., Biotechnol. Bioeng. 86:19-26 (2004); Yin, G. and Swartz, J. R., Biotechnol. Bioeng. 86:188-95 (2004); Jewett, M. C. and Swartz, J. R., Biotechnol. Bioeng. 87:465-72 (2004); Voloshin, A. M. and Swartz, J. R., Biotechnol. Bioeng. 91:516-21 (2005).

Once incorporated into the T-Cell-MP, epitopes and/or payload bearing groups reactive with the incorporated selenocysteine or non-natural aa are brought into contact with the T-Cell-MP under suitable conditions to form a covalent bond. By way of example, the keto group of the pAcPhe is reactive towards alkoxyamines, and via oxime coupling can be conjugated directly to alkoxyamine containing epitopes and/or payloads or indirectly to epitopes and payloads via an alkoxyamine containing linker. Selenocysteine reacts with, for example, primary alkyl iodides (e.g., iodoacetamide which can be used as a linker), maleimides, and methylsulfone phenyloxadiazole groups. Accordingly, epitopes and/or payloads bearing those groups or bound to linkers bearing those groups can be covalently bound to polypeptide chains bearing selenocysteines.

As discussed above for other chemical conjugation sites, selenocysteines and/or non-natural aas may be incorporated into any desired location in the T-Cell-MP. In an embodiment, selenocysteines and/or non-natural aas may be added in (e.g., at or near the terminus of) any T-Cell-MP element, including the MHC-H or β2M polypeptide sequences or any linker sequence joining them (the L3 linker). Selenocysteines and/or non-natural aas may also be added to the scaffold polypeptide (e.g., the Ig Fc) or any of the linkers present in the T-Cell-MP (e.g., L1 to L6).

Selenocysteines and non-natural aas may be incorporated into, or attached to (e.g., via a peptide linker), a β2M polypeptide in a T-Cell-MP with a sequence having at least 85% (e.g., at least 90%, 95%, 98% or 99%, or even 100%) aa sequence identity to at least 50 (e.g., at least 60, 70, 80, 90, 96, 97, or 98 or all) contiguous aas of a mature β2M polypeptide sequence shown in FIG. 4 (e.g., the sequences shown in FIG. 4 starting at aa 21 and ending at their C-terminus). The mature human β2M polypeptide sequence in FIG. 4, may be selected for incorporation of the selenocysteines and non-natural aas. Sequence identity to the β2M polypeptides is determined relative to the corresponding portion of a β2M polypeptide in FIG. 4 without consideration of the added selenocysteines, non-natural aas, or any linker or other sequences present.

In an embodiment, a selenocysteine(s) or non-natural aa(s) may be incorporated into a β2M polypeptide sequence having 1 to 15 (e.g., 1, 2, 3, 4, 5, 6, 7, 8, 9, 10, 11, 12, 13, 14, or 15) aa deletions, insertions and/or changes compared with a sequence shown in FIG. 4 (either an entire sequence shown in FIG. 4, or the sequence of a mature polypeptide starting at aa 21 and ending at its C-terminus). Changes are assessed without consideration of the selenocysteine(s), non-natural aa(s), and any linker sequences present. In one such embodiment, a selenocysteines or non-natural aa may be placed and/or be inserted within aas 1-15, 15-35, 35-55, 40-50, or 50-70 of a mature β2M sequence, such as those shown in FIG. 4. In one embodiment, selenocysteines or non-natural aas may be located between aas 35-55 (e.g., 40 to 50) of the human mature β2M polypeptide sequence of FIG. 4 and may have 0 to 15 aa substitutions.

A selenocysteine or non-natural aa may be incorporated into, or attached to (e.g., via a peptide linker), a MHC Class I heavy chain polypeptide sequence having at least 85% (e.g., at least 90%, 95%, 98% or 99%, or even 100%) aa sequence identity to at least 150, 175, 200, or 225 contiguous aas of a MHC-H sequence shown in FIGS. 3A to 3I before the addition of the selenocysteines or non-natural aas.

In an embodiment, the added selenocysteine(s) or non-natural aa(s) is attached to the N- or C-terminus of a T-Cell-MP or, if present, attached to or within a linker located at the N- or C-terminus of the T-Cell-MP. In one such embodiment they may be utilized as sites for the conjugation of, for example, epitopes, targeting sequences (e.g., polypeptides comprising a targeting sequence), and/or payloads conjugated to the T-Cell-MP either directly or indirectly through a peptide or chemical linker.

e. Amino Acid Chemical Conjugation Sites

Any of the variety of functionalities (e.g., —SH, —NH₃, —OH, —COOH and the like) present in the side chains of naturally occurring amino acids, or at the termini of polypeptides, can be used as chemical conjugation sites. This includes the side chains of lysine and cysteine, which are readily modifiable by reagents including N-hydroxysuccinimide and maleimide functionalities, respectively. The main disadvantages of utilizing such amino acid residues is the potential variability and heterogeneity of the products. For example, an IgG has over 80 lysines, with over 20 at solvent-accessible sites. See, e.g., McComb and Owen, AAPS J. 117(2): 339-351. Cysteines tend to be less widely distributed; they tend to be engaged in disulfide bonds, and may be inaccessible (e.g., not accessible by solvent or to molecules used to modify the cysteines), and not located where it is desirable to place a chemical conjugation site. It is, however, possible to selectively modify T-Cell-MP polypeptides to provide naturally occurring and, as discussed above, non-naturally occurring amino acids at the desired locations for placement of a chemical conjugation site. Modification may take the form of direct chemical synthesis of the polypeptides (e.g., by coupling appropriately blocked amino acids) and/or by modifying the sequence of a nucleic acid encoding the polypeptide following expression in a cell or cell-free system. Accordingly, this disclosure includes and provides for the preparation of the T-Cell-MP polypeptides by transcription/translation systems capable of incorporating a non-natural aa or natural aa (including selenocysteine) to be used as a chemical conjugation site for epitope or payload conjugation.

This disclosure includes and provides for the preparation of a portion of a T-Cell-MP by transcription/translation systems and joining to its C- or N-terminus a polypeptide bearing a non-natural aa or natural aa (including selenocysteine) prepared by, for example, chemical synthesis. The polypeptide, which may include a linker, may be joined by any suitable method including the use of a sortase as described above for peptide epitopes. In an embodiment, the polypeptide may comprise a sequence of 2, 3, 4, or 5 alanines or glycines that may serve for sortase conjugation and/or as part of a linker sequence.

A naturally occurring aa (e.g., a cysteine) to be used as a chemical conjugation site may be provided at any desired location of a T-Cell-MP. In an embodiment, the naturally occurring aa may be provided in (e.g., at or near the terminus of) any T-Cell-MP element, including the MHC-H or β2M polypeptide sequences or any linker sequence joining them (the L3 linker). Naturally occurring aa(s) may also be provided in the scaffold polypeptide (e.g., the Ig Fc) or any of the linkers present in the T-Cell-MP (e.g., L1 to L6).

A naturally occurring aa (e.g., a cysteine) may also be provided in (e.g., via protein engineering), or attached to (e.g., via a peptide linker), a β2M polypeptide in a T-Cell-MP with a sequence having at least 85% (e.g., at least 90%, 95%, 98% or 99%, or even 100%) aa sequence identity to at least 50 (e.g., at least 60, 70, 80, 90, 96, 97, or 98 or all) contiguous aas of a mature β2M polypeptide sequence shown in FIG. 4 (e.g., the sequences shown in FIG. 4 starting at aa 21 and ending at their C-terminus). The mature human β2M polypeptide sequence in FIG. 4 may be selected for incorporation of the naturally occurring aa. Sequence identity to the β2M polypeptides is determined relative to the corresponding portion of a β2M polypeptide in FIG. 4 without consideration of the added naturally occurring aa, any linker, or any other sequences present.

In an embodiment, a naturally occurring aa (e.g., a cysteine) may be provided, e.g., via protein engineering in a β2M polypeptide sequence having 1 to 15 (e.g., 1, 2, 3, 4, 5, 6, 7, 8, 9, 10, 11, 12, 13, 14, or 15) aa deletions, insertions and/or changes compared with a sequence shown in FIG. 4 (either an entire sequence shown in FIG. 4, or the sequence of a mature polypeptide starting at aa 21 and ending at its C-terminus). Changes are assessed without consideration of the naturally occurring aa that will be used as a conjugation site, any linker, or other sequences present. In one such embodiment a naturally occurring aa (e.g., a cysteine) may be engineered (e.g., using the techniques of molecular biology) within aas 1-15, 15-35, 35-55, 40-50, or 50-70 of a mature β2M sequence, such as those shown in FIG. 4. In one embodiment, a naturally occurring aa (e.g., a cysteine) may be provided between aas 35-55 (e.g., between 40 and 50, between 42 and 48, between 43 and 45, or at aa 44) of the human mature β2M polypeptide sequence of FIG. 4 and may have 0 to 15 aa substitutions.

A naturally occurring aa (e.g., a cysteine) may be provided in, or attached to (e.g., via a peptide linker), a MHC Class I heavy chain polypeptide sequence having at least 85% (e.g., at least 90%, 95%, 98% or 99%, or even 100%) aa sequence identity to at least 150, 175, 200, or 225 contiguous aas of a MHC-H sequence shown in FIGS. 3A to 3I before the addition of the naturally occurring aa.

In an embodiment, the naturally occurring aa (e.g., a cysteine) may be attached to the N- or C-terminus of a T-Cell-MP, or attached to or within a linker, if present, located at the N- or C-terminus of the T-Cell-MP.

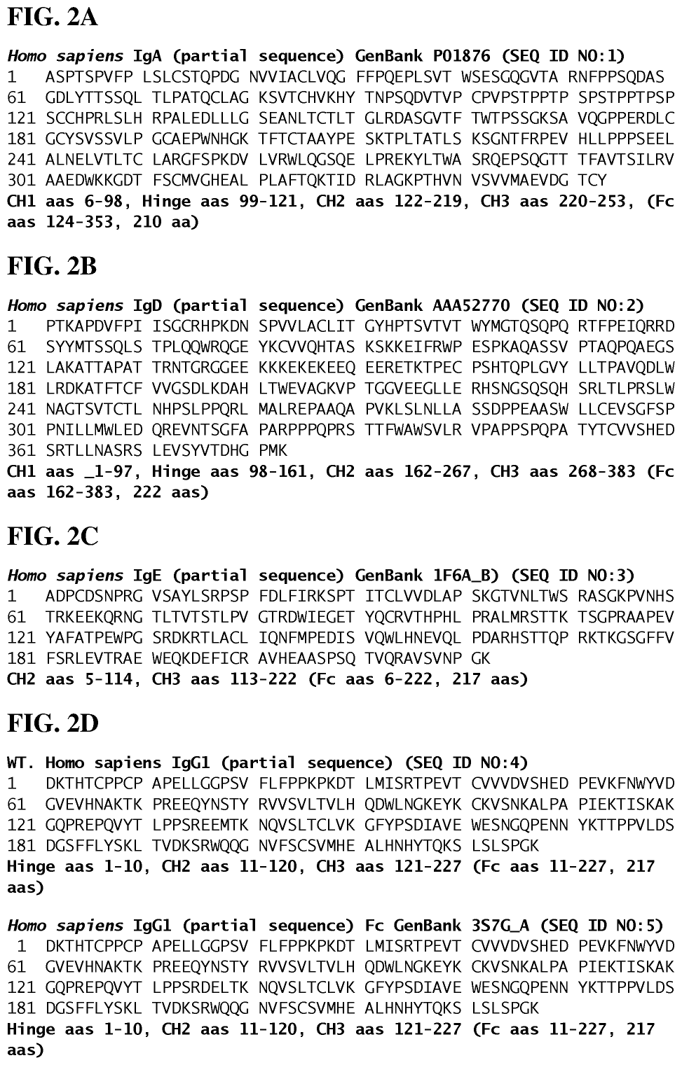

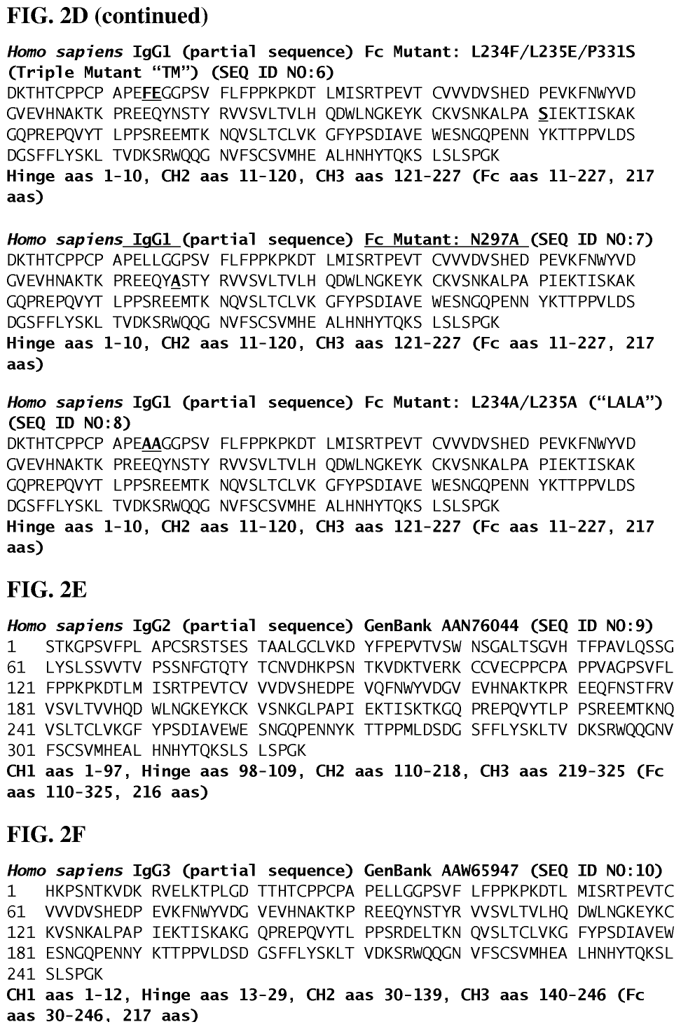

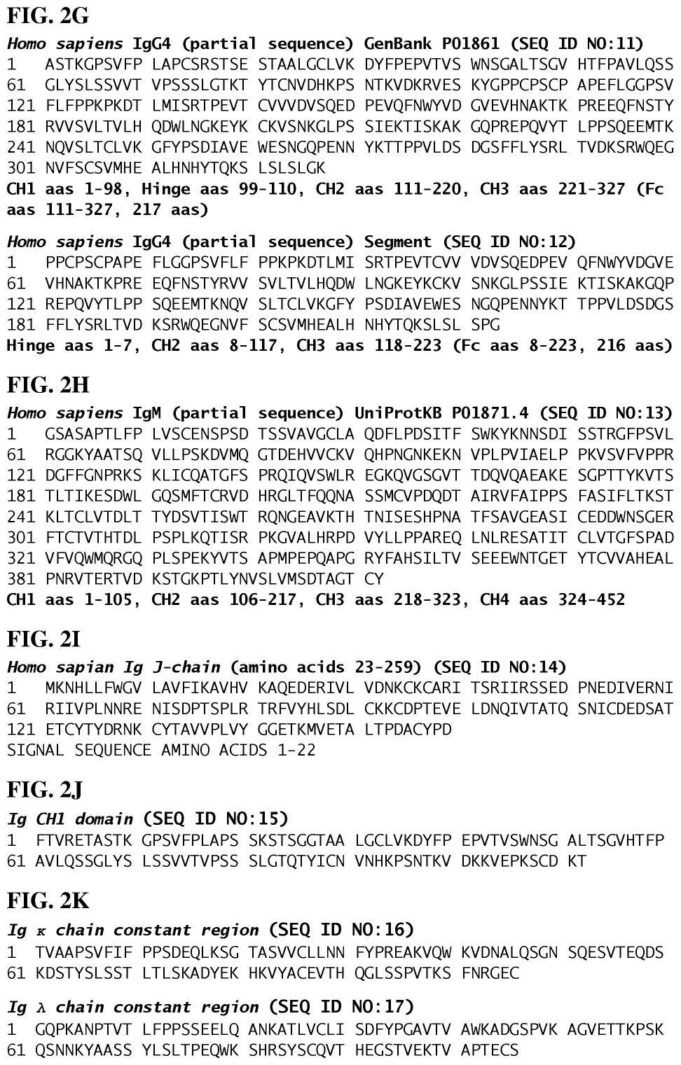

In one embodiment, a T-Cell-MP contains at least one naturally occurring aa (e.g., a cysteine) to be used as a chemical conjugation site provided, e.g., via protein engineering, in a β2M sequence as shown in FIG. 4, an Ig Fc sequence as shown in any of FIGS. 2A-G, or a MHC Class I heavy chain polypeptide as shown in FIGS. 3A-3I. In an embodiment, at least one naturally occurring aa to be used as a chemical conjugation site is provided in a polypeptide having at least 85% (e.g., at least 90%, 95%, 98% or 99%, or even 100%) aa sequence identity to at least 50 (e.g., at least 60, 70, 80, 90, 96, 97, or 98 or all) contiguous aas of a mature β2M sequence as shown in FIG. 4, an Ig Fc sequence as shown in FIG. 2, or at least 85% (e.g., at least 90%, 95%, 98% or 99%, or even 100%) aa sequence identity to at least 150, 175, 200, or 225 contiguous aas of a MHC Class I heavy chain polypeptide as shown in any of FIGS. 3A-3I. At least one naturally occurring aa (e.g., a cysteine) may be provided as a chemical conjugation site in a T-Cell-MP β2M aa sequence having at least 90% (e.g., at least 93%, 95%, 98% or 99%, or even 100%) aa sequence identity with at least the amino terminal 10, 20, 30, 40, 50, 60 or 70 aas of a mature β2M sequence as shown in FIG. 4. At least one naturally occurring aa (e.g., a cysteine) may be provided as a chemical conjugation site in a T-Cell-MP Ig Fc sequence (e.g., as shown in any of FIGS. 2A-2G). At least one naturally occurring aa (e.g., a cysteine) may be provided as a chemical conjugation site in a T-Cell-MP MHC Class I heavy chain polypeptide sequence having at least 85% (e.g., at least 90%, 95%, 98% or 99%, or even 100%) aa sequence identity to at least 150, 175, 200, or 225 contiguous aas of a MHC H polypeptide sequence provided in any of FIGS. 3A to 3I. In another embodiment, at least one naturally occurring aa to be used as a chemical conjugation site is provided in a T-Cell-MP polypeptide comprising at least 30, 40, 50, 60, 70, 80, 90, or 100 contiguous aas having 100% aa sequence identity to a MHC Class I heavy chain sequence as shown in any of FIGS. 3A to 3I or a mature β2M sequence as shown in FIG. 4.

In any of the embodiments mentioned above where a naturally occurring aa is provided, e.g., via protein engineering, in a polypeptide, the aa may be selected from the group consisting of arginine, lysine, cysteine, serine, threonine, glutamic acid, glutamine, aspartic acid, and asparagine. Alternatively, the aa provided as a conjugation site is selected from the group consisting of lysine, cysteine, serine, threonine, and glutamine. The aa provided as a conjugation site may also be selected from the group consisting of lysine, glutamine, and cysteine. In one instance, the provided aa is cysteine. In another instance, the provided aa is lysine. In still another instance, the provided aa is glutamine.

Any method known in the art may be used to couple payloads or epitopes to amino acids provided in an unconjugated T-Cell-MP. By way of example, maleimides may be utilized to couple to sulfhydryls, N-hydroxysuccinimide may be utilized to couple to amine groups, acid anhydrides or chlorides may be used to couple to alcohols or amines, and dehydrating agents may be used to couple alcohols or amines to carboxylic acid groups. Accordingly, using such chemistry an epitope or payload may be coupled directly, or indirectly through a linker (e.g., a homo- or hetero-bifunctional crosslinker), to a location on an unconjugated T-Cell-MP polypeptide. A number of bifunctional crosslinkers may be utilized, including, but not limited to, those described for linking a payload to a T-Cell-MP described herein below. For example, a peptide epitope (or a peptide-containing payload) including a maleimide group attached by way of a homo- or hetero-bifunctional linker (see, e.g., FIG. 9) or a maleimide amino acid can be conjugated to a sulfhydryl of a chemical conjugation site (e.g., a cysteine residue) that is naturally occurring or provided in a T-Cell-MP.

Maleimido amino acids can be incorporated directly into peptides (e.g., peptide epitopes) using a Diels-Alder/retro-Diels-Alder protecting scheme as part of a solid phase peptide synthesis. See, e.g., Koehler, Kenneth Christopher (2012), “Development and Implementation of Clickable Amino Acids,” Chemical & Biological Engineering Graduate Theses & Dissertations, 31, https://scholar.colorado.edu/chbe_gradetds/31.

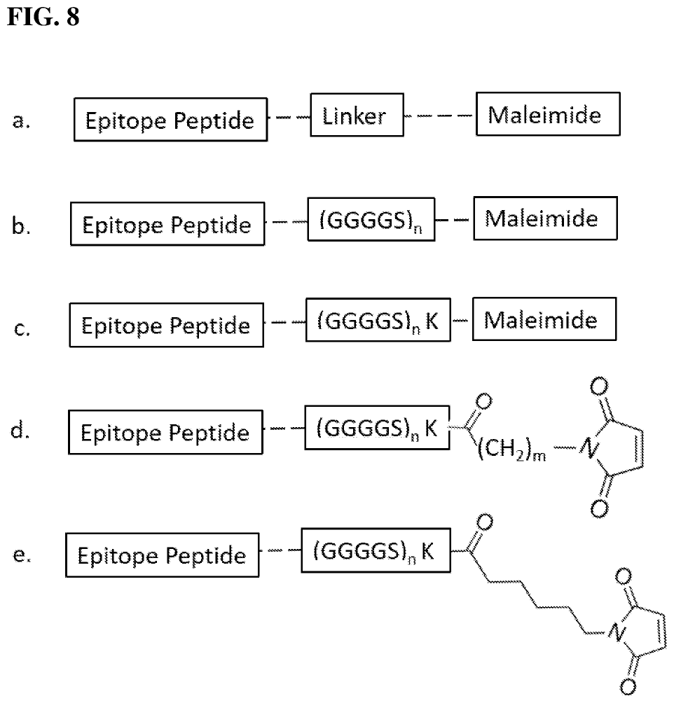

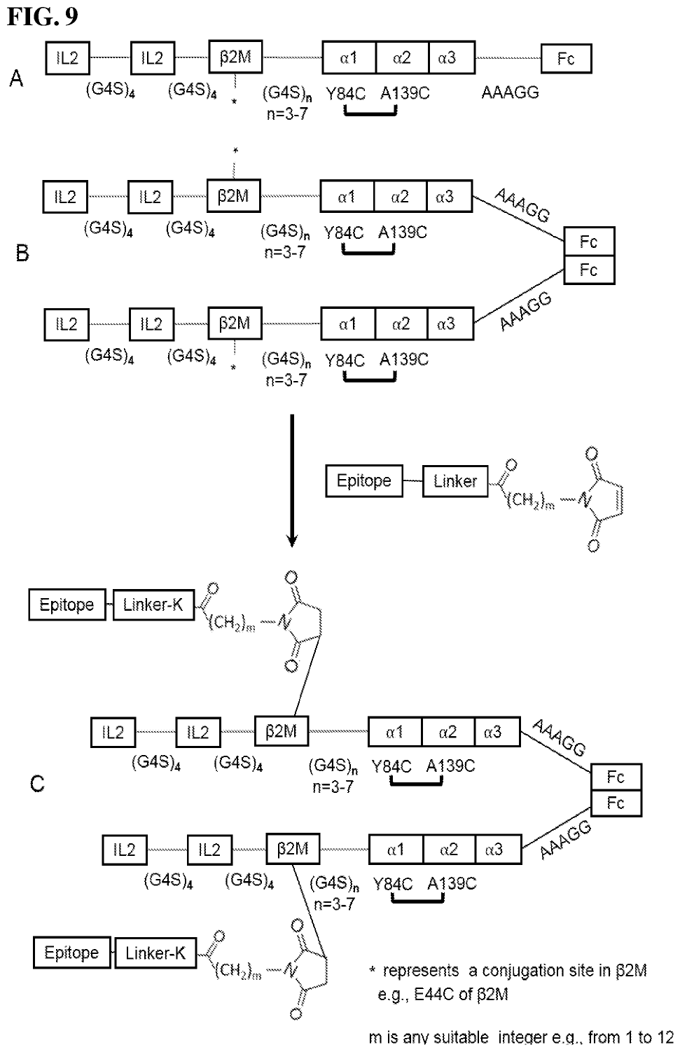

A maleimide group may also be appended to an epitope (e.g., a peptide epitope) using a homo- or hetero-bifunctional linker (sometimes referred to as a crosslinker) that attaches a maleimide directly (or indirectly, e.g., through an intervening linker that may comprise additional aas bound to the epitope) to the epitope (e.g., peptide epitope). For example, a heterobifunctional N-hydroxysuccinimide-maleimide crosslinker can attach maleimide to an amine group of a peptide lysine. Some specific crosslinkers include molecules with a maleimide functionality and either a N-hydroxysuccinimide ester (NHS) or N-succinimidyl group that can attach a maleimide to an amine (e.g., an epsilon amino group of lysine). Examples of such crosslinkers include, but are not limited to, NHS-PEG4-maleimide, γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); and N-(α-maleimidoacetoxy)-succinimide ester (AMAS), which offer different lengths and properties for peptide immobilization. Other amine reactive crosslinkers that incorporate a maleimide group include N-succinimidyl 4-(2-pyridyldithio) butanoate (SPDB). Additional crosslinkers (bifunctional agents) are recited below. In an embodiment the epitopes coupled to the T-Cell-MP have a maleimido alkyl carboxylic acid coupled to the peptide by an optional linker (see, e.g., FIGS. 8 and 9), coupled, for example, by an amide formed with the epsilon amino group of a lysine. The maleimido carboxylic acid can be, for example, a maleimido ethanoic, propanoic, butanoic, pentanoic, hexanoic, heptanoic, or octanoic acid.

A peptide epitope may be coupled to a naturally occurring cysteine present or provided in (e.g., engineered into), for example, the binding pocket of a T-Cell-MP through a bifunctional linker comprising a maleimide or a maleimide amino acid incorporated into the peptide, thereby forming a T-Cell-MP-epitope conjugate. A peptide epitope may be conjugated (e.g., by one or two maleimide amino acids or at least one maleimide containing bifunctional linker) to a MHC heavy chain having cysteine residues at any one or more locations within or adjacent to the MHC-H binding pocket. By way of example, a peptide epitope comprising maleimido amino acids or bearing a maleimide group as part of a crosslinker attached to the peptide may be covalently attached at 1 or 2 aas (e.g., cysteines) at MHC-H positions 2, 5, 7, 59, 84, 116, 139, 167, 168, 170, and/or 171 (e.g., Y7C, Y59C, Y116C, A139C, W167C, L168C, R170C, and Y171C substitutions) with the numbering as in FIGS. 3D-3I. A peptide epitope may also be conjugated (e.g., by one or two maleimide amino acids or at least one maleimide containing bifunctional linker) to a MHC heavy chain having cysteine residues at any one or more (e.g., 1 or 2) aa positions selected from positions 7 and/or 116, (e.g., Y7C and Y116C substitutions) with the numbering as in FIGS. 3D-3H. Cysteine substitution at positions 116 (e.g., Y116C) and/or 167 (e.g., W167C), with the numbering as in FIGS. 3D-3H, may be used separately or in combination to anchor epitopes (e.g., peptide epitopes) with one or two bonds formed through maleimide groups (e.g., at one or both of the ends of the epitope containing peptide).

Peptide epitopes may also be coupled to a naturally occurring cysteine present or provided in (e.g., engineered into) a β2M polypeptide sequence having at least 85% (e.g., at least 90%, 95% 97% or 100%) sequence identity to at least 60 contiguous amino acids (e.g., at least 70, 80, 90 or all contiguous aas) of a mature β2M polypeptide sequence set forth in FIG. 4. Some solvent accessible positions of mature β2M polypeptides that may be substituted by a cysteine to create a chemical conjugation site include: 2, 14, 16, 34, 36, 44, 45, 47, 48, 50, 58, 74, 77, 85, 88, 89, 91, 94, and 98 (Gln 2, Pro 14, Glu 16, Asp 34, Glu 36, Glu 44, Arg 45, Glu 47, Arg 48, Glu 50, Lys 58, Glu 74, Glu 77, Val 85, Ser 88, Gln 89, Lys 91, Lys 94, and Asp 98) of the mature peptide from NP_004039.1, or their corresponding amino acids in other β2M sequences (see the sequence alignment in FIG. 4). For example, epitopes may be conjugated to cysteines at positions 2, 44, 50, 77, 85, 88, 91, or 98 of the mature β2M polypeptides (aas 22, 64, 70, 97, 105, 108, 111, or 118 of the mature β2M sequences as shown in FIG. 4). Accordingly, the β2M sequences of a T-Cell-MP may contain cysteine chemical conjugation sites provided (e.g., by protein engineering) in the mature β2M sequence selected from Q2C, E44C, E50C, E77C, V85C, S88C, K91C, and D98C. The cysteine chemical conjugation sites in β2M sequences may also be combined with MHC-H Y84C and A139C substitutions made to stabilize the MHC H by forming an intrachain disulfide bond between MHC-H sequences. In one instance, the cysteine chemical conjugation site provided in the mature β2M is located at E44 (an E44C substitution). In another instance, the cysteine chemical conjugation site provided in the mature β2M is located at E44 (an E44C substitution) and the β2M sequence also comprises MHC-H Y84C and A139C substitutions that form an intrachain disulfide bond.

Where conjugation of an epitope, targeting sequence (e.g., a polypeptide comprising a targeting sequence), and/or payload is to be conducted through a cysteine chemical conjugation site present in an unconjugated T-cell-MP (e.g., using a maleimide modified epitope or payload) a variety of process conditions may affect the conjugation efficiency and the quality (e.g., the amount/fraction of unaggregated duplex T-Cell-MP-epitope conjugate resulting from the reaction) of conjugated T-Cell-MP resulting from the conjugation reaction. Conjugation process conditions that may be individually optimized include but are not limited to (i) prior to conjugation unblocking of cysteine sulfhydryls (e.g., potential blocking groups may be present and removed), (ii) the ratio of the T-Cell-MP to the epitope or payload, (iii) the reaction pH, (iv) the buffer employed, (v) additives present in the reaction, (vi) the reaction temperature, and (vii) the reaction time.

Prior to conjugation T-Cell-MPs may be treated with a disulfide reducing agent such as dithiothreitol (DTT), mercaptoethanol, or tris(2-carboxyethyl) phosphine (TCEP) to reduce and free cysteine sulfhydryls that may be blocked. Treatment may be conducted using relatively low amounts of reducing agent, for example from about 0.5 to 2.0 reducing equivalents per cysteine conjugation site for relatively short periods, and the cysteine chemical conjugation site of the unconjugated T-Cell-MP may be available as a reactive nucleophile for conjugation from about 10 minutes to about 1 hour, or from about 1 hour to 5 hours.

The ratio of the unconjugated T-Cell-MP to the epitope or payload being conjugated may be varied from about 1:2 to about 1:100, such as from about 1:2 to about 1:3, from about 1:3 to about 1:10, from about 1:10 to about 1:20, from about 1:20 to about 1:40, or from about 1:40 to about 1:100. The use of sequential additions of the reactive epitope or payload may be made to drive the coupling reaction to completion (e.g., multiple does of maleimide or N-hydroxy succinimide modified epitopes may be added to react with the T-Cell-MP).

As previously indicated, the conjugation reaction may be affected by the buffer, its pH, and additives that may be present. For maleimide coupling to reactive cysteines present in a T-Cell-MP the reactions are typically carried out from about pH 6.5 to about pH 8.5 (e.g., from about pH 6.5 to about pH 7.0, from about pH 7.0 to about pH 7.5, from about pH 7.5 to about pH 8.0, or from about pH 8.0 to about pH 8.5). Any suitable buffer not containing active nucleophiles (e.g., reactive thiols) and preferably degassed to avoid reoxidation of the sulfhydryl may be employed for the reaction. Some suitable traditional buffers include phosphate buffered saline (PBS), Tris-HCl, and (4-(2-hydroxyethyl)-1-piperazineethanesulfonic acid) HEPES. As an alternative to traditional buffers, maleimide conjugation reactions may be conducted in buffers/reaction mixtures comprising amino acids such as arginine, glycine, lysine, or histidine. The use of high concentrations of amino acids, e.g., from about 0.1 M (molar) to about 1.5 M (e.g., from about 0.1 to about 0.25, from about 0.25 to about 0.5 from about 0.3 to about 0.6, from about 0.4 to about 0.7, from about 0.5 to about 0.75, from about 0.75 to about 1.0, from about 1.0 to about 1.25 M, or from about 1.25 to about 1.5 M) may stabilize the conjugated and/or unconjugated T-Cell-MP.

Additives useful for maleimide and other conjugation reactions include, but are not limited to: protease inhibitors; metal chelators (e.g., EDTA) that can block unwanted side reactions and inhibit metal dependent proteases if they are present; detergents (e.g., polysorbate 80 sold as TWEEN 80®, or nonylphenoxypolyethoxyethanol sold under the names NP40 and Tergitol™ NP); and polyols such as sucrose or glycerol that can add to protein stability.

Conjugation of T-Cell-MPs with epitopes, targeting sequences (e.g., polypeptides comprising a targeting sequence), and/or payloads, and particularly conjugation at cysteines using maleimide chemistry, can be conducted over a range of temperatures, such as 0° to 40° C. For example, conjugation reactions, including cysteine-maleimide reactions, can be conducted from about 0° to about 10° C., from about 10° to about 20° C., from about 20° to about 30° C., from about 25° to about 37° C., or from about 30° to about 40° C. (e.g., at about 20° C., at about 30° C. or at about 37° C.).

Where a pair of sulfhydryl groups are present, they may be employed simultaneously for chemical conjugation to a T-Cell-MP. In such an embodiment, an unconjugated T-Cell-MP that has a disulfide bond, or that has two cysteines (or selenocysteines) provided at locations proximate to each other, may be utilized as a chemical conjugation site by incorporation of bis-thiol linkers. Bis-thiol linkers, described by Godwin and co-workers, avoid the instability associated with reducing a disulfide bond by forming a bridging group in its place and at the same time permit the incorporation of another molecule, which can be an epitope or payload. See, e.g., t G, et al., (2014), Bioconjug Chem., 25 (6): 1124-36, entitled Bridging disulfides for stable and defined antibody drug conjugates, describing the use of bis-sulfone reagents, which incorporate a hydrophilic linker (e.g., PEG (polyethylene glycol) linker).

Generally, stoichiometric or near stoichiometric amounts of dithiol reducing agents (e.g., dithiothreitol) are employed to reduce the disulfide bond and allow the bis-thiol linker to react with both cysteine and/or selenocysteine residues. Where multiple disulfide bonds are present, the use of stoichiometric or near stoichiometric amounts of reducing agents may allow for selective modification at one site. See, e.g., Brocchini, et al., Adv. Drug. Delivery Rev. (2008) 60:3-12. Where a T-Cell-MP or duplexed T-Cell-MP does not comprise a pair of cysteines and/or selenocysteines (e.g., a selenocysteine and a cysteine), they may be provided in the polypeptide (by introducing one or both of the cysteines or selenocysteines) to provide a pair of residues that can interact with a bis-thiol linker. The cysteines and/or selenocysteines should be located such that a bis-thiol linker can bridge them (e.g., at a location where two cysteines could form a disulfide bond). Any combination of cysteines and selenocysteines may be employed (i.e. two cysteines, two selenocysteines, or a selenocysteine and a cysteine). The cysteines and/or selenocysteines may both be present on a T-Cell-MP. Alternatively, in a duplex T-Cell-MP the first cysteine and/or selenocysteine is present in the first T-Cell-MP of the duplex and a second cysteine and/or selenocysteine is present in the second T-Cell-MP of the duplex, with the bis-thiol linker acting as a covalent bridge between the duplexed T-Cell-MPs.

In an embodiment, a pair of cysteine and/or selenocysteine residues is incorporated into a β2M sequence of a T-Cell-MP having at least 85% (e.g., at least 90%, 95%, 98% or 99%, or even 100%) aa sequence identity to at least 50 (e.g., at least 60, 70, 80, 90, 96, 97, or 98 or all) contiguous aas of a mature β2M polypeptide sequence shown in FIG. 4 before the addition of the pair of cysteines and/or selenocysteines, and/or into an L2 or L3 peptide linker attached to one of those sequences. In one such embodiment the pair of cysteines and/or selenocysteines may be utilized as a bis-thiol linker coupling site for the conjugation of an epitope and/or payload through a peptide or chemical linker attached to the bis-thiol group.

In another embodiment, a pair of cysteines and/or selenocysteines is incorporated into a MHC-H polypeptide sequence of a T-Cell-MP as a chemical conjugation site. In an embodiment, a pair of cysteines and/or selenocysteines is incorporated into a polypeptide comprising a sequence having at least 85% (e.g., at least 90%, 95%, 98% or 99%, or even 100%) aa sequence identity to a sequence having at least 150, 175, 200, or 225 contiguous aas of a MHC-H sequence shown in any of FIGS. 3A-3I before the addition of a pair of cysteines or selenocysteines, or into a peptide linker attached to one of those sequences. In one such embodiment the pair of cysteines and/or selenocysteines may be utilized as a bis-thiol linker coupling site for the conjugation of an epitope and/or payload through a peptide or chemical linker attached to the bis-thiol linker. Where the MHC-H sequence includes a Y84C and A139C substitutions the bis-thiol linker may be used to form a covalent bridge between those sites for the covalent coupling of an epitope (e.g., a peptide epitope).

In another embodiment, a pair of cysteines and/or selenocysteines is incorporated into an Ig Fc sequence of a T-Cell-MP to provide a chemical conjugation site. In an embodiment a pair of cysteines and/or selenocysteines is incorporated into a polypeptide comprising an Ig Fc sequence having at least 85% (e.g., at least 90%, 95%, 98% or 99%, or even 100%) aa sequence identity to a sequence shown in any of the Fc sequences of FIGS. 2A-2G before the addition of the pair of cysteines or selenocysteines. In one such embodiment the pair of cysteines and/or selenocysteines is utilized as a bis-thiol linker coupling site for the conjugation of an epitope and/or payload through a peptide or chemical linker attached to the bis-thiol group. The bis-thiol linker may be used to form a covalent bridge between scaffold polypeptides of a duplex T-Cell-MP. In such a case the cysteines of the lower hinge region that form interchain disulfide bonds, if present in the Ig Fc scaffold polypeptide sequence, may be used to insert the bis-thiol linker.

f. Other Chemical Conjugation Sites

### (i) Carbohydrate Chemical Conjugation Sites

Many proteins prepared by cellular expression contain added carbohydrates (e.g., oligosaccharides of the type added to antibodies expressed in mammalian cells). Accordingly, where a T-Cell-MP is prepared by cellular expression, carbohydrates may be present and available as selective chemical conjugation sites in, for example, glycol-conjugation reactions, particularly where the T-Cell-MP comprises an Ig Fc scaffold. McCombs and Owen, AAPS Journal, (2015) 17(2): 339-351, and references cited therein, describe the use of carbohydrate residues for glycol-conjugation of molecules to antibodies.

The addition and modification of carbohydrate residues may also be conducted in vitro, through the use of chemicals that alter the carbohydrates (e.g., periodate, which introduces aldehyde groups), or by the action of enzymes (e.g., fucosyltransferases) that can incorporate chemically reactive carbohydrates or carbohydrate analogs for use as chemical conjugation sites. In an embodiment, the incorporation of an Ig Fc scaffold with known glycosylation sites may be used to introduce site specific chemical conjugation sites.

This disclosure includes and provides for T-Cell-MPs having carbohydrates as chemical conjugation (e.g., glycol-conjugation) sites.

The disclosure also includes and provides for the use of such molecules in forming conjugates with epitopes and with other molecules such as polypeptide comprising targeting sequences, drugs, and diagnostic agent payloads.

### (ii) Nucleotide Binding Sites

Nucleotide binding sites offer site-specific functionalization through the use of a UV-reactive moiety that can covalently link to the binding site. Bilgicer et al., Bioconjug Chem. (2014) 25(7):1198-202, reported the use of an indole-3-butyric acid (IBA) moiety that can be covalently linked to an IgG at a nucleotide binding site. By incorporation of the sequences required to form a nucleotide binding site, chemical conjugates of T-Cell-MP with suitably modified epitopes and/or other molecules (e.g., payload drugs or diagnostic agents) bearing a reactive nucleotide may be employed to prepare T-Cell-MP-epitope conjugates. The epitope or payload may be coupled to the nucleotide binding site through the reactive entity (e.g., an IBA moiety) either directly or indirectly through an interposed linker.

This disclosure includes and provides for T-Cell-MPs having nucleotide binding sites as chemical conjugation sites. The disclosure also includes and provides for the use of such molecules in forming conjugates with epitopes and with other molecules such as drugs and diagnostic agents, and the use of those molecules in methods of treatment and diagnosis.

3 MHC polypeptides of T-Cell-MPs

As noted above, T-Cell-MPs include MHC polypeptides. For the purposes of the instant disclosure, the term “major histocompatibility complex (MHC) polypeptides” is meant to include MHC Class I polypeptides of various species, including human MHC (also referred to as human leukocyte antigen (HLA)) polypeptides, rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.), and the like. The term “MHC polypeptide” is meant to include Class I MHC polypeptides (e.g., β-2 microglobulin and MHC Class I heavy chain and/or portions thereof). Both the β2M and MHC-H chain sequences in a T-Cell-MP (may be of human origin. Unless expressly stated otherwise, the T-Cell-MPs and the T-Cell-MP-epitope conjugates described herein are not intended to include membrane anchoring domains (transmembrane regions) of a MHC-H chain, or a part of that molecule sufficient to anchor a T-Cell-MP, or a peptide thereof, to a cell (e.g., eukaryotic cell such as a mammalian cell) in which it is expressed. In addition, the MHC-H chain present in T-Cell-MPs does not include a signal peptide, a transmembrane domain, or an intracellular domain (cytoplasmic tail) associated with a native MHC Class I heavy chain. Thus, e.g., in some cases, the MHC-H chain present in a T-Cell-MP includes only the α1, α2, and α3 domains of a MHC Class I heavy chain. The MHC Class I heavy chain present in a T-Cell-MP may have a length of from about 270 amino acids (aa) to about 290 aa. The MHC Class I heavy chain present in a T-Cell-MP may have a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

The Class I HLA heavy chain polypeptides of T-Cell-MPs may comprise polypeptide sequences having at least 95%, or at least 98% aa sequence identity to all or part, for example at least about 200 (e.g., at least about 225, at least about 250, or at least about 260) contiguous aas, of the sequence of any of the human HLA heavy chain polypeptides depicted in FIGS. 3A to 3I (e.g., the sequences encompassing the α1, α2, and α3 domains). The Class I HLA heavy chain polypeptides of T-Cell-MPs may comprise polypeptide sequences having at least 95%, or at least 98% aa sequence identity to at least about 200 (e.g., at least about 225, at least about 250, or at least about 260) contiguous aas of the sequence of any of the human HLA heavy chain polypeptides depicted in FIGS. 3A to 3I (e.g., the sequences encompassing the α1, α2, and α3 domains). The Class I HLA heavy chain polypeptides of T-Cell-MPs may comprise polypeptide sequences having at least 95%, or at least 98% aa sequence identity to all or part, for example at least about 220 or at least about 240 contiguous aas, of the sequence of any of the human HLA heavy chain polypeptides depicted in FIGS. 3A to 3I (e.g., the sequences encompassing the α1, α2, and α3 domains). When calculating the percent aa sequence identity of a sequence (a “test sequence”) to any consensus sequence provided herein, aa residues of the test sequence at a position that aligns with variable residue and contain an amino acid recited among the amino acids present at variable residue in the consensus sequence may be counted as having identity. By way of example, where a test sequence recites a glu residue at a position corresponding to variable position X1 of an MHC sequence, where X1 is glu or lys, the test sequence glu residue may be counted as having identity.

The Class I HLA heavy chain polypeptides may comprise 1-30, 1-5, 5-10, 10-15, 15-20, 20-25 or 25-30 aa insertions, deletions, and/or substitutions (in addition to those locations indicated as being variable in the heavy chain consensus sequences of FIGS. 3E to 3I).

As an example, a MHC Class I heavy chain polypeptide of a multimeric polypeptide may comprise an aa sequence having at least 85%, at least 90%, at least 95%, at least 98%, at least 99%, or 100% aa sequence identity to aas 25-300 (lacking all, or substantially all, of the leader, transmembrane and cytoplasmic sequences) or aas 25-365 (lacking the leader) of a human HLA-A heavy chain polypeptides depicted in FIGS. 3A, 3B and/or 3C. An MHC Class I heavy chain polypeptide of a multimeric polypeptide may comprise an aa sequence having at least 95%, or at least 98%, aa sequence identity to aas 25-300 of an HLA heavy chain polypeptides depicted in FIG. 3A, 3B, or 3C. An MHC Class I heavy chain polypeptide of a multimeric polypeptide may comprise an aa sequence having at least 99% or 100%, aa sequence identity to aas 25-300 of an HLA heavy chain polypeptides depicted in FIG. 3A, 3B, or 3C.

a. MHC Class I Heavy Chains

Class I human MHC polypeptides may be drawn from the classical HLA alleles (HLA-A, B, and C), or the non-classical HLA alleles (e.g., HLA-E, F and G). The following are non-limiting examples of MHC-H alleles and variants of those alleles that may be incorporated into T-Cell-MPs and their epitope conjugates.

### (i) HLA-A Heavy Chains

The HLA-A heavy chain peptide sequences, or portions thereof, that may be incorporated into a T-Cell-MP include, but are not limited to, the alleles: A*0101, A*0201, A*0301, A*1101, A*2301, A*2402, A*2407, A*3303, and A*3401, which are aligned without all, or substantially all, of the leader, transmembrane and cytoplasmic sequences in FIG. 3E. Any of those alleles may further comprise a substitution at one or more of positions 84 and/or 139 (as shown in FIG. 3E) selected from: a tyrosine to alanine at position 84 (Y84A); a tyrosine to cysteine at position 84 (Y84C); and an alanine to cysteine at position 139 (A139C). In addition, an HLA-A sequence having at least 85% (e.g., at least about 90%; at least about 95%; at least about 98%; or at least about 99%) or 100% aa sequence identity to all or part (e.g., 100, 150, 200, 225, 250, or 260 contiguous aas) of the sequence of those HLA-A alleles may also be incorporated into a T-Cell-MP (e.g., it may comprise 1-30, 1-5, 5-10, 10-15, 15-20, 20-25, or 25-30 aa insertions, deletions, and/or substitutions). The HLA-A heavy chain polypeptide sequence of a T-Cell-MP may comprise Y84C and A139C substitutions that may form a disulfide bond.

### (a) HLA-A*0101 (HLA-A*01:01:01:01)

A MHC Class I heavy chain polypeptide of a T-Cell-MP or a T-Cell-MP-epitope conjugate may comprise the aa sequence of HLA-A*01:01:01:01 (HLA-A*0101, or HLA-A*01:01 listed as HLA-A in FIG. 3D (SEQ ID NO:24) and in FIG. 3E), or a sequence having at least 85% (e.g., at least about 90%; at least about 95%; at least about 98%; or at least about 99%) or 100% aa sequence identity to all or part, for example at least 200 (e.g., at least 225, at least 250, or at least 260) contiguous aas of that sequence. For example, the sequence may comprise 1-30, 1-5, 5-10, 10-15, 15-20, 20-25, or 25-30 aa insertions, deletions, and/or substitutions). In an embodiment, where the HLA-A heavy chain polypeptide of a T-Cell-MP has less than 100% identity to the sequence labeled HLA-A in FIG. 3D, it may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-A*0101 heavy chain polypeptide sequence of a T-Cell-MP may comprise Y84C and A139C substitutions that may form a disulfide bond.

### (b) HLA-A*0201 (HLA-A*02:01)

A MHC Class I heavy chain polypeptide of a T-Cell-MP or a T-Cell-MP-epitope conjugate may comprise an aa sequence of HLA-A*0201 (SEQ ID NO:27) provided in FIG. 3D or FIG. 3E, or a sequence having at least 85% (e.g., at least about 90%; at least about 95%; at least about 98%; or at least about 99%) or 100% aa sequence identity to all or part, for example at least 200 (e.g., at least 225, at least 250, or at least 260) contiguous aas of that sequence. For example, the sequence may comprise 1-30, 1-5, 5-10, 10-15, 15-20, 20-25, or 25-30 aa insertions, deletions, and/or substitutions). In an embodiment, where the HLA-A*0201 heavy chain polypeptide of a T-Cell-MP has less than 100% identity to the sequence labeled HLA-A*0201 in FIG. 3D or 3E, it may comprise a substitution at one or more of positions 84 and/or 139selected from: a tyrosine to alanine at position 84 (Y84A); a tyrosine to cysteine at position 84 (Y84C); and an alanine to cysteine at position 139 (A139C). The HLA-A*0201 heavy chain polypeptide sequence of a T-Cell-MP may comprise Y84C and A139C substitutions that may form a disulfide bond.

### (c) HLA-A*1101 (HLA-A*11:01)

A MHC Class I heavy chain polypeptide of a T-Cell-MP or a T-Cell-MP-epitope conjugate may comprise an aa sequence of HLA-A*1101 (SEQ ID NO:32) provided in FIG. 3D or 3E, or a sequence having at least 85% (e.g., at least about 90%; at least about 95%; at least about 98%; or at least about 99%) or 100% aa sequence identity to all or part, for example at least 200 (e.g., at least 225, at least 250, or at least 260) contiguous aas of that sequence. For example, the sequence may comprise 1-30, 1-5, 5-10, 10-15, 15-20, 20-25, or 25-30 aa insertions, deletions, and/or substitutions). The HLA-A*1101 heavy chain allele may be prominent in Asian populations, including populations of individuals of Asian descent.

In an embodiment, where the HLA-A*1101 heavy chain polypeptide of a T-Cell-MP has less than 100% identity to the sequence labeled HLA-A*1101 in FIG. 3D or 3E, it may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-A*1101 heavy chain polypeptide sequence of a T-Cell-MP may comprise Y84C and A139C substitutions that may form a disulfide bond.

### (d) HLA-A*2402 (HLA-A*24:02)

A MHC Class I heavy chain polypeptide of a T-Cell-MP or a T-Cell-MP-epitope conjugate may comprise an aa sequence of HLA-A*2402 (SEQ ID NO:33) provided in FIG. 3D or 3E, or a sequence having at least 85% (e.g., at least about 90%; at least about 95%; at least about 98%; or at least about 99%) or 100% aa sequence identity to all or part, for example at least 200 (e.g., at least 225, at least 250, or at least 260) contiguous aas of that sequence. For example, the sequence may comprise 1-30, 1-5, 5-10, 10-15, 15-20, 20-25, or 25-30 aa insertions, deletions, and/or substitutions). The HLA-A*2402 heavy chain allele may be prominent in Asian populations, including populations of individuals of Asian descent.

In an embodiment, where the HLA-A*2402 heavy chain polypeptide of a T-Cell-MP has less than 100% identity to the sequence labeled HLA-A*2402 in FIG. 3D or 3E, it may comprise a substitution at one or more of positions 84 and/or selected from: a tyrosine to alanine at position 84 (Y84A); a tyrosine to cysteine at position 84 (Y84C); and an alanine to cysteine at position 139 (A139C). The HLA-A*2402 heavy chain polypeptide sequence of a T-Cell-MP may comprise Y84C and A139C substitutions that may form a disulfide bond.

### (e) HLA-A*3303 (HLA-A*33:03) or HLA-A*3401 (HLA-A*34:01)

A MHC Class I heavy chain polypeptide of a T-Cell-MP or a T-Cell-MP-epitope conjugate may comprise an aa sequence of HLA-A*3303 (SEQ ID NO:34) or HLA-A*3401 (SEQ ID NO:38) provided in FIG. 3D or 3E, or a sequence having at least 85% (e.g., at least about 90%; at least about 95%; at least about 98%; or at least about 99%) or 100% aa sequence identity to all or part, for example at least 200 (e.g., at least 225, at least 250, or at least 260) contiguous aas of either of those sequences. 1-30, 1-5, 5-10, 10-15, 15-20, 20-25, or 25-30 aa insertions, deletions, and/or substitutions). The HLA-A*3303 heavy chain allele may be prominent in Asian populations, including populations of individuals of Asian descent.

In an embodiment, where the HLA-A*3303 or HLA-A*3401 heavy chain polypeptide of a T-Cell-MP has less than 100% identity to the sequence labeled HLA-A*3303 in FIG. 3D, it may comprise a substitution at one or more of positions 84 and/or139 selected from: a tyrosine to alanine at position 84 (Y84A); a tyrosine to cysteine at position 84 (Y84C); and an alanine to cysteine at position 139 (A139C). The HLA-A*3303 or HLA-A*3401 heavy chain polypeptide sequence of a T-Cell-MP may comprise Y84C and A139C substitutions that may form a disulfide bond.

### (ii) HLA-B Heavy Chains.

The HLA-B heavy chain peptide sequences, or portions thereof, that may be incorporated into a T-Cell-MP include, but are not limited to, the alleles: B*0702, B*0801, B*1501, B*1502, B*2705, B*03501, B*3802, B*4001, B*4402, B*4403, B*4601, B*5301, and B*5801, some of which are aligned without all, or substantially all, of the leader, transmembrane and cytoplasmic sequences in FIG. 3F. Any of those alleles may comprise a substitution at one or more of positions 84 and/or 139 (as shown in FIG. 3F) selected from: a tyrosine to alanine at position 84 (Y84A); a tyrosine to cysteine at position 84 (Y84C); and an alanine to cysteine at position 139 (A139C). In addition, an HLA-B sequence having at least 85% (e.g., at least about 90%; at least about 95%; at least about 98%; or at least about 99%) or 100% sequence identity to all or part (e.g., 100, 150, 200, 225, 250, or 260 contiguous aas) of the sequence of those HLA-B alleles may also be incorporated into a T-Cell-MP (e.g., it may comprise 1-25, 1-5, 5-10, 10-15, 15-20, 20-25, or 25-30 aa insertions, deletions, and/or substitutions). The HLA-B heavy chain polypeptide sequence of a T-Cell-MP may comprise Y84C and A139C substitutions that may form a disulfide bond.

### (a) HLA-B*0702 (HLA-B*07:02)

A MHC Class I heavy chain polypeptide of a T-Cell-MP or a T-Cell-MP-epitope conjugate may comprise an aa sequence of HLA-B*0702 (SEQ ID NO:25) in FIG. 3D (labeled HLA-B in FIG. 3D), or a sequence having at least 85% (e.g., at least about 90%, at least about 95%, at least about 98%, or at least about 99%) or 100% aa sequence identity to all or part, for example at least 200 (e.g., at least 225, at least 250, or at least 260) contiguous aas of that sequence. For example, the sequence may comprise 1-30, 1-5, 5-10, 10-15, 15-20, 20-25, or 25-30 aa insertions, deletions, and/or substitutions). In an embodiment, where the HLA-B heavy chain polypeptide of a T-Cell-MP has less than 100% identity to the sequence labeled HLA-B in FIG. 3D, it may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-B*0702 heavy chain polypeptide sequence of a T-Cell-MP may comprise Y84C and A139C substitutions.

### (b) HLA-B*1501 (HLA-B*15:01)

A MHC Class I heavy chain polypeptide of a T-Cell-MP or a T-Cell-MP-epitope conjugate may comprise an aa sequence of HLA-B*1501: GSHSMRYFYTAMSRPGRGEPRFIAVGYVDDTQFVRFDSDAASPRMAPRAPWIE QEGPEYWDRETQISKTNTQTYRESLRNLRGYYNQSEAGSHTLQRMYGCDVGPDGRLLRGHDQSAYDGKDYIALNE DLSSWTAADTAAQITQRKWEAAREAEQWRAYLEGLCVEWLRRYLENGKETLQRADPPKTHVTHHPISDHEATLRCW ALGFYPAEITLTWQRDGEDQTQDTELVETRPAGDRTFQKWAAVVVPSGEEQRYTCHVQHEGLPKPLTLRWEP (shown lacking its signal sequence and transmembrane/intracellular regions SEQ ID NO:2041), or a sequence having at least 85% (e.g., at least about 90%, at least about 95%, at least about 98%, or at least about 99%) or 100% aa sequence identity to all or part, for example at least 200 (e.g., at least 225, at least 250, or at least 260) contiguous aas of that sequence. For example, the sequence may comprise 1-30, 1-5, 5-10, 10-15, 15-20, 20-25, or 25-30 aa insertions, deletions, and/or substitutions). In an embodiment, the sequence may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-B*3501 heavy chain polypeptide sequence of a T-Cell-MP may comprise Y84C and A139C substitutions.

### (c) HLA-B*2705 (HLA-B*27:05)

A MHC Class I heavy chain polypeptide of a T-Cell-MP or a T-Cell-MP-epitope conjugate may comprise an aa sequence of HLA-B*2705: GSHSMRYFHTSVSRPGRGEPRFITVGYVDDTLFVRFDSDAASPREEPRAPWIEQEGPEY WDRETQICKAKAQTDREDLRTLLRYYNQSEAGSHTLQNMYGCDVGPDGRLLRGYHQDAYDGKDYIALNEDLSSWT AADTAAQITQRKWEAARVAEQLRAYLEGECVEWLRRYLENGKETLQRADPPKTHVTHHPISDHEATLRCWALGFYP AEITLTWQRDGEDQTQDTELVETRPAGDRTFQKWAAVVVPSGEEQRYTCHVQHEGLPKPLTLRWEP (shown lacking its signal sequence and transmembrane/intracellular regions SEQ ID NO:2042), or a sequence having at least 85% (e.g., at least about 90%, at least about 95%, at least about 98%, or at least about 99%) or 100% aa sequence identity to all or part, for example at least 200 (e.g., at least 225, at least 250, or at least 260) contiguous aas of that sequence. For example, the sequence may comprise 1-30, 1-5, 5-10, 10-15, 15-20, 20-25, or 25-30 aa insertions, deletions, and/or substitutions). In an embodiment, the sequence may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-B*3501 heavy chain polypeptide sequence of a T-Cell-MP may comprise Y84C and A139C substitutions.

### (d) HLA-B*3501 (HLA-B*35:01)

A MHC Class I heavy chain polypeptide of a T-Cell-MP or a T-Cell-MP-epitope conjugate may comprise an aa sequence of HLA-B*3501: GSHSMRYFYTAMSRPGRGEPRFIAVGYVDDTQFVRFDSDAASPRTEPRAPWIEQ EGPEYWDRNTQIFKTNTQTYRESLRNLRGYYNQSEAGSHIIQRMYGCDLGPDGRLLRGHDQSAYDGKDYIALNEDL SSWTAADTAAQITQRKWEAARVAEQLRAYLEGLCVEWLRRYLENGKETLQRADPPKTHVTHHPVSDHEATLRCWAL GFYPAEITLTWQRDGEDQTQDTELVETRPAGDRTFQKWAAVVVPSGEEQRYTCHVQHEGLPKPLTLRWEP (shown lacking its signal sequence and transmembrane/intracellular regions SEQ ID NO:80), or a sequence having at least 85% (e.g., at least about 90%, at least about 95%, at least about 98%, or at least about 99%) or 100% aa sequence identity to all or part, for example at least 200 (e.g., at least 225, at least 250, or at least 260) contiguous aas of that sequence. For example, the sequence may comprise 1-30, 1-5, 5-10, 10-15, 15-20, 20-25, or 25-30 aa insertions, deletions, and/or substitutions). In an embodiment, the sequence may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-B*3501 heavy chain polypeptide sequence of a T-Cell-MP may comprise Y84C and A139C substitutions.

### (e) HLA-B*4402 (HLA-B*44:02)

A MHC Class I heavy chain polypeptide of a T-Cell-MP or a T-Cell-MP-epitope conjugate may comprise an aa sequence of HLA-B*4402: GSHSMRYFYTAMSRPGRGEPRFITVGYVDDTLFVRFDSDATSPRKEPRAPWIEQE GPEYWDRETQISKTNTQTYRENLRTALRYYNQSEAGSHIIQRMYGCDVGPDGRLLRGYDQDAYDGKDYIALNEDLSS WTAADTAAQITQRKWEAARVAEQDRAYLEGLCVESLRRYLENGKETLQRADPPKTHVTHHPISDHEVTLRCWALGF YPAEITLTWQRDGEDQTQDTELVETRPAGDRTFQKWAAVVVPSGEEQRYTCHVQHEGLPKPLTLRWEP (shown lacking its signal sequence and transmembrane/intracellular regions SEQ ID NO:81), or a sequence having at least 85% (e.g., at least about 90%, at least about 95%, at least about 98%, or at least about 99%) or 100% aa sequence identity to all or part, for example at least 200 (e.g., at least 225, at least 250, or at least 260) contiguous aas of that sequence. For example, the sequence may comprise 1-30, 1-5, 5-10, 10-15, 15-20, 20-25, or 25-30 aa insertions, deletions, and/or substitutions). In an embodiment, the sequence may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-B*4402 heavy chain polypeptide sequence of a T-Cell-MP may comprise Y84C and A139C substitutions.

### (f) HLA-B*4403 (HLA-B*44:03)

A MHC Class I heavy chain polypeptide of a T-Cell-MP or a T-Cell-MP-epitope conjugate may comprise an aa sequence of HLA-B*4403: GSHSMRYFYTAMSRPGRGEPRFITVGYVDDTLFVRFDSDATSPRKEPRAPWIEQ EGPEYWDRETQISKTNTQTYRENLRTALRYYNQSEAGSHIIQRMYGCDVGPDGRLLRGYDQDAYDGKDYIALNEDLS SWTAADTAAQITQRKWEAARVAEQLRAYLEGLCVESLRRYLENGKETLQRADPPKTHVTHHPISDHEVTLRCWALGF YPAEITLTWQRDGEDQTQDTELVETRPAGDRTFQKWAAVVVPSGEEQRYTCHVQHEGLPKPLTLRWEP (shown lacking its signal sequence and transmembrane/intracellular regions SEQ ID NO:82), or a sequence having at least 85% (e.g., at least about 90%, at least about 95%, at least about 98%, or at least about 99%) or 100% aa sequence identity to all or part, for example at least 200 (e.g., at least 225, at least 250, or at least 260) contiguous aas of that sequence. For example, the sequence may comprise 1-30, 1-5, 5-10, 10-15, 15-20, 20-25, or 25-30 aa insertions, deletions, and/or substitutions). In an embodiment, the sequence may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-B*4403 heavy chain polypeptide sequence of a T-Cell-MP may comprise Y84C and A139C substitutions.

### (g) HLA-B*5801 (HLA-B*58:01)

A MHC Class I heavy chain polypeptide of a T-Cell-MP or a T-Cell-MP-epitope conjugate may comprise an aa sequence of HLA-B*58:01: GSHSMRYFYTAMSRPGRGEPRFIAVGYVDDTQFVRFDSDAASPRTEPRAPWIEQ EGPEYWDGETRNMKASAQTYRENLRIALRYYNQSEAGSHIIQRMYGCDLGPDGRLLRGHDQSAYDGKDYIALNEDL SSWTAADTAAQITQRKWEAARVAEQLRAYLEGLCVEWLRRYLENGKETLQRADPPKTHVTHHPVSDHEATLRCWAL GFYPAEITLTWQRDGEDQTQDTELVETRPAGDRTFQKWAAVVVPSGEEQRYTCHVQHEGLPKPLTLRWEP (shown lacking its signal sequence and transmembrane/intracellular regions SEQ ID NO:83), or a sequence having at least 85% (e.g., at least about 90%, at least about 95%, at least about 98%, or at least about 99%) or 100% aa sequence identity to all or part, for example at least 200 (e.g., at least 225, at least 250, or at least 260) contiguous aas of that sequence. For example, the sequence may comprise 1-30, 1-5, 5-10, 10-15, 15-20, 20-25, or 25-30 aa insertions, deletions, and/or substitutions). In an embodiment, the sequence may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-B*5901 heavy chain polypeptide sequence of a T-Cell-MP may comprise Y84C and A139C substitutions.

(iii) HLA-C Heavy Chains

The HLA-C heavy chain peptide sequences, or portions thereof, that may be incorporated into a T-Cell-MP include, but are not limited to, the alleles: C*0102, C*0303, C*0304, C*0401, C*0602, C*0701, C*0702, C*0801, and C*1502, which are aligned without all, or substantially all, of the leader, transmembrane and cytoplasmic sequences in FIG. 3G. Any of those alleles may comprise a substitution at one or more of positions 84, 139 and/or 236 (as shown in FIG. 3G) selected from: a tyrosine to alanine at position 84 (Y84A); a tyrosine to cysteine at position 84 (Y84C); and an alanine to cysteine at position 139 (A139C). In addition, an HLA-C sequence having at least 85% (e.g., at least about 90%, at least about 95%, at least about 98%, or at least about 99%) or 100% aa sequence identity to all or part, for example at least 200 (e.g., at least 225, at least 250, or at least 260) contiguous aas of any of those sequences. For example, the sequence may comprise 1-30, 1-5, 5-10, 10-15, 15-20, 20-25, or 25-30 aa insertions, deletions, and/or substitutions). The HLA-C heavy chain polypeptide sequence of a T-Cell-MP may comprise Y84C and A139C substitutions that may form a disulfide bond.

### (a) HLA-C*701 (HLA-C*07:01) and HLA-C*702 (HLA-C*07:02)

A MHC Class I heavy chain polypeptide of a T-Cell-MP or a T-Cell-MP-epitope conjugate may comprise an aa sequence of HLA-C*701 (SEQ ID NO:23) or HLA-C*702 (SEQ ID NO:54) in FIG. 3G (labeled HLA-C in FIG. 3D), or a sequence having at least 85% (e.g., at least about 90%, at least about 95%, at least about 98%, or at least about 99%) or 100% aa sequence identity to all or part, for example at least 200 (e.g., at least 225, at least 250, or at least 260) contiguous aas of any of those sequences. For example, the sequence may comprise 1-30, 1-5, 5-10, 10-15, 15-20, 20-25, or 25-30 aa insertions, deletions, and/or substitutions relative to those sequences). In an embodiment, where the HLA-C heavy chain polypeptide of a T-Cell-MP has less than 100% identity to the sequence labeled HLA-C in FIG. 3D, it may comprise a substitution at one or more of positions 84 and/or 139 selected from: a tyrosine to alanine at position 84 (Y84A); a tyrosine to cysteine at position 84 (Y84C); and an alanine to cysteine at position 139 (A139C). The HLA-C*701 or HLA-C*0702 heavy chain polypeptide sequence of a T-Cell-MP may comprise Y84C and A139C substitutions that may form a disulfide bond.

### (iv) Non-Classical HLA-E, F and G Heavy Chains

The non-classical HLA heavy chain peptide sequences, or portions thereof, that may be incorporated into a T-Cell-MP include, but are not limited to, those of the HLA-E, F, and/or G alleles. Sequences for those alleles, (and the HLA-A, B and C alleles) may be found on the World Wide Web at, for example, hla.alleles.org/nomenclature/index.html, the European Bioinformatics Institute (www.ebi.ac.uk), which is part of the European Molecular Biology Laboratory (EMBL), and the National Center for Biotechnology Information (www.ncbi.nlm.nih.gov).

Some suitable HLA-E alleles include, but are not limited to, HLA-E*0101 (HLA-E*01:01:01:01), HLA-E*01:03 (HLA-E*01:03:01:01), HLA-E*01:04, HLA-E*01:05, HLA-E*01:06, HLA-E*01:07, HLA-E*01:09, and HLA-E*01:10. Some suitable HLA-F alleles include, but are not limited to, HLA-F*0101 (HLA-F*01:01:01:01), HLA-F*01:02, HLA-F*01:03 (HLA-F*01:03:01:01), HLA-F*01:04, HLA-F*01:05, and HLA-F*01:06. Some suitable HLA-G alleles include, but are not limited to, HLA-G*0101 (HLA-G*01:01:01:01), HLA-G*01:02, HLA-G*01:03 (HLA-G*01:03:01:01), HLA-G*01:04 (HLA-G*01:04:01:01), HLA-G*01:06, HLA-G*01:07, HLA-G*01:08, HLA-G*01:09: HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, and HLA-G*01:22. Consensus sequences for those HLA-E, -F, and -G alleles without all, or substantially all, of the leader, transmembrane and cytoplasmic sequences are provided in FIG. 3H, and aligned with consensus sequences of the above-mentioned HLA-A, -B, and -C alleles provided in FIGS. 3E-3G and in FIG. 3I.

Any of the above-mentioned HLA-E, F and/or G alleles may comprise a substitution at one or more of positions 84 and/or 139 as shown in FIG. 3I for the consensus sequences. In an embodiment, the substitutions may be selected from: a position 84 tyrosine to alanine (Y84A) or cysteine (Y84C), or in the case of HLA-F a R84A or R84C substitution; and/or a position 139 alanine to cysteine (A139C), or in the case of HLA-F a V139C substitution. In addition, HLA-E, -F, and/or -G sequences having at least 85% (e.g., at least about 90%; at least about 95%; at least about 98%; or at least about 99%) or 100% aa sequence identity to all or part (e.g., 100, 150, 200, 225, 250, or 260 contiguous aas) of any of the consensus sequences set forth in FIG. 3I may also be employed (e.g., the sequences may comprise 1-30, 1-5, 5-10, 10-15, 15-20, 20-25, or 25-30 aa insertions, deletions, and/or substitutions in addition to changes at variable residues listed therein). The HLA-E, F, or G heavy chain polypeptide sequence of a T-Cell-MP may comprise a cysteine at both position 84 and 139.

### (v) Mouse H2K

A MHC Class I heavy chain polypeptide of a T-Cell-MP or a T-Cell-MP-epitope conjugate may comprise an aa sequence of MOUSE H2K (SEQ ID NO:28) (MOUSE H2K in FIG. 3D), or a sequence having at least 75%, at least 80%, at least 85%, at least 90%, at least 95%, at least 98%, at least 99%, or 100% aa sequence identity to all or part (e.g., 50, 75, 100, 150, 200, 225, 250, or 260 contiguous aas) of that sequence (e.g., it may comprise 1-30, 1-5, 5-10, 10-15, 15-20, 20-25, or 25-30 aa insertions, deletions, and/or substitutions). In an embodiment, where the MOUSE H2K heavy chain polypeptide of a T-Cell-MP has less than 100% identity to the sequence labeled MOUSE H2K in FIG. 3D, it may comprise a substitution at one or more of positions 84 and/or139 selected from: a tyrosine to alanine at position 84 (Y84A); a tyrosine to cysteine at position 84 (Y84C); and an alanine to cysteine at position 139 (A139C). The mouse H2K heavy chain polypeptide sequence of a T-Cell-MP may comprise Y84C and A139C substitutions that may form a disulfide bond.

### (vi) The Effect of Amino Acid Substitutions in MHC Polypeptides on T-Cell-MPs

### (a) Substitutions at Positions 84 and 139

Substitution of position 84 of the MHC H chain (see FIG. 3I), particularly when it is a tyrosine residue, with a small amino acid such as alanine (Y84A) tends to open one end of the MHC binding pocket, allowing a linker (e.g., attached to a peptide epitope) to “thread” through the end of the pocket, and accordingly, permits a greater variation in the size of the epitope (e.g., longer peptides bearing epitope sequences) that can fit into the MHC pocket and be presented by the T-Cell-MP. Alternatively, the MHC-H (e.g., HLA-heavy chain) of a T-Cell-MP may be substituted with cysteines to form an intrachain disulfide bond between a cysteine substituted into the carboxyl end portion of the α1 helix and a cysteine in the amino end portion of the α2-1 helix (e.g., amino acids 84 and 139). Such disulfide bonds stabilize the MHC-H polypeptide sequence of a T-Cell-MP, and permit its translation, cellular processing, and excretion from eukaryotic cells in the absence of a bound peptide epitope (or null peptide). Any combination of substitutions provided in the table provide below at residues 84 and 130 may be combined with any combination of substitutions in the epitope binding cleft, such as those described at positions 116 and 167.

### (b) Substitutions at Positions 116 and 167

Any MHC Class I heavy chain sequences (including those disclosed above for: the HLA-A*0101; HLA-A*0201; HLA-A*1101; HLA-A*2402; HLA-A*3303; HLA-B; HLA-C; Mouse H2K, or any of the other HLA-A, B, C, E, F, and/or G sequence disclosed herein) may further comprise a cysteine substitution at position 116 (e.g., Y116C) or at position 167.

As with aa position 84 substitutions that open one end of the MHC-H binding pocket (e.g., Y84A or its equivalent), substitution of an alanine or glycine at position 167 (e.g., a W167A substitution or its equivalent) opens the other end of the MHC binding pocket, creating a groove that permits greater variation (e.g., longer length) of the peptide epitopes that may be presented by the T-Cell-MP-epitope conjugates. Substitutions at positions 84 and/or 167, or their equivalent (e.g., Y84A in combination with W167A or W167G) may be used in combination to modify the binding pocket of MHC-H chains. A cysteine substitution at positions 116 (e.g., Y116C) and/or 167 (e.g., W167C) may be used separately or in combination to anchor epitopes (e.g., peptide epitopes) in one or two locations (e.g., the ends of the epitope containing peptide). Substitutions at positions 116 and/or 167 may be combined with substitutions including those at positions 84 and/or 139 described above.

The Table below lists some MHC heavy chain sequence modifications that may be incorporated into a T-Cell-MPs.

Some Combinations of MHC Class 1 Heavy Chain Sequence Modifications that May be Incorporated into a T-Cell-MP or its Epitope Conjugate

| | HLA Heavy Chain | | | |
|---|---|---|---|---|
| | Sequence | Sequence | Substitutions | Substitutions |
| | From | Identity | at aa positions | at positions |
| Entry | FIGS. 3D-H | Range [symbol] | 84 and/or 139 | 116 and/or 167 |
| 1 | HLA-A | 75%-99.8%, 80%-99.8%, 85%-99.8%, | None; Y84C; Y84A; | None; Y116C; |
| | Consensus | 90%-99.8%, 95%-99.8%, 98%-99.8%, or | A139C; or (Y84C & | W167A; W167C; or |
| | FIG. 3E | 99%-99.8%; or 1-25, 1-5, 5-10, 10-15, 15- | A139C) | (Y116C & |
| | | 20, or 20-25 aa insertions, deletions, | | W167C) |
| | | and/or substitutions (not counting variable | | |
| | | residues) | | |
| 2 | A*0101, A*0201, | 75%-99.8%, 80%-99.8%, 85%-99.8%, | None; Y84C; Y84A; | None; Y116C; |
| | A*0301, A*1101, | 90%-99.8%, 95%-99.8%, 98%-99.8%, or | A139C; or (Y84C & | W167A; W167C; or |
| | A*2402, A*2301, | 99%-99.8%; or 1-25, 1-5, 5-10, 10-15, 15- | A139C) | (Y116C & |
| | A*2402, A*2407, | 20, or 20-25 aa insertions, deletions, and/or | | W167C) |
| | A*3303, or A*3401 | substitutions | | |
| 3 | HLA-B | 75%-99.8%, 80%-99.8%, 85%-99.8%, | None; Y84C; Y84A; | None; Y116C; |
| | Consensus | 90%-99.8%, 95%-99.8%, 98%-99.8%, or | A139C; or (Y84C & | W167A; W167C; or |
| | FIG. 3F | 99%-99.8%; or 1-25, 1-5, 5-10, 10-15, 15- | A139C) | (Y116C & |
| | | 20, or 20-25 aa insertions, deletions, and/or | | W167C) |
| | | substitutions (not counting variable | | |
| | | residues) | | |
| 4 | B*0702, B*0801, | 75%-99.8%, 80%-99.8%, 85%-99.8%, | None; Y84C; Y84A; | None; Y116C; |
| | B*1502, B*3501, | 90%-99.8%, 95%-99.8%, 98%-99.8%, or | A139C; or (Y84C & | W167A; W167C; or |
| | B*3802, B*4001, | 99%-99.8%; or 1-25, 1-5, 5-10, 10-15, 15- | A139C) | (Y116C & |
| | B*4402, B*4403, | 20, or 20-25 aa insertions, deletions, and/or | | W167C) |
| | B*4601, B*5301, or | substitutions | | |
| | B*5801 | | | |
| 5 | HLA-C | 75%-99.8%, 80%-99.8%, 85%-99.8%, | None; Y84C; Y84A; | None; Y116C; |
| | Consensus | 90%-99.8%, 95%-99.8%, 98%-99.8%, or | A139C; or (Y84C & | W167A; W167C; or |
| | FIG. 3G | 99%-99.8%; or 1-25, 1-5, 5-10, 10-15, 15- | A139C) | (Y116C & |
| | | 20, or 20-25 aa insertions, deletions, and/or | | W167C) |
| | | substitutions (not counting variable | | |
| | | residues) | | |
| 6 | C*0102, C*0303, | 75%-99.8%, 80%-99.8%, 85%-99.8%, | None; Y84C; Y84A; | None; Y116C; |
| | C*0304, C*0401, | 90%-99.8%, 95%-99.8%, 98%-99.8%, or | A139C; or (Y84C & | W167A; W167C; or |
| | C*0602, C*0701, | 99%-99.8%; or 1-25, 1-5, 5-10, 10-15, 15- | A139C) | (Y116C & |
| | C*702, C*0801, or | 20, or 20-25 aa insertions, deletions, and/or | | W167C) |
| | C*1502 | substitutions | | |
| 7 | HLA-E, F, or G | 75%-99.8%, 80%-99.8%, 85%-99.8%, | None; Y84C; Y84A; | None; Y116C; |
| | Consensus | 90%-99.8%, 95%-99.8%, 98%-99.8%, or | A139C; or (Y84C & | W167A; W167C; or |
| | FIG. 3H | 99%-99.8%; or 1-25, 1-5, 5-10, 10-15, 15- | A139C) | (Y116C & |
| | | 20, or 20-25 aa insertions, deletions, and/or | | W167C) |
| | | substitutions (not counting variable | | |
| | | residues) | | |
| 8 | MOUSE H2K | 75%-99.8%, 80%-99.8%, 85%-99.8%, | None; Y84C; Y84A; | None; Y116C; |
| | | 90%-99.8%, 95%-99.8%, 98%-99.8%, or | A139C; or (Y84C & | W167A; W167C; or |
| | | 99%-99.8%; or 1-25, 1-5, 5-10, 10-15, 15- | A139C) | (Y116C & |
| | | 20, or 20-25 aa insertions, deletions, and/or | | W167C) |
| | | substitutions | | |

[symbol]

The Sequence Identity Range is the permissible range in sequence identity of a MHC-H polypeptide sequence incorporated into a T-Cell-MP relative to the corresponding portion of the sequences listed in FIG. 3D-3H not counting the variable residues when the consensus sequences are used for the comparison.

b. MHC Class I β2-Microglobins and Combinations with MHC-H Polypeptides

A β2M polypeptide of a T-Cell-MP can be a human β2M polypeptide, a non-human primate β2M polypeptide, a murine β2M polypeptide, and the like. In some instances, a β2M polypeptide comprises an aa sequence having at least 75%, at least 80%, at least 85%, at least 90%, at least 95%, at least 98%, at least 99%, or 100% aa sequence identity to a β2M aa sequence (e.g., a mature β2M sequence) depicted in FIG. 4. The β2M polypeptide of a T-Cell-MP may comprise an aa sequence having at least 75%, at least 80%, at least 85%, at least 90%, at least 95%, at least 98%, at least 99%, or 100% aa sequence identity to aas 21 to 119 of a β2M aa sequence depicted in FIG. 4, which may include a cysteine or other aa substitution or insertion as a chemical conjugation site for epitope attachment (e.g., and E44C substitution) when the identity is less than 100%. Chemical conjugation sites may be located at, for example, solvent accessible locations in the β2M polypeptide sequence.

The β2M polypeptide sequence of a T-Cell-MP may have at least 90% (e.g., at least 95% or at least 98%) or 100% sequence identity to at least 70 (e.g., at least 80, at least 90, at least 96, at least 97, at least 98) or all contiguous aas of a mature human β2M polypeptide (e.g., aas 21-119 of NCBI accession number NP_004039.1 provided in FIG. 4). By way of example, a β2M polypeptide sequence of a T-Cell-MP may have up to six (e.g., 1, 2, 3, 4, 5, or 6) aa substitutions within an aa segment of at least 70 (e.g., at least 80, at least 90, at least 96, at least 97, or at least 98) or all contiguous aas of a mature human β2M polypeptide (e.g., aas 21-119 of NCBI accession number NP_004039.1 provided in FIG. 4), and may comprise the chemical conjugation site for attachment of an epitope (e.g., an E44C substitution in the mature peptide). As noted above, in such β2M polypeptide sequences the chemical conjugation sites of epitopes may be located at a variety of locations including solvent accessible aa positions. For example, a cysteine or other amino acid substitution or insertion at a solvent accessible amino acid position can provide a chemical conjugation site for direct or indirect (e.g., through a peptide linker) attachment of an epitope.

Some solvent accessible positions of mature β2M polypeptides lacking their leader sequence include aa positions 2, 14, 16, 34, 36, 44, 45, 47, 48, 50, 58, 74, 77, 85, 88, 89, 91, 94, and 98 (Gln 2, Pro 14, Glu 16, Asp 34, Glu 36, Glu 44, Arg 45, Glu 47, Arg 48, Glu 50, Lys 58, Glu 74, Glu 77, Val 85, Ser 88, Gln 89, Lys 91, Lys 94, and Asp 98) of the mature peptide from NP_004039.1, or their corresponding amino acids in other β2M sequences (see the sequence alignment in FIG. 4). The solvent accessible locations for chemical conjugation sites (e.g., a cysteine or another reactive aa substitution) may be selected from positions 2, 44, 50, 77, 85, 88, 91, or 98 of a mature β2M polypeptide sequence such as NP_004039.1, or the corresponding aa positions in other β2M sequences such as those in FIG. 4. The solvent accessible locations for chemical conjugation sites (e.g., a cysteine or another reactive aa substitution) may also be selected from positions 2, 44, 50, or 98 of a mature β2M polypeptide sequence such as NP_004039.1, or the corresponding aa positions in other β2M sequences such as those in FIG. 4. The solvent accessible locations for chemical conjugation sites (e.g., a cysteine or another reactive aa substitution) may be selected from positions 2 or 44 (Glu 2 or Glu 44) of a mature β2M polypeptide sequence such as NP_004039.1, or the corresponding aa positions in other β2M sequences such as those in FIG. 4.

A β2M polypeptide sequence may comprise a single cysteine substituted into a wt. β2M polypeptide (e.g., a β2M sequence in FIG. 4). Such cysteine residues, when present in a T-Cell-MP polypeptide, can act as a chemical conjugation site for the covalent coupling of an epitope (either directly or indirectly through a linker). The covalent attachment may be in the form of a bond made to a reactive group in or attached to the epitope, such as a maleimide group incorporated into the epitope or a linker attached to the peptide epitope, or in the form of a disulfide bond. For example, in some cases, one of amino acids 43, 44, or 45 of the mature β2M lacking its signal sequence (residues 63, 64, and 65 of the unprocessed proteins as shown with their signal sequences in FIG. 4) may be substituted with a cysteine residue. The aa of β2M substituted with a cysteine may be at position 44 of a mature β2M aa sequence. For example, an E44C substitution of the mature human β2M protein NP_004039.1 has the sequence IQRTPKIQVYSRHPAENGKSNFLNCYVSGFHPSDIEVDLLKNGCRIEKVEHSDLSFSKDWSFYLLYY TEFTPTEKDEYACRVNHVTLSQPKIVKWDRDM (SEQ ID NO:2092), with the cysteine at position 44 bolded and italicized. A corresponding aa substitution in other β2M sequences, such as those in FIG. 4 may be used for a conjugation site. Alternatively, the aa position substituted with a cysteine may be position 2 (e.g., a Q44C substitution of the mature human protein NP_004039.1 or a corresponding aa substitution in a β2M sequence such as those in FIG. 4).

c. Some Combinations of Substitutions in the MHC-H and the β2M Polypeptide Sequences

Separately, or in addition to, any cysteine residues inserted into the MHC-H or β2M polypeptide sequence of a T-Cell-MP that may function as a chemical conjugation site for an epitope or a payload (e.g., an E44C substitution in a β2M polypeptide sequence that provides a chemical conjugation site for an epitope), a T-Cell-MP may comprise an intrachain disulfide bond between a cysteine substituted into the carboxyl end portion of the α1 helix and a cysteine in the amino end portion of the α2-1 helix (e.g., amino acids at aa positions 84 and 139, such as Y84C and A139C). The carboxyl end portion of the α1 helix is from about aa position 79 to about aa position 89 and the amino end portion of the α2-1 helix is from about aa position 134 to about aa position 144 of the MHC-H chain (the aa positions are determined based on the sequence of the heavy chains without their leader sequence (see, e.g., FIGS. 3D-3H). Accordingly, a disulfide bond may be between a cysteine located at positions 83, 84, or 85 and a cysteine located at any of positions 138, 139 or 140 of the MHC-H polypeptide sequence. For example, in a T-Cell-MP a disulfide bond may be formed between a cysteine inserted at position 84 and a cysteine inserted at any of positions 138, 139 or 140 of the MHC-H polypeptide sequence. In one aspect, the MHC-H intrachain disulfide bond is between cysteines substituted at positions 84 and 139 of any of the heavy chain sequences set forth in FIGS. 3D-3H.

A T-Cell-MP may comprise a combination of: (i) a mature β2M polypeptide sequence having at least 90% (e.g., at least 95% or at least 98%) sequence identity to at least 70 (e.g., at least 80, at least 90, at least 96, at least 97, at least 98 or all) of aas 21-119 of NP_004039.1 with an E44C (or another cysteine substitution) as a chemical conjugation site for an epitope; and (ii) an HLA Class I heavy chain polypeptide sequence having at least 90% sequence identity (e.g., at least 95%, at least 98%, or 100% sequence identity) excluding variable aa clusters (aac) 1-4 to: GSHSMRYFFTSVSRPGRGEPRFIAVGYVDDTQFVRFDSDAASQRMEPRAPWIEQEGPEYWDGETRKVKAH SQTHRVDL(aa cluster 1){C}(aa cluster 2)AGSHTVQRMYGCDVGSDWRFLRGYHQYAYDGKDYIALKEDLRSW(aa cluster 3){C}(aa cluster 4)HKWEAAHVAEQLRAYLEGTCVEWLRRYLENGKETLQRTDAPKTHMTHHAVSDHEATLRC WALSFYPAEITLTWQRDGEDQTQDTELVETRPAGDGTFQKWAAVVVPSGQEQRYTCHVQHEGLPKPLTLRWEP (SEQ ID NO:84); where the cysteine residues indicated as {C} form a disulfide bond between the α1 and α2-1 helices.

Each occurrence of aa cluster 1, aa cluster 2, aa cluster 3, aa cluster 4, aa cluster 5, and aa cluster 6 is independently selected to be 1-5 aa residues, wherein the aa residues are each selected independently from i) any naturally occurring (proteinogenic) aa or ii) any naturally occurring aa except proline or glycine. The MHC-H polypeptide sequence may be an HLA-A chain, wherein:
aa cluster 1 may be the amino acid sequence GTLRG (SEQ ID NO:85) or that sequence with one or two aas deleted or substituted with other naturally occurring aas (e.g., L replaced by I, V, A or F);
aa cluster 2 may be the amino acid sequence YNQSE (SEQ ID NO:86) or that sequence with one or two aas deleted or substituted with other naturally occurring aas (e.g., N replaced by Q, Q replaced by N, and/or E replaced by D);
aa cluster 3 may be the amino acid sequence TAADM (SEQ ID NO:87) or that sequence with one or two aas deleted or substituted with other naturally occurring aas (e.g., T replaced by S, A replaced by G, D replaced by E, and/or M replaced by L, V, or); and/or aa cluster 4 may be the amino acid sequence AQTTK (SEQ ID NO: 88) or that sequence with one or two aas deleted or substituted with other naturally occurring aas (e.g., A replaced by G, Q replaced by N, or T replaced by S, and or K replaced by R or Q).

As noted above, any of the MHC-H intrachain disulfide bonds, including a disulfide bond between cysteines at 84 and 139 (a Y84C and A139C disulfide), may be combined with substitutions that permit incorporation of a peptide epitope into a T-Cell-MP. Accordingly, the present disclosure includes and provides for T-Cell-MPs and their higher order complexes (e.g., duplexes) comprising one or more T-Cell-MP polypeptides having a MHC-H polypeptide sequence with an intrachain Y84C A139C disulfide bond and an E44C substitution in the β2M polypeptide sequence. T-Cell-MPs and their higher order complexes (e.g., duplexes) may comprise: (i) a mature β2M polypeptide sequence with an E44C substitution having at least 90% (e.g., at least 95% or at least 98%) sequence identity to at least 70 (e.g., at least 80, 90, 96, 97, 98 or all) of aas 21-119 of any one of NP_004039.1, NP_001009066.1, NP_001040602.1, NP_776318.1, or NP_033865.2 (SEQ ID NOs: 61 to 65, see FIG. 4); and (ii) a MHC-H sequence with Y84C and A139C substitutions (that form a disulfide bond) may have at least 85% (e.g., at least 90%, at least 95% or at least 98%) or 100% sequence identity to at least 200 (e.g., at least 225, at least 250, at least 260, or at least 275) contiguous aas of the α1, α2, and α3 domains an HLA-A, -B, -C, -E, -F, or -G sequences in FIGS. 3D-3H. The MHC-H polypeptide sequence may be an HLA-A*0101, HLA-A*0201, HLA-A*1101, HLA-A*2402, HLA-A*3303, or HLA-A*3401polypeptide sequence having Y84C and A139C substitutions (see FIG. 3E). The MHC-H polypeptide sequence may be an HLA-B*0702, HLA-B*0801, HLA-B*1502, B27 (subtypes HLA-B*2701-2759), HLA-B*3802, HLA-B*4001, HLA-B*4601, or HLA-B*5301 polypeptide sequence having Y84C and A139C substitutions (see, e.g., FIG. 3F). The MHC-H polypeptide sequence may be an HLA-C*0102, HLA-C*0303, HLA-C*0304, HLA-C*0401, HLA-C*0602, HLA-C*0701, HLA-C*0702, HLA-C*0801, or HLA-C*1502 polypeptide sequence having Y84C and A139C substitutions (see, e.g., FIG. 3G).

### 4 Scaffold Polypeptides

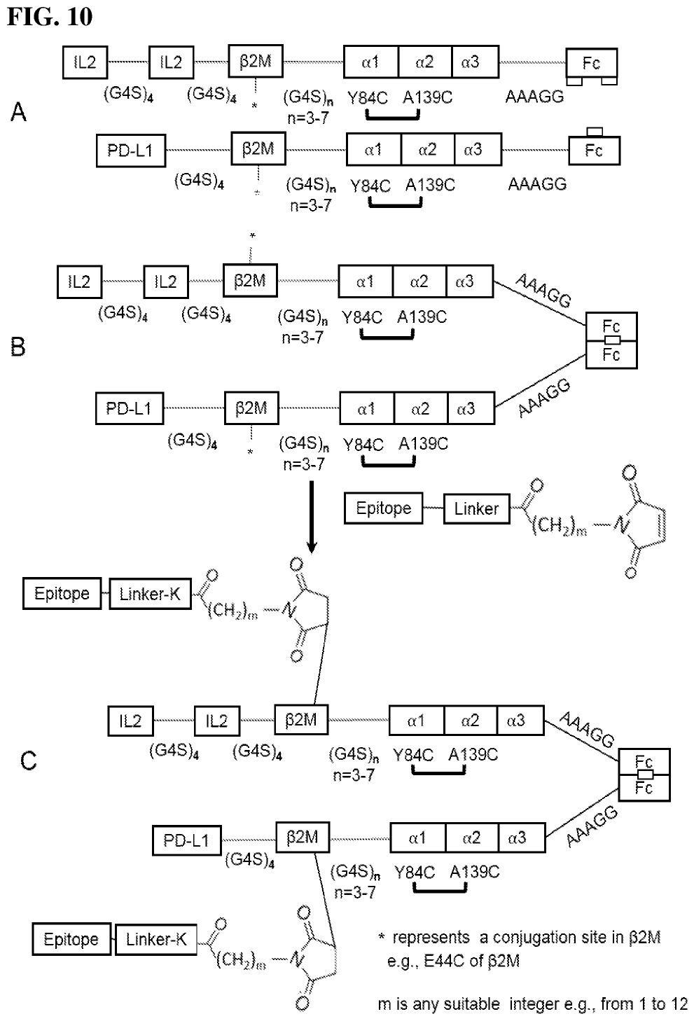

T-Cell-MPs and T-Cell-MP-epitope conjugates may comprise an Ig heavy chain constant region (“Ig Fc” or “Fc”) polypeptide, or may comprise another suitable scaffold polypeptide. Where scaffold polypeptide sequences are identical and pair or multimerize (e.g., some Ig Fc sequences or leucine zipper sequences), they can form symmetrical pairs or multimers (e.g., homodimers, see e.g., FIG. 9 with an Fc scaffold). In contrast, where an asymmetric pairing between two T-Cell-MP molecules is desired (e.g., to produce a duplex T-Cell-MP with each bearing one or more different MODs), the scaffold polypeptides present in the T-Cell-MP may comprise interspecific binding sequences. Interspecific binding sequences are non-identical polypeptide sequences that selectively interact with their specific complementary counterpart sequence to form asymmetric pairs (heterodimers, see e.g., FIG. 10 with an interspecific Fc scaffold). Interspecific binding sequences may in some instances form some amount of homodimers, but preferentially dimerize by binding more strongly) with their counterpart interspecific binding sequence. Accordingly, specific heterodimers tend to be formed when an interspecific dimerization sequence and its counterpart interspecific binding sequence are incorporated into a pair of polypeptides. By way of example, where an interspecific dimerization sequence and its counterpart are incorporated into a pair of polypeptides they may selectively form greater than 70%, 80%, 90%, 95%, 98% or 99% heterodimers when an equimolar mixture of the polypeptides are combined. The remainder of the polypeptides may be present as monomers or homodimers, which may be separated from the heterodimer. Moreover, because interspecific sequences are selective for their counterpart sequence, they can limit the interaction with other proteins expressed by cells (e.g., in culture or in a subject) particularly where the interspecific sequences are not naturally occurring or are variants of naturally occurring protein sequences.

Scaffold polypeptide sequences generally may be less than 300 aa (e.g., about 100 to about 300 aa). Scaffold polypeptide sequences may be less than 250 aa (e.g., about 75 to about 250 aa). Scaffold polypeptide sequences may be less than 200 aa (e.g., about 60 to about 200 aa). Scaffold polypeptide sequences may be less than 150 aa (e.g., about 50 to about 150 aa).

Scaffold polypeptide sequences include, but are not limited to, interspecific and non-interspecific Ig Fc polypeptide sequences, however, polypeptide sequences other than Ig Fc polypeptide sequences (non-Ig sequences) may be used as scaffolds.

a. Non-Immunoglobulin Fc Scaffold Polypeptides

Non-Ig Fc scaffold polypeptides include, but are not limited to: albumin, XTEN (extended recombinant); transferrin; Fc receptor, elastin-like; albumin-binding; silk-like (see, e.g., Valluzzi et al. (2002) Philos Trans R Soc Lond B Biol Sci. 357:165); a silk-elastin-like (SELP; see, e.g., Megeed et al. (2002) Adv Drug Deliv Rev. 54:1075) polypeptides; and the like. Suitable XTEN polypeptides include, e.g., those disclosed in WO 2009/023270, WO 2010/091122, WO 2007/103515, US 2010/0189682, and US 2009/0092582; see, also, Schellenberger et al. (2009) Nat Biotechnol. 27:1186). Suitable albumin polypeptides include, e.g., human serum albumin. Suitable elastin-like polypeptides are described, for example, in Hassouneh et al. (2012) Methods Enzymol. 502:215.

Other non-Ig Fc scaffold polypeptide sequences include but are not limited to: polypeptides of the collectin family (e.g., ACRP30 or ACRP30-like proteins) that contain collagen domains consisting of collagen repeats Gly-Xaa-Yaa and/or Gly-Xaa-Pro (which may be repeated from 10-40 times); coiled-coil domains; leucine-zipper domains; Fos/Jun binding pairs; Ig CH1 and light chain constant region C_{L} sequences (Ig CH1/C_{L} pairs such as a Ig CH1 sequence paired with a Ig C_{L} κ or C_{L} λ light chain constant region sequence).

Non-Ig Fc scaffold polypeptides can be interspecific or non-interspecific in nature. For example, both Fos/Jun binding pairs and Ig CH1 polypeptide sequences and light chain constant region C_{L} sequences form interspecific binding pairs. Coiled-coil sequences, including leucine zipper sequences, can be either interspecific leucine zipper or non-interspecific leucine zipper sequences. See e.g., Zeng et al., (1997) PNAS (USA) 94:3673-3678; and Li et al., (2012), Nature Comms. 3:662.

The scaffold polypeptides of a duplex T-Cell-MP may each comprise a leucine zipper polypeptide sequence. The leucine zipper polypeptides bind to one another to form a dimer. Non-limiting examples of leucine-zipper polypeptides include a peptide comprising any one of the following aa sequences: RMKQIEDKIEEILSKIYHIE NEIARIKKLIGER (SEQ ID NO:89); LSSIEKKQEEQTSW LIWISNELTLIRNELAQS (SEQ ID NO:90); LSSIEKKLEEIT SQLIQISNELTLIRNELAQ (SEQ ID NO:91); LSSIEKKLEEITSQLIQIRNELTLIRNELAQ (SEQ ID NO:92); LSSIEKKLE EITSQLQQIRN ELTLIRNELAQ (SEQ ID NO:93); LSSLEKKLEELTSQLIQLRNELTLLRNELAQ (SEQ ID NO:94); ISSLEKKIEELTSQIQQLRNEITLLRNEIAQ (SEQ ID NO:95). In some cases, a leucine zipper polypeptide comprises the following aa sequence: LEIEAAFLERENTALETRVAELRQRVQRLRNRV SQYRTRYGPLGGGK (SEQ ID NO:96). Additional leucine-zipper polypeptides are known in the art, a number of which are suitable for use as scaffold polypeptide sequences.

The scaffold polypeptide of a T-Cell-MP may comprise a coiled-coil polypeptide sequence that forms a dimer. Non-limiting examples of coiled-coil polypeptides include, for example, a peptide of any one of the following aa sequences: LKSVENRLAVVENQLKTVIEELKTVKDLLSN (SEQ ID NO:97); LARIEEKLKTIKAQLSEIASTLN MIREQLAQ (SEQ ID NO:98); VSRLEEKVKTLKSQVTELASTVS LLREQVAQ (SEQ ID NO:99); IQSEKKIEDISSLIG QIQSEITLIRNEIAQ (SEQ ID NO:100); and LMSLEKKLEELTQTLMQLQNELSMLKNELAQ (SEQ ID NO:101).

The T-Cell-MPs of a T cell MP duplex may comprise a pair of scaffold polypeptide sequences that each comprise at least one cysteine residue that can form a disulfide bond permitting homodimerization or heterodimerization of those polypeptides stabilized by an interchain disulfide bond between the cysteine residues. Examples of such aa sequences include: VDLEGSTSNGRQCAGIRL (SEQ ID NO:102); EDDVTTTEELAPALVPPP KGTCAGWMA (SEQ ID NO:103); and GHDQETTTQ GPGVLLPLPKGACTGQMA (SEQ ID NO:104).

Some scaffold polypeptide sequences permit formation of T-Cell-MP complexes of higher order than duplexes, such as triplexes, tetraplexes, pentaplexes or hexaplexes. Such aa sequences include, but are not limited to, IgM constant regions (discussed below). Collagen domains, which form trimers, can also be employed. Collagen domains may comprise the three aa sequence Gly-Xaa-Xaa and/or GlyXaaYaa, where Xaa and Yaa are independently any aa, with the sequence appear or are repeated multiple times (e.g., from 10 to 40 times). In Gly-Xaa-Yaa sequences, Xaa and Yaa are frequently proline and hydroxyproline respectively in greater than 25%, 50%, 75%, 80% 90% or 95% of the Gly-Xaa-Yaa occurrences, or in each of the Gly-Xaa-Yaa occurrences. In some cases, a collagen domain comprises the sequence Gly-Xaa-Pro repeated from 10 to 40 times. A collagen oligomerization peptide can comprise the following aa sequence: VTAFSNMDDMLQKAHLVIEGTFIYLRDSTEFFIRVRDGWK KLQLGELIPIPADSPPPPALSSNP (SEQ ID NO:105).

b. Immunoglobulin Fc Scaffold Polypeptides

### (i) Non-Interspecific Immunoglobulin Fc Scaffold Polypeptides

The scaffold polypeptide sequences of a T-Cell-MP or its corresponding T-Cell-MP-epitope conjugate may comprise an immunoglobulin (Ig) Fc polypeptide. The Fc polypeptide of a T-Cell-MP or T-Cell-MP-epitope conjugate can be, for example, from an IgA, IgD, IgE, IgG, or IgM, any of which may be a human polypeptide sequence, a humanized polypeptide sequence, a Fc region polypeptide of a synthetic heavy chain constant region, or a consensus heavy chain constant region. In embodiments, the Fc polypeptide can be from a human IgG1 Fc, a human IgG2 Fc, a human IgG3 Fc, a human IgG4 Fc, a human IgA Fc, a human IgD Fc, a human IgE Fc, a human IgM Fc, etc. An Fc polypeptide may comprise an aa sequence having at least about 85% (e.g., at least about 90%; at least about 95%; at least about 98%; or at least about 99%), or 100% aa sequence identity to at least 175 contiguous aas (e.g., at least 200, or at least 210 contiguous aas), or all aas of an aa sequence of a Fc region depicted in FIGS. 2A-2H. Such Ig sequences can interact forming a duplex or higher order structure from T-Cell-MP molecules. In some instances, the Fc scaffold polypeptide sequences include naturally occurring cysteine residues (or non-naturally occurring cysteine residues provided by protein engineering) that are capable of forming interchain disulfide bonds covalently linking two T-Cell-MP polypeptides together. Unless stated otherwise, the Fc polypeptides used in the T-Cell-MPs and their epitope conjugates do not comprise a transmembrane anchoring domain or a portion thereof sufficient to anchor the T-Cell-MP to a cell membrane.

An Ig Fc sequence, or any one or more of the CH1, CH2, and CH3 domains present in a T-Cell-MP may have at least about 80%, at least about 90%, at least about 95%, at least about 98%, at least about 99%, or 100% aa sequence identity to an IgFc polypeptide sequence provided in any of FIGS. 2A-2H. The Ig Fc may comprise a sequence having at least about 95%, at least about 98%, aa sequence identity to sequence provided in any of FIGS. 2A-2H. In those instances where the IgFc region is located at the C-terminus of the T-Cell-MP, the C-terminal lysine provided in some of the sequences provided in FIGS. 2A-2H (e.g., the IgG sequences in FIGS. 2D, 2E, 2F, and 2G) may be removed during cellular processing of MAPPs, and may not be present on some or all of the T-Cell-MP molecules as expressed. See, e.g., van den Bremer et al. (2015) mAbs 7:4; and Sissolak et al. (2019) J. Industrial Microbiol. & Biotechnol. 46:1167. The terminal lysine may also be intentionally removed, thereby avoiding the clipping in cellular processing to insure protein homogeneity.

Most Ig Fc scaffold polypeptides, particularly those comprising only or largely wt. sequences, may spontaneously link together via disulfide bonds to form homodimers resulting in duplex T-Cell-MPs. For example, IgG1 cysteine residues (e.g., cysteine residues at positions 6 and 9 of SEQ ID NO:4, or the corresponding cysteine residues of other IgG1 sequences) may each form an interchain disulfide bond covalently linking a pair of IgG1 sequences by two disulfide bonds. In the case of IgM heavy chain constant regions, in the presences of a J-chains, higher order complexes may be formed. Scaffold polypeptides may comprise an aa sequence having 100% aa sequence identity to the wt. human IgG1 Fc polypeptide depicted in FIG. 2D. A scaffold polypeptide may comprise an aa sequence having at least about 85% (e.g., at least about 90%; at least about 95%; at least about 98%; or at least about 99%) or 100% aa sequence identity to at least 175 contiguous aas (e.g., at least 200, or at least 210 contiguous aas), or all aas, of the wt. human IgG1 Fc polypeptide depicted in FIG. 2D. Such scaffold sequences may include a substitution of N297 (N77 as numbered in FIG. 2D, SEQ ID NO:4) with an aa other than asparagine. In one case, N297 is substituted by alanine, (N297A). Substitutions at N297 lead to the removal of carbohydrate modifications and result antibody sequences with reduced complement component 1q (“C1q”) binding compared to the wt. protein, and accordingly a reduction in complement-dependent cytotoxicity (CDC). K322 (e.g., K322A) substitutions shows a substantial reduction in reduction in FcγR binding affinity and ADCC, with the C1q binding and CDC functions substantially or completely eliminated. Hezareh et al., (2001) J. Virol. 75:12161-168.

Amino acid L234 and other aas in the lower hinge region (e.g., aas 234 to 239, such as L235, G236, G237, P238, S239) which correspond to aas 14-19 of SEQ ID NO:8) of IgG are involved in binding to the Fc gamma receptor (FcγR), and accordingly, mutations at that location reduce binding to the receptor (relative to the wt. protein) and resulting in a reduction in antibody-dependent cellular cytotoxicity (ADCC). Hezareh et al., (2001) have demonstrated that the double mutant (L234A, L235A) does not effectively bind either FcγR or C1q, and both ADCC and CDC functions were substantially or completely abolished. A scaffold polypeptide with a substitution in the lower hinge region may comprise an aa sequence having at least about 85% (e.g., at least about 90%; at least about 95%; at least about 98%; or at least about 99%) aa sequence identity to at least 175 contiguous aas (e.g., at least 200, or at least 210 contiguous aas), or all aas, of the wt. human IgG1 Fc polypeptide depicted in FIG. 2D, that includes a substitution of L234 (L14 of the aa sequence depicted in FIG. 2D) with an aa other than leucine.

A scaffold polypeptide with a substitution in the lower hinge region may comprise an aa sequence having at least about 85% (e.g., at least about 90%; at least about 95%; at least about 98%; or at least about 99%) aa sequence identity to at least 175 contiguous aas (e.g., at least 200, or at least 210 contiguous aas), or all aas, of the wt. human IgG1 Fc polypeptide depicted in FIG. 2D, that includes a substitution of L235 (L15 of the aa sequence depicted in FIG. 2D) with an aa other than leucine. In some cases, the scaffold polypeptide present in a T-Cell-MP with substitutions in the lower hinge region includes L234A and L235A (“LALA”) substitutions (the positions corresponding to positions 14 and 15 of the wt. aa sequence depicted in FIG. 2D; see, e.g., SEQ ID NO:8).

A scaffold polypeptide with a substitution in the lower hinge region may comprise an aa sequence having at least about 85% (e.g., at least about 90%; at least about 95%; at least about 98%; or at least about 99%) aa sequence identity to at least 175 contiguous aas (e.g., at least 200, or at least 210 contiguous aas), or all aas of the wt. human IgG1 Fc polypeptide depicted in FIG. 2D, that includes a substitution of P331 (P111 of the aa sequence depicted in FIG. 2D) with an aa other than proline. Substitutions at P331, like those at N297, lead to reduced binding to C1q relative to the wt. protein, and thus a reduction in complement dependent cytotoxicity. In one embodiment, the substitution is a P331S substitution. In another embodiment, the substitution is a P331A substitution.

A scaffold polypeptide may comprise an aa sequence having at least about 85% (e.g., at least about 90%; at least about 95%; at least about 98%; or at least about 99%) aa sequence identity to at least 175 contiguous aas (e.g., at least 200, or at least 210 contiguous aas), or all aas, of the wt. human IgG1 Fc polypeptide depicted in FIG. 2D, and include substitutions of D270, K322, and/or P329 (corresponding to D50, K102, and P109 of SEQ ID NO:4 in FIG. 2D) that reduce binding to C1q protein relative to the wt. proteins.

A scaffold polypeptide may comprise an aa sequence having at least about 85% (e.g., at least about 90%; at least about 95%; at least about 98%; or at least about 99%) aa sequence identity to at least 175 contiguous aas (e.g., at least 200, or at least 210 contiguous aas), or all aas, of the wt. human IgG1 Fc polypeptide depicted in FIG. 2D, including substitutions at L234 and/or L235 (L14 and/or L15 of the aa sequence depicted in FIG. 2D) with aas other than leucine (such as L234A and L235A substitutions), and a substitution of P331 (P111 of the aa sequence depicted in FIG. 2D) with an aa other than proline such as P331S. In one instance, a scaffold polypeptide present in a T-Cell-MP comprises the “Triple Mutant” aa sequence (SEQ ID NO:6) depicted in FIG. 2D (human IgG1 Fc) having L234F, L235E, and P331S substitutions (corresponding to aa positions 14, 15, and 111 of the aa sequence depicted in FIG. 2D).

The scaffold Fc polypeptide of a T-Cell-MP may comprise an aa sequence having at least about 85% (e.g., at least about 90%; at least about 95%; at least about 98%; or at least about 99%), or 100% aa, sequence identity to at least 175 contiguous aas (e.g., at least 200, or at least 210 contiguous aas), or all aas, of a human IgG2 Fc polypeptide depicted in FIG. 2E. The scaffold Fc polypeptide of a T-Cell-MP may comprise an aa sequence having at least about 85%, (e.g., at least about 90%; at least about 95%; at least about 98%; or at least about 99%), or 100% aa, sequence identity to at least 175 contiguous aas (e.g., at least 200, or at least 210 contiguous aas), or all aas, of a human IgG3 Fc polypeptide depicted in FIG. 2F. The scaffold Fc polypeptide of a T-Cell-MP may comprise an aa sequence having at least about 85% (e.g., at least about 90%; at least about 95%; at least about 98%; or at least about 99%), or 100% aa, sequence identity to at least 175 contiguous aas (e.g., at least 200, or at least 210 contiguous aas), or all aas, of a human IgG4 Fc polypeptide depicted in FIG. 2G. The scaffold Fc polypeptide of a T-Cell-MP may comprise an aa sequence having at least about 85% (e.g., at least about 90%; at least about 95%; at least about 98%; or at least about 99%), or 100% aa sequence identity to at least 175 contiguous aas (e.g., at least 200, or at least 210 contiguous aas e.g., aas 99 to 327 or 111 to 327), or all of the GenBank P01861 human IgG4 Fc polypeptide depicted in FIG. 2G.

The scaffold Fc polypeptide of a T-Cell-MP may comprise IgM heavy chain constant regions (see e.g., FIG. 2H), which forms hexamer, or pentamers (particularly when combined with a mature j-chain peptide lacking a signal sequence such as that provided in FIG. 2I.

### (ii) Interspecific Immunoglobulin Fc Scaffold Polypeptides

Where an asymmetric pairing between two T-Cell-MP molecules is desired (e.g., to produce a duplex T-Cell-MP with different MODs), a scaffold polypeptide present in a T-Cell-MP may comprise, consist essentially of, or consist of an interspecific Ig Fc polypeptides) sequence variants. Such interspecific polypeptide sequences include, but are not limited to, knob-in-hole without (KiH) or with (KiHs-s) a stabilizing disulfide bond, HA-TF, ZW-1, 7.8.60, DD-KK, EW-RVT, EW-RVTs-s, and A107 sequences. One interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. See Xu et al. mAbs 7:1, 231-242 (2015). Another interspecific binding pair has a first Ig Fc polypeptide with Y349C, T366S, L368A, and Y407V substitutions and a second Ig Fc polypeptide with S354C, and T366W substitutions (disulfide bonds can form between the Y349C and the S354C). See e.g., Brinkmann and Konthermann, mAbs 9:2, 182-212 (2015). Ig Fc polypeptide sequences, either with or without knob-in-hole modifications, can be stabilized by the formation of disulfide bonds between the Ig Fc polypeptides (e.g., the hinge region disulfide bonds). Several interspecific binding sequences based upon Ig sequences are summarized in the table that follows (Table 1), with cross reference to the numbering of the aa positions as they appear in the wt. IgG1 sequence (SEQ ID NO:4) set forth in FIG. 2D shown in brackets “{ }”.

TABLE 1
Interspecific Ig sequences and their cognate counterpart interspecific sequences

| | Substitutions in the first | Substitutions in the second | |
|---|---|---|---|
| Interspecific | interspecific polypeptide | (counterpart) interspecific | |
| Pair Name | sequence | polypeptide sequence | Comments |
| KiH | T366W | T366S/L368A/Y407V | Hydrophobic/steric |
| | {T146W} | {T146S/L148A/Y187V} | complementarity |
| KiHs-s | T366W/S354C* | T366S/L368A/Y407V/Y349C | KiH + inter-CH3 |
| | {T146W/S134C*} | {T146S/L148A/Y187V/Y129C} | domain S—S bond |
| HA-TF | S364H/F405A | Y349T/T394F | Hydrophobic/steric |
| | {S144H/F185A} | {Y129T/T174F} | complementarity |
| ZW1 | T350V/L351Y/F405A/Y407V | T350V/T366L/K392L/T394W | Hydrophobic/steric |
| | {T130V/L131Y/F185A/Y187V} | {T130V/T146L/K172L/T174W} | complementarity |
| 7.8.60 | K360D/D399M/Y407A | E345R/Q347R/T366V/K409V | Hydrophobic/steric |
| | {K140D/D179M/Y187A} | {E125R/Q127R/T146V/K189V} | complementarity + |
| | | | electrostatic |
| | | | complementarity |
| DD-KK | K409D/K392D | D399K/E356K | Electrostatic |
| | {K189D/K172D} | {D179K/E136K} | complementarity |
| EW-RVT | K360E/K409W | Q347R/D399V/F405T | Hydrophobic/steric |
| | {K140E/K189W} | {Q127R/D179V/F185T} | complementarity & |
| | | | long-range |
| | | | electrostatic |
| | | | interaction |
| EW-RVTs-s | K360E/K409W/Y349C* | Q347R/D399V/F405T/S354C | EW-RVT + inter-CH3 |
| | {K140E/K189W/Y129C*} | {Q127R/D179V/F185T/S134C} | domain S—S bond |
| A107 | K370E/K409W | E357N/D399V/F405T | Hydrophobic/steric |
| | {K150E/K189W} | {E137N/D179V/F185T} | complementarity + |
| | | | hydrogen bonding |
| | | | complementarity |

Table 1 is modified from Ha et al., Frontiers in Immunol. 7: 1-16 (2016).
*aa forms a stabilizing disulfide bond.

In addition to the interspecific pairs of sequences in Table 1, scaffold polypeptides may include interspecific “SEED” sequences having 45 residues derived from IgA in an IgG1 CH3 domain of the interspecific sequence, and 57 residues derived from IgG1 in the IgA CH3 in its counterpart interspecific sequence. See Ha et al., Frontiers in Immunol. 7:1-16 (2016).

Interspecific Ig sequences my include substitutions described above for non-interspecific Ig sequences that inhibit binding either or both of the FcγR or C1q, and reduce or abolish ADCC and CDC function.

In an embodiment, a scaffold polypeptide found in a T-Cell-MP may comprise an interspecific binding sequence or its counterpart interspecific binding sequence selected from the group consisting of: knob-in-hole (KiH); knob-in-hole with a stabilizing disulfide (KiHs-s); HA-TF; ZW-1; 7.8.60; DD-KK; EW-RVT; EW-RVTs-s; A107; or SEED sequences.

In an embodiment, a T-Cell-MP comprises a scaffold polypeptide comprising an IgG1 sequence with a T146W KiH sequence substitution, and its counterpart interspecific binding partner polypeptide comprises an IgG1 sequence having T146W, L148A, and Y187V KiH sequence substitutions, where the scaffold polypeptides comprises a sequence having at least 85%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D. Scaffold polypeptides optionally comprise substitutions at one of more of: L234 and L235 (e.g., L234A/L235A “LALA” or L234F/L235E); N297 (e.g., N297A); P331 (e.g. P331S); L351 (e.g., L351K); T366 (e.g., T366S); P395 (e.g., P395V); F405 (e.g., F405R); Y407 (e.g., Y407A); and K409 (e.g., K409Y). Those substitutions appear at: L14 and L15 (e.g., L14A/L 15A “LALA” or L14F/L15E); N77 (e.g., N77A); P111 (e.g. P111S) L131 (e.g., L131K); T146 (e.g., T146S); P175 (e.g., P175V); F185 (e.g., F185R); Y187 (e.g., Y187A); and K189 (e.g., K189Y) in the wt. IgG1 sequence of FIG. 2D.

In an embodiment, a T-Cell-MP or duplex T-Cell-MP comprises a scaffold polypeptide comprising an IgG1 sequence with a T146W KiH sequence substitution, and its counterpart interspecific binding partner polypeptide comprises an IgG1 sequence having T146S, L148A, and Y187V KiH sequence substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a T146W and S134C KiHs-s substitutions, and its counterpart interspecific binding partner polypeptide comprises an IgG1 sequence having T146S, L148A, Y187V and Y129C KiHs-s substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) sequences may comprise additional substitutions such as L14 and/or L15 substitutions (e.g., “LALA” substitutions L234A and L235A), and/or N77 (N297 e.g., N297A or N297G).

In an embodiment, a T-Cell-MP comprises a scaffold polypeptide comprising an IgG1 sequence with a S144H and F185A HA-TF substitutions, and its counterpart interspecific binding partner polypeptide comprises an IgG1 sequence having Y129T and T174F HA-TF substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a T130V, L131Y, F185A, and Y187V ZW1 substitutions, and its counterpart interspecific binding partner polypeptide comprises an IgG1 sequence having T130V, T146L, K172L, and T174W ZW1 substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a K140D, D179M, and Y187A 7.8.60 substitutions, and its counterpart interspecific binding partner polypeptide comprises an IgG1 sequence having T130V E125R, Q127R, T146V, and K189V 7.8.60 substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a K189D, and K172D DD-KK substitutions, and its counterpart interspecific binding partner polypeptide comprises an IgG1 sequence having T130V D179K and E136K DD-KK substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a K140E and K189W EW-RVT substitutions, its counterpart interspecific binding partner polypeptide comprises an IgG1 sequence having T130V Q127R, D179V, and F185T EW-RVT substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a K140E, K189W, and Y129C EW-RVTs-s substitutions, its counterpart interspecific binding partner polypeptide comprises an IgG1 sequence having T130V Q127R, D179V, F185T, and S134C EW-RVTs-s substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

In an embodiment, a T-Cell-MP or duplex T-Cell-MP comprises a scaffold polypeptide comprising an IgG1 sequence with a K150E and K189W A107 substitutions, its counterpart interspecific binding partner polypeptide comprises an IgG1 sequence having T130V E137N, D179V, and F185T A107 substitutions, where the scaffold polypeptides comprise a sequence having at least 80%, at least 90%, at least 95%, or at least 97% sequence identity to at least 100 (e.g., at least 125, 150, 170, 180, 190, 200, 210, 220, or all 227) contiguous aas of the wt. IgG1 of FIG. 2D; where one or both (in the case of duplex T-Cell-MP) scaffold polypeptide sequence(s) may comprise additional substitutions such as L14 and/or L15 substitutions (e.g., “LALA” substitutions L234A and L235A), and/or N77 (N297 e.g., N297A or N297G).

As an alternative to the use of Ig CH2 and CH3 heavy chain constant regions as scaffold sequences, Ig light chain constant regions (see FIG. 2K) can be paired with Ig CH1 sequences (See, e.g., FIG. 2J) as interspecific scaffold sequences.

In an embodiment, a T-Cell-MP scaffold polypeptide comprises an Ig CH1 domain (e.g., the polypeptide of FIG. 2J), and the sequence with which it will form a complex (its counterpart binding partner) comprises is an Ig κ chain constant region sequence, where the scaffold polypeptide comprise a sequence having at least 80%, 85%, 90%, 95%, 98%, 99%, or 100% sequence identity to at least 70, at least 80, at least 90, at least 100, or at least 110 contiguous aas of SEQ ID NOs: 16 and/or 17. See FIG. 2K. The Ig CH1 and Ig κ sequences may be modified to increase their affinity for each other, and accordingly the stability of any heterodimer formed utilizing them. Among the substitutions that increase the stability of CH1-Ig κ heterodimers are those identified as the MD13 combination in Chen et al., MAbs, 8 (4): 761-774 (2016). In the MD13 combination two substitutions are introduced into to each of the IgCH1 and Ig κ sequences. The Ig CH1 sequence is modified to contain S64E and S66V substitutions (S70E and S72V of the sequence shown in FIG. 2J). The Ig κ sequence is modified to contain S69L and T71S substitutions (S68L and T70S of the sequence shown in FIG. 2K).

In another embodiment, a scaffold polypeptide of a T-Cell-MP comprises an Ig CH1 domain (e.g., the polypeptide of FIG. 2J, SEQ ID NO:15), and its counterpart sequence comprises an Ig λ chain constant region sequence such as is shown in FIG. 2K (SEQ ID NO:17), where the scaffold polypeptide comprises a sequence having at least 80%, 85%, 90%, 95%, 98%, 99%, or 100% sequence identity to at least 70 (e.g., at least 80, at least 90, or at least 100) contiguous aas of the sequences shown in FIG. 2K.

c. Effects on Stability and Half-Life

Suitable scaffold polypeptides (e.g., those with an Ig Fc scaffold sequence) will in some cases extend the half-life of T-Cell-MP polypeptides and their higher order complexes. In some cases, a suitable scaffold polypeptide increases the in vivo half-life (e.g., the serum half-life) of the T-Cell-MP or duplex T-Cell-MP, compared to a control T-Cell-MP or duplex T-Cell-MP lacking the scaffold polypeptide or comprising a control scaffold polypeptide. For example, in some cases, a scaffold polypeptide increases the in vivo half-life (e.g. serum half-life) of a conjugated or unconjugated T-Cell-MP or duplex T-Cell-MP, compared to an otherwise identical control lacking the scaffold polypeptide, or having a control scaffold polypeptide, by at least about 10%, at least about 20%, at least about 30%, at least about 50%, at least about 2-fold, at least about 5-fold, at least about 10-fold, at least about 25-fold, at least about 50-fold, at least about 100-fold, or more than 100-fold.

### 5 Immunomodulatory Polypeptides (“MODs”)

T-Cell-MPs may comprise, for example one or more aa sequences of MOD polypeptides. A Cell MP may comprises two or three aa sequences of MOD polypeptides that may be the same (identical sequences) or different (e.g., encoding different MOD polypeptide sequences). Different MOD polypeptide sequence include both different variants of a MOD (e.g., a wt. IL-2 and variant IL-2 polypeptide sequence) and MOD sequence that are unrelated (e.g., having different CoMOD receptors such as an IL-2 MOD and a CD86 MOD). Where the more than one MOD polypeptide is present they may be located in tandem or in series, optionally joined to each other by independently selected aa linker sequences without any other elements of the T-Cell-MP (e.g., β2M, MHC-H, or scaffold polypeptide) between the MOD polypeptide sequences. In some cases, a MOD present in a T-Cell-MP is a wt. MOD. In other cases, a MOD present in a T-Cell-MP is a variant MOD, e.g., a variant MOD that has reduced affinity for a Co-MOD, compared to the affinity of a corresponding wt. MOD for the Co-MOD. Suitable variant MODs for incorporation into a T-Cell-MP can be identified by, for example, mutagenesis, such as scanning mutagenesis (e.g., alanine, serine, or glycine scanning mutagenesis).

Exemplary pairs of MODs suitable for incorporation into a T-Cell-MP and their cognate Co-MODs include, but are not limited to, entries (a) to (t) listed in the following table:

### Exemplary Pairs of MODs and Co-MODs

| | |
|---|---|
| a) | 4-1BBL (MOD) and 4-1BB (Co-MOD); |
| b) | PD-L1 (MOD) and PD1 (Co-MOD); |
| c) | IL-2 (MOD) and IL-2 receptor (Co-MOD); |
| d) | CD80 (MOD) and CD28 (Co-MOD); |
| e) | CD86 (MOD) and CD28 (Co-MOD); |
| f) | OX40L (CD252) (MOD) and OX40 (CD134) (Co-MOD); |
| g) | Fas ligand (MOD) and Fas (Co-MOD); |
| h) | ICOS-L (MOD) and ICOS (Co-MOD); |
| i) | ICAM (MOD) and LFA-1 (Co-MOD); |
| j) | CD30L (MOD) and CD30 (Co-MOD); |
| k) | CD40 (MOD) and CD40L (Co-MOD); |
| l) | CD83 (MOD) and CD83L (Co-MOD); |
| m) | HVEM (CD270) (MOD) and CD 160 (Co-MOD); |
| n) | JAG1 (CD339) (MOD) and Notch (Co-MOD); |
| o) | JAG1 (CD339) (MOD) and CD46 (Co-MOD); |
| p) | CD70 (MOD) and CD27 (Co-MOD); |
| q) | CD80 (MOD) and CTLA4 (Co-MOD); |
| r) | CD86 (MOD) and CTLA4 (Co-MOD); |
| s) | PD-L1(MOD) and CD-80 (Co-MOD); and |
| t) | TGF-β1, TGF-β2, and/or TGF-β3 (MODs) and |
| | TGF-β Receptor (e.g., TGFBR1 and/or |
| | TGFBR2) (Co-MOD) |

Generally speaking, the MOD(s) present in a T-Cell-MP will be MODs that provide activating immunomodulatory signals to the T cell, including, e.g., signals that cause an increase in the number of epitope-specific T cells. Such MODs include, but are not limited to, wt. and variants of: IL-2; 4-1BBL; CD80; and CD86,

a. MODS and Variant MODs with Reduced Affinity

Suitable immunomodulatory domains that exhibit reduced affinity for a co-immunomodulatory domain can have from 1 aa to 20 aa differences from a wt. immunomodulatory domain. For example, in some cases, a variant MOD present in a T-Cell-MP differs in aa sequence by 1 aa to 10 aa, or by 11 aa to 20 aa from a corresponding wt. MOD. A variant MOD present in a T-Cell-MP may include a single aa substitution compared to a corresponding reference (e.g., wt.) MOD. A variant MOD present in a T-Cell-MP may include 2 aa substitutions compared to a corresponding reference (e.g., wt.) MOD. A variant MOD present in a T-Cell-MP may include 3 aa substitutions compared to a corresponding reference (e.g., wt.) MOD. A variant MOD present in a T-Cell-MP may include 4 aa substitutions compared to a corresponding reference (e.g., wt.) MOD. A variant MOD present in a T-Cell-MP may include 5 aa substitutions compared to a corresponding reference (e.g., wt.) MOD. A variant MOD present in a T-Cell-MP may include 6 aa or 7 aa substitutions compared to a corresponding reference (e.g., wt.) MOD. A variant MOD present in a T-Cell-MP may include 8 aa, 9 aa, or 10 aa substitutions compared to a corresponding reference (e.g., wt.) MOD. A variant MOD present in a T-Cell-MP may include 11, 12, 13, 14, or 15 aa substitutions compared to a corresponding reference (e.g., wt.) MOD. A variant MOD present in a T-Cell-MP may include 16, 17, 18, 19, or 20 aa substitutions compared to a corresponding reference (e.g., wt.) MOD.

As discussed above, a variant MOD suitable for inclusion in a T-Cell-MP may exhibit reduced affinity for a cognate Co-MOD, compared to the affinity of a corresponding wt. MOD for the cognate Co-MOD. Similarly, a T-Cell-MP that comprises a variant MOD exhibits reduced affinity for the MOD's cognate Co-MOD as compared to the binding affinity of the T-Cell-MP with a wild-type MOD for its cognate co-MOD.

Alternatively, or in addition to, reduced affinity binding, the MOD may be a variant that exhibits selective binding to a Co-MOD. In one aspect, where a MOD can bind to more than one Co-MOD, a variant may be chosen that selectively binds to at least one Co-MOD. For example, wt. PD-L1 binds to both PD-1 and CD80 (also known as B7-1). In such case, a variant PD-L1 MOD may be chosen that selectively (preferentially) binds either to PD-1 or CD80. Likewise, where a wt. MOD may bind to multiple polypeptides within a Co-MOD, a variant may be chosen to selectively bind to only the desired polypeptides with the Co-MOD. For example, IL-2 binds to the alpha, beta and gamma chains of IL-2R. A variant of IL-2 can be chosen that either binds with reduced affinity, or does not bind, to one of the polypeptides, e.g., the alpha chain of IL-2R, or even to two of the chains. For example, an IL-2 variant may have reduced binding to both the alpha chain and the beta chain.

### (i) Determining Binding Affinity

Binding affinity between a MOD and its cognate Co-MOD can be determined by bio-layer interferometry (BLI) using purified MOD and purified cognate Co-MOD. Binding affinity between a T-Cell-MP and its cognate Co-MOD can also be determined by BLI using purified T-Cell-MP and the cognate Co-MOD. BLI methods are well known to those skilled in the art. See, e.g., Lad et al. (2015) J. Biomol. Screen. 20 (4): 498-507; and Shah and Duncan (2014) J. Vis. Exp. 18: e51383. The specific and relative binding affinities described in this disclosure between a Co-MOD and a MOD, or between a Co-MOD and a T-Cell-MP (or its epitope conjugate), can be determined using the procedures described or assessment of TMP molecules in PCT/US2018/049756 published as WO 2019/051091. See e.g., paragraphs [0052] to [0064].

Unless otherwise stated herein, the affinity of a T-Cell-MP-epitope conjugate for a Co-MOD, or the affinity of a control T-Cell-MP-epitope conjugate (where a control T-Cell-MP-epitope conjugate comprises a wt. MOD) for a Co-MOD, is determined using BLI, as described above. Likewise, the affinity of a MOD and its Co-MOD polypeptide can be determined using BLI as described above.

A variant MOD present in a T-Cell-MP may bind to its Co-MOD with an affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the affinity of a corresponding wt. MOD for the Co-MOD.

The combination of the reduced affinity of the MOD for its Co-MOD and the affinity of the epitope for a TCR provides for enhanced selectivity of a T-Cell-MP-epitope conjugate, while still allowing for activity of the MOD. Thus, a T-Cell-MP-epitope conjugate may bind selectively to a first T cell that displays both: i) a TCR specific for the epitope present in the T-Cell-MP-epitope conjugate; and ii) a Co-MOD that binds to the MOD present in the T-Cell-MP-epitope conjugate, compared to binding to a second T cell that displays: i) a TCR specific for an epitope other than the epitope present in the T-Cell-MP-epitope conjugate; and ii) a Co-MOD that binds to the MOD present in the T-Cell-MP-epitope conjugate.

b. Wild-Type and Variant CD80 MODs

In some cases, a variant MOD polypeptide present in a T-Cell-MP is a variant CD80 polypeptide. Wild-type CD80 binds to CD28.

A wild-type amino acid sequence of the ectodomain of human CD80 can be as follows: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:106).

A wild-type CD28 amino acid sequence can be as follows: MLRLLLALNL FPSIQVTGNK ILVKQSPMLV AYDNAVNLSC KYSYNLFSRE FRASLHKGLD SAVEVCVVYG NYSQQLQVYS KTGFNCDGKL GNESVTFYLQ NLYVNQTDIY FCKIEVMYPP PYLDNEKSNG TIIHVKGKHL CPSPLFPGPS KPFWVLVVVG GVLACYSLLV TVAFIIFWVR SKRSRLLHSD YMNMTPRRPG PTRKHYQPYA PPRDFAAYRS (SEQ ID NO:107). In some cases, where a T-Cell-MP comprises a variant CD80 polypeptide, a Co-MOD is a CD28 polypeptide comprising the amino acid sequence of SEQ ID NO: 107.

A wild-type CD28 amino acid sequence can also be as follows: MLRLLLALNL FPSIQVTGNK ILVKQSPMLV AYDNAVNLSW KHLCPSPLFP GPSKPFWVLV VVGGVLACYS LLVTVAFIIF WVRSKRSRLL HSDYMNMTPR RPGPTRKHYQ PYAPPRDFAA YRS (SEQ ID NO:108).

A wild-type CD28 amino acid sequence can be as follows: MLRLLLALNL FPSIQVTGKH LCPSPLFPGP SKPFWVLVVV GGVLACYSLL VTVAFIIFWV RSKRSRLLHS DYMNMTPRRP GPTRKHYQPY APPRDFAAYR S (SEQ ID NO:109).

In some cases, a variant CD80 polypeptide exhibits reduced binding affinity to CD28, compared to the binding affinity of a CD80 polypeptide comprising the amino acid sequence set forth in SEQ ID NO:107 for CD28. For example, in some cases, a variant CD80 polypeptide binds CD28 with a binding affinity that is at least 10% less (e.g., at least: 15% less, 20% less, 25% less, 30% less, 35% less, 40% less, 45% less, 50% less, 55% less, 60% less, 65% less, 70% less, 75% less, 80% less, 85% less, 90% less, 95% less, or more than 95% less) than the binding affinity of a CD80 polypeptide comprising the amino acid sequence set forth in SEQ ID NO: 107 for CD28 (e.g., a CD28 polypeptide comprising the amino acid sequence set forth in one of SEQ ID NOs: 107, 108, or 109) when assayed under the same conditions.

In some cases, a variant CD80 polypeptide has a single amino acid insertion, deletion, or substitution compared to the CD80 amino acid sequence set forth in SEQ ID NO:106. In some cases, a variant CD80 polypeptide has from 2 to 10 aa insertions, deletions, or substitutions compared to the CD80 amino acid sequence set forth in SEQ ID NO:106. In some cases, a variant CD80 polypeptide has 2, 3, 4, 5, 6, 7, 8, 9, or 10 amino acid insertions, deletions, and/or substitutions compared to the CD80 amino acid sequence set forth in SEQ ID NO: 106.

Suitable CD80 variants are described in published PCT Application WO 2019/051091, published 14 Mar. 2019 (Applicant Cue Biopharma, Inc.). See paragraphs [00170]-[00196], the disclosure of which is expressly incorporated herein by reference.

c. Wild-Type and Variant CD86 MODs

In some cases, a variant MOD polypeptide present in a T-Cell-MP is a variant CD86 polypeptide. Wild-type CD86 binds to CD28.

The amino acid sequence of the full ectodomain of a wild-type human CD86 can be as follows:

(SEQ ID NO: 110)
APLKIQAYFNETADLPCQFANSQNQSLSELVVFWQDQENLVLNEVYLGKE
KFDSVHSKYMNRTSFDSDSWTLRLHNLQIKDKGLYQCIIHHKKPTGMIRI
HQMNSELSVLANFSQPEIVPISNITENVYINLTCSSIHGYPEPKKMSVLL
RTKNSTIEYDGIMQKSQDNVTELYDVSISLSVSFPDVTSNMTIFCILETD
KTRLLSSPFSIELEDPQPPPDHIP.

The amino acid sequence of the IgV domain of a wild-type human CD86 can be as follows:

(SEQ ID NO: 111)
APLKIQAYFNETADLPCQFANSQNQSLSELVVFWQDQENLVLNEVYLGKE
KFDSVHSKYMNRTSFDSDSWTLRLHNLQIKDKGLYQCIIHHKKPTGMIRI
HQMNSELSVL.

In some cases, a variant CD86 polypeptide exhibits reduced binding affinity to CD28, compared to the binding affinity of a CD86 polypeptide comprising the amino acid sequence set forth in SEQ ID NO:110 or SEQ ID NO:111 for CD28. For example, in some cases, a variant CD86 polypeptide binds CD28 with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less than the binding affinity of a CD86 polypeptide comprising the amino acid sequence set forth in SEQ ID NO:110 or SEQ ID NO: 111 for CD28 (e.g., a CD28 polypeptide comprising the amino acid sequence set forth in one of SEQ ID NOs: 107, 108, or 109) when assayed under the same conditions.

In some cases, a variant CD86 polypeptide has a single aa insertion, deletion, or substitutions compared to the CD86 amino acid sequence set forth in SEQ ID NO: 110. In some cases, a variant CD86 polypeptide has from 2 to 10 amino acid insertions, deletions, and/or substitutions compared to the CD86 amino acid sequence set forth in SEQ ID NO:110. In some cases, a variant CD86 polypeptide has 2, 3, 4, 5, 6, 7, 8, 9, or 10 aa insertions, deletions, and/or substitutions compared to the CD86 amino acid sequence set forth in SEQ ID NO: 110.

Suitable CD86 variants are described in published PCT Application WO 2019/051091, published 14 Mar. 2019 (Applicant Cue Biopharma, Inc.). See paragraphs [00197]-[00228], the disclosure of which is expressly incorporated herein by reference.

d. Wild-Type and Variant 4-1BBL MODs

In some cases, a variant MOD polypeptide present in a T-Cell-MP is a variant 4-1BBL polypeptide. Wild-type 4-1BBL binds to 4-1BB (CD137).

A wild-type 4-1BBL amino acid sequence can be as follows: MEYASDASLD PEAPWPPAPR ARACRVLPWA LVAGLLLLLL LAAACAVFLA CPWAVSGARA SPGSAASPRL REGPELSPDD PAGLLDLRQG MFAQLVAQNV LLIDGPLSWY SDPGLAGVSL TGGLSYKEDT KELVVAKAGV YYVFFQLELR RWVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:112) NCBI Reference Sequence: NP_003802.1, where aas 29-49 are a transmembrane region.

In some cases, a variant 4-1BBL polypeptide is a variant of the tumor necrosis factor (TNF) homology domain (THD) of human 4-1BBL.

A wild-type amino acid sequence of the THD of human 4-1BBL can be, e.g., one of SEQ ID NOs: 113-115, as follows: PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:113); D PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:114); or D PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPA (SEQ ID NO:115).

A wild-type 4-1BB amino acid sequence can be as follows: MGNSCYNIVA TLLLVLNFER TRSLQDPCSN CPAGTFCDNN RNQICSPCPP NSFSSAGGQR TCDICRQCKG VFRTRKECSS TSNAECDCTP GFHCLGAGCS MCEQDCKQGQ ELTKKGCKDC CFGTENDQKR GICRPWTNCS LDGKSVLVNG TKERDVVCGP SPADLSPGAS SVTPPAPARE PGHSPQIISF FLALTSTALL FLLFFLTLRF SVVKRGRKKL LYIFKQPFMR PVQTTQEEDG CSCRFPEEEE GGCEL (SEQ ID NO:116). In some cases, where a T-Cell-MP comprises a variant 4-1BBL polypeptide, a Co-MOD is a 4-1BB polypeptide comprising the amino acid sequence of SEQ ID NO:116.

Variant 4-1BBL polypeptides exhibit reduced binding affinity to 4-1BB, compared to the binding affinity of a 4-1BBL polypeptide comprising the amino acid sequence set forth in one of SEQ ID NOs: 112-115. For example, in some cases, a variant 4-1BBL polypeptide binds 4-1BB with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less than the binding affinity of a 4-1BBL polypeptide comprising the amino acid sequence set forth in one of SEQ ID NOs: 112-115 for a 4-1BB polypeptide (e.g., a 4-1BB polypeptide comprising the amino acid sequence set forth in SEQ ID NO:116), when assayed under the same conditions.

4-1BBL variants suitable for use as a MOD in a T-Cell-MP include those comprising a sequence with at least one aa substitution and having at least 90%, at least 95%, at least 98%, or at least 99% aa sequence identity to SEQ ID NOs: 113, 114 or 115. 4-1BBL variants suitable for use as a MOD in a T-Cell-MP include those comprising a sequence with at least two aa substitutions and having at least 90%, at least 95%, at least 98%, or at least 99% aa sequence identity to SEQ ID NOs: 113, 114 or 115.

4-1BBL variants suitable for inclusion in a T-Cell-MP include those comprising a sequence with at least one aa substitution (e.g., two, three, or four insertions, deletions, and/or substitutions) include those having at least 90%, at least 95%, at least 98%, or at least 99% aa sequence identity to at least 140 (e.g., at least 160, 175, 180, or 181) contiguous aas of SEQ ID NO:113.

Suitable 4-1BBL variants are described in published PCT Application WO 2019/051091, published 14 Mar. 2019 (Applicant Cue Biopharma, Inc.). See paragraphs [00229]-[00324], the disclosure of which is expressly incorporated herein by reference.

e. Wild-Type and Variant IL-2 MODs

In some cases, a variant MOD polypeptide present in a T-Cell-MP is a variant IL-2 polypeptide. Wild-type IL-2 binds to IL-2 receptor (IL-2R), i.e., a heterotrimeric polypeptide comprising IL-2Rα, IL-2Rβ, and IL-2Rγ.

A wild-type IL-2 amino acid sequence can be as follows: APTSSSTKKT QLQLEHLLLD LQMILNGINN YKNPKLTRML TFKFYMPKKA TELKHLQCLEEELKPLEEVL NLAQSKNFHL RPRDLISNIN VIVLELKGSE TTFMCEYADE TATIVEFLNRWITFCQSIIS TLT (UniProt, P60568, SEQ ID NO: 117).

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122) and a gamma chain (IL-2Rγ; also referred to as CD132). Amino acid sequences of human IL-2Rα, IL2Rβ, and IL-2Rγ are provided in the accompanying sequence listing as SEQ ID NO:118, SEQ ID NO:119, and SEQ ID NO: 120, and are also provided in, for example, U.S. Patent Pub. No. 20200407416.

Suitable variant IL-2 polypeptide sequences include polypeptide sequences comprising an aa sequence having at least 80% (e.g., at least 85%, at least 90%, at least 95%, at least 98%, at least 99%, or 100%) aa sequence identity to at least 80 (e.g., 90, 100, 110, 120, 130 or 133) contiguous aas of SEQ ID NO: 117. Potential amino acids where substitutions may be introduced include one or more of the following positions:
(i) position 15, where the aa is other than E (e.g., A);
(ii) position 16, where the aa is other than H (e.g., A, T, E, D, N, C, Q, M, V or W);
(iii) position 20 is an aa other than D (e.g., A);
(iv) position 42, where the aa is other than F (e.g., A, M, P, S, T, Y, V or H);
(v) position 45, where the aa is other than Y (e.g., A);
(vi) position 88, where the aa is other than N (e.g., A or R);
(vii) position 126, where the aa is other than Q (e.g., A); SEQ ID NO:2086.

Combinations of the above substitutions include (H16X, F42X), (D20X, F42X), (E15X, D20X, F42X), (an H16X, D20X, F42X), (H16X, F42X, R88X), (H16X, F42X, Q126X), (D20X, F42X, Q126X), (D20X, F42X, and Y4X), (H16X, D20X, F42X, and Y45X), (D20X, F42X, Y45X, Q126X), (H16X, D20X, F42X, Y45X, Q126X), where X is the substituted aa, optionally chosen from the following: positions 15, 20, 45, 126—A; position 16—A or T, or also N, C, Q, M, V or W; position 42—A, or also M, P, S, T, Y, V or H; position 88—A or R.

Suitable variant IL-2 polypeptide sequences include polypeptide sequences comprising at least one insertion, deletion, or substitution and comprise an aa sequence having at least 90% (e.g., at least 95%, at least 98%, at least 99%, or 100%) aa sequence identity to at least 90 (e.g., 95, 100, 110, 120, 130 or 133) contiguous aas of SEQ ID NO: 117.

IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO: 117, wherein the aa at position 16 is an aa other than H. In one case, the position of H16 is substituted by Asn, Cys, Gln, Met, Val, or Trp. In one case, the position of H16 is substituted by Ala. In another case, the position of H16 is substituted by Thr. Additionally, or alternatively, IL-2 variants include polypeptides having at least 90% (e.g., at least 95%, 98%, or 99%) aa sequence identity to at least 80 (e.g., at least 90, 100, 110, 120, or 130) contiguous aas of SEQ ID NO:117, wherein the aa at position 42 is an aa other than F. In one case, the position of F42 is substituted by Met, Pro, Ser, Thr, Trp, Tyr, Val, or His. In one case, the position of F42 is substituted by Ala.

In some cases, an IL-2 variant MOD of this disclosure exhibits decreased binding to IL-2Rα, thereby minimizing or substantially reducing the activation of Tregs by the IL-2 variant. Alternatively, or additionally, in some cases, an IL-2 variant MOD of this disclosure exhibits decreased binding to IL-2Rβ and/or IL-2Rγ such that the IL-2 variant MOD exhibits an overall reduced affinity for IL-2R. In some cases, an IL-2 variant MOD of this disclosure exhibits both properties, i.e., it exhibits decreased or substantially no binding to IL-2Rα, and also exhibits decreased binding to IL-2Rβ and/or IL-2Rγ such that the IL-2 variant polypeptide exhibits an overall reduced affinity for IL-2R. For example, IL-2 variants having substitutions at H16 and F42 have shown decreased binding to IL-2Rα and IL-2Rβ. See, Quayle et al., Clin Cancer Res; 26 (8) Apr. 15, 2020, which discloses that the binding affinity of an IL-2 polypeptide with H16A and F42A substitutions for human IL-2Rα and IL-2Rβ was decreased 110- and 3-fold, respectively, compared with wild-type IL2 binding, predominantly due to a faster off-rate for each of these interactions. TMPs comprising such variants, including variants that exhibit decreased binding to IL-2Rα and IL-2Rβ, have shown the ability to preferentially bind to and activate IL-2 receptors on T cells that contain the target TCR that is specific for the peptide epitope on the TMP, and are thus less likely to deliver IL-2 to non-target T cells, i.e., T cells that do not contain a TCR that specifically binds the peptide epitope on the TMP. That is, the binding of the IL-2 variant MOD to its costimulatory polypeptide on the T cell is substantially driven by the binding of the MHC-epitope moiety rather than by the binding of the IL-2. IL-2 variants thus include polypeptides comprising an aa sequence comprising all or part of human IL-2 having a substation at position H16 and/or F42 (e.g., H16A and/or F42A substitutions).

IL-2 variants include polypeptides having at least 90% (e.g., at least 95%, 98%, or 99%) aa sequence identity to at least 80 (e.g., at least 100, 110, 120, or 130) contiguous aas of SEQ ID NO: 117, wherein the aa at position 16 is an aa other than H and the aa at position 42 is other than F (SEQ ID NO:2087). In one case, the position of H16 is substituted by Ala or Thr and the position of F42 is substituted by Ala or Thr. In one case, the position of H16 is substituted by Ala and the position of F42 is substituted by Ala (an H16A and F42A variant SEQ ID NO: 2088). In a second case, the position of H16 is substituted by Thr and the position of F42 is substituted by Ala (an H16T and F42A variant). In a third case, the position of H16 is substituted by Ala and the position of F42 is substituted by Thr (an H16A and F42T variant). In a fourth case, the position of H16 is substituted by Thr and the position of F42 is substituted by Ala (an H16D and F42A variant). In each of the foregoing pairs of substitutions, the F42A substitution could be replaced by an F42T substitution. As noted above, such variants will exhibit reduced binding to both the human IL-2Rα chain and IL2Rβ chain.

In any of the wild-type or variant IL-2 sequences provided herein, the cysteine at position 125 may be substituted with an alanine (a C125A substitution). In addition to any stability provided by the substitution, it may be employed where, for example, an epitope containing peptide or payload is to be conjugated to a cysteine residue elsewhere in a T-Cell-MP first or second polypeptide, thereby avoiding competition from the C125 of the IL-2 MOD sequence.

f. Wild-Type and Variant PD-L1 MODs

As noted above, the MOD(s) that will be present in a T-Cell-MP generally will be MODs that provide activating immunomodulatory signals to the T cell, including, e.g., signals that cause an increase in the number of epitope-specific T cells. In some cases, however, it may be desirable to include a MOD that can provide an inhibitory/suppressing immunomodulatory signal to T cells, or may have an activating effect to T cell under some conditions, e.g., a PD-L1.

Suitable wild-type PD-L1 and PD-L1 variants are described in published PCT Application WO 2019/051091, published 14 Mar. 2019 (Applicant Cue Biopharma, Inc.). See paragraphs [00157]-[00169], the disclosure of which is expressly incorporated herein by reference. Other inhibitory/suppressing immunomodulatory, e.g., FasL also are known and may be included where an inhibitory/suppressing immunomodulatory signal is desired.

### 6 Linkers

T-Cell-MPs (and their T-Cell-MP-epitope conjugates) can include one or more independently selected linker polypeptide sequences interposed between, for example, any one or more elements of a T-Cell-MP, for example:
i) two MOD polypeptides located on the N-terminal side of the β2M polypeptide sequence (referred to as an L 1linker or position);
(ii) between a MOD and a β2M polypeptide sequence (referred to as an L2 linker or position);
(iii) between a β2M polypeptide sequence and a MHC-H polypeptide sequence (referred to as an L3 linker or position);
(iv) between a MHC-H polypeptide sequence and a scaffold polypeptide sequence (referred to as an L4 linker or position);
(iv) at the carboxyl end of the scaffold or between a scaffold polypeptide sequence and a MOD polypeptide sequence placed carboxy terminal to it (referred to as an L5 linker or position); or
(vi) between two MOD polypeptide sequences placed on the carboxy side of the scaffold (referred to as an L6 linker or position).
See, e.g., FIG. 5.

Chemical conjugation sites for coupling epitopes (e.g., peptide epitopes) may be incorporated into linkers (e.g., L1-L6 linkers) including the L3 between the MHC-H and β2M polypeptide sequences. Accordingly, chemical conjugation sites including, but not limited to: sulfatase, sortase, transglutaminase, selenocysteine, non-natural amino acids, and naturally occurring proteinogenic amino acids (e.g., cysteine residues) etc. may be incorporated into linkers, including the L3 linker. Polypeptide linkers placed at either the N- or C-termini provide locations to couple additional polypeptides (e.g., histidine tags), payloads and the like, and to protect the polypeptide from exoproteases.

Linkers may also be utilized between the peptide epitope and any reactive chemical moiety (group) used to couple the peptide epitope to the chemical conjugation site of an unconjugated T-Cell-MP (see e.g., FIG. 10). Linkers utilized between epitope (e.g., peptide epitope) and a reactive chemical moiety may be peptide/polypeptide linkers, and/or other chemical linkers (e.g., non-peptide linkers in the form of homo or hetero bifunctional linkers that comprise an alkyl group as a spacer, see e.g., FIG. 10 at entries d and e).

Suitable polypeptide linkers (also referred to as “spacers”) can be readily selected and can be of any of a number of suitable lengths, such as from 1 aa to 50 aa, from 1aa to 5 aa, from 1 aa to 15 aa, from 2 aa to 15 aa, from 2 aa to 25 aa, from 3 aa to 12 aa, from 4 aa to 10 aa, from 4 aa to 35 aa, from 5 aa to 35 aa, from 5 aa to 10 aa, from 5 aa to 20 aa, from 6 aa to 25 aa, from 7 aa to 35 aa, from 8aa to 40 aa, from 9 aa to 45 aa, from 10 to 15 aa, from 10 aa to 50 aa, from 15 to 20 aa, from 20 to 40 aa, or from 40 to 50 aa. Suitable polypeptide linkers in the range from 10 to 50 aas in length may be from 10 to 20, from 10 to 25, from 15 to 25, from 20 to 30, from 25 to 35, from 25 to 50 30 to 35, from 35 to 45, or from 40 to 50). In embodiments, a suitable linker can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 aa in length. A polypeptide linker may have a length of from 15 aa to 50 aa, e.g., from 20 to 35, from 25 to 30, from 25 to 45, from 30 to 35, from 35 to 40, from 40 to 45, or from 45 to 50 aa in length. Linkers can be generally classified into three groups, i.e., flexible, rigid and cleavable. See, e.g., Chen et al. (2013) Adv. Drug Deliv. Rev. 65:1357; and Klein et al. (2014) Protein Engineering, Design & Selection 27:325. Unless stated otherwise, the linkers employed in the T-Cell-MPs of this disclosure are not the cleavable linkers generally known in the art.

Polypeptide linkers in the T-Cell-MP may include, for example, polypeptides that comprise, consist essentially of, or consists of: i) Gly and/or Ser; ii) Ala and Ser; iii) Gly, Ala, and Ser; iv) Gly, Ser, and Cys (e.g., a single Cys residue); v) Ala, Ser, and Cys (e.g., a single Cys residue); and vi) Gly, Ala, Ser, and Cys (e.g., a single Cys residue). Exemplary linkers may comprise glycine polymers, glycine-serine polymers, glycine-alanine polymers; alanine-serine polymers (including, for example polymers comprising the sequences GA, AG, AS, SA, GS, GSGGS (SEQ ID NO: 121) or GGGS (SEQ ID NO:122), any of which may be repeated from 1 to 10 times (e.g., repeated 1, 2, 3, 4, 5, 6, 7, 8, 9, or 10 times); and other flexible linkers known in the art. Glycine and glycine-serine polymers can both be used as both Gly and Ser are relatively unstructured and therefore can serve as a neutral tether between components. Glycine polymers access significantly more phi-psi space than even alanine polymers, and are much less restricted than residues with longer side chains (see Scheraga, Rev. Computational Chem. 11173-142 (1992). Exemplary linkers may also comprise an aa sequence comprising, but not limited to, GGSG (SEQ ID NO:123), GGSGG (SEQ ID NO:124), GSGSG (SEQ ID NO:125), GSGGG (SEQ ID NO:126), GGGSG (SEQ ID NO:127), GSSSG (SEQ ID NO:128), any of which may be repeated from 1 to 15 times (e.g., repeated 1, 2, 3, 4, 5, 6, 7, 8, 9, 10, 11, 12, 13, 14, or 15 times), or combinations thereof, and the like. Linkers can also comprise the sequence Gly(Ser)₄ (SEQ ID NO:129) or (Gly)₄Ser (SEQ ID NO: 130), either of which may be repeated from 1 to 10 times (e.g., 1, 2, 3, 4, 5, 6, 7, 8, 9, or 10 times). In an embodiment, the linker comprises the X1-X2-X3-X4-X5 where X1-X5 are selected from glycine and serine, and one of which may be a leucine, cysteine, methionine, or alanine (SEQ ID NO: 131). In one embodiment the linker comprises the aa sequence AAAGG (SEQ ID NO: 132), which may be repeated from 1 to 10 times.

Rigid polypeptide linkers comprise a sequence of amino acids that effectively separates protein domains by maintaining a substantially fixed distance/spatial separation between the domains, thereby reducing or substantially eliminating unfavorable interactions between such domains. Rigid polypeptide linkers thus may be employed where it is desired to minimize the interaction between the domains of the T-Cell-MP at any of linker positions L1-L6. For example, when the T-Cell-MP comprises one or more C-terminal MODs, then a rigid linker may be employed at position L5 or L6 to provide spacing between the T-Cell-MP scaffold and the MOD(s). Rigid peptide linkers include peptide linkers rich in proline, and peptide linkers having an inflexible helical structure, such as an α-helical structure. Examples of rigid peptide linkers include, e.g., (EAAAK)n (SEQ ID NO:133), A (EAAAK)nA (SEQ ID NO: 134), A (EAAAK)nALEA (EAAAK)nA (SEQ ID NO:135), (Lys-Pro)n, (Glu-Pro)n, (Thr-Pro-Arg)n, and (Ala-Pro)n where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid linkers comprising EAAAK (SEQ ID NO:136) include EAAAK (SEQ ID NO:136), (EAAAK)2 (SEQ ID NO:137), (EAAAK)3 (SEQ ID NO:138), A(EAAAK)4ALEA (EAAAK)4A (SEQ ID NO:139), and AEAAAKEAAAKA (SEQ ID NO: 140). Non-limiting examples of suitable rigid linkers comprising (AP)n include APAP (SEQ ID NO:141; also referred to herein as “(AP)2”); APAPAPAP (SEQ ID NO:142; also referred to herein as “(AP)4”); APAPAPAPAPAP (SEQ ID NO:143; also referred to herein as “(AP)6”); APAPAPAPAPAPAPAP (SEQ ID NO: 144; also referred to herein as “(AP)8”); and APAPAPAPAPAPAPAPAPAP (SEQ ID NO: 145; also referred to herein as “(AP)10”). Non-limiting examples of suitable rigid linkers comprising (KP) n include KPKP (SEQ ID NO:146; also referred to herein as “(KP) 2”); KPKPKPKP (SEQ ID NO:147; also referred to herein as “(KP) 4”); KPKPKPKPKPKP (SEQ ID NO:148; also referred to herein as “(KP) 6”); KPKPKPKPKPKPKPKP (SEQ ID NO:149; also referred to herein as “(KP) 8”); and KPKPKPKPKPKPKPKPKPKP (SEQ ID NO:150; also referred to herein as “(KP) 10”). Non-limiting examples of suitable rigid linkers comprising (EP) n include EPEP (SEQ ID NO:151; also referred to herein as “(EP) 2”); EPEPEPEP (SEQ ID NO:152; also referred to herein as “(EP) 4”); EPEPEPEPEPEP (SEQ ID NO: 153; also referred to herein as “(EP) 6”); EPEPEPEPEPEPEPEP (SEQ ID NO:154; also referred to herein as “(EP) 8”); and EPEPEPEPEPEPEPEPEPEP (SEQ ID NO:155; also referred to herein as “(EP) 10”).

In some cases, a linker polypeptide present in a T-Cell-MP includes a cysteine residue that can form a disulfide bond with a cysteine residue present elsewhere in the T-Cell-MP, in another T-Cell-MP, or act as a chemical conjugation site for the coupling of an epitope (e.g., via reaction with a maleimide). In some cases, for example, the linker comprises Gly, Ser and a single Cys, such as in the aa sequence GCGGS (G4S) (SEQ ID NO:156) where the G₄S unit may be repeated from 1 to 10 times (e.g., repeated 1, 2, 3, 4, 5, 6, 7, 8, 9, or 10 times), GCGASGGGGSGGGGS (SEQ ID NO:157), GCGGSGGGGSGGGGSGGGGS (SEQ ID NO:158) or GCGGSGGGGSGGGGS (SEQ ID NO:159).

A linker may comprise the aa sequence (GGGGS) (SEQ ID NO: 130, that may also be represented as Gly₄Ser or G₄S), which may be repeated from 1 to 10 times (e.g., 1, 2, 3, 4, 5, 6, 7, 8, 9, or 10 times). In some embodiments a linker comprising G₄S repeats has one glycine or serine residue replaced by a leucine or methionine. A first T-Cell-MP comprising a Gly₄Ser containing linker polypeptide that includes a cysteine residue may, when duplexed with a second T-Cell-MP, form a disulfide bond with a cysteine residue present in the second T-Cell-MP of the duplex T-Cell-MP. Such cysteine residues present in linkers (particularly the L3 linker) may also be utilized as a chemical conjugation site for the attachment of an epitope (e.g., a peptide epitope), such as by reaction with a maleimide functionality that is part of, or indirectly connected by a linker to, the epitope. In some cases, for example, the linker comprises the aa sequence GCGGS (G₄S) (SEQ ID NO: 156) where the G₄S unit may be repeated from 1 to 10 times (e.g., repeated 1, 2, 3, 4, 5, 6, 7, 8, 9, or 10 times), GCGASGGGGSGGGGS (SEQ ID NO:157), the sequence GCGGSGGGGSGGGGSGGGGS (SEQ ID NO: 158) or the sequence GCGGSGGGGSGGGGS (SEQ ID NO: 159). Non-peptide linkers that may be used to covalently attach epitopes, targeting sequences (e.g., polypeptides comprising a targeting sequence), and/or payloads (e.g., a drug or labeling agent) to a T-Cell-MP (including its peptide linkers) may take a variety of forms, including, but not limited to, alkyl, poly(ethylene glycol), disulfide groups, thioether groups, acid labile groups, photolabile groups, peptidase labile groups, and esterase labile groups. The non-peptide linkers (or “crosslinkers”) may also be, for example, homobifunctional or heterobifunctional linkers that comprise reactive end groups such as N-hydroxysuccinimide esters, maleimide, iodoacetate esters, and the like. Examples of suitable cross-linkers include: N-succinimidyl-[(N-maleimidopropionamido)-tetraethyleneglycol]ester (NHS-PEG4-maleimide); N-succinimidyl 4-(2-pyridyldithio) butanoate (SPDB); N-succinimidyl 4-(2-pyridyldithio) 2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); κ-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); N-(α-maleimidoacetoxy)-succinimide ester (AMAS); succinimidyl-6-(β-maleimidopropionamide) hexanoate (SMPH); N-succinimidyl 4-(p-maleimidophenyl) butyrate (SMPB); N-(p-maleimidophenyl) isocyanate (PMPI); N-succinimidyl 4 (2-pyridylthio) pentanoate (SPP); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexane-carboxylate (SMCC); N-succinimidyl-4-(N-maleimidomethyl)-cyclohexane-1-carboxy-(6-amidocaproate), a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido) propionate (SBAP).

### 7 Additional Polypeptide Sequences

A polypeptide chain of a T-Cell-MP (either an unconjugated T-Cell-MPs or a T-Cell-MP-epitope conjugate) can include one or more polypeptides in addition to those described above. Suitable additional polypeptides include epitope tags, affinity domains, and fluorescent protein sequences (e.g., green fluorescent protein). The one or more additional polypeptide(s) can be included as part of a polypeptide translated by cell or cell-free system at the N-terminus of a polypeptide chain of a multimeric polypeptide, at the C-terminus of a polypeptide chain of a multimeric polypeptide, or internally within a polypeptide chain of a multimeric polypeptide.

a. Epitope Tags and Affinity Domains

Suitable epitope tags include, but are not limited to, hemagglutinin (HA; e.g., YPYDVPDYA (SEQ ID NO: 160)); c-myc (e.g., EQKLISEEDL; SEQ ID NO: 161)), and the like.

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel SEPHAROSE®. Exemplary affinity domains include His5 (HHHHH) (SEQ ID NO:162), HisX6 (HHHHHH) (SEQ ID NO:163), C-myc (EQKLISEEDL) (SEQ ID NO:161), Flag (DYKDDDDK) (SEQ ID NO:164, StrepTag (WSHPQFEK) (SEQ ID NO:165), hemagglutinin (e.g., HA Tag (YPYDVPDYA) (SEQ ID NO:160)), glutathione-S-transferase (GST), thioredoxin, cellulose binding domain, RYIRS (SEQ ID NO:166), FHHT (SEQ ID NO:167), chitin binding domain, S-peptide, T7 peptide, SH2 domain, C-end RNA tag, WEAAAREACCRECCARA (SEQ ID NO: 168), metal binding domains (e.g., zinc binding domains or calcium binding domains such as those from calcium-binding proteins such as calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin), inteins, biotin, streptavidin, MyoD, Id, leucine zipper sequences, and maltose binding protein.

b. Targeting Sequences and Their Targets

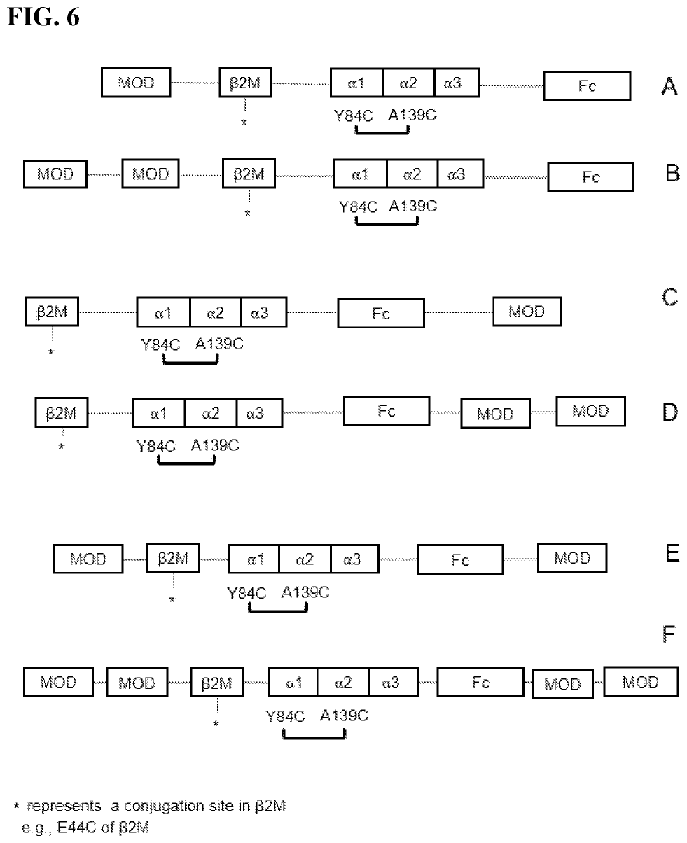

T-Cell-MPs or T-Cell-MP-epitope conjugates may include one or more targeting sequences, which are moieties, typically polypeptides or proteins, that can bind to a target such as an antigen on a cancerous cell. Targeting sequences may be located anywhere within the T-Cell-MP polypeptide, for example within, at, or near the carboxyl terminal end of a scaffold peptide (e.g., translated with the scaffold in place of a C-terminal MOD in FIG. 5 or 6 or attached to an L5 linker). In some instances the targeting sequence is part of the T-Cell-MP fusion protein and is a Fab, scFv, nanobody or the like directed to a suitable antigen. Alternatively, a targeting sequence, such as an antibody antigen-binding fragment (Fab), may be covalently or non-covalently attached to a T-Cell-MP. Covalent attachment of a targeting sequence may be made at a chemical conjugation site (e.g., a chemical conjugation site in a scaffold polypeptide), where the targeting sequence effectively becomes a payload-like molecule attached to the T-Cell-MP.

Although targeting sequences may be part of a T-Cell-MP as translated (a fusion protein) or covalently linked via a crosslinker, targeting sequences may also be non-covalently bound to a T-Cell-MP (e.g., a T-Cell-MP having a biotin labeled scaffold may be non-covalently attached to an avidin labeled targeting antibody, Fab, nanobody or the like directed to a suitable antigen). A bispecific antibody (e.g., a bispecific IgG or humanized antibody) having a first antigen binding site directed to a part of the T-Cell-MP (e.g., the scaffold) may also be employed to non-covalently attach a T-Cell-MP to a targeting sequence. The targeting sequence may then be the second bispecific antibody binding site that targets, for example, a cancer antigen. Alternatively, the second bispecific antibody binding site may be directed to, for example, the Fc domain of an antibody against a cancer antigen. Targeting sequences serve to bind or “localize” T-Cell-MPs to cells and/or tissues displaying the protein (or other molecule) to which the targeting sequence binds. In some instances, targeting sequence may be an antibody or antigen binding fragment thereof (scFv or nanobody). A targeting sequence may also be a single-chain T cell receptor (scTCR).

There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b. Antibodies of any or all of those classes or subclasses may be used as targeting sequences, and where necessary an antibody that is from a non-human may be humanized to form a humanized immunoglobulin. The term “humanized immunoglobulin” as used herein refers to an immunoglobulin comprising portions of immunoglobulins of different origin, wherein at least one portion comprises amino acid sequences of human origin. Chimeric or CDR-grafted single chain antibodies are also encompassed by the term humanized immunoglobulin.

The terms “antibodies”, “antibody” and “immunoglobulin” include antibodies or immunoglobulins of any isotype, fragments of antibodies that retain specific binding to antigen, including, but not limited to, Fab, F(ab′)₂, Fv, scFv, and Fd fragments, chimeric antibodies, humanized antibodies, single-chain antibodies (scAb), single chain camelid (e.g., llama) antibodies, single domain antibodies (dAb), single domain heavy chain antibodies, a single domain light chain antibodies, nanobodies, diabodies, bi-specific antibodies, multi-specific antibodies, and fusion proteins comprising an antigen-binding (also referred to herein as antigen binding) portion of an antibody and a non-antibody protein. In some instances in this disclosure, specific types of antibody fragments may be recited along with the term “antibody” but it is to be understood that the terms “antibodies” and “antibody” are intended to include all such fragments.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (1996) Nature Structural Biol. 3:803; and Desmyter et al. (2015) Curr. Opin. Struct. Biol. 32:1).

“Fv” is the minimum antibody fragment that contains a complete antigen-recognition and -binding site. This region consists of a dimer of one heavy- and one light-chain variable domain in tight, non-covalent association.

“Single-chain Fv” (“sFv” or “scFv”) antibody fragments comprise the V_{H} and V_{L} domains of antibody, wherein these domains are present in a single polypeptide chain. In some embodiments, the Fv polypeptide further comprises a polypeptide linker between the VH and VL domains, which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “CDR” or “complementarity determining region” is intended to mean the non-contiguous antigen combining sites found within the variable region of both heavy and light chain polypeptides. CDRs have been described by Kabat et al (1977) J. Biol. Chem. 252:6609; Kabat et al., U.S. Dept. of Health and Human Services, “Sequences of proteins of immunological interest” (1991) (also referred to herein as Kabat 1991); by Chothia et al. (1987) J. Mol. Biol. 196:901 (also referred to herein as Chothia 1987); and MacCallum et al. (1996) J. Mol. Biol. 262:732, where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs, as defined by each of the above cited references are set forth in the CDR-table below as a comparison.

CDR Table

| | Kabat¹ | Chothia² | MacCallum³ |
|---|---|---|---|
| V_{H} CDR-1 | 31-35 | 26-32 | 30-35 |
| V_{H} CDR-2 | 50-65 | 53-55 | 47-58 |
| V_{H} CDR-3 | 95-102 | 96-101 | 93-101 |
| V_{L} CDR-1 | 24-34 | 26-32 | 30-36 |
| V_{L} CDR-2 | 50-56 | 50-52 | 46-55 |
| V_{L} CDR-3 | 89-97 | 91-96 | 89-96 |

¹Residue numbering follows the nomenclature of Kabat et al., 1991, supra
²Residue numbering follows the nomenclature of Chothia et al., supra
³Residue numbering follows the nomenclature of MacCallum et al., supra

As used herein, the terms “CDR-L1”, “CDR-L2”, and “CDR-L3” refer, respectively, to the first, second, and third CDRs in a light chain variable region. The terms “CDR-L1”, “CDR-L2”, and “CDR-L3” may be used interchangeably with “VL CDR1,” “VL CDR2,” and “VL CDR3,” respectively. As used herein, the terms “CDR-H1”, “CDR-H2”, and “CDR-H3” refer, respectively, to the first, second, and third CDRs in a heavy chain variable region. The terms “CDR-H1”, “CDR-H2”, and “CDR-H3” may be used interchangeably with “VH CDR1,” “VH CDR2,” and “VH CDR3,” respectively. As used herein, the terms “CDR-1”, “CDR-2”, and “CDR-3” refer, respectively, to the first, second and third CDRs of either chain's variable region.

T cell receptors, or portions thereof such as the TCR beta chains, also may be used as targeting sequences. A single chain TCR (“scTCR”) may also be utilized as a targeting sequence. scTCRs include an alpha chain variable region (Vα) and a beta chain variable region (Vβ) covalently linked through a suitable peptide linker sequence. For example, the Vα can be covalently linked to the VB through a suitable peptide linker (L) sequence fused to the C-terminus of the Vα and the N-terminus of the Vβ. A scTCR can have the structure Vα-L-Vβ. A scTCR can have the structure Vβ-L-Vα. A scTCR can also comprise a constant domain (also referred to as constant region). In some cases, a scTCR comprises, in order from N-terminus to C-terminus: i) a TCR α chain variable domain polypeptide; ii) a peptide linker; iii) a TCR β chain variable domain polypeptide; and iv) a TCR β chain constant region extracellular domain polypeptide. In some cases, a scTCR comprises, in order from N-terminus to C-terminus: i) a TCR β chain variable domain polypeptide; ii) a peptide linker; iii) a TCR α chain variable domain polypeptide; and iv) a TCR α chain constant region extracellular domain polypeptide.

As discussed above, a T-Cell-MP or T-Cell-MP-epitope conjugate may include, or have attached to the T-Cell-MP, a targeting sequence such as a targeting sequence specific for a cancer-associated epitope (i.e., a cancer targeting polypeptide or “CTP”). For example, the first and/or the second polypeptide of a duplex T-Cell-MP-epitope conjugate may have independently selected targeting sequences such as CTPs attached. A “cancer-associated” epitope is an epitope that is present in a cancer-associated antigen. In some cases, a CTP is an antibody. In some cases, a CTP is a T cell receptor (e.g., a TCR beta chain or a TCR single-chain T cell receptor (scTCR)). Alternatively, instead of targeting a cancer antigen, a targeting sequence present in a T-Cell-MP may target an antigen of an infecting coronavirus (e.g., MERS-CoV, Bat-SL-CoV, SARS-CoV, and SARS-CoV-2 (COVID-19)) protein that is expressed on the surface of an infected cell. Non-limiting examples of targeting antibodies (or antigen binding fragments thereof) that can be included in a T-Cell-MP include, but are not limited to, antibodies against the coronavirus structural proteins, spike, nucleocapsid, membrane and envelope, which have homologues in all coronaviruses. Tan et al., Antiviral, 65 (2): 69-78 (2005).

For example, some coronavirus spike protein may be present on the surface of infected cells according to Heald-Sargent and Gallager, Viruses 4:557-580 (2012). Even if not present on an infected cell surface due to expression within a cell, the portion of a viral protein that is exposed on the coronavirus may be targeted as the virus binds to a cell protein as part of its entry into a cell, particularly when therapeutics inhibit endocytosis of coronavirus are co-administered. For example, heparin sulfate proteoglycan (HAPG)-assisted endocytosis of coronaviruses may be inhibited by mitoxantrone, which targets HSPGs directly, and Sunitinib and BNTX disrupt the actin network to impair HSPG-assisted viral entry. While the use of targeting sequences that bind to proteins of the viral envelope may cause scavenging of some portion of the T-Cell-MP-epitope conjugate, binding to the targeting sequence may effectively neutralize the virus, and the T-Cell-MP-epitope conjugate may still act to present epitope and MOD(s) to target specific T cells. Examples of monoclonal antibodies that may be utilized for such targeting include bamlanivimab, etesevimab, casirivimab, imdevimab, and sotrovimab all directed to the spike protein of Covid-19.

Where it is desirable to specifically direct the T-Cell-MP to a virus infected cell, such as with a redirected T-Cell-MP-epitope conjugate, the targeting sequence may be directed to a coronavirus peptide epitope-MHC complex (peptide epitope-HLA complex). Such MHC-epitope complexes may be targeted by, for example, a single-chain TCR, a monoclonal antibody, a scFv, a nanobody targeting sequence, or the like. The peptide of the peptide epitope-MHC complex may be an epitope of a coronavirus structural protein such as the spike, nucleocapsid, membrane, or envelope proteins.

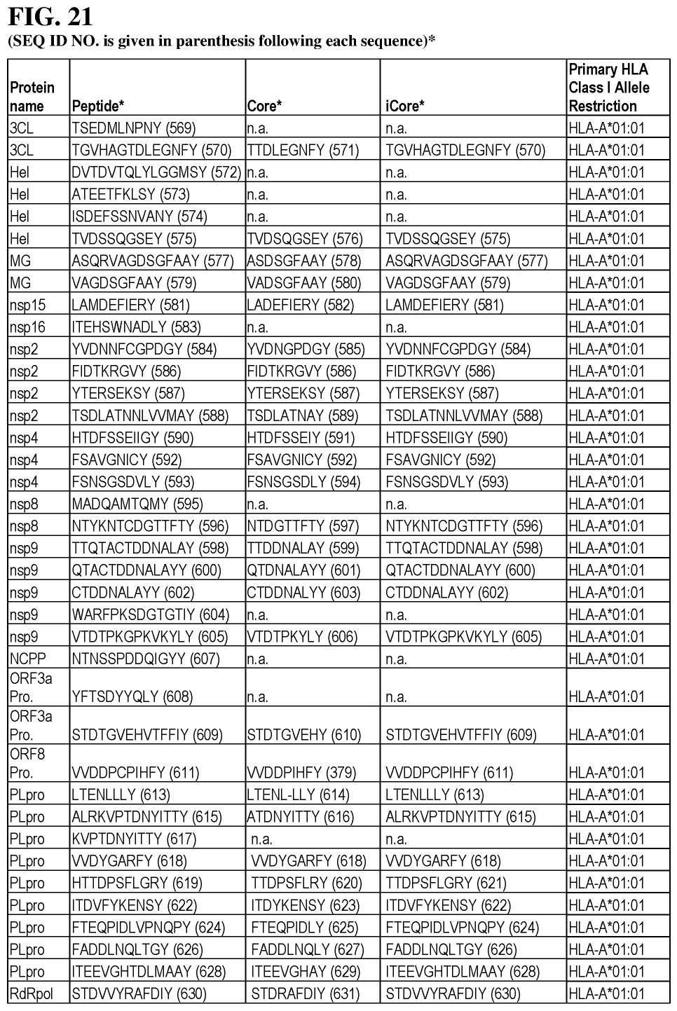

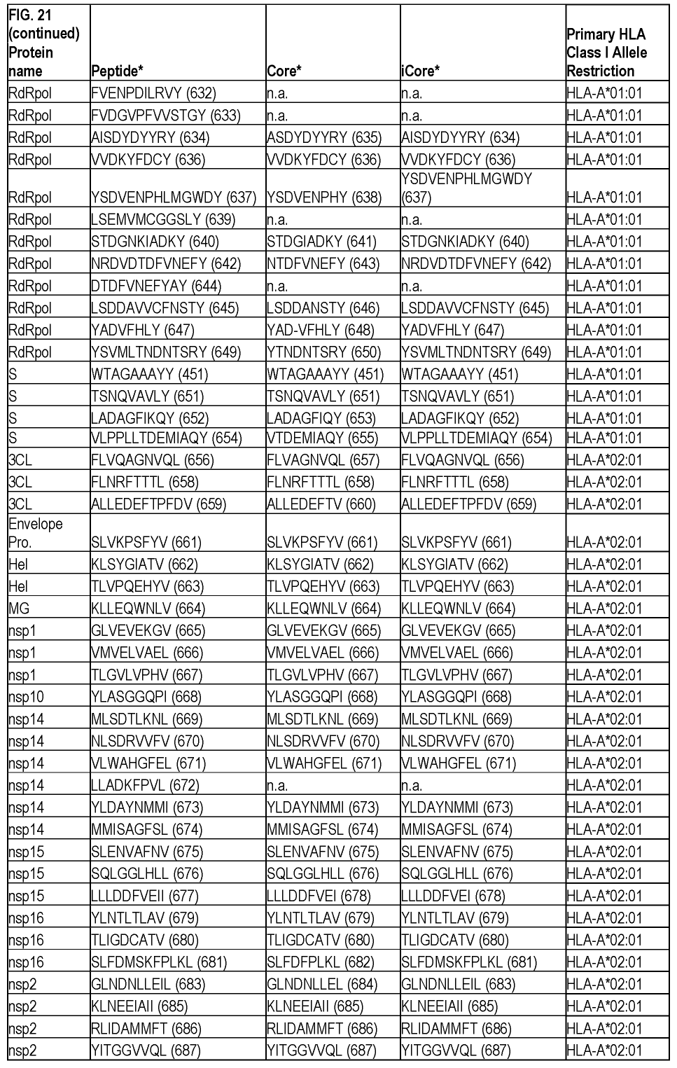

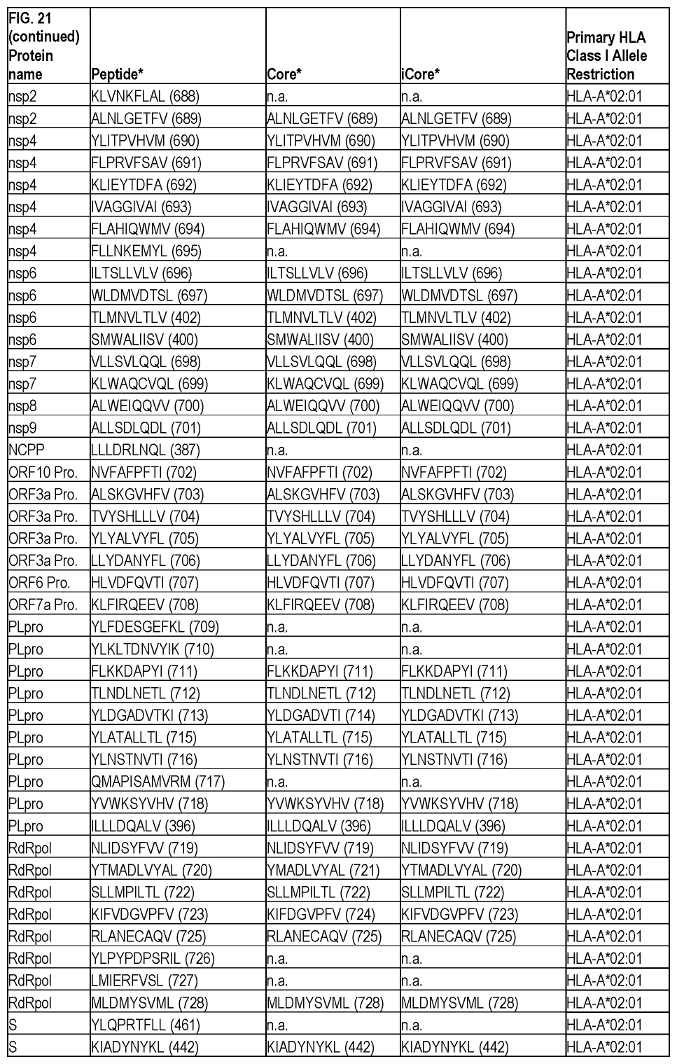

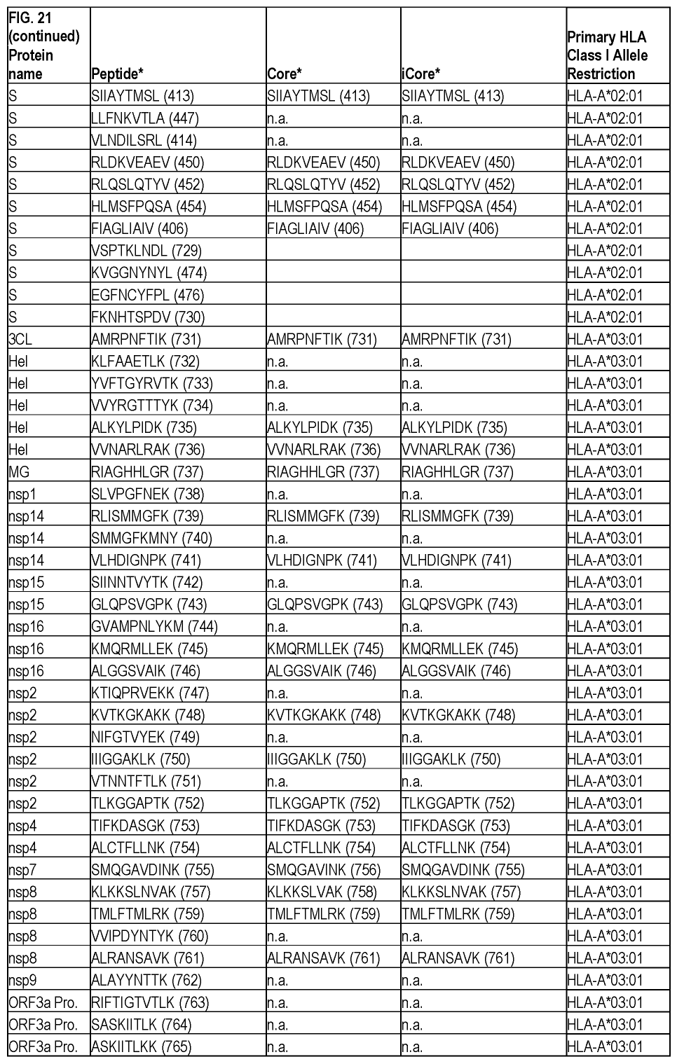

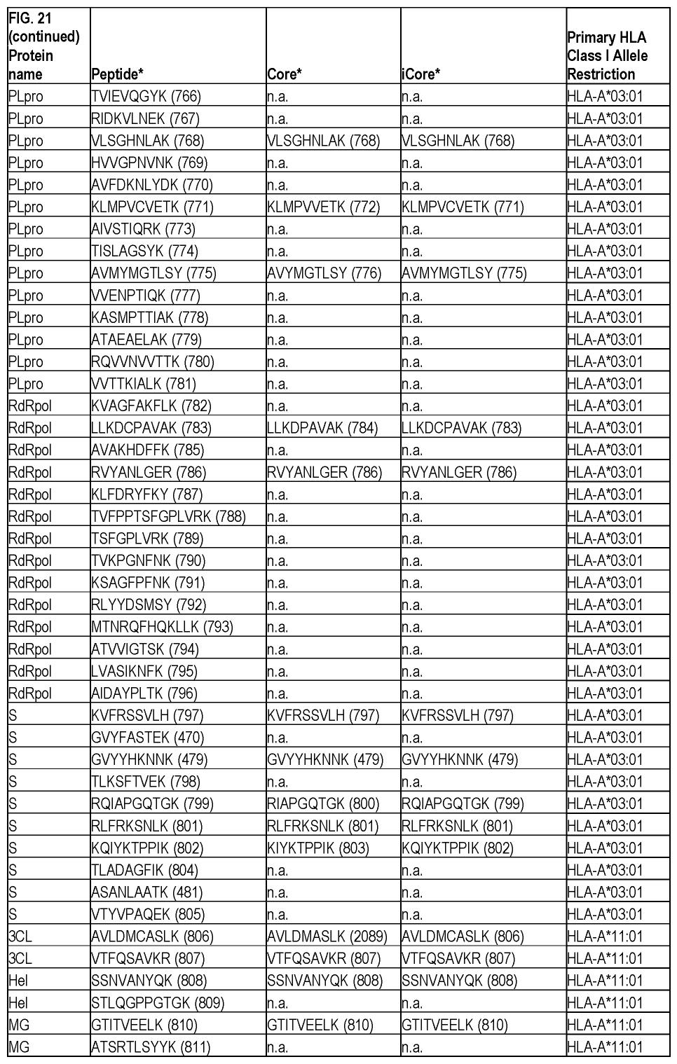

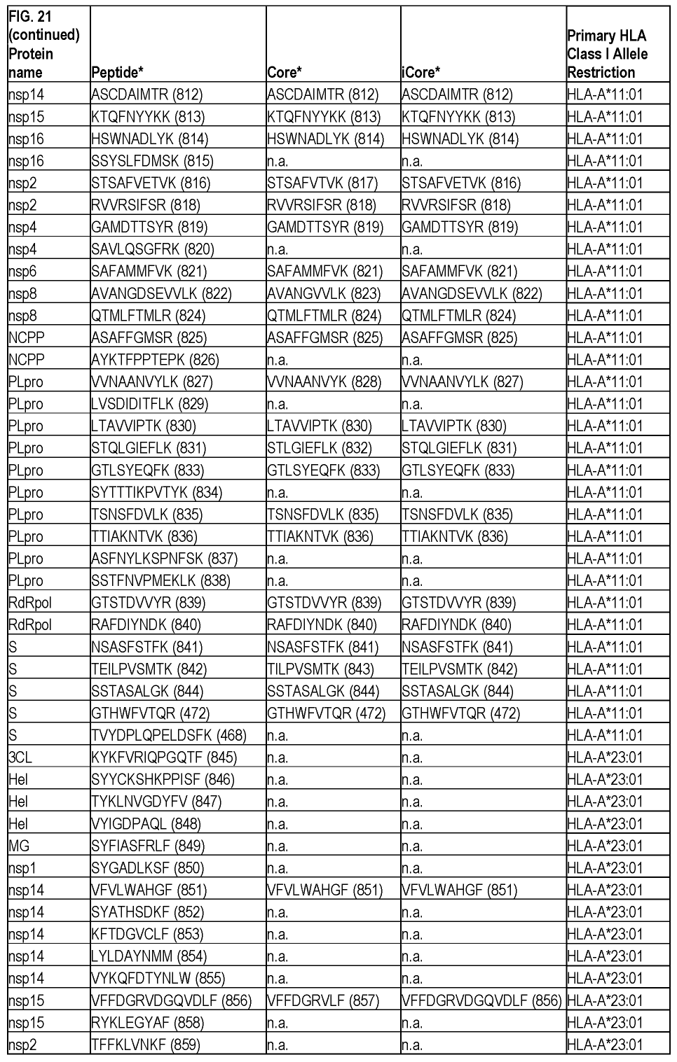

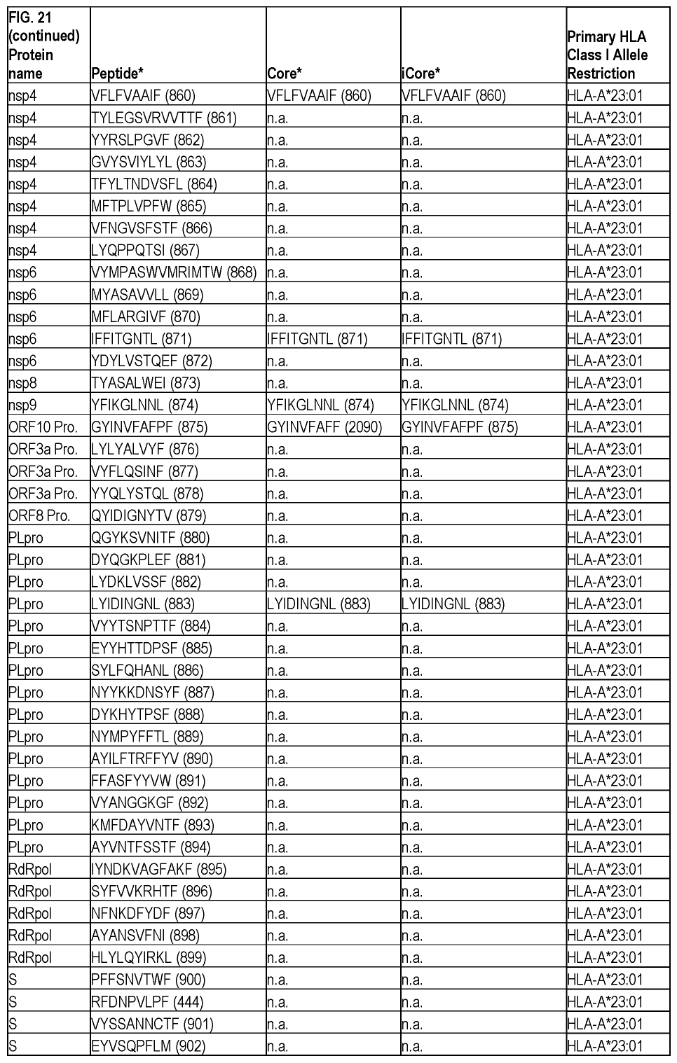

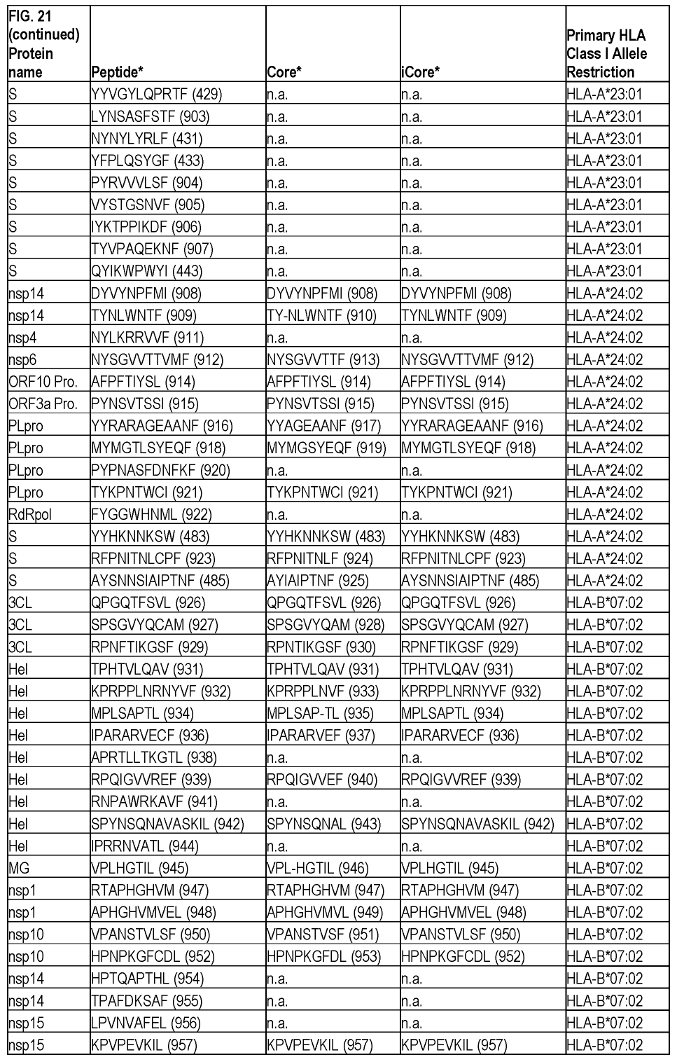

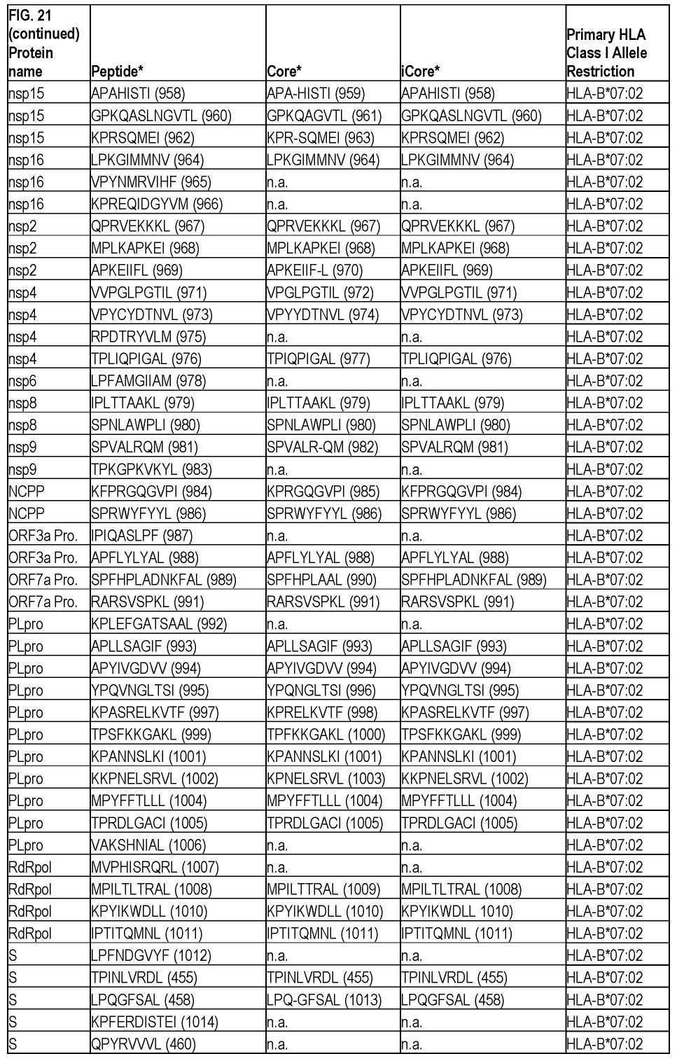

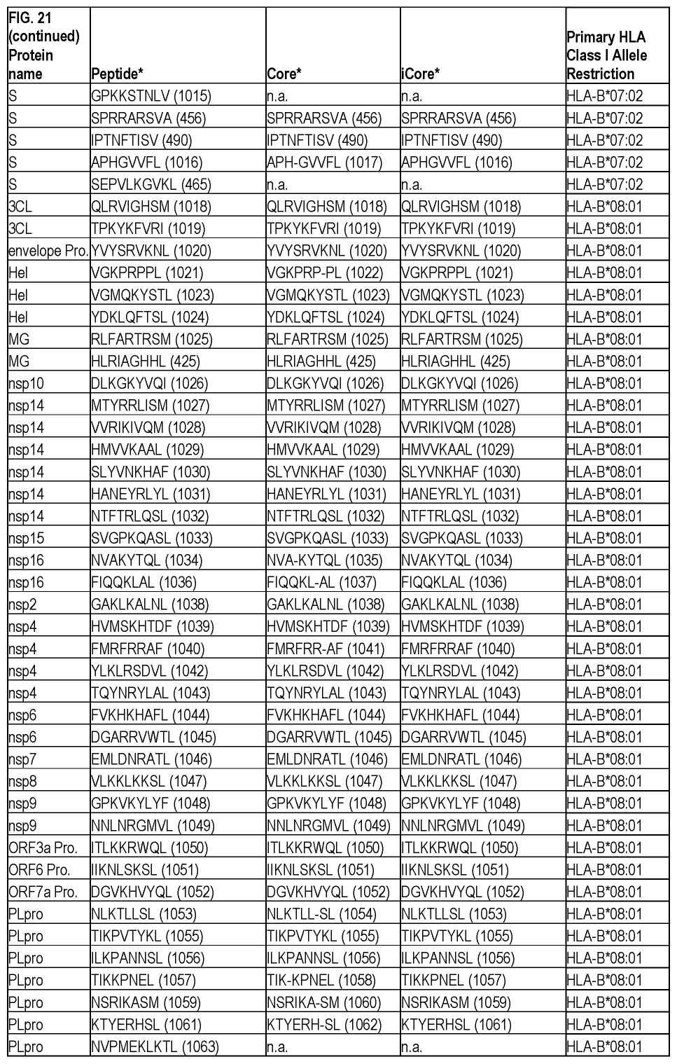

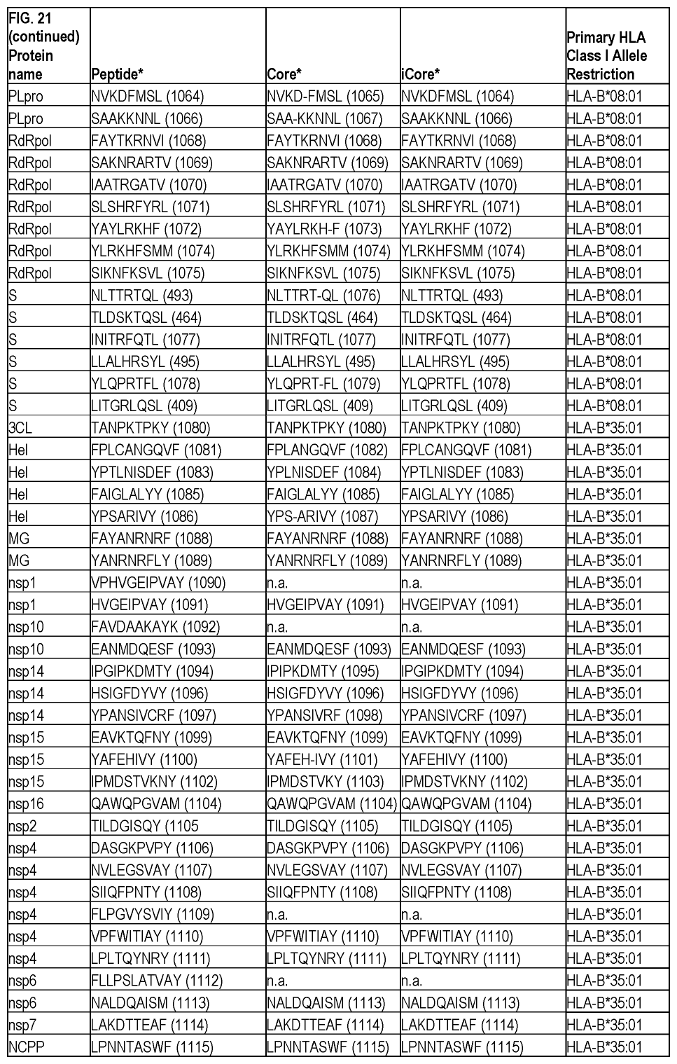

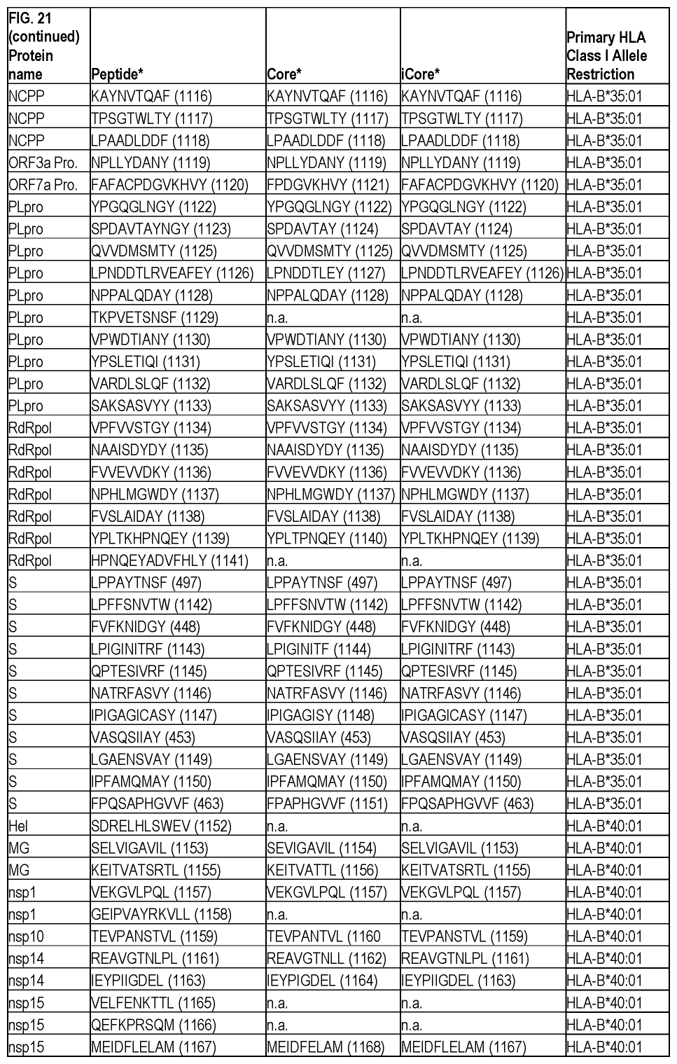

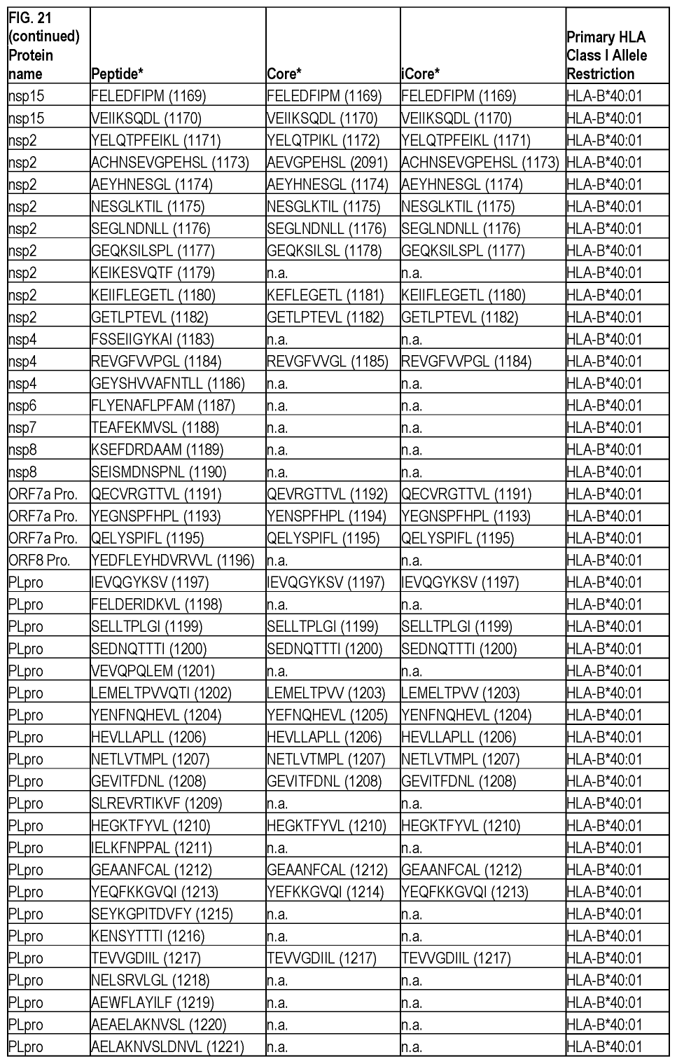

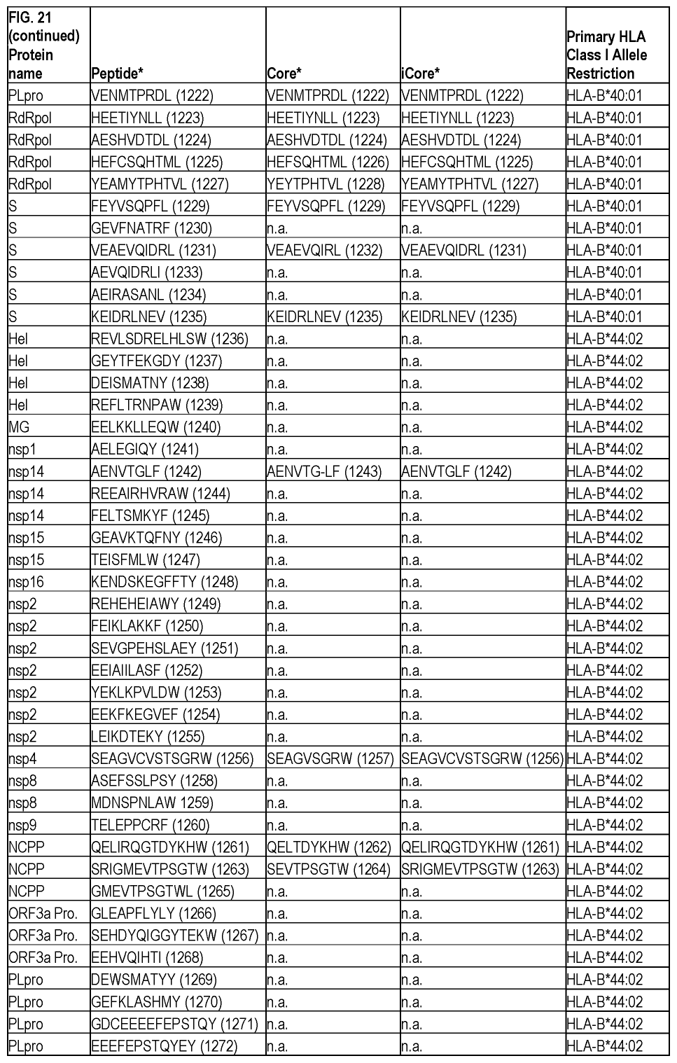

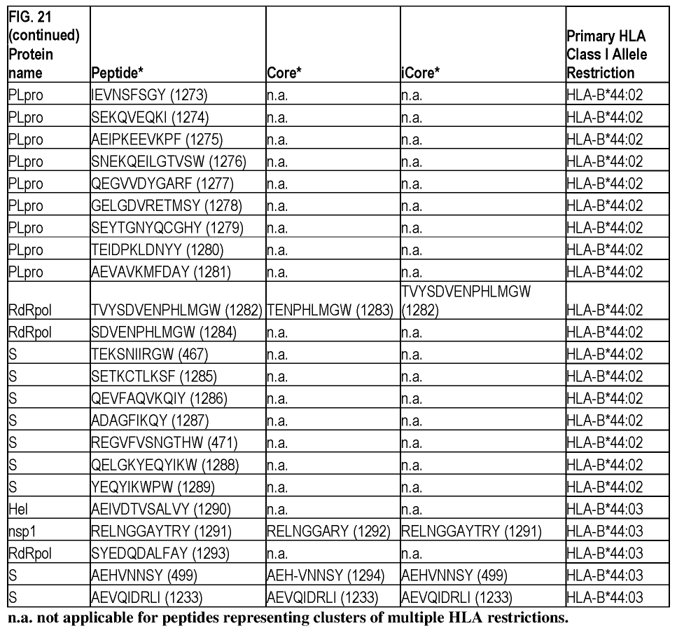

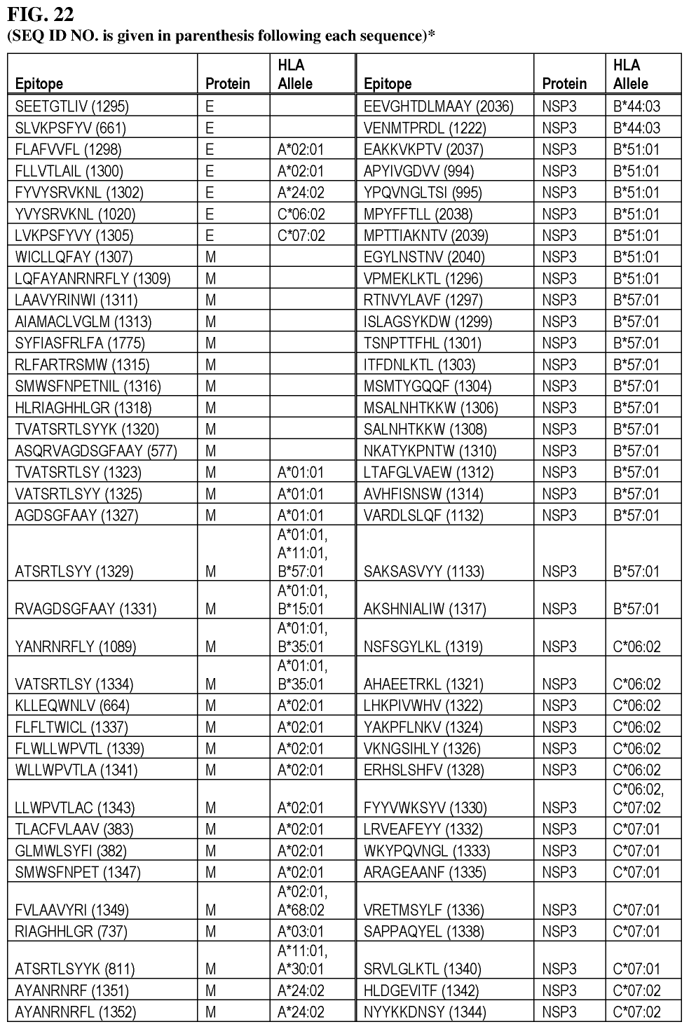

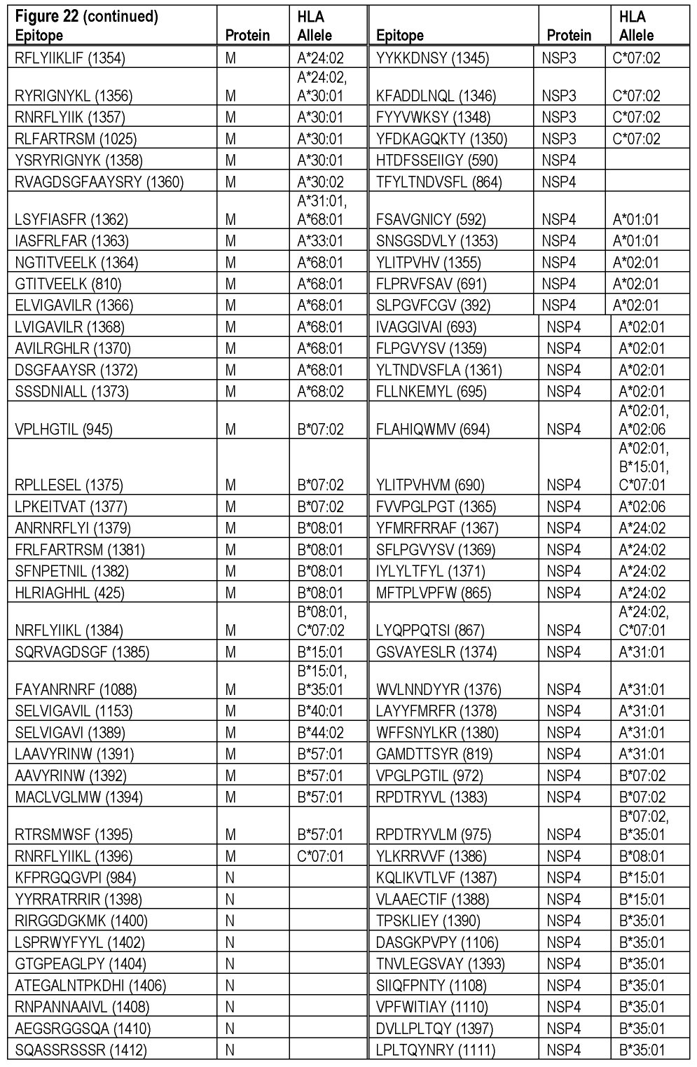

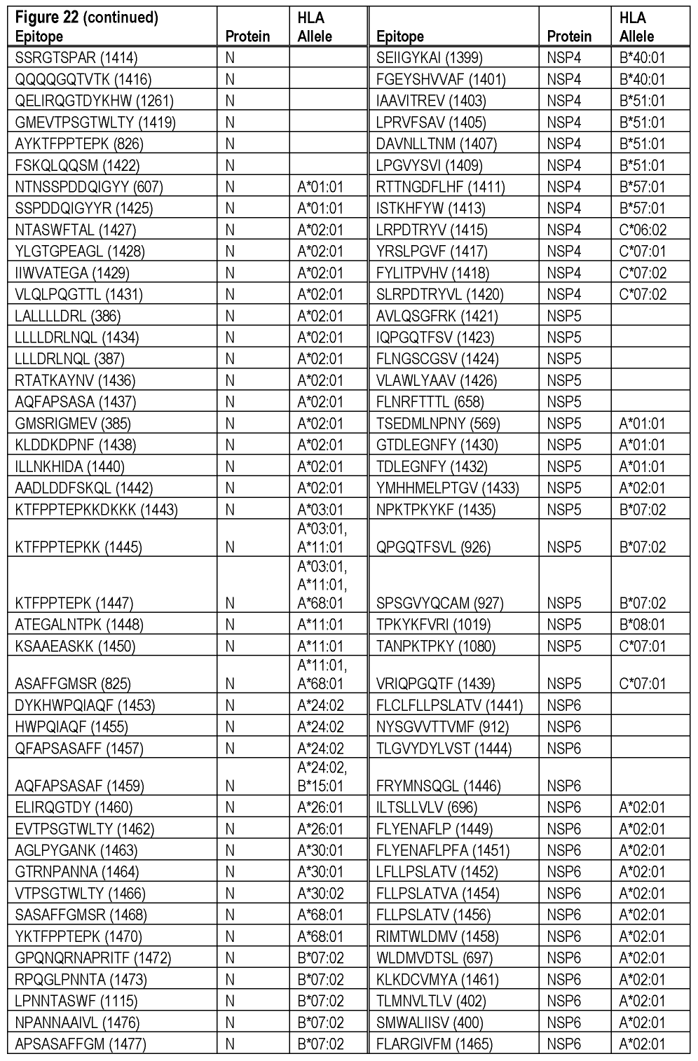

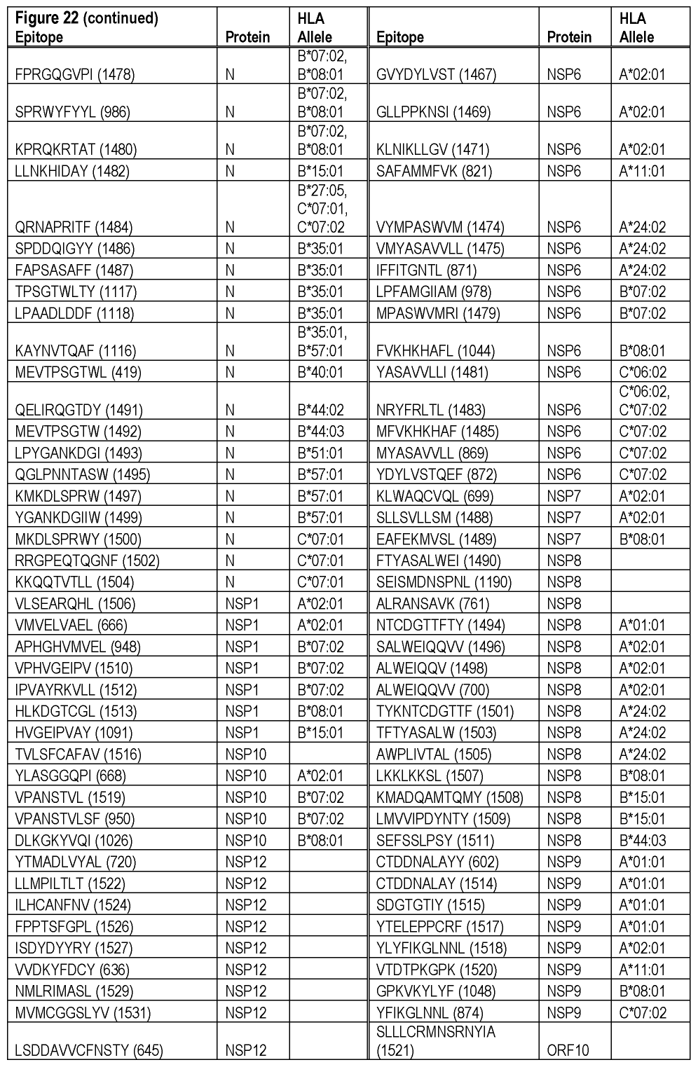

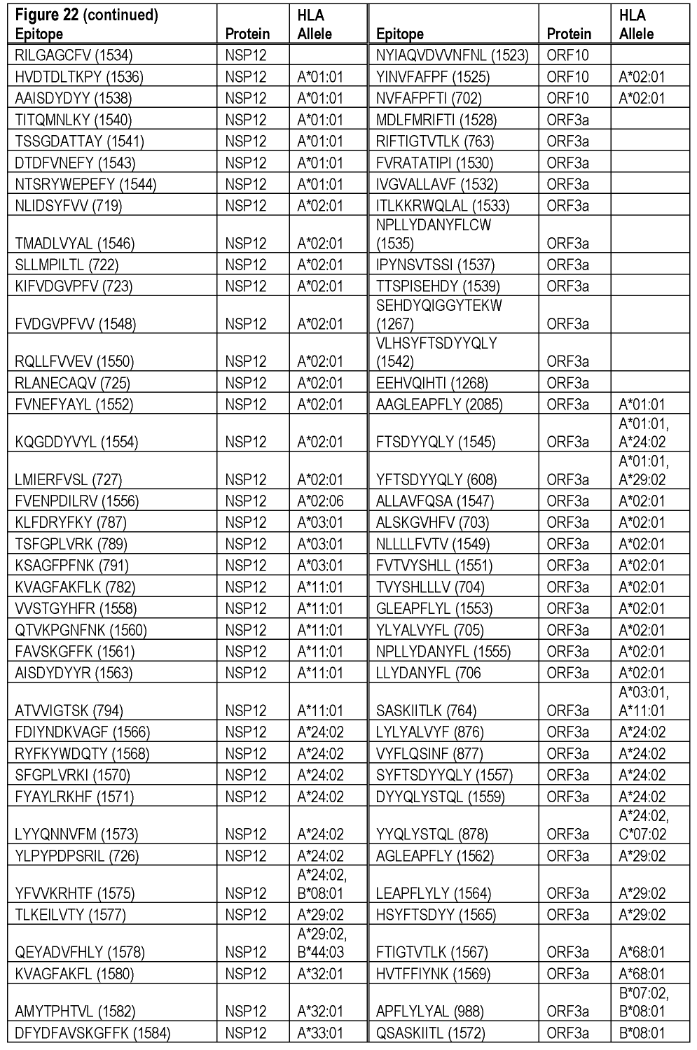

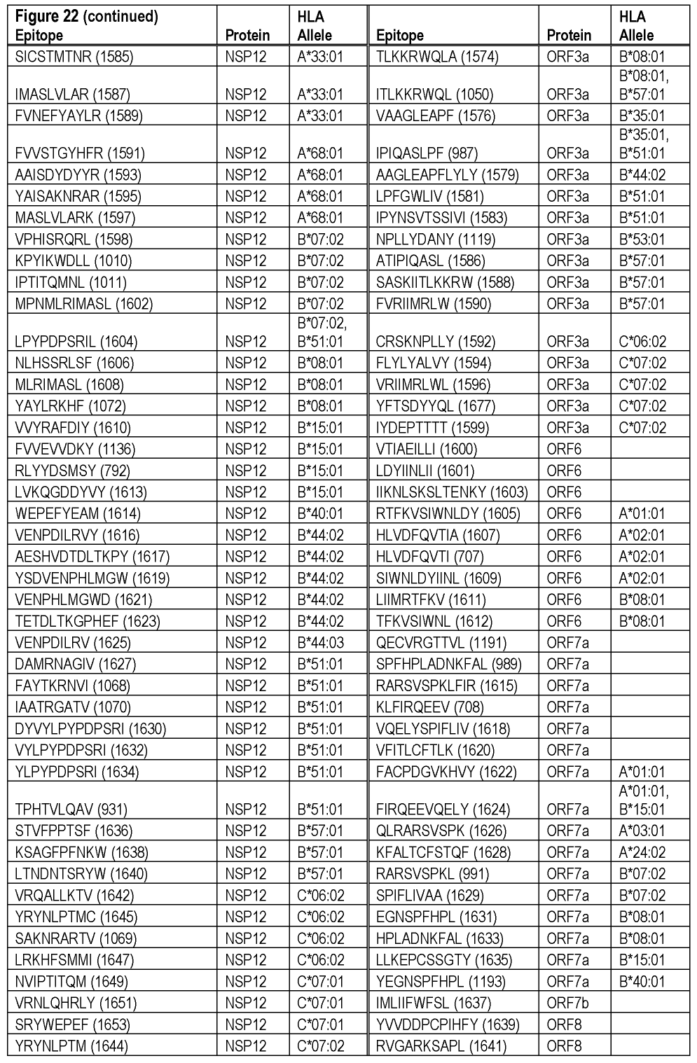

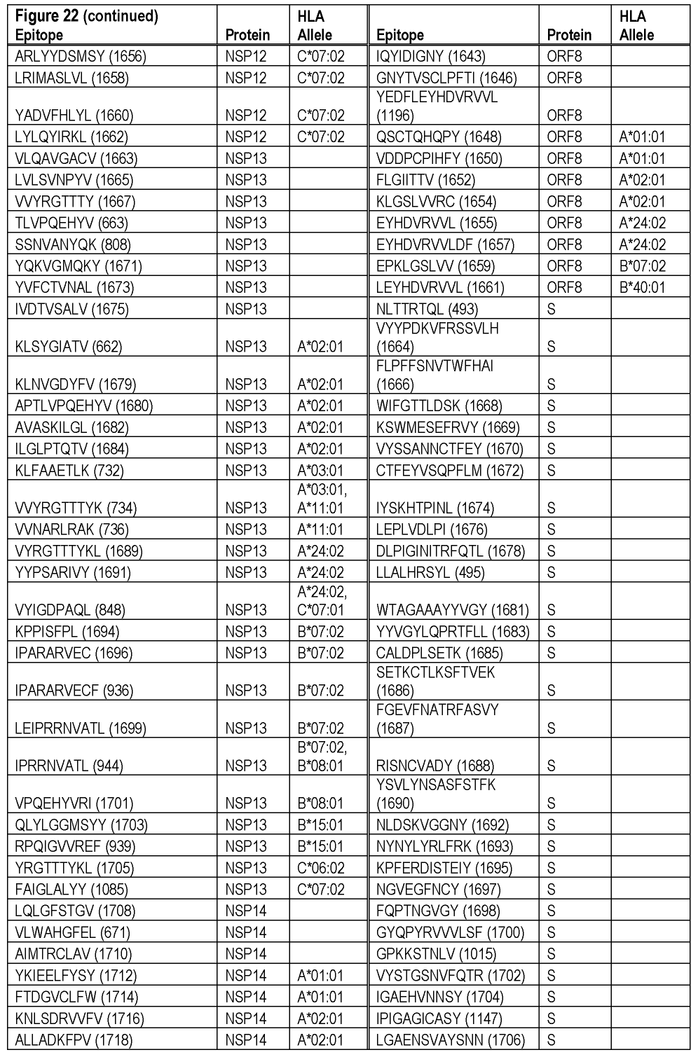

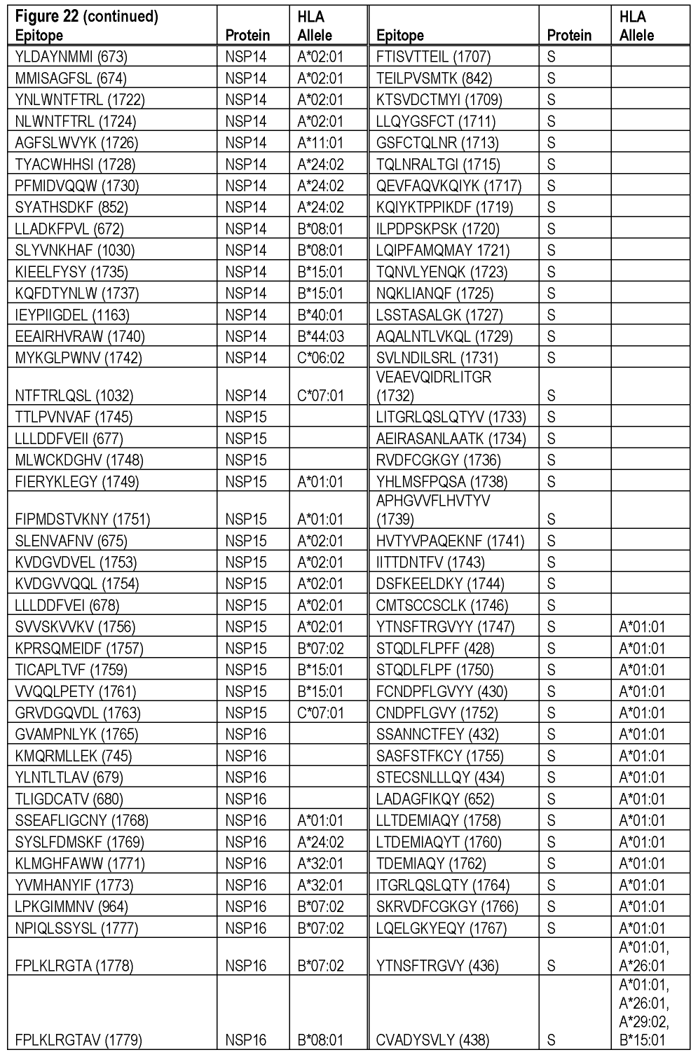

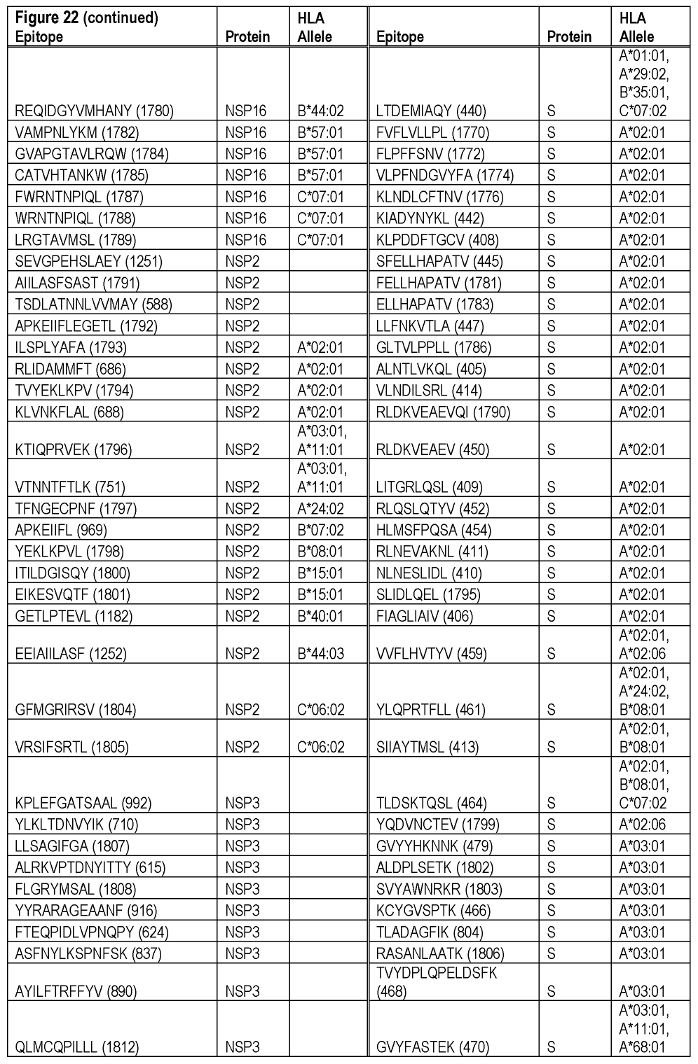

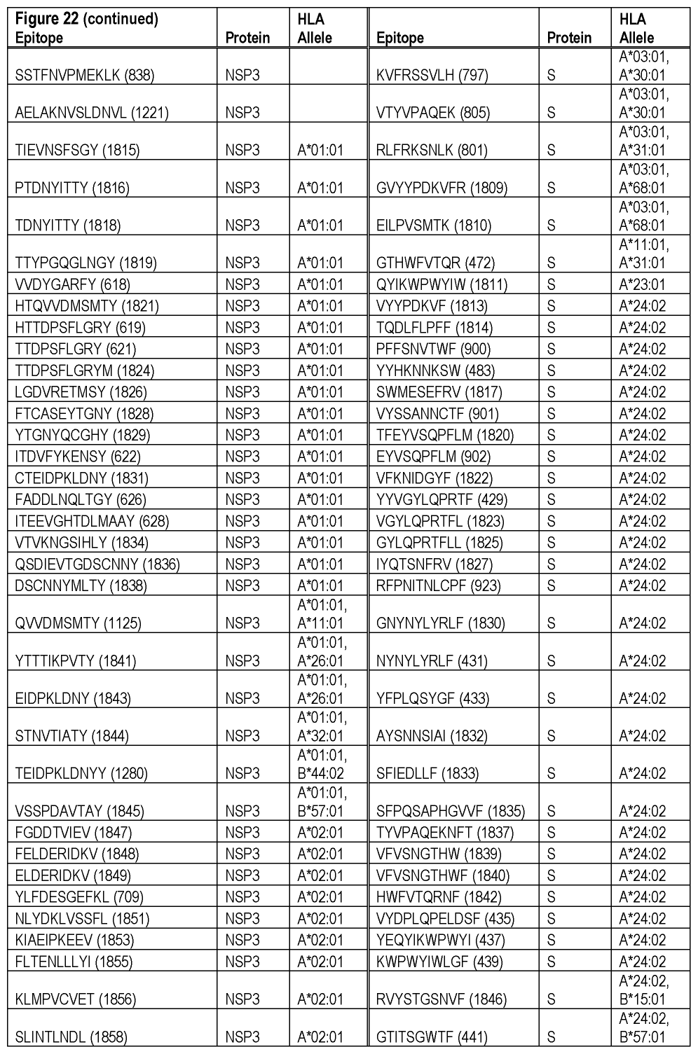

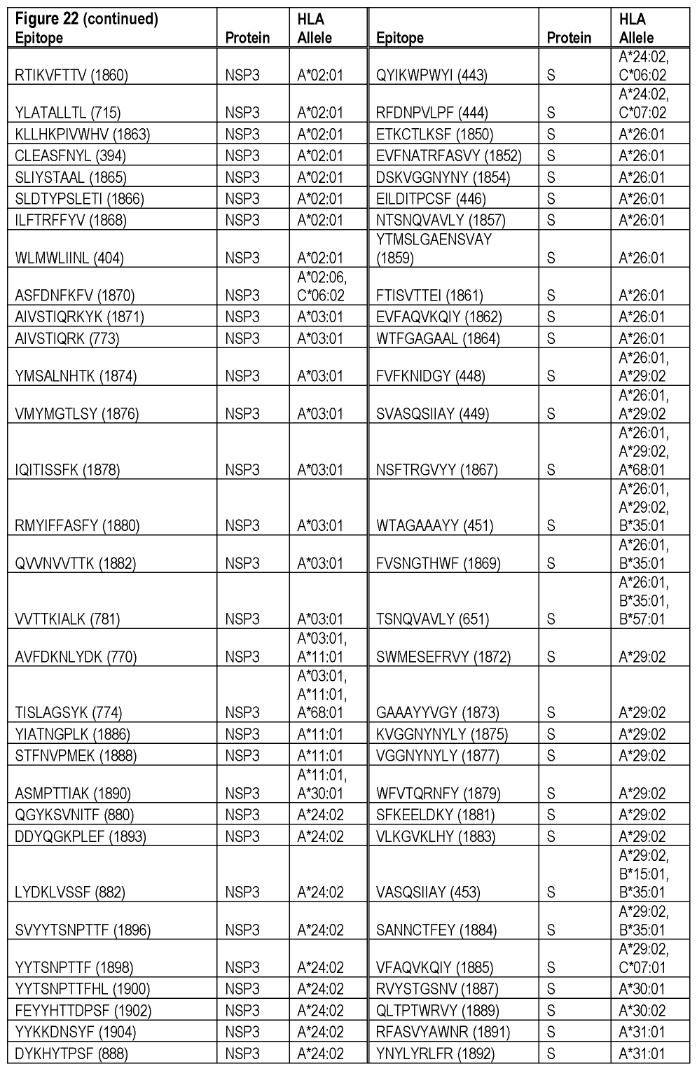

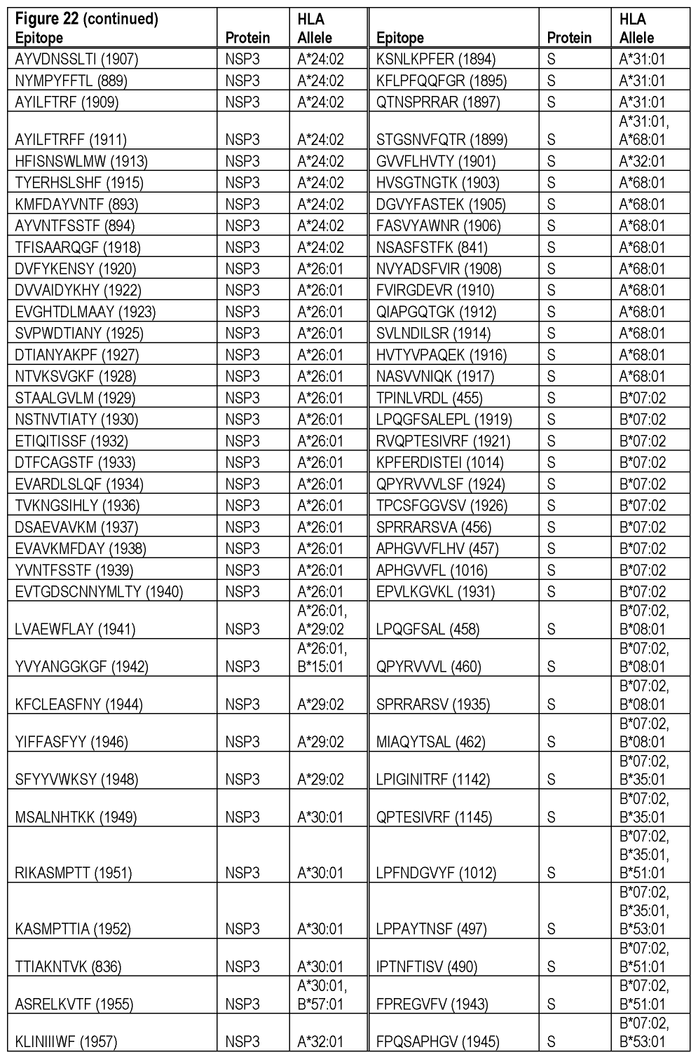

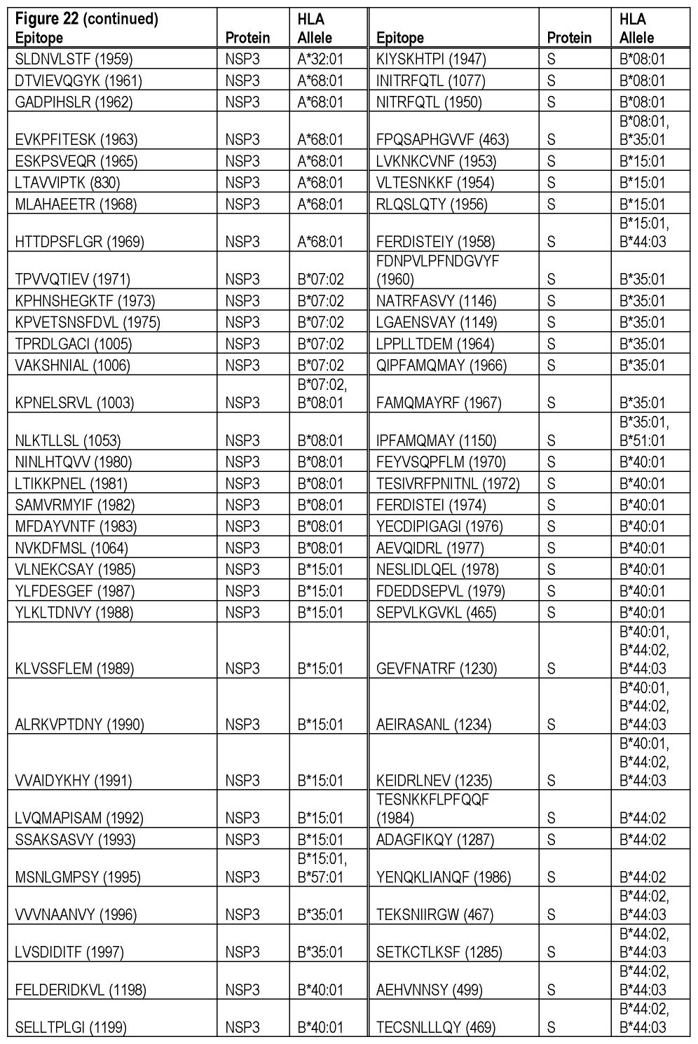

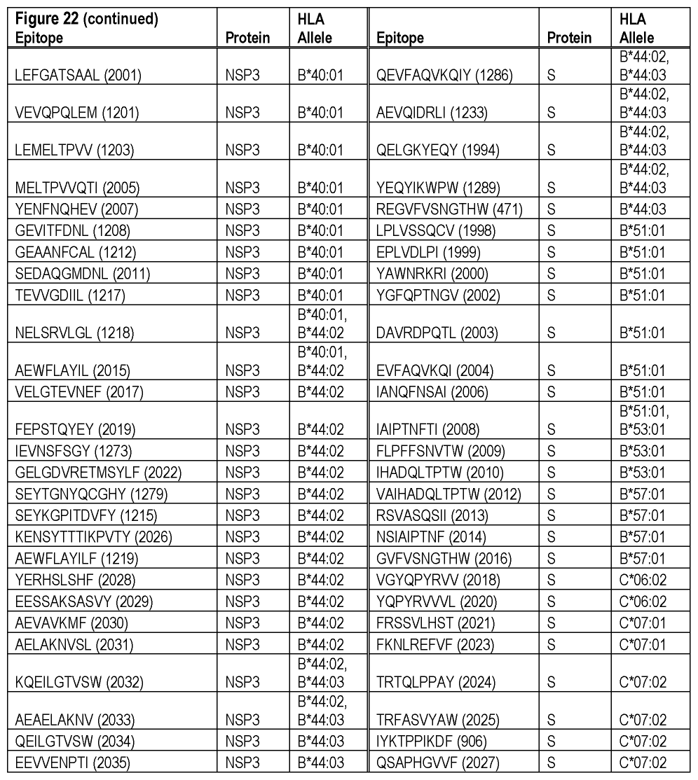

Some suitable epitopes of spike and other coronavirus proteins that may be directly targeted with, for example, monoclonal antibodies or antigen binding fragments thereof, or are provided in FIGS. 21 and 22 and in Tables 2 and 3. Antibodies or antigen binding fragments thereof may also be used to target HLA peptide complexes comprising the peptide epitopes in FIGS. 21 and 22 and in Tables 2 and 3.

As an alternative, the targeting sequence of T-Cell-MPs may be directed to an antigen present in tissues likely to be infected by coronaviruses such as alveolar epithelial cells. Alveolar epithelial cells are generally divided into alveolar epithelial type 1 (ATI) and alveolar epithelial type II cells (ATII). Upon injury to lung epithelium ATII cells have the ability to act as progenitor cells giving rise to ATI cells and additional ATII cells Gonzales et al. J Histochem Cytochem. 58 (10): 891-901 (2010). Suitable apical plasma membrane proteins of ATI cells that may be targeted include, for example, hTIa-2, gp36 and Human type I cell 56-kDa protein (McElroy and Kasper, Eur Respir J, 24:664-673 (2004). Apical plasma membrane proteins of ATII cells include, for example, HTII-280 (Gonzales loc. cit.).

### (i) Targets

In some cases, a targeting sequence is a CTP that targets a cancer-associated antigen. In some cases, the target of a CTP is a peptide/HLA (pHLA) complex on the surface of a cancer cell, where the peptide can be a cancer-associated peptide (e.g., a peptide fragment of a cancer-associated antigen). See, e.g., Tebentafusp, which targets gp 100 peptide (positions 280-288) that is presented on HLA-A*02:01 with picomolar affinity (gp 100 is a lineage melanocytic antigen expressed in melanocytes and melanoma).

### (a) Cancer-Associated Antigens

Cancer-associated antigens that can be targeted with a CTP include, e.g., NY-ESO (New York Esophageal Squamous Cell Carcinoma 1 or 2), melanoma antigen recognized by T cells or “MART” (e.g., MART-1, also known as Melan-A, or MELANA), HPV (human papilloma virus) E6, BCMA (B-cell maturation antigen), CD123, CD133, CD171, CD19, CD20, CD22, CD30, CD33, CD38, CD138, CEA (carcinoembryonic antigen), EGFR (epidermal growth factor receptor), EGFRvIII (epidermal growth factor receptor variant III), EpCAM (epithelial cell adhesion molecule), EphA2 (ephrin type-A receptor 2), disialoganglioside GD2, GPC3 (glypican-3), HER2, IL 13Ralpha2 (Interleukin 13 receptor subunit alpha-2), LeY (a difucosylated type 2 blood group-related antigen), melanoma-associated antigen(s) or “MAGE” (e.g., MAGE A3 or MAGE A4), melanoma glycoprotein, mesothelin, MUC1 (mucin 1), MUC16 (mucin-16), myelin, NKG2D (Natural Killer Group 2D) ligands, PSMA (prostate specific membrane antigen), and ROR1 (type I receptor tyrosine kinase-like orphan receptor).

Cancer-associated antigens that can be targeted with a CTP present in a T-Cell-MP include, but are not limited to, 17-1A-antigen, alpha-fetoprotein (AFP), alpha-actinin-4, A3, antigen specific for A33 antibody, ART-4, B7, Ba 733, BAGE, bcl-2, bcl-6, BCMA, BrE3-antigen, CA125, CAMEL, CAP-1, carbonic anhydrase IX (CAIX), CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CEA, CEACAM5, CEACAM6, claudin (e.g., claudin-1, claudin-10, claudin-18 (e.g., claudin-18, isoform 2)), complement factors (such as C3, C3a, C3b, C5a and C5), colon-specific antigen-p (CSAp), c-Met, CTLA-4, CXCR4, CXCR7, CXCL12, DAM, Dickkopf-related protein (DKK), ED-B fibronectin, epidermal growth factor receptor (EGFR), EGFRvIII, EGP-1 (TROP-2), EGP-2, ELF2-M, Ep-CAM, EphA2, EphA3, fibroblast activation protein (FAP), fibroblast growth factor (FGF), Flt-1, Flt-3, folate binding protein, folate receptor, G250 antigen, gangliosides (such as GC2, GD3 and GM2), GAGE, GD2, gp100, GPC3, GRO-13, HLA-DR, HM1.24, human chorionic gonadotropin (HCG) and its subunits, HER2, HER3, HMGB-1, hypoxia inducible factor (HIF-1), HIF-1a, HSP70-2M, HST-2, Ia, IFN-gamma, IFN-alpha, IFN-beta, IFN-X, IL-4R, IL-6R, IL-13R, IL13Ralpha2, IL-15R, IL-17R, IL-18R, IL-2, IL-6, IL-8, IL-12, IL-15, IL-17, IL-18, IL-23, IL-25, ILGF, ILGF-1R, insulin-like growth factor-1 (IGF-1), IGF-1R, integrin αVβ3, integrin α5β1, KC4-antigen, killer-cell Ig-like receptor (KIR), Kras, KS-1-antigen, KS1-4, LDR/FUT, Le^{gamma}, macrophage migration inhibitory factor (MIF), MAGE (e.g., MAGE A1, MAGE A2, MAGE A2B, MAGE A3, MAGE A4, MAGE A6, MAGE A8, MAGE A9, MAGE A9B, MAGE A10, MAGE A11, MAGE A12, MAGE C1, MAGE C2 in Human Genome Nomenclature Committee or “HGNC” abbreviations), MART-1, MART-2, mCRP, MCP-1, melanoma glycoprotein, mesothelin, MIP-1A, MIP-1B, MIF, mucins (such as MUC1, MUC2, MUC3, MUC4, MUC5ac, MUC13, MUC16, MUM-1/2 and MUM-3), NCA66, NCA95, NCA90, Nectin-4, NY-ESO-1, NY-ESO2, PAM4 antigen, pancreatic cancer mucin, PD-1, PD-L1, PD-1 receptor, placental growth factor, p53, PLAGL2, prostatic acid phosphatase, PSA, PRAME, PSMA, P1GF, RSS, RANTES, SAGE, 5100, survivin, survivin-2B, T101, TAC, TAG-72, tenascin, Thomson-Friedenreich antigens, Tn antigen, TNF-alpha, tumor necrosis antigens, TRAG-3, TRAIL receptors, vascular endothelial growth factor (VEGF), VEGF receptor (VEGFR) and WT-1.

In some cases, the cancer-associated antigen is an antigen associated with a hematological cancer. Examples of such antigens include, but are not limited to, BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-L1, TRAILR1, TRAILR2, transferrin receptor, and VEGF. In some cases, the cancer-associated antigen is an antigen expressed by malignant B cells, such as CD19, CD20, CD22, CD25, CD38, CD40, CD45, CD74, CD80, CTLA-4, IGF-R1, IL6, PD-1, TRAILR2, or VEGF.

In some cases, the cancer-associated antigen is MAGE A4 or NY-ESO-1 (e.g., NY-ESO-1B).

In some cases, the cancer-associated antigen is an antigen associated with a solid tumor. Examples of such antigens include, but are not limited to, CAIX, cadherins, CEA, c-MET, CTLA-4, EGFR family members, EpCAM, EphA3, FAP, folate-binding protein, FR-alpha, gangliosides (such as GC2, GD3 and GM2), HER2, HER3, IGF-1R, integrin αVβ3, integrin α5β1, Le^{gamma}, Liv1, mesothelin, mucins, NaPi2b, PD-1, PD-L1, PD-1 receptor, pgA33, PSMA, RANKL, ROR1, TAG-72, tenascin, TRAILR1, TRAILR2, VEGF, VEGFR, and others listed above.

### (1) Peptide/HLA Complexes

In some cases, the target of a CTP is a peptide/HLA (pHLA) complex on the surface of a cancer cell, where the peptide can be a cancer-associated peptide (e.g., a peptide fragment of a cancer-associated antigen). Cancer-associated peptides are known in the art. In some cases, a cancer-associated peptide is bound to an HLA complex comprising an HLA-A*0201 heavy chain and a β2M polypeptide.

In some cases, the epitope present in the pHLA on the surface of a cancer cell is bound to an HLA complex comprising an HLA heavy chain such as HLA-A*0101, A*0201, A*0301, A*1101, A*2301, A*2402, A*2407, A*3303, and/or A*3401. In some cases, the epitope present in the pHLA on the surface of a cancer cell is bound to an HLA complex comprising an HLA heavy chain such as HLA-B*0702, B*0801, B*1502, B*3802, B*4001, B*4601, and/or B*5301. In some cases, the epitope present in the pHLA on the surface of a cancer cell is bound to an HLA complex comprising an HLA heavy chain such as C*0102, C*0303, C*0304, C*0401, C*0602, C*0701, C*702, C*0801, and/or C*1502.

In some cases, the epitope is a cancer-associated epitope of any one of the following cancer-associated antigens: a MUC1 polypeptide, an LMP2 polypeptide, an epidermal growth factor receptor (EGFR) vIII polypeptide, a HER-2/neu polypeptide, a melanoma associated antigen-1 (MAGE A1) polypeptide, a melanoma associated antigen family A3 (MAGE A3) polypeptide, a melanoma associated antigen family A4 (MAGE A4) polypeptide, a p53 polypeptide, a mutant p53 polypeptide, an NY-ESO-1 or 2 polypeptide, a folate hydrolase (prostate-specific membrane antigen; PSMA) polypeptide, a carcinoembryonic antigen (CEA) polypeptide, a claudin polypeptide (e.g., claudin-1, claudin-10, claudin-18 (e.g., claudin-18, isoform 2)), a Nectin-4 polypeptide, a melanoma antigen recognized by T-cells (melanA/MART1) polypeptide, a Ras polypeptide, a gp 100 polypeptide, a proteinase3 (PR1) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFB) polypeptide, a MAD-CT-2 polypeptide, a Fos-related antigen-1 (FOSL) polypeptide; a human papilloma virus (HPV) antigen; an alpha-fetoprotein (AFP) antigen; and a Wilms tumor-1 (WT1) antigen.

For example, in some cases, a CTP binds to: a) a WT-1 peptide bound to an HLA complex comprising an HLA heavy chain (e.g., an HLA-A*0201 heavy chain or an HLA-A*2402 heavy chain) and a β2M polypeptide; b) an HPV peptide bound to an HLA complex comprising a class I HLA heavy chain and a β2M polypeptide; c) a mesothelin peptide bound to an HLA complex comprising a class I HLA heavy chain and a β2M polypeptide; d) a Her2 peptide bound to an HLA complex comprising a class I HLA heavy chain and a β2M polypeptide; or e) a BCMA peptide bound to an HLA complex comprising a class I HLA heavy chain and a β2M polypeptide.

In some cases, a cancer-associated peptide is a peptide of a mesothelin polypeptide having at least 90%, at least 95%, at least 98%, at least 99%, or 100%, amino acid sequence identity to the following mesothelin amino acid sequence:

LAGE TGQEAAPLDG VLANPPNISS LSPRQLLGFP CAEVSGLSTE RVRELAVALA QKNVKLSTEQ LRCLAHRLSE PPEDLDALPL DLLLFLNPDA FSGPQACTRF FSRITKANVD LLPRGAPERQ RLLPAALACW GVRGSLLSEA DVRALGGLAC DLPGRFVAES AEVLLPRLVS CPGPLDQDQQ EAARAALQGG GPPYGPPSTW SVSTMDALRG LLPVLGQPII RSIPQGIVAA WRQRSSRDPS WRQPERTILR PRFRREVEKT ACPSGKKARE IDESLIFYKK WELEACVDAA LLATQMDRVN AIPFTYEQLD VLKHKLDELY PQGYPESVIQ HLGYLFLKMS PEDIRKWNVT SLETLKALLE VNKGHEMSPQ VATLIDRFVK GRGQLDKDTL DTLTAFYPGY LCSLSPEELS SVPPSSIWAV RPQDLDTCDP RQLDVLYPKA RLAFQNMNGS EYFVKIQSFL GGAPTEDLKA LSQQNVSMDL ATFMKLRTDA VLPLTVAEVQ KLLGPHVEGL KAEERHRPVR DWILRQRQDD LDTLGLGLQG GIPNGYLVLD LSMQEALSGT PCLLGPGPVL TVLALLLAST LA (SEQ ID NO:169). For example, a mesothelin peptide present in a pHLA complex can be: i) KLLGPHVEGL (SEQ ID NO:170); ii) AFYPGYLCSL (SEQ ID NO:171), which can bind HLA-A*2402/32M; iii) VLPLTVAEV (SEQ ID NO:172); iv) ELAVALAQK (SEQ ID NO:173); v) ALQGGGPPY (SEQ ID NO: 174); vi) FYPGYLCSL (SEQ ID NO:175); vii) LYPKARLAF (SEQ ID NO:176); viii) LLFLLFSLGWVGPSR (SEQ ID NO: 177); ix) VNKGHEMSPQAPRRP (SEQ ID NO:178); x) FMKLRTDAVLPLTVA (SEQ ID NO:179); or xi) DAALLATQMD (SEQ ID NO:180).

In some cases, a cancer-associated peptide is a peptide of a Her2 polypeptide having at least 90%, at least 95%, at least 98%, at least 99%, or 100%, amino acid sequence identity to the following Her2 (receptor tyrosine-protein kinase erbB2) amino acid sequence:

(SEQ ID NO: 181)

MELAALCRWG LLLALLPPGA ASTQVCTGTD MKLRLPASPE
THLDMLRHLY QGCQVVQGNL ELTYLPTNAS LSFLQDIQEV
QGYVLIAHNQ VRQVPLQRLR IVRGTQLFED NYALAVLDNG
DPLNNTTPVT GASPGGLREL QLRSLTEILK GGVLIQRNPQ
LCYQDTILWK DIFHKNNQLA LTLIDTNRSR ACHPCSPMCK
GSRCWGESSE DCQSLTRTVC AGGCARCKGP LPTDCCHEQC
AAGCTGPKHS DCLACLHFNH SGICELHCPA LVTYNTDTFE
SMPNPEGRYT FGASCVTACP YNYLSTDVGS CTLVCPLHNQ
EVTAEDGTQR CEKCSKPCAR VCYGLGMEHL REVRAVTSAN
IQEFAGCKKI FGSLAFLPES FDGDPASNTA PLQPEQLQVF
ETLEEITGYL YISAWPDSLP DLSVFQNLQV IRGRILHNGA
YSLTLQGLGI SWLGLRSLRE LGSGLALIHH NTHLCFVHTV
PWDQLFRNPH QALLHTANRP EDECVGEGLA CHQLCARGHC
WGPGPTQCVN CSQFLRGQEC VEECRVLQGL PREYVNARHC
LPCHPECQPQ NGSVTCFGPE ADQCVACAHY KDPPFCVARC
PSGVKPDLSY MPIWKFPDEE GACQPCPINC THSCVDLDDK
GCPAEQRASP LTSIISAVVG ILLVVVLGVV FGILIKRRQQ
KIRKYTMRRL LQETELVEPL TPSGAMPNQA QMRILKETEL
RKVKVLGSGA FGTVYKGIWI PDGENVKIPV AIKVLRENTS
PKANKEILDE AYVMAGVGSP YVSRLLGICL TSTVQLVTQL
MPYGCLLDHV RENRGRLGSQ DLLNWCMQIA KGMSYLEDVR
LVHRDLAARN VLVKSPNHVK ITDFGLARLL DIDETEYHAD
GGKVPIKWMA LESILRRRFT HQSDVWSYGV TVWELMTFGA
KPYDGIPARE IPDLLEKGER LPQPPICTID VYMIMVKCWM
IDSECRPRFR ELVSEFSRMA RDPQRFVVIQ NEDLGPASPL
DSTFYRSLLE DDDMGDLVDA EEYLVPQQGF FCPDPAPGAG
GMVHHRHRSS STRNM.

In some cases, a cancer-associated peptide is a peptide of a BCMA polypeptide having at least 90%, at least 95%, at least 98%, at least 99%, or 100%, amino acid sequence identity to the following BCMA amino acid seQuence:

(SEQ ID NO: 182)

MLQMAGQCSQ NEYFDSLLHA CIPCQLRCSS NTPPLTCQRY
CNASVTNSVK GTNAILWTCL GLSLIISLAV FVLMFLLRKI
SSEPLKDEFK NTGSGLLGMA NIDLEKSRTG DEIILPRGLE
YTVEECTCED CIKSKPKVDS DHCFPLPAME EGATILVTTK
TNDYCKSLPA ALSATEIEKS ISAR.

In some cases, a cancer-associated peptide is a peptide of a WT-1 polypeptide having at least 90%, at least 95%, at least 98%, at least 99%, or 100%, amino acid sequence identity to the following WT-1 amino acid sequence:

(SEQ ID NO: 183)

MDFLLLQDPA STCVPEPASQ HTLRSGPGCL QQPEQQGVRD
PGGIWAKLGA AEASAERLQG RRSRGASGSE PQQMGSDVRD
LNALLPAVPS LGGGGGCALP VSGAAQWAPV LDFAPPGASA
YGSLGGPAPP PAPPPPPPPP PHSFIKQEPS WGGAEPHEEQ
CLSAFTVHFS GQFTGTAGAC RYGPFGPPPP SQASSGQARM
FPNAPYLPSC LESQPAIRNQ GYSTVTFDGT PSYGHTPSHH
AAQFPNHSFK HEDPMGQQGS LGEQQYSVPP PVYGCHTPTD
SCTGSQALLL RTPYSSDNLY QMTSQLECMT WNQMNLGATL
KGHSTGYESD NHTTPILCGA QYRIHTHGVF RGIQDVRRVP
GVAPTLVRSA SETSEKRPFM CAYPGCNKRY FKLSHLQMHS
RKHTGEKPYQ CDFKDCERRF SRSDQLKRHQ RRHTGVKPFQ
CKTCQRKFSR SDHLKTHTRT HTGEKPFSCR WPSCQKKFAR
SDELVRHHNM HQRNMTKLQL AL.

Non-limiting examples of WT-1 peptides include RMFPNAPYL (SEQ ID NO: 184), CMTWNQMN (SEQ ID NO: 185), CYTWNQMNL (SEQ ID NO: 186), CMTWNQMNLGATLKG (SEQ ID NO: 187), WNQMNLGATLKGVAA (SEQ ID NO: 188), CMTWNYMNLGATLKG (SEQ ID NO:189), WNYMNLGATLKGVAA (SEQ ID NO:190), MTWNQMNLGATLKGV (SEQ ID NO:191), TWNQMNLGATLKGVA (SEQ ID NO:192), CMTWNLMNLGATLKG (SEQ ID NO: 193), MTWNLMNLGATLKGV (SEQ ID NO:194), TWNLMNLGATLKGVA (SEQ ID NO:195), WNLMNLGATLKGVAA (SEQ ID NO: 196), MNLGATLK (SEQ ID NO:197), MTWNYMNLGATLKGV (SEQ ID NO: 198), TWNYMNLGATLKGVA (SEQ ID NO:199), CMTWNQMNLGATLKGVA (SEQ ID NO:200), CMTWNLMNLGATLKGVA (SEQ ID NO:201), CMTWNYMNLGATLKGVA (SEQ ID NO:202), GYLRNPTAC (SEQ ID NO: 203), GALRNPTAL (SEQ ID NO:204), YALRNPTAC (SEQ ID NO:205), GLLRNPTAC (SEQ ID NO:206), RYRPHPGAL (SEQ ID NO:207), YQRPHPGAL (SEQ ID NO:208), RLRPHPGAL (SEQ ID NO:209), RIRPHPGAL (SEQ ID NO:210), QFPNHSFKHEDPMGQ (SEQ ID NO:211), HSFKHEDPY (SEQ ID NO:212), QFPNHSFKHEDPM (SEQ ID NO:213), QFPNHSFKHEDPY (SEQ ID NO:214), KRPFMCAYPGCNK (SEQ ID NO:215), KRPFMCAYPGCYK (SEQ ID NO:216), FMCAYPGCY (SEQ ID NO:217), FMCAYPGCK (SEQ ID NO:218), KRPFMCAYPGCNKRY (SEQ ID NO:219), SEKRPFMCAYPGCNK (SEQ ID NO:220), KRPFMCAYPGCYKRY (SEQ ID NO: 221), NLMNLGATL (SEQ ID NO:222), and NYMNLGATL (SEQ ID NO:223).

In some cases, a cancer-associated peptide is a peptide of a human papillomavirus (HPV) polypeptide having at least 90%, at least 95%, at least 98%, at least 99%, or 100%, amino acid sequence identity to an HPV polypeptide. An HPV peptide can be a peptide of an HPV E6 polypeptide or an HPV E7 polypeptide. The HPV epitope can be an epitope of HPV of any of a variety of genotypes, including, e.g., HPV16, HPV18, HPV31, HPV33, HPV35, HPV39, HPV45, HPV51, HPV52, HPV56, HPV58, HPV59, HPV68, HPV73, or HPV82. Non-limiting examples of HPV peptides include: E6 18-26 (KLPQLCTEL; SEQ ID NO:224); E6 26-34 (LQTTIHDII; SEQ ID NO: 225); E6 49-57 (VYDFAFRDL; SEQ ID NO:226); E6 52-60 (FAFRDLCIV; SEQ ID NO:227); E6 75-83 (KFYSKISEY; SEQ ID NO:228); E6 80-88 (ISEYRHYCY; SEQ ID NO:229); E7 7-15 (TLHEYMLDL; SEQ ID NO:230); E7 11-19 (YMLDLQPET; SEQ ID NO:231); E7 44-52 (QAEPDRAHY; SEQ ID NO:232); E7 49-57 (RAHYNIVTF (SEQ ID NO:233); E7 61-69 (CDSTLRLCV; SEQ ID NO:234); E7 67-76 (LCVQSTHVDI; SEQ ID NO:235); E7 82-90 (LLMGTLGIV; SEQ ID NO:236); E7 86-93 (TLGIVCPI; SEQ ID NO:237); and E7 92-93 (LLMGTLGIVCPI; SEQ ID NO: 238).

In some cases, a cancer-associated peptide is a peptide of a claudin polypeptide having at least 90%, at least 95%, at least 98%, at least 99%, or 100%, amino acid sequence identity to the following claudin-18 (isoform 2) (CLDN 18.2) amino acid sequence:

(SEQ ID NO: 239)

MAVTACQGLG FWVSLIGIAG IIAATCMDQW STQDLYNNPV
TAVFNYQGLW RSCVRESSGF TECRGYFTLL GLPAMLQAVR
ALMIVGIVLG AIGLLVSIFA LKCIRIGSME DSAKANMTLT
SGIMFIVSGL CAIAGVSVFA NMLVTNFWMS TANMYTGMGG
MVQTVQTRYT FGAALFVGWV AGGLTLIGGV MMCIACRGLA
PEETNYKAVS YHASGHSVAY KPGGFKASTG FGSNTKNKKI
YDGGARTEDE VQSYPSKHDY V.

In some cases, a cancer-associated peptide is a peptide of a claudin polypeptide having the amino acid sequence TEDEVQSYPSKHDYV (SEQ ID NO:240) (and having a length of about 15 amino acids) or EVQSYPSKHDYV (SEQ ID NO: 241) (and having a length of about 12 amino acids.

In some cases, a cancer-associated peptide is a peptide of a trophoblast cell-surface antigen-2 (Trop-2) polypeptide. Trop-2 (also known as epithelial glycoprotein-1, gastrointestinal tumor-associated antigen GA733-1, membrane component chromosome 1 surface marker-1, and tumor-associated calcium signal transducer-2) is a transmembrane glycoprotein that is upregulated in numerous cancer types, and is the protein product of the TACSTD2 gene. In some cases, a cancer-associated peptide is a peptide of a TROP-2 polypeptide having at least 90%, at least 95%, at least 98%, at least 99%, or 100%, amino acid sequence identity to the following TROP-2 amino acid sequence: QDNCTCPTNK MTVCSPDGPG GRCQCRALGS GMAVDCSTLT SKCLLLKARM SAPKNARTLV RPSEHALVDN DGLYDPDCDP EGRFKARQCN QTSVCWCVNS VGVRRTDKGD LSLRCDELVR THHILIDLRH RPTAGAFNHS DLDAELRRLF RERYRLHPKF VAAVHYEQPT IQIELRQNTS QKAAGDVDIG DAAYYFERDI KGESLFQGRG GLDLRVRGEP LQVERTLIYY LDEIPPKFSM KRLTAGLIAV IVVVVVALVA GMAVLVITNR RKSGKYKKVE IKELGELRKE PSL (SEQ ID NO:242).

### (ii) Antibodies as Targeting Sequences

As noted above, in some cases, a targeting sequence such as a CTP is an antibody. In some cases, the CTP is an antibody specific for a peptide/HLA complex on the surface of a cancer cell, where the peptide can be a cancer-associated peptide (e.g., a peptide of a cancer-associated antigen).

Non-limiting examples of cancer-associated antigen-targeted antibodies include, but are not limited to, abituzumab (anti-CD51), LL1 (anti-CD74), LL2 or RFB4 (anti-CD22), veltuzumab (hA20, anti-CD20), obinutuzu (anti-CD20), obinutuzumab (GA101, anti-CD20), daratumumab (anti-CD38), lambrolizumab (anti-PD-1 receptor), nivolumab (anti-PD-1 receptor), ipilimumab (anti-CTLA-4), RS7 (anti-TROP-2), PAM4 or KC4 (both anti-mucin), MN-14 (anti-CEA), MN-15 or MN-3 (anti-CEACAM6), Mu-9 (anti-colon-specific antigen-p), Immu 31 (anti-alpha-fetoprotein), R1 (anti-IGF-1R), A19 (anti-CD19), TAG-72 (e.g., CC49), Tn, J591 or HuJ591 (anti-PSMA), AB-PG1-XG1-026 (anti-PSMA dimer), D2/B (anti-PSMA), G250 (anti-carbonic anhydrase IX), L243 (anti-HLA-DR) alemtuzumab (anti-CD52), oportuzumab (anti-EpCAM), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (also known as clivatuzumab; anti-mucin), trastuzumab (anti-HER2), pertuzumab (anti-HER2), polatuzumab (anti-CD79b), and anetumab (anti-mesothelin).

In some cases, the targeting sequence (e.g., CTP) comprises a single-chain antibody. In some cases, the targeting sequence (e.g., CTP) is a scFv. In some cases, the targeting sequence (e.g., CTP) is a nanobody (also referred to as a single domain antibody (sdAb). In some cases, t targeting sequence (e.g., CTP) is a heavy chain nanobody. In some cases, the targeting sequence (e.g., CTP) is a light chain nanobody.

VH and VL amino acid sequences of various tumor antigen-binding antibodies are known in the art, as are the light chain and heavy chain CDRs of such antibodies. See, e.g., Ling et al. (2018) Frontiers Immunol. 9:469; WO 2005/012493; US 2019/0119375; US 2013/0066055. The following are non-limiting examples of tumor antigen-binding antibodies.

### (1) Anti-Her2

In some cases, an anti-Her2 antibody comprises: a) a light chain comprising an amino acid sequence having at least 90%, at least 95%, at least 98%, at least 99%, or 100%, amino acid sequence identity to the following amino acid sequence: DIQMTQSPSSLSASVGDRVTITCRASQDVNTAVAWYQQKPGKAPKLLIYSASFLYSGVPSRF SGSRSGTDFTLTISSLQPEDFATYYCQQHYTTPPTFGQGTKVEIKRTVAAPSVFIFPPSDEQLKSGTASVVCLLNNFYP REAKVQWKVDNALQSGNSQESVTEQDSKDSTYSLSSTLTLSKADYEKHKVYACEVTHQGLSSPVTKSFNRGEC (SEQ ID NO:243); and b) a heavy chain comprising an amino acid sequence having at least 90%, at least 95%, at least 98%, at least 99%, or 100%, amino acid sequence identity to the following amino acid sequence: EVOLVESGG GLVQPGGSLRLSCAASGFNIKDTYIHWVRQAPGKGLEWVARIYPTNGYTRYADSVKGRFTISADTSKNTAYLQMNSL RAEDTAVYYCSRWGGDGFYAMDYWGQGTLVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDKKVEPKSCDKTHTCPPCPAPELL GGPSVFLFPPKPKDTLMISRTPEVTCVVVDVSHEDPEVKFNWYVDGVEVHNAKTKPREEQYNSTYRVVSVLTVLHQ DWLNGKEYKCKVSNKALPAPIEKTISKAKGQPREPQVYTLPPSREEMTKNQVSLTCLVKGFYPSDIAVEWESNGQPE NNYKTTPPVLDSDGSFFLYSKLTVDKSRWQQGNVFSCSVMHEALHNHYTQKSLSLSPGK (SEQ ID NO:244).

In some cases, an anti-Her2 antibody comprises a light chain variable region (VL) present in the light chain amino acid sequence provided above; and a heavy chain variable region (VH) present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSG VPSRFSGSRSGTDFTLTISSLQPEDFATYYCQQHYTTPPTFGQGTKVEIK (SEQ ID NO:245); and b) a VH comprising an amino acid sequence having at least 90%, at least 95%, at least 98%, at least 99%, or 100%, amino acid sequence identity to the amino acid sequence: EVOLVESGGGLVQPGGSLRLSCAASGFNIKDTYIHWVRQ APGKGLEWVARIYPTNGYTRYADSVKGRFTISADTSKNTAYLQMNSLRAEDTAVYYCSRWGGDGFYAMDYWGQGT LVTVSS (SEQ ID NO:246). In some cases, an anti-Her2 antibody comprises, in order from N-terminus to C-terminus: a) a VH comprising an amino acid sequence having at least 90%, at least 95%, at least 98%, at least 99%, or 100%, amino acid sequence identity to the amino acid sequence: EVOLVESGGGLVQPGGSLRLSCAASGFNIKDTYIHW VRQAPGKGLEWVARIYPTNGYTRYADSVKGRFTISADTSKNTAYLQMNSLRAEDTAVYYCSRWGGDGFYAMDYWG QGTLVTVSS (SEQ ID NO:246); b) a linker; and c) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSGVPSRFSGSRSGTDFTLTISSL QPEDFATYYCQQHYTTPPTFGQGTKVEIK (SEQ ID NO:245). Suitable linkers are described elsewhere herein and include, e.g., GGGGS (SEQ ID NO:130), consecutive repeats of which may appear from 2 to 10 times as a linker (e.g., 1, 2, 3, 4, 5, 6, 7, 8, 9, or 10 times).

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the VH and VL CDRs are as defined by Kabat (see, e.g., Table 1, above; and Kabat 1991). In some cases, the VH and VL CDRs are as defined by Chothia (see, e.g., Table 1, above; and Chothia 1987).

For example, an anti-Her2 antibody can comprise a VL CDR1 having the amino acid sequence RASQDVNTAVA (SEQ ID NO:247); a VL CDR2 having the amino acid sequence SASFLY (SEQ ID NO:248); a VL CDR3 having the amino acid sequence QQHYTTPP (SEQ ID NO:249); a VH CDR1 having the amino acid sequence GFNIKDY (SEQ ID NO:250); a VH CDR2 having the amino acid sequence IYPTNGYT (SEQ ID NO:251); and a VH CDR3 having the amino acid sequence SRWGGDGFYAMDY (SEQ ID NO:252).

In some cases, an anti-Her2 antibody is a scFv antibody. For example, an anti-Her2 scFv can comprise an amino acid sequence having at least 90%, at least 95%, at least 98%, at least 99%, or 100%, amino acid sequence identity to the following amino acid sequence: EVQLVESGGGLVQPGGSLRLSCAASGFNIKDTYIHWVRQAPGKGLE WVARIYPTNGYTRYADSVKGRFTISADTSKNTAYLQMNSLRAEDTAVYYCSRWGGDGFYAMDYWGQGTLVTVSSG GGGSGGGGSGGGGSDIQMTQSPSSLSASVGDRVTITCRASQDVNTAVAWYQQKPGKAPKLLIYSASFLYSGVPSRF SGSRSGTDFTLTISSLQPEDFATYYCQQHYTTPPTFGQGTKVEIK (SEQ ID NO:253).

As another example, in some cases, an anti-Her2 antibody comprises: a) a light chain variable region (VL) comprising an amino acid sequence having at least 90%, at least 95%, at least 98%, at least 99%, or 100%, amino acid sequence identity to the following amino acid sequence: DIQMTQSPSSLSASVGDRVTITCKASQDVSIGVAWYQ QKPGKAPKLLIYSASYRYTGVPSRFSGSGSGTDFTLTISSLQPEDFATYYCQQYYIYPYTFGQGTKVEIKRTVAAPSVF IFPPSDEQLKSGTASVVCLLNNFYPREAKVQWKVDNALQSGNSQESVTEQDSKDSTYSLSSTLTLSKADYEKHKVYA CEVTHQGLSSPVTKSFNRGEC (SEQ ID NO:254); and b) a heavy chain variable region (VH) comprising an amino acid sequence having at least 90%, at least 95%, at least 98%, at least 99%, or 100%, amino acid sequence identity to the following amino acid sequence: EVOLVESGGGLVQPGGSLRLSCAASGFTFTDYTMDWVRQAPGKGLEWVA DVNPNSGGSIYNQRFKGRFTLSVDRSKNTLYLQMNSLRAEDTAVYYCARNLGPSFYFDYWGQGTLVTVSSASTKGP SVFPLAPSSKSTSGGTAALGCLVKDYFPEPVTVSWNSGALTSGVHTFPAVLQSSGLYSLSSVVTVPSSSLGTQTYICN VNHKPSNTKVDKKVEPKSCDKTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPIEKTISKAKGQPREPQVYTLPPSR EEMTKNQVSLTCLVKGFYPSDIAVEWESNGQPENNYKTTPPVLDSDGSFFLYSKLTVDKSRWQQGNVFSCSVMHEA LHNHYTQKSLSLSPG (SEQ ID NO:255).

In some cases, an anti-Her2 antibody comprises a VL present in the light chain amino acid sequence provided above; and a VH present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRV TITCKASQDVSIGVAWYQQKPGKAPKLLIYSASYRYTGVPSRFSGSGSGTDFTLTISSLQPEDFATYYCQQYYIYPYTF GQGTKVEIK (SEQ ID NO:256); and b) a VH comprising an amino acid sequence having at least 90%, at least 95%, at least 98%, at least 99%, or 100%, amino acid sequence identity to the amino acid sequence: EVOLVESGGGLVQ PGGSLRLSCAASGFTFTDYTMDWVRQAPGKGLEWVADVNPNSGGSIYNQRFKGRFTLSVDRSKNTLYLQMNSLRA EDTAVYYCARNLGPSFYFDYWGQGTLVTVSS (SEQ ID NO:257).

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the VH and VL CDRs are as defined by Kabat (see, e.g., Table 1, above; and Kabat 1991). In some cases, the VH and VL CDRs are as defined by Chothia (see, e.g., Table 1, above; and Chothia 1987).

For example, an anti-HER2 antibody can comprise a VL CDR1 having the amino acid sequence KASQDVSIGVA (SEQ ID NO:258); a VL CDR2 having the amino acid sequence SASYRY (SEQ ID NO:259); a VL CDR3 having the amino acid sequence QQYYIYPY (SEQ ID NO:260); a VH CDR1 having the amino acid sequence GFTFTDYTMD (SEQ ID NO:261); a VH CDR2 having the amino acid sequence ADVNPNSGGSIYNQRFKG (SEQ ID NO: 262); and a VH CDR3 having the amino acid sequence ARNLGPSFYFDY (SEQ ID NO:263).

In some cases, an anti-Her2 antibody is a scFv. For example, in some cases, an anti-Her2 scFv comprises an amino acid sequence having at least 90%, at least 95%, at least 98%, at least 99%, or 100%, amino acid sequence identity to the following amino acid sequence: EVOLVESGGGLVQPGGSLRLSCAASGFNIKDTYIHWVRQ APGKGLEWVARIYPTNGYTRYADSVKGRFTISADTSKNTAYLQMNSLRAEDTAVYYCSRWGGDGFYAMDYWGQGT LVTVSSGGGGSGGGGSGGGGSDIQMTQSPSSLSASVGDRVTITCRASQDVNTAVAWYQQKPGKAPKLLIYSASFLY SGVPSRFSGSRSGTDFTLTISSLQPEDFATYYCQQHYTTPPTFGQGTKVEIK (SEQ ID NO:253).

### (2) Anti-CD19

Anti-CD19 antibodies are known in the art; and the VH and VL, or the VH and VL CDRs, of any anti-CD19 antibody can be used in a T-Cell-MP or T-Cell-MP-epitope conjugate. See e.g., WO 2005/012493.

In some cases, an anti-CD19 antibody includes a VL CDR1 comprising the amino acid sequence KASQSVDYDGDSYLN (SEQ ID NO:264); a VL CDR2 comprising the amino acid sequence DASNLVS (SEQ ID NO: 265); and a VL CDR3 comprising the amino acid sequence QQSTEDPWT (SEQ ID NO:266). In some cases, an anti-CD19 antibody includes a VH CDR1 comprising the amino acid sequence SYWMN (SEQ ID NO:267); a VH CDR2 comprising the amino acid sequence QIWPGDGDTNYNGKFKG (SEQ ID NO:268); and a VH CDR3 comprising the amino acid sequence RETTTVGRYYYAMDY (SEQ ID NO:269). In some cases, an anti-CD19 antibody includes a VL CDR1 comprising the amino acid sequence KASQSVDYDGDSYLN (SEQ ID NO:264); a VL CDR2 comprising the amino acid sequence DASNLVS (SEQ ID NO:265); a VL CDR3 comprising the amino acid sequence QQSTEDPWT (SEQ ID NO:266); a VH CDR1 comprising the amino acid sequence SYWMN (SEQ ID NO: 267); a VH CDR2 comprising the amino acid sequence QIWPGDGDTNYNGKFKG (SEQ ID NO:268); and a VH CDR3 comprising the amino acid sequence RETTTVGRYYYAMDY (SEQ ID NO:269).

In some cases, an anti-CD19 antibody is a scFv. For example, in some cases, an anti-CD19 scFv comprises an amino acid sequence having at least 90%, at least 95%, at least 98%, at least 99%, or 100%, amino acid sequence identity to the following amino acid sequence: DIQLTQSPASLAVSLGQRATISCKASQSVDYDGDSY LNWYQQIPGQPPKLLIYDASNLVSGIPPRFSGSGSGTDFTLNIHPVEKVDAATYHCQQSTEDPWTFGGGTKLEIKGG GGSGGGGSGGGGSQVQLQQSGAELVRPGSSVKISCKASGYAFSSYWMNWVKQRPGQGLEWIGQIWPGDGDTNY NGKFKGKATLTADESSSTAYMQLSSLASEDSAVYFCARRETTTVGRYYYAMDYWGQGTTVTVS (SEQ ID NO:270).

### (3) Anti-mesothelin

Anti-mesothelin antibodies are known in the art; and the VH and VL, or the VH and VL CDRs, of any anti-mesothelin antibody can be used in a T-Cell-MP or T-Cell-MP-epitope conjugate. See, e.g., U.S. 2019/0000944; WO 2009/045957; WO 2014/031476; U.S. Pat. No. 8,460,660; US 2013/0066055; and WO 2009/068204. In some cases, a CTP is an anti-mesothelin scFv or an anti-mesothelin nanobody.

In some cases, an anti-mesothelin antibody comprises: a) a light chain comprising an amino acid sequence having at least 90%, at least 95%, at least 98%, at least 99%, or 100%, amino acid sequence identity to the following amino acid sequence: DIALTQPASVSGSPGQSITISCTGTSSDIGGYNSVSWYQQHPGKAPKLMIYGV NNRPSGVSNRFSGSKSGNTASLTISGLQAEDEADYYCSSYDIESATPVFGGGTKLTVLGQPKAAPSVTLFPPSSEEL QANKATLVCLISDFYPGAVTVAWKGDSSPVKAGVETTTPSKQSNNKYAASSYLSLTPEQWKSHRSYSCQVTHEGST VEKTVAPTESS (SEQ ID NO:271); and

b) a heavy chain comprising an amino acid sequence having at least 90%, at least 95%, at least 98%, at least 99%, or 100%, amino acid sequence identity to the following amino acid sequence: QVELVQSGAEVKKPGES LKISCKGSGYSFTSYWIGWVRQAPGKGLEWMGIIDPGDSRTRYSPSFQGQVTISADKSISTAYLQWSSLKASDTAMY YCARGQLYGGTYMDGWGQGTLVTVSSASTKGPSVFPLAPSSKSTSGGTAALGCLVKDYFPEPVTVSWNSGALTSG VHTFPAVLQSSGLYSLSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVSVLTVLHQDWLNGKEYK CKVSNKALPAPIEKTISKAKGQPREPQVYTLPPSRDELTKNQVSLTCLVKGFYPSDIAVEWESNGQPENNYKTTPPVL DSDGSFFLYSKLTVDKSRWQQGNVFSCSVMHEALHNHYTQKSLSLSPGK (SEQ ID NO:272).

In some cases, an anti-mesothelin antibody comprises a VL present in the light chain amino acid sequence provided above; and a VH present in the heavy chain amino acid sequence provided above. For example, an anti-mesothelin antibody can comprise: a) a VL comprising an amino acid sequence having at least 90%, at least 95%, at least 98%, at least 99%, or 100%, amino acid sequence identity to the amino acid sequence: DIALTQPASV SGSPGQSITISCTGTSSDIGGYNSVSWYQQHPGKAPKLMIYGVNNRPSGVSNRFSGSKSGNTASLTISGLQAEDEAD YYCSSYDIESATPVFGGGTK (SEQ ID NO:273); and b) a VH comprising an amino acid sequence having at least 90%, at least 95%, at least 98%, at least 99%, or 100%, amino acid sequence identity to the amino acid sequence:

(SEQ ID NO: 274)
QVELVQSGAEVKKPGESLKISCKGSGYSFTSYWIGWVRQA
PGKGLEWMGIIDPGDSRTRYSPSFQGQVTISADKSISTAY
LQWSSLKASDTAMYYCARGQLYGGTYMDGWGQGTLVTVSS.

In some cases, an anti-mesothelin antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-mesothelin antibody can comprise a VL CDR1 having the amino acid sequence TGTSSDIGGYNSVS (SEQ ID NO:275); a VL CDR2 having the amino acid sequence LMIYGVNNRPS (SEQ ID NO: 276); a VL CDR3 having the amino acid sequence SSYDIESATP (SEQ ID NO:277); a VH CDR1 having the amino acid sequence GYSFTSYWIG (SEQ ID NO:278); a VH CDR2 having the amino acid sequence WMGIIDPGDSRTRYSP (SEQ ID NO:279); and a VH CDR3 having the amino acid sequence GQLYGGTYMDG (SEQ ID NO:280).

An anti-mesothelin antibody can be a scFv. As one non-limiting example, an anti-mesothelin scFv can comprise the following amino acid sequence:

(SEQ ID NO: 281)
QVQLQQSGAEVKKPGASVKVSCKASGYTFTGYYMHWVRQAPGQGLEWMGR
INPNSGGTNYAQKFQGRVTMTRDTSISTAYMELSRLRSEDTAVYYCARGR
YYGMDVWGQGTMVTVSSGGGGSGGGGSGGGGSGGGGSEIVLTQSPATLSL
SPGERATISCRASQSVSSNFAWYQQRPGQAPRLLIYDASNRATGIPPRFS
GSGSGTDFTLTISSLEPEDFAAYYCHQRSNWLYTFGQGTKVDIK,

where VH CDR1, CDR2, and CDR3 are underlined; and VL CDR1, CDR2, and CDR3 are bolded and underlined.

As one non-limiting example, an anti-mesothelin scFv can comprise the following amino acid sequence:

(SEQ ID NO: 282)
QVQLVQSGAEVKKPGASVKVSCKASGYTFTGYYMHWVRQAPGQGLEWMGW
INPNSGGTNYAQKFQGRVTMTRDTSISTAYMELSRLRSDDTAVYYCARDL
RRTVVTPRAYYGMDVWGQGTTVTVSSGGGGGGGGSGGGGSGGGGSDIQLT
QSPSTLSASVGDRVTITCQASQDISNSLNWYQQKAGKAPKLLIYDASTLE
TGVPSRFSGSGSGTDFSFTISSLQPEDIATYYCQQHDNLPLTFGQGTKVE
IK,

where VH CDR1, CDR2, and CDR3 are underlined; and VL CDR1, CDR2, and CDR3 are bolded and underlined.

In some cases, an anti-mesothelin antibody comprises: a) VL CDR1, VL CDR2, and VL CDR3 present in a light chain variable region (VL) comprising the following amino acid sequence: EIVLTQSP GTLSLSPGERATLSCRASQSVSSSYLAWYQQKPGQAPRLLIYGASSRATGIPDRFSGSGSGTDFTLTISRLEPEDFAV YYCQQYGSSPIFTFGPGTKVDIK (SEQ ID NO:283); and b) VH CDR1, CDR2, and CDR3 present in a heavy chain variable region (VH) comprising the following amino acid sequence: QMQLVESGGGVVQPGRSLRLSCTASGFTFS NNGMHWVRQAPGKGLEWVAVIWFDGMNKFYVDSVKGRFTISRDNSKNTLYLEMNSLRAEDTAIYYCAREGDGSGIY YYYGMDVWGQGTTVTVSS (SEQ ID NO:284). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). See, e.g., BMS6A5.

In some cases, an anti-mesothelin antibody comprises: a) a VL region comprising an amino acid sequence having at least 90%, at least 95%, at least 98%, at least 99%, or 100%, amino acid sequence identity to the following amino acid sequence: EIVLTQSPGTLSLSPGERATLSCRASQSVSSSYLAWYQQKPGQAPRLLIYGASSRATGIPDRF SGSGSGTDFTLTISRLEPEDFAVYYCQQYGSSPIFTFGPGTKVDIK (SEQ ID NO:283); and b) a VH region comprising an amino acid sequence having at least 90%, at least 95%, at least 98%, at least 99%, or 100%, amino acid sequence identity to the following amino acid sequence: QMQLVESGGGVVQPGRSLRLSCTASGFTFSNNGMH WVRQAPGKGLEWVAVIWFDGMNKFYVDSVKGRFTISRDNSKNTLYLEMNSLRAEDTAIYYCAREGDGSGIYYYYGM DVWGQGTTVTVSS (SEQ ID NO:284).

In some cases, an anti-mesothelin antibody is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QMQLVESGGGVVQPGRSLRLSCTASG FTFSNNGMHWVRQAPGKGLEWVAVIWFDGMNKFYVDSVKGRFTISRDNSKNTLYLEMNSLRAEDTAIYYCAREGDG SGIYYYYGMDVWGQGTTVTVSS (SEQ ID NO:284); b) a peptide linker; and c) a VL region comprising an amino acid sequence having at least 90%, at least 95%, at least 98%, at least 99%, or 100%, amino acid sequence identity to the following amino acid sequence: EIVLTQSPGTLSLSPGERATLSCRASQSVSSSYLAWYQQKPGQAPRLLI YGASSRATGIPDRFSGSGSGTDFTLTISRLEPEDFAVYYCQQYGSSPIFTFGPGTKVDIK (SEQ ID NO:283). In some cases, the peptide linker comprises the amino acid sequence GGGGS (SEQ ID NO:130), which may be repeated 2-10 times as a linker (e.g., 1, 2, 3, 4, 5, 6, 7, 8, 9, or 10 times). In some cases, the peptide linker comprises the amino acid sequence GGGGSGGGGSGGGGS (SEQ ID NO:285) and has a length of 15 amino acids.

In some cases, an anti-mesothelin antibody is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: EIVLTQSPGTLSLSPGERATLSCRASQS VSSSYLAWYQQKPGQAPRLLIYGASSRATGIPDRFSGSGSGTDFTLTISRLEPEDFAVYYCQQYGSSPIFTFGPGTKV DIK (SEQ ID NO:283); b) a peptide linker; and c) a VH region comprising an amino acid sequence having at least 90%, at least 95%, at least 98%, at least 99%, or 100%, amino acid sequence identity to the following amino acid sequence: QMQLVESGGGVVQPGRSLRLSCTASGFTFSNNGMHWVRQAPGKGLEWVAVIWFDGMNKFYVDSVK GRFTISRDNSKNTLYLEMNSLRAEDTAIYYCAREGDGSGIYYYYGMDVWGQGTTVTVSS (SEQ ID NO:284). In some cases, the peptide linker comprises the amino acid sequence GGGGS (SEQ ID NO:130), which may be repeated 2-10 times as a linker (e.g., 1, 2, 3, 4, 5, 6, 7, 8, 9, or 10 times). In some cases, the peptide linker comprises the amino acid sequence GGGGSGGGGSGGGGS (SEQ ID NO:285) and has a length of 15 amino acids.

In some cases, an anti-mesothelin antibody comprises: a) VL CDR1, VL CDR2, and VL CDR3 present in a light chain variable region (VL) comprising the following amino acid sequence: DIELTQSPAIMSASPGEKVTMTCSA SSSVSYMHWYQQKSGTSPKRWIYDTSKLASGVPGRFSGSGSGNSYSLTISSVEAEDDATYYCQQWSKHPLTFGSG TKVEIK (SEQ ID NO:286); and b) VH CDR1, CDR2, and CDR3 present in a heavy chain variable region (VH) comprising the following amino acid sequence: QVQLQQSGPELEKPGASVKISCKASGYSFTGYTMNWVKQSH GKSLEWIGLITPYNGASSYNQKFRGKATLTVDKSSSTAYMDLLSLTSEDSAVYFCARGGYDGRGFDYWGSGTPVTV SS (SEQ ID NO:287). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). See, e.g., Amatuximab.

In some cases, an anti-mesothelin antibody comprises: a) a VL region comprising an amino acid sequence having at least 90%, at least 95%, at least 98%, at least 99%, or 100%, amino acid sequence identity to the following amino acid sequence: DIELTQSPAIMSASPGEKVTMTCSASSSVSYMHWYQQKSGTSPKRWIYDTSKLASGVPGRFS GSGSGNSYSLTISSVEAEDDATYYCQQWSKHPLTFGSGTKVEIK (SEQ ID NO:286); and b) a VH region comprising an amino acid sequence having at least 90%, at least 95%, at least 98%, at least 99%, or 100%, amino acid sequence identity to the following amino acid sequence: QVQLQQSGPELEKPGASVKISCKASGYSFTGYTMNWVK QSHGKSLEWIGLITPYNGASSYNQKFRGKATLTVDKSSSTAYMDLLSLTSEDSAVYFCARGGYDGRGFDYWGSGTP VTVSS (SEQ ID NO:287).

In some cases, an anti-mesothelin antibody is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIELTQSPAIMSASPGEKVTMTCSASSS VSYMHWYQQKSGTSPKRWIYDTSKLASGVPGRFSGSGSGNSYSLTISSVEAEDDATYYCQQWSKHPLTFGSGTKV EIK (SEQ ID NO:286); b) a peptide linker; and c) a VH region comprising an amino acid sequence having at least 90%, at least 95%, at least 98%, at least 99%, or 100%, amino acid sequence identity to the following amino acid sequence: QVQLQQSGPELEKPGASVKISCKASGYSFTGYTMNWVKQSHGKSLEWIGLITPYNGASSYNQKFRGKA TLTVDKSSSTAYMDLLSLTSEDSAVYFCARGGYDGRGFDYWGSGTPVTVSS (SEQ ID NO:287). In some cases, the peptide linker comprises the amino acid sequence GGGGS (SEQ ID NO:130), which may be repeated 2-10 times as a linker (e.g., 1, 2, 3, 4, 5, 6, 7, 8, 9, or 10 times). In some cases, the peptide linker comprises the amino acid sequence GGGGSGGGGSGGGGS (SEQ ID NO:285) and has a length of 15 amino acids.

In some cases, an anti-mesothelin antibody is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLQQSGPELEKPGASVKISCKASG YSFTGYTMNWVKQSHGKSLEWIGLITPYNGASSYNQKFRGKATLTVDKSSSTAYMDLLSLTSEDSAVYFCARGGYD GRGFDYWGSGTPVTVSS (SEQ ID NO:287); b) a peptide linker; and c) a VL region comprising an amino acid sequence having at least 90%, at least 95%, at least 98%, at least 99%, or 100%, amino acid sequence identity to the following amino acid sequence: DIELTQSPAIMSASPGEKVTMTCSASSSVSYMHWYQQKSGTSPKRWIYDTSK LASGVPGRFSGSGSGNSYSLTISSVEAEDDATYYCQQWSKHPLTFGSGTKVEIK (SEQ ID NO:286). In some cases, the peptide linker comprises the amino acid sequence GGGGS (SEQ ID NO: 130), which may be repeated 2-10 times as a linker (e.g., 1, 2, 3, 4, 5, 6, 7, 8, 9, or 10 times). In some cases, the peptide linker comprises the amino acid sequence GGGGSGGGGSGGGGS (SEQ ID NO:285) and has a length of 15 amino acids.

In some cases, an anti-mesothelin antibody comprises: a) VL CDR1, VL CDR2, and VL CDR3 present in a light chain variable region (VL) comprising the following amino acid sequence: DIALTQPASVSGSPGQSITISCT GTSSDIGGYNSVSWYQQHPGKAPKLMIYGVNNRPSGVSNRFSGSKSGNTASLTISGLQAEDEADYYCSSYDIESATP VFGGGTKLTVLG (SEQ ID NO:288); and b) VH CDR1, CDR2, and CDR3 present in a heavy chain variable region (VH) comprising the following amino acid sequence: QVELVQSGAEVKKPGESLKISCKGSGYSFTSYWIGWVRQAP GKGLEWMGIIDPGDSRTRYSPSFQGQVTISADKSISTAYLQWSSLKASDTAMYYCARGQLYGGTYMDGWGQGTLVT VSS (SEQ ID NO:274). In some cases, the VH and VL CDRs are as defined by Kabat (see, e.g., Table 1, above; and Kabat 1991). In some cases, the VH and VL CDRs are as defined by Chothia (see, e.g., Table 1, above; and Chothia 1987).

In some cases, an anti-mesothelin antibody comprises: a) a VL region comprising an amino acid sequence having at least 90%, at least 95%, at least 98%, at least 99%, or 100%, amino acid sequence identity to the following amino acid sequence: DIALTQPASVSGSPGQSITISCTGTSSDIGGYNSVSWYQQHPGKAPKLMIYGVN NRPSGVSNRFSGSKSGNTASLTISGLQAEDEADYYCSSYDIESATPVFGGGTKLTVLG (SEQ ID NO:288); and b) a VH region comprising an amino acid sequence having at least 90%, at least 95%, at least 98%, at least 99%, or 100%, amino acid sequence identity to the following amino acid sequence: QVELVQSGAEVKKPGESLKISCKGSG YSFTSYWIGWVRQAPGKGLEWMGIIDPGDSRTRYSPSFQGQVTISADKSISTAYLQWSSLKASDTAMYYCARGQLY GGTYMDGWGQGTLVTVSS (SEQ ID NO:274).

In some cases, an anti-mesothelin antibody is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIALTQPASVSGSPGQSITISCTGTSSDI GGYNSVSWYQQHPGKAPKLMIYGVNNRPSGVSNRFSGSKSGNTASLTISGLQAEDEADYYCSSYDIESATPVFGGG TKLTVLG (SEQ ID NO:288); b) a peptide linker; and c) a VH region comprising an amino acid sequence having at least 90%, at least 95%, at least 98%, at least 99%, or 100%, amino acid sequence identity to the following amino acid sequence: QVELVQSGAEVKKPGESLKISCKGSGYSFTSYWIGWVRQAPGKGLEWMGIIDPGDSRTRYSPSFQ GQVTISADKSISTAYLQWSSLKASDTAMYYCARGQLYGGTYMDGWGQGTLVTVSS (SEQ ID NO:274). In some cases, the peptide linker comprises the amino acid sequence GGGGS (SEQ ID NO: 130), which may be repeated 2-10 times as a linker (e.g., 1, 2, 3, 4, 5, 6, 7, 8, 9, or 10 times). In some cases, the peptide linker comprises the amino acid sequence GGGGSGGGGSGGGGS (SEQ ID NO:285) and has a length of 15 amino acids.

In some cases, an anti-mesothelin antibody is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVELVQSGAEVKKPGESLKISCKGSGY SFTSYWIGWVRQAPGKGLEWMGIIDPGDSRTRYSPSFQGQVTISADKSISTAYLQWSSLKASDTAMYYCARGQLYG GTYMDGWGQGTLVTVSS (SEQ ID NO:274); b) a peptide linker; and c) a VL region comprising an amino acid sequence having at least 90%, at least 95%, at least 98%, at least 99%, or 100%, amino acid sequence identity to the following amino acid sequence: DIALTQPASVSGSPGQSITISCTGTSSDIGGYNSVSWYQQHPGKAPKLMIYGV NNRPSGVSNRFSGSK SGNTASLTISGLQAEDEADYYCSSYDIESATPVFGGGTKLTVLG (SEQ ID NO:288). In some cases, the peptide linker comprises the amino acid sequence GGGGS (SEQ ID NO:130), which may be repeated 2-10 times as a linker (e.g., 1, 2, 3, 4, 5, 6, 7, 8, 9, or 10 times). In some cases, the peptide linker comprises the amino acid sequence GGGGSGGGGSGGGGS (SEQ ID NO:285) and has a length of 15 amino acids.

In some cases, an anti-mesothelin antibody comprises: a) VL CDR1, VL CDR2, and VL CDR3 present in a light chain variable region (VL) comprising the following amino acid sequence: DIQMTQSPSSLSASVGDRVTIT CSASSSVSYMHWYQQKSGKAPKLLIYDTSKLASGVPSRFSGSGSGTDFTLTISSLQPEDFATYYCQQWSKHPLTFG QGTKLEIK (SEQ ID NO:289); and b) VH CDR1, CDR2, and CDR3 present in a heavy chain variable region (VH) comprising the following amino acid sequence: QVQLVQSGAEVKKPGASVKVSCKASGYSFTGYTMNWVRQAPGQ GLEWMGLITPYNGASSYNQKFRGKATMTVDTSTSTVYMELSSLRSEDTAVYYCARGGYDGRGFDYWGQGTLVTVS S (SEQ ID NO:290). In some cases, the VH and VL CDRs are as defined by Kabat (see, e.g., Table 1, above; and Kabat 1991). In some cases, the VH and VL CDRs are as defined by Chothia (see, e.g., Table 1, above; and Chothia 1987). See, e.g., RG7787.

In some cases, an anti-mesothelin antibody comprises: a) a VL region comprising an amino acid sequence having at least 90%, at least 95%, at least 98%, at least 99%, or 100%, amino acid sequence identity to the following amino acid sequence: DIQMTQSPSSLSASVGDRVTITCSASSSVSYMHWYQQKSGKAPKLLIY DTSKLASGVPSRFSGSGSGTDFTLTISSLQPEDFATYYCQQWSKHPLTFGQGTKLEIK (SEQ ID NO:289); and b) a VH region comprising an amino acid sequence having at least 90%, at least 95%, at least 98%, at least 99%, or 100%, amino acid sequence identity to the following amino acid sequence: QVQLVQSGAEVKKPGASVKVSCKASG YSFTGYTMNWVRQAPGQGLEWMGLITPYNGASSYNQKFRGKATMTVDTSTSTVYMELSSLRSEDTAVYYCARGGY DGRGFDYWGQGTLVTVSS (SEQ ID NO:290).

In some cases, an anti-mesothelin antibody is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIQMTQSPSSLSASVGDRVTITCSASSS VSYMHWYQQKSGKAPKLLIYDTSKLASGVPSRFSGSGSGTDFTLTISSLQPEDFATYYCQQWSKHPLTFGQGTKLEI K (SEQ ID NO:289); b) a peptide linker; and c) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYSFTGYTMNWVRQAPGQGLEWMGLITPYNGASSYNQKFRGK ATMTVDTSTSTVYMELSSLRSEDTAVYYCARGGYDGRGFDYWGQGTLVTVSS (SEQ ID NO:290). In some cases, the peptide linker comprises the amino acid sequence (GGGGS (SEQ ID NO: 130), which may be repeated 2-10 times as a linker (e.g., 1, 2, 3, 4, 5, 6, 7, 8, 9, or 10 times). In some cases, the peptide linker comprises the amino acid sequence GGGGSGGGGSGGGGS (SEQ ID NO:285) and has a length of 15 amino acids.

In some cases, an anti-mesothelin antibody is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLVQSGAEVKKPGASVKVSCKASG YSFTGYTMNWVRQAPGQGLEWMGLITPYNGASSYNQKFRGKATMTVDTSTSTVYMELSSLRSEDTAVYYCARGGY DGRGFDYWGQGTLVTVSS (SEQ ID NO:290); b) a peptide linker; and c) a VL region comprising an amino acid sequence having at least 90%, at least 95%, at least 98%, at least 99%, or 100%, amino acid sequence identity to the following amino acid sequence: DIQMTQSPSSLSASVGDRVTITCSASSSVSYMHWYQQKSGKAPKLLIYDTSKLA SGVPSRFSGSGSGTDFTLTISSLQPEDFATYYCQQWSKHPLTFGQGTKLEIK (SEQ ID NO:289/). In some cases, the peptide linker comprises the amino acid sequence (GGGGS) n (SEQ ID NO:130), GGGGS (SEQ ID NO:130), which may be repeated 2-10 times as a linker sequence (e.g., 1, 2, 3, 4, 5, 6, 7, 8, 9, or 10 times). In some cases, the peptide linker comprises the amino acid sequence GGGGSGGGGSGGGGS (SEQ ID NO:285) and has a length of 15 amino acids.

In some cases, an anti-mesothelin scFv comprises the following amino acid sequence:

(SEQ ID NO: 291)
QMQLVESGGGVVQPGRSLRLSCTASGFTFSNNGMHWVRQAPGKGL
EWVAVIWFDGMNKFYVDSVKGRFTISRDNSKNTLYLEMNSLRAED
TAIYYCAREGDGSGIYYYYGMDVWGQGTTVTVSSGGGGSGGGGSG
GGGSEIVLTQSPGTLSLSPGERATLSCRASQSVSSSYLAWYQQKP
GQAPRLLIYGASSRATGIPDRFSGSGSGTDFTLTISRLEPEDFAV
YYCQQYGSSPIFTFGPGTKVDIK,

where the VH sequence is italicized, the (GGGGS)3 linker is bolded and underlined, and the VL sequence is underlined.

In some cases, an anti-mesothelin scFv comprises the following amino acid sequence

(SEQ ID NO: 292
EIVLTQSPGTLSLSPGERATLSCRASQSVSSSYLAWYQQKPGQAP
RLLIYGASSRATGIPDRESGSGSGTDFTLTISRLEPEDFAVYYCQ
QYGSSPIFTFGPGTKVDIKGGGGSGGGGSGGGGSQMQLVESGGGV
VQPGRSLRLSCTASGFTFSNNGMHWVRQAPGKGLEWVAVIWFDGM
NKFYVDSVKGRFTISRDNSKNTLYLEMNSLRAEDTAIYYCAREGD
GSGIYYYYGMDVWGQGTTVTVSS,

where the VL sequence is underlined, the (GGGGS)3 linker is bolded and underlined, and the VL sequence is italicized.

In some cases, an anti-mesothelin scFv comprises the following amino acid sequence:

(SEQ ID NO: 293)
QVQLQQSGPELEKPGASVKISCKASGYSFTGYTMNWVKQSHGKSL
EWIGLITPYNGASSYNQKFRGKATLTVDKSSSTAYMDLLSLTSED
SAVYFCARGGYDGRGFDYWGSGTPVTVSSGGGGSGGGGSGGGGSD
IELTQSPAIMSASPGEKVTMTCSASSSVSYMHWYQQKSGTSPKRW
IYDTSKLASGVPGRFSGSGSGNSYSLTISSVEAEDDATYYCQQWS
KHPLTFGSGTKVEIK,

where the VH sequence is italicized, the (GGGGS)3 linker is bolded and underlined, and the VL sequence is underlined.

In some cases, an anti-mesothelin scFv comprises the following amino acid sequence:

(SEQ ID NO: 294)
DIELTQSPAIMSASPGEKVTMTCSASSSVSYMHWYQQKSGTSPKR
WIYDTSKLASGVPGRFSGSGSGNSYSLTISSVEAEDDATYYCQQW
SKHPLTFGSGTKVEIKGGGGSGGGGSGGGGSQVQLQQSGPELEKP
GASVKISCKASGYSFTGYTMNWVKQSHGKSLEWIGLITPYNGASS
YNQKFRGKATLTVDKSSSTAYMDLLSLTSEDSAVYFCARGGYDGR
GFDYWGSGTPVTVSS,

where the VL sequence is underlined, the (GGGGS)3 linker is bolded and underlined, and the VL sequence is italicized.

In some cases, an anti-mesothelin scFv comprises the following amino acid sequence:

(SEQ ID NO: 295)
QVELVQSGAEVKKPGESLKISCKGSGYSFTSYWIGWVRQAPGKGL
EWMGIIDPGDSRTRYSPSFQGQVTISADKSISTAYLQWSSLKASD
TAMYYCARGQLYGGTYMDGWGQGTLVTVSSGGGGSGGGGSGGGGS
DIALTQPASVSGSPGQSITISCTGTSSDIGGYNSVSWYQQHPGKA
PKLMIYGVNNRPSGVSNRFSGSKSGNTASLTISGLQAEDEADYYC
SSYDIESATPVFGGGTKLTVLG,

where the VH sequence is italicized, the (GGGGS)3 linker is bolded and underlined, and the VL sequence is underlined.

In some cases, an anti-mesothelin scFv comprises the following amino acid sequence:

(SEQ ID NO: 296)
DIALTQPASVSGSPGQSITISCTGTSSDIGGYNSVSWYQQHPGKA
PKLMIYGVNNRPSGVSNRFSGSKSGNTASLTISGLQAEDEADYYC
SSYDIESATPVFGGGTKLTVLGGGGGSGGGGSGGGGSQVELVQSG
AEVKKPGESLKISCKGSGYSFTSYWIGWVRQAPGKGLEWMGIIDP
GDSRTRYSPSFQGQVTISADKSISTAYLQWSSLKASDTAMYYCAR
GQLYGGTYMDGWGQGTLVTVSS,

where the VL sequence is underlined, the (GGGGS)3 linker is bolded and underlined, and the VL sequence is italicized.

In some cases, an anti-mesothelin scFv comprises the following amino acid sequence:

(SEQ ID NO: 297)
QVQLVQSGAEVKKPGASVKVSCKASGYSFTGYTMNWVRQAPGQGL
EWMGLITPYNGASSYNQKFRGKATMTVDTSTSTVYMELSSLRSED
TAVYYCARGGYDGRGFDYWGQGTLVTVSSGGGGSGGGGSGGGGSD
IQMTQSPSSLSASVGDRVTITCSASSSVSYMHWYQQKSGKAPKLL
IYDTSKLASGVPSRFSGSGSGTDFTLTISSLQPEDFATYYCQQWS
KHPLTFGQGTKLEIK,

where the VH sequence is italicized, the (GGGGS)3 linker is bolded and underlined, and the VL sequence is underlined.

In some cases, an anti-mesothelin scFv comprises the following amino acid sequence:

(SEQ ID NO: 298)
DIQMTQSPSSLSASVGDRVTITCSASSSVSYMHWYQQKSGKAPKL
LIYDTSKLASGVPSRFSGSGSGTDFTLTISSLQPEDFATYYCQQW
SKHPLTFGQGTKLEIKGGGGSGGGGSGGGGSQVQLVQSGAEVKKP
GASVKVSCKASGYSFTGYTMNWVRQAPGQGLEWMGLITPYNGASS
YNQKFRGKATMTVDTSTSTVYMELSSLRSEDTAVYYCARGGYDGR
GFDYWGQGTLVTVSS,

where the VL sequence is underlined, the (GGGGS)3 (SEQ ID NO:285) linker is bolded and underlined, and the VL sequence is italicized.

### (4) Anti-PSMA

Prostate-specific membrane antigen (PSMA) (also known as folate hydrolase 1 (FOLH1); membrane glutamate carboxypeptidase, and N-Acetylated-Alpha-Linked Acidic Dipeptidase 1) that is up-regulated in cancerous cells in the prostate and is used as a diagnostic and prognostic indicator of prostate cancer.

Anti-PSMA antibodies are known in the art; and the VH and VL, or the VH and VL CDRs, of any anti-PSMA antibody can be used in a T-Cell-MP or T-Cell-MP-epitope conjugate. See, e.g., U.S. Pat. No. 10,179,819 and U.S. Patent Publication No. 2021/0277141.

### (5) Anti-CD22

CD22 (also known as B-Lymphocyte Cell Adhesion Molecule, Sialic Acid-Binding Ig-Like Lectin 2, or SIGLEC2) is a sialic acid-binding adhesion molecule largely restricted to the B cell lineage and expressed on most B-lineage malignancies.

Anti-CD22 antibodies are known in the art; and the VH and VL, or the VH and VL CDRs, of any anti-CD22 antibody can be used in a T-Cell-MP or T-Cell-MP-epitope conjugate. See, e.g., Xiao et al. (2009) Mabs 1:297 (describing the fully human anti-CD22 m971 scFv); and U.S. Patent Publication No. 2020/0147134. Examples of anti-CD22 antibodies include epratuzumab and inotuzumab. See, e.g., Lenoard et al. (2007) Oncogene 26:3704 and U.S. Pat. No. 5,789,554 (describing epratuzumab); and DiJoseph et al. (2007) Leukemia 21:2240 (describing inotuzumab).

For example, an anti-CD22 antibody can comprise: i) a heavy chain variable region (VH) CDR1 having the amino acid sequence: GDSVSSNSAA (SEQ ID NO:299); ii) a VH CDR2 having the amino acid sequence: TYYRSKWYN (SEQ ID NO:300); iii) a VH CDR3 having the amino acid sequence: AREVTGDLEDAFDI (SEQ ID NO: 301); iv) a light chain variable region (VL) CDR1 having the amino acid sequence: QTIWSY (SEQ ID NO:302); v) a VL CDR2 having the amino acid sequence: AAS (Ala-Ala-Ser); and vi) a VL CDR3 having the amino acid sequence: QQSYSIPQT (SEQ ID NO:303).

### (6) Anti-TROP-2

Trophoblast cell surface antigen 2 (Trop-2) (also known as epithelial glycoprotein-1, gastrointestinal tumor-associated antigen GA733-1, membrane component chromosome 1 surface marker-1, and tumor-associated calcium signal transducer-2) is a transmembrane glycoprotein that is upregulated in numerous cancer types, and is the protein product of the TACSTD2 gene.

In some cases, a CTP is an anti-TROP-2 scFv or an anti-TROP-2 nanobody.

Anti-TROP-2 antibodies are known in the art; and the VH and VL, or the VH and VL CDRs, of any anti-TROP-2 antibody can be used in a T-Cell-MP or T-Cell-MP-epitope conjugate. See, e.g., U.S. Pat. No. 7,238,785). In some cases, an anti-TROP-2 antibody comprises: i) light chain CDR sequences CDR1 (KASQDVSIAVA; SEQ ID NO: 304); CDR2 (SASYRYT; SEQ ID NO:305); and CDR3 (QQHYITPLT; SEQ ID NO:306); and ii) heavy chain CDR sequences CDR1 (NYGMN; SEQ ID NO:307); CDR2 (WINTYTGEPTYTDDFKG; SEQ ID NO:308); and CDR3 (GGFGSSYWYFDV; SEQ ID NO:309).

In some cases, an anti-TROP-2 antibody comprises: i) heavy chain CDR sequences CDR1 (TAGMQ; SEQ ID NO: 310); CDR2 (WINTHSGVPKYAEDFKG; (SEQ ID NO:311); and CDR3 (SGFGSSYWYFDV; SEQ ID NO:312); and ii) light chain CDR sequences CDR1 (KASQDVSTAVA; SEQ ID NO:313); CDR2 (SASYRYT; SEQ ID NO:305); and CDR3 (QQHYITPLT; SEQ ID NO:306).

In some cases, an anti-TROP2 antibody comprises: a) VL CDR1, VL CDR2, and VL CDR3 present in a light chain variable region (VL) comprising the following amino acid sequence: DIQLTQSPSSLSASVGDRVSITCKAS QDVSIAVAWYQQKPGKAPKLLIYSASYRYTGVPDRFSGSGSGTDFTLTISSLQPEDFAVYYCQQHYITPLTFGAGTKV EIK (SEQ ID NO:314); and b) VH CDR1, CDR2, and CDR3 present in a heavy chain variable region (VH) comprising the following amino acid sequence: QVQLQQSGSELKKPGASVKVSCKASGYTFTNYGMNWVKQAPGQGLKWMGWI NTYTGEPTYTDDFKGRFAFSLDTSVSTAYLQISSLKADDTAVYFCARGGFGSSYWYFDVWGQGSLVTVSS (SEQ ID NO: 315). In some cases, the VH and VL CDRs are as defined by Kabat (see, e.g., Table 1, above; and Kabat 1991). In some cases, the VH and VL CDRs are as defined by Chothia (see, e.g., Table 1, above; and Chothia 1987).

In some cases, an anti-TROP-2 antibody comprises: a) a VL region comprising an amino acid sequence having at least 90%, at least 95%, at least 98%, at least 99%, or 100%, amino acid sequence identity to the following amino acid sequence: DIQLTQSPSSLSASVGDRVSITCKASQDVSIAVAWYQQKPGKAPKLLIYSASYRYTGVPD RFSGSGSGTDFTLTISSLQPEDFAVYYCQQHYITPLTFGAGTKVEIK (SEQ ID NO:314); and b) a VH region comprising an amino acid sequence having at least 90%, at least 95%, at least 98%, at least 99%, or 100%, amino acid sequence identity to the following amino acid sequence: QVQLQQSGSELKKPGASVKVSCKASGYTFTNYG MNWVKQAPGQGLKWMGW INTYTGEPTYTDDFKGRFAFSLDTSVSTAYLQISSLKADDTAVYFCARGGFGS SYWYFDVWGQGSLVTVSS (SEQ ID NO:315).

In some cases, an anti-TROP-2 antibody is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIQLTQSPSSLSASVGDRVSITCKASQD VSIAVAWYQQKPGKAPKLLIYSASYRYTGVPDRFSGSGSGTDFTLTISSLQPEDFAVYYCQQHYITPLTFGAGTKVEIK (SEQ ID NO:314); b) a peptide linker; and c) a VH region comprising an amino acid sequence having at least 90%, at least 95%, at least 98%, at least 99%, or 100%, amino acid sequence identity to the following amino acid sequence: QVQLQQSGSELKKPGASVKVSCKASGYTFTNYGMNWVKQAPGQGLKWMGWINTYTGEPTYTDDFKG RFAFSLDTSVSTAYLQISSLKADDTAVYFCARGGFGSSYWYFDVWGQGSLVTVSS (SEQ ID NO:315). In some cases, the peptide linker comprises the amino acid sequence GGGGS (SEQ ID NO:130), which may be repeated 2-10 times as the linker (e.g., 1, 2, 3, 4, 5, 6, 7, 8, 9, or 10 times). In some cases, the peptide linker comprises the amino acid sequence GGGGSGGGGSGGGGS (SEQ ID NO:285) and has a length of 15 amino acids.

In some cases, an anti-TROP-2 antibody is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLQQSGSELKKPGASVKVSCKASG YTFTNYGMNWVKQAPGQGLKWMGWINTYTGEPTYTDDFKGRFAFSLDTSVSTAYLQISSLKADDTAVYFCARGGFG SSYWYFDVWGQGSLVTVSS (SEQ ID NO:315); b) a peptide linker; and c) a VL region comprising an amino acid sequence having at least 90%, at least 95%, at least 98%, at least 99%, or 100%, amino acid sequence identity to the following amino acid sequence: DIQLTQSPSSLSASVGDRVSITCKASQDVSIAVAWYQQKPGKAPKLLIYS ASYRYTGVPDRFSGSGSGTDFTLTISSLQPEDFAVYYCQQHYITPLTFGAGTKVEIK (SEQ ID NO:314). In some cases, the peptide linker comprises the amino acid sequence GGGGS (SEQ ID NO:130), which may be repeated 2-10 times as a linker sequence (e.g., 1, 2, 3, 4, 5, 6, 7, 8, 9, or 10 times). In some cases, the peptide linker comprises the amino acid sequence GGGGSGGGGSGGGGS (SEQ ID NO:285) and has a length of 15 amino acids.

In some cases, an anti-TROP2 antibody comprises: a) VL CDR1, VL CDR2, and VL CDR3 present in a light chain variable region (VL) comprising the following amino acid sequence: DIQMTQSP SSLSASVGDRVTITCK ASQDVSTAVAWYQQKPGKAPKLLIYSASYRYTGVPSRFSGSGSGTDFTLTISSLQPEDFAVYYCQQHYITPLTFGQG TKLEIK (SEQ ID NO:316); and b) VH CDR1, CDR2, and CDR3 present in a heavy chain variable region (VH) comprising the following amino acid sequence: QVQLVQSGAEVKKPGASVKVSCKASGYTFTTAGMQWVRQAPGQ GLEWMGWINTHSGVPKYAEDFKGRVTISADTSTSTAYLQLSSLKSEDTAVYYCARSGFGSSYWYFDVWGQGTLVTV SS (SEQ ID NO:317). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

In some cases, an anti-TROP-2 antibody comprises: a) a VL region comprising an amino acid sequence having at least 90%, at least 95%, at least 98%, at least 99%, or 100%, amino acid sequence identity to the following amino acid sequence: DIQMTQSPSSLSASVGDRVTITCKASQDVSTAVAWYQQKPGKAPKLLIYSASYRYTGVPSRF SGSGSGTDFTLTISSLQPEDFAVYYCQQHYITPLTFGQGTKLEIK (SEQ ID NO:316); and b) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYTFTTAGMQWVR QAPGQGLEWMGWINTHSGVPKYAEDFKGRVTISADTSTSTAYLQLSSLKSEDTAVYYCARSGFGSSYWYFDVWGQ GTLVTVSS (SEQ ID NO:317).

In some cases, an anti-TROP-2 antibody is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIQMTQSPSSLSASVGDRVTITCKASQ DVSTAVAWYQQKPGKAPKLLIYSASYRYTGVPSRFSGSGSGTDFTLTISSLQPEDFAVYYCQQHYITPLTFGQGTKLE IK (SEQ ID NO:316); b) a peptide linker; and c) a VH region comprising an amino acid sequence having at least 90%, at least 95%, at least 98%, at least 99%, or 100%, amino acid sequence identity to the following amino acid sequence: QVQLVQSGA EVKKPGASVKVSCKASGYTFTTAGMQWVRQAPGQGLEWMGWINTHSGVPKYAEDFKGRVTISADTSTSTAYLQLS SLKSEDTAVYYCARSGFGSSYWYFDVWGQGTLVTVSS (SEQ ID NO:317). In some cases, the peptide linker comprises the amino acid sequence GGGGS (SEQ ID NO: 130), which may be repeated 2-10 times as a linker sequence (e.g., 1, 2, 3, 4, 5, 6, 7, 8, 9, or 10 times). In some cases, the peptide linker comprises the amino acid sequence GGGGSGGGGSGGGGS (SEQ ID NO:285) and has a length of 15 amino acids.

In some cases, an anti-TROP-2 antibody is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLVQSGAEVKKPGASVKVSCKASG YTFTTAGMQWVRQAPGQGLEWMGWINTHSGVPKYAEDFKGRVTISADTSTSTAYLQLSSLKSEDTAVYYCARSGFG SSYWYFDVWGQGTLVTVSS (SEQ ID NO:317); b) a peptide linker; and c) a VL region comprising an amino acid sequence having at least 90%, at least 95%, at least 98%, at least 99%, or 100%, amino acid sequence identity to the following amino acid sequence: DIQMTQSPSSLSASVGDRVTITCKASQDVSTAVAWYQQKPGKAPKLLIYSASYR YTGVPSRFSGSGSGTDFTLTISSLQPEDFAVYYCQQHYITPLTFGQGTKLEIK (SEQ ID NO:316). In some cases, the peptide linker comprises the amino acid sequence GGGGS (SEQ ID NO: 130), which may be repeated 2-10 times as a linker sequence (e.g., 1, 2, 3, 4, 5, 6, 7, 8, 9, or 10 times). In some cases, the peptide linker comprises the amino acid sequence GGGGSGGGGSGGGGS (SEQ ID NO:285) and has a length of 15 amino acids.

### (7) Anti-BCMA

Anti-BCMA (B-cell maturation antigen) antibodies are known in the art; and the VH and VL, or the VH and VL CDRs, of any anti-BCMA antibody can be used in a T-Cell-MP or T-Cell-MP-epitope conjugate. See, e.g., WO 2014/089335; US 2019/0153061; and WO 2017/093942.

In some cases, an anti-BCMA antibody comprises: a) a light chain comprising an amino acid sequence having at least 90%, at least 95%, at least 98%, at least 99%, or 100%, amino acid sequence identity to the following amino acid sequence: QSVLTQPPSASGTPGQRVTISCSGSSSNIGSNTVNWYQQLPGTAPKLLIFNYHQRPSGVPDR FSGSKSGSSASLAISGLQSEDEADYYCAAWDDSLNGWVFGGGTKLTVLGQPKAAPSVTLFPPSSEELQANKATLVCL ISDFYPGAVTVAWKADSSPVKAGVETTTPDSKQSNNKYAASSYLSLTPEQWKSHRSYSCQVTHEGSTVEKTVAPTE CS (SEQ ID NO:318); and b) a heavy chain comprising an amino acid sequence having at least 90%, at least 95%, at least 98%, at least 99%, or 100%, amino acid sequence identity to the following amino acid sequence: EVOLVES GGGLVKPGGSLRLSCAASGFTFGDYALSWFRQAPGKGLEWVGVSRSKAYGGTTDYAASVKGRFTISRDDSKSTAYL QMNSLKTEDTAVYYCASSGYSSGWTPFDYWGQGTLVTVSSASTKGPSVFPLAPSSKSTSGGTAALGCLVKDYFPEP VTVSWNSGALTSGVHTFPAVLQSSGLYSLSSVVTVPSSSLGTQTYICNVNHKPSNTKVDKKVEPKSCDKTHTCPPCP APELLGGPSVFLFPPKPKDTLMISRTPEVTCVVVDVSHEDPEVKFNWYVDGVEVHNAKTKPREEQYNSTYRVVSVLT VLHQDWLNGKEYKCKVSNKALPAPIEKTISKAKGQPREPQVYTLPPSREEMTKNQVSLTCLVKGFYPSDIAVEWESN GQPENNYKTTPPVLDSDGSFFLYSKLTVDKSRWQQGNVFSCSVMHEALHNHYTQKSLSLSPGK (SEQ ID NO:319).

In some cases, an anti-BCMA antibody comprises a VL present in the light chain amino acid sequence provided above; and a VH present in the heavy chain amino acid sequence provided above. For example, an anti-BCMA antibody can comprise: a) a VL comprising an amino acid sequence having at least 90%, at least 95%, at least 98%, at least 99%, or 100%, amino acid sequence identity to the amino acid sequence: QSVLTQPPSASGT PGQRVTISCSGSSSNIGSNTVNWYQQLPGTAPKLLIFNYHQRPSGVPDRFSGSKSGSSASLAISGLQSEDEADYYCA AWDDSLNGWVFGGGTKLTVLG (SEQ ID NO:320); and b) a VH comprising an amino acid sequence having at least 90%, at least 95%, at least 98%, at least 99%, or 100%, amino acid sequence identity to the amino acid sequence: EVOLVESGGGLVKPGGSLRLSCAASGFTFGDYALSWFRQAPGKGLEWVGVSRSKAYGGTTDYAASVKGRFT ISRDDSKSTAYLQMNSLKTEDTAVYYCASSGYSSGWTPFDYWGQGTLVTVSSASTKGPSV (SEQ ID NO:321).

In some cases, an anti-BCMA antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-BCMA antibody can comprise a VL CDR1 having the amino acid sequence SSNIGSNT (SEQ ID NO:322), a VL CDR2 having the amino acid sequence NYH, a VL CDR3 having the amino acid sequence AAWDDSLNGWV (SEQ ID NO:323)), a VH CDR1 having the amino acid sequence GFTFGDYA (SEQ ID NO: 324), a VH CDR2 having the amino acid sequence SRSKAYGGTT (SEQ ID NO:325), and a VH CDR3 having the amino acid sequence ASSGYSSGWTPFDY (SEQ ID NO:326).

An anti-BCMA antibody can be a scFv. As one non-limiting example, an anti-BCMA scFv can comprise the following amino acid sequence: QVQLVQSGAEVKKPGSSVKVSCKASGGTFSNYWMHWVRQAPGQGLEWMGATY RGHSDTYYNQKFKGRVTITADKSTSTAYMELSSLRSEDTAVYYCARGAIYNGYDVLDNWGQGTLVTVSSGGGGSGG GGSGGGGSGGGGSDIQMTQSPSSLSASVGDRVTITCSASQDISNYLNWYQQKPGKAPKLLIYYTSNLHSGVPSRFS GSGSGTDFTLTISSLQPEDFATYYCQQYRKLPWTFGQGTKLEIKR (SEQ ID NO:327).

As another example, an anti-BCMA scFv can comprise the following amino acid sequence: DIQMTQSPSS LSASVGDRVTITCSASQDISNYLNWYQQKPGKAPKLLIYYTSNLHSGVPSRFSGSGSGTDFTLTISSLQPEDFATYYC QQYRKLPWTFGQGTKLEIKRGGGGSGGGGSGGGGSGGGGSQVQLVQSGAEVKKPGSSVKVSCKASGGTFSNYW MHWVRQAPGQGLEWMGATYRGHSDTYYNQKFKGRVTITADKSTSTAYMELSSLRSEDTAVYYCARGAIYNGYDVL DNWGQGTLVTVSS (SEQ ID NO:328).

In some cases, an anti-BCMA antibody can comprise a VL CDR1 having the amino acid sequence SASQDISNYLN (SEQ ID NO:329); a VL CDR2 having the amino acid sequence YTSNLHS (SEQ ID NO:330); a VL CDR3 having the amino acid sequence QQYRKLPWT (SEQ ID NO:331); a VH CDR1 having the amino acid sequence NYWMH (SEQ ID NO:332); a VH CDR2 having the amino acid sequence ATYRGHSDTYYNQKFKG (SEQ ID NO: 333); and a VH CDR3 having the amino acid sequence GAIYNGYDVLDN (SEQ ID NO:334).

In some cases, an anti-BCMA antibody comprises: a) a light chain comprising an amino acid sequence having at least 90%, at least 95%, at least 98%, at least 99%, or 100%, amino acid sequence identity to the following amino acid sequence: DIQMTQSPSSLSASVGDRVTITCSASQDISNYLNWYQQKPGKAPKLLIYYTSNLHSGVPSRFS GSGSGTDFTLTISSLQPEDFATYYCQQYRKLPWTFGQGTKLEIKR (SEQ ID NO:335).

In some cases, an anti-BCMA antibody comprises: a) a heavy chain comprising an amino acid sequence having at least 90%, at least 95%, at least 98%, at least 99%, or 100%, amino acid sequence identity to the following amino acid sequence: QVQLVQSGAEVKKPGSSVKVSCKASGGTFSNYWM HWVRQAPGQGLEWMGATYRGHSDT YYNQKFKGRVTITADKSTSTAYMELSSLRSEDTAVYYCARGAIYDGYDVLDNWGQGTLVTVSS (SEQ ID NO:336).

In some cases, an anti-BCMA antibody (e.g., an antibody referred to in the literature as belantamab) comprises a light chain comprising the amino acid sequence: DIQMTQSPSSLSASVGDRVTITCSASQDISNYLNWYQ QKPGKAPKLLIYYTSNLHSGVPSRFSGSGSGTDFTLTISSLQPEDFATYYCQQYRKLPWTFGQGTKLEIKR (SEQ ID NO: 335); and a heavy chain comprising the amino acid sequence: QVQLVQSGAEVKKPGSSVKVSCKASGGTFSNY WMHWVRQAPGQGLEWMGATYRGHSDTYYNQKFKGRVTITADKSTSTAYMELSSLRSEDTAVYYCARGAIYDGYDV LDNWGQGTLVTVSS (SEQ ID NO:336).

In some cases, the anti-BCMA antibody has a cancer chemotherapeutic agent linked to the antibody. For example, in some cases, the anti-BCMA antibody is GSK2857916 (belantamab-mafodotin), where monomethyl auristatin F (MMAF) is linked via a maleimidocaproyl linker to the anti-BCMA antibody belantamab.

### (8) Anti-MUC1

A CTP may be an antibody specific for MUC1. For example, a CTP can be specific for a MUC1 polypeptide present on a cancer cell. In some cases, the CTP is specific for the cleaved form of MUC1; see, e.g., Fessler et al. (2009) Breast Cancer Res. Treat. 118:113. A CTP may be an antibody specific for a glycosylated MUC1 peptide; see, e.g., Naito et al. (2017) ACS Omega 2:7493; and U.S. Pat. No. 10,017,580.

As one non-limiting example, a CTP can be a single-chain Fv specific for MUC1. See, e.g., Singh et al. (2007) Mol. Cancer Ther. 6:562; Thie et al. (2011) PloSOne 6: e15921; Imai et al. (2004) Leukemia 18:676; Posey et al. (2016) Immunity 44:1444; EP3130607; EP3164418; WO 2002/044217; and US 2018/0112007. A CTP may be an scFv specific for the MUC1 peptide VTSAPDTRPAP GSTAPPAHG (SEQ ID NO:337). A CTP may be an scFv specific for the MUC1 peptide SNIKFRPGSVVVQLTLAFREGTINVHDVETQFNQYKTEAASRY (SEQ ID NO:338). A CTP may be an scFv specific for the MUC1 peptide SVVVQLTLAFREGTINVHDVETQFNQYKTEAASRY (SEQ ID NO: 339). A CTP may be an scFv specific for the MUC1 peptide LAFREGTINVHDV ETQFNQY (SEQ ID NO:340). In some cases, a CTP is a scFv specific for the MUC1 peptide SNIKFRPGSVVVQLTLAAFREGTIN (SEQ ID NO:341).

As an example, an anti-MUC1 antibody can comprise: a VH CDR1 having the amino acid sequence RYGMS (SEQ ID NO:342); a VH CDR2 having the amino acid sequence TISGGGTYIYY PDSVKG (SEQ ID NO: 343); a VH CDR3 having the amino acid sequence DNYGRNYDYGMDY (SEQ ID NO:344); a VL CDR1 having the amino acid sequence SATSSVSYIH (SEQ ID NO:345); a VL CDR2 having the amino acid sequence STSNLAS (SEQ ID NO:346); and a VL CDR3 having the amino acid sequence QQRSSSPFT (SEQ ID NO:347). See, e.g., US 2018/0112007.

As another example, an anti-MUC1 antibody can comprise a VH CDR1 having the amino acid sequence GYAMS (SEQ ID NO:348); a VH CDR2 having the amino acid sequence TISSGGTYIYYP DSVKG (SEQ ID NO:349); a VH CDR3 having the amino acid sequence LGGDNYYEYFDV (SEQ ID NO:350); a VL CDR1 having the amino acid sequence RASKSVSTSGYSYMH (SEQ ID NO:351); a VL CDR2 having the amino acid sequence LASNLES (SEQ ID NO:352); and a VL CDR3 having the amino acid sequence QHSRELPFT (SEQ ID NO:353). See, e.g., US 2018/0112007.

As another example, an anti-MUC1 antibody can comprise a VH CDR1 having the amino acid sequence DYAMN (SEQ ID NO:354); a VH CDR2 having the amino acid sequence VISTFSGNINFN QKFKG (SEQ ID NO:355); a VH CDR3 having the amino acid sequence SDYYGPYFDY (SEQ ID NO:356); a VL CDR1 having the amino acid sequence RSSQTIVHSNGNTYLE (SEQ ID NO:357); a VL CDR2 having the amino acid sequence KVSNRFS (SEQ ID NO: 358); and a VL CDR3 having the amino acid sequence FQGSHVPFT (SEQ ID NO:359). See, e.g., US 2018/0112007.

As another example, an anti-MUC1 antibody can comprise a VH CDR1 having the amino acid sequence GYAMS (SEQ ID NO:348); a VH CDR2 having the amino acid sequence TISSGGTYIYYP DSVKG (SEQ ID NO:349); a VH CDR3 having the amino acid sequence LGGDNYYEY (SEQ ID NO:360); a VL CDR1 having the amino acid sequence TASKSVSTSGYSYMH (SEQ ID NO:361); a VL CDR2 having the amino acid sequence LVSNLES (SEQ ID NO: 362); and a VL CDR3 having the amino acid sequence QHIRELTRSE (SEQ ID NO:363). See, e.g., US 2018/0112007.

### (9) Anti-MUC16

In some cases, a CTP is an antibody specific for MUC16 (also known as CA125). See, e.g., Yin et al. (2002) Int. J. Cancer 98:737. For example, a CTP can be specific for a MUC16 polypeptide present on a cancer cell. See, e.g., US 2018/0118848; and US 2018/0112008. In some cases, a MUC16-specific CTP is a scFv. In some cases, a MUC16-specific CTP is a nanobody.

As one example, an anti-MUC16 antibody can comprise a VH CDR1 having the amino acid sequence GFTFSNYY (SEQ ID NO:364); a VH CDR2 having the amino acid sequence ISGRGSTI (SEQ ID NO:365); a VH CDR3 having the amino acid sequence VKDRGGYSPY (SEQ ID NO:366); a VL CDR1 having the amino acid sequence QSISTY (SEQ ID NO:367); a VL CDR2 having the amino acid sequence TAS; and a VL CDR3 having the amino acid sequence QQSYSTPPIT (SEQ ID NO:368). See, e.g., US 2018/0118848.

### (10) Anti-Claudin-18.2

In some cases, a CTP is an antibody specific for claudin-18 isoform 2 (“claudin-18.2”). See, e.g., WO 2013/167259. In some cases, a claudin-18.2-specific CTP is a scFv. In some cases, a claudin-18.2-specific CTP is a nanobody. A CTP may be an antibody specific for TEDEVQSYP SKHDYV (SEQ ID NO:240) or EVQSYPSKHDYV (SEQ ID NO:241).

As one example, an anti-claudin-18.2 antibody can comprise a VH CDR1 having the amino acid sequence GYTFTDYS (SEQ ID NO:369); a VH CDR2 having the amino acid sequence INTETGVP (SEQ ID NO:370); a VH CDR3 having the amino acid sequence ARRTGFDY (SEQ ID NO:371); a VL CDR1 having the amino acid sequence KNLLHSDGITY (SEQ ID NO:372); a VL CDR2 having the amino acid sequence RVS; and a VL CDR3 having the amino acid sequence VQVLELPFT (SEQ ID NO:373).

As another example, an anti-claudin-18.2 antibody can comprise a VH CDR1 having the amino acid sequence GFTFSSYA (SEQ ID NO:374); a VH CDR2 having the amino acid sequence ISDGGSYS (SEQ ID NO: 375); a VH CDR3 having the amino acid sequence ARDSYYDNSYVRDY (SEQ ID NO:376); a VL CDR1 having the amino acid sequence QDINTF (SEQ ID NO:377); a VL CDR2 having the amino acid sequence RTN; and a VL CDR3 having the amino acid sequence LQYDEFPLT (SEQ ID NO:378).

8 Epitopes and their Assessment

The chemical conjugation sites and chemistries described herein permit the incorporation of a molecule presenting a coronavirus epitope into an unconjugated T-Cell-MP to form a T-Cell-MP-coronavirus epitope conjugate. Molecules that may be conjugated to an unconjugated T-Cell-MP include those presenting a peptide epitope, phosphopeptide epitope, or glycopeptide epitope (such as those from the spike glycoprotein, nucleoprotein, membrane protein, replicase protein, non-structural protein (nsp) and the like); collectively an “epitope.” Epitopes of a T-Cell-MP conjugate are not part of the first or second polypeptide as translated from mRNA, but may be added to a T-Cell-MP at a chemical conjugation site. Selection of candidate MHC allele and peptide (e.g., phosphopeptide, lipopeptides or glycopeptide) epitope combinations for effective presentation to a TCR by a T-Cell-MP-epitope conjugate can be accomplished using any of a number of well-known methods to determine if the free peptide has affinity for the specific HLA allele used to construct the T-Cell-MP in which it will be presented as part of the epitope conjugate. It is also possible to determine if the peptide in combination with the specific heavy chain allele and β2M can affect the T cell in the desired manner (e.g., induction of cell activation, proliferation, anergy, or apoptosis). Applicable methods include binding assays and T cell activation assays.

It is possible to determine if an epitope in combination with the specific heavy chain allele and β2M sequence can affect the T cell in the desired manner (e.g., induction of proliferation, granule mediated responses, anergy, or apoptosis). Applicable methods include binding assays and T cell activation assays including BLI assays utilized for assessing binding affinity of T-Cell-MPs with wt. and variant MODs discussed above. See, e.g., published PCT Application WO 2020/243315 (Cue Biopharma, Inc.) at to [00347].

a. Epitopes

An epitope present in a T-Cell-MP-coronavirus epitope conjugate may be bound in an epitope-specific manner by a T cell (i.e., the epitope is specifically bound by an epitope-specific T cell whose TCR recognizes the peptide). An epitope-specific T cell binds an epitope having a reference aa sequence in the context of a specific MHC-H allele polypeptide/β2M complex, but does not substantially bind an epitope that differs from the reference aa sequence presented in the same context. For example, an epitope-specific T cell may bind an epitope in the context of a specific MHC-H allele polypeptide/β2M complex having a reference aa sequence, and may bind an epitope that differs from the reference aa sequence presented in the same context, if at all, with an affinity that is less than 10⁻⁶ M, less than 10⁻⁵ M, or less than 10⁻⁴ M. An epitope-specific T cell may bind an epitope (e.g., a peptide presenting an epitope of interest) for which it is specific with an affinity of at least 10⁻⁷ M, at least 10⁻⁸ M, at least 10⁻⁹ M, or at least 10⁻¹⁰ M.

In some cases, the peptide epitope present in a T-Cell-MP-coronavirus epitope conjugate presents an epitope-specific to an HLA-A, -B, -C, -E, -F or -G allele. In an embodiment, the peptide epitope present in a T-Cell-MP presents an epitope restricted to HLA-A*0101, A*0201, A*0301, A*1101, A*2301, A*2402, A*2407, A*3303, and/or A*3401. In an embodiment, the peptide epitope present in a T-Cell-MP presents an epitope restricted to HLA-B*0702, B*0801, B*1502, B3501, B*3802, B*4001, B*4402, B*4601, and/or B*5301. In an embodiment, the peptide epitope present in a T-Cell-MP presents an epitope restricted to C*0102, C*0303, C*0304, C*0401, C*0602, C*0701, C*702, C*0801, and/or C*1502. In an embodiment, the peptide epitope present in a T-Cell-MP presents an epitope restricted to HLA-E, e.g., HLA-E*0101 (HLA-E*01:01:01:01), HLA-E*01:03 (HLA-E*01:03:01:01), HLA-E*01:04, HLA-E*01:05, HLA-E*01:06, HLA-E*01:07, HLA-E*01:09, and HLA-E*01:10. Of these, isoforms HLA-E*0101 and HLA-E*01.03 are of particular note since these are highly prevalent alleles, and differ by only 1 aa (Arg or Gly at position 107).

Among the epitopes that may be bound and presented to a TCR by a T-Cell-MP with a class I MHC-H and a β2M polypeptide sequence are antigens from infectious agents (e.g., viral or bacterial agents), particularly coronaviruses. Particular coronavirus antigens are selected from structural proteins or surface protein antigens, particularly antigens (or epitopes) derived from spike (S protein or surface glycoprotein), membrane protein (M or membrane glycoprotein), envelope protein (E) or nucleocapsid (N) protein. Coronavirus antigens may also be non-structural proteins (NSPs).

### (i) Epitopes Present in Coronavirus-Associated Antigens

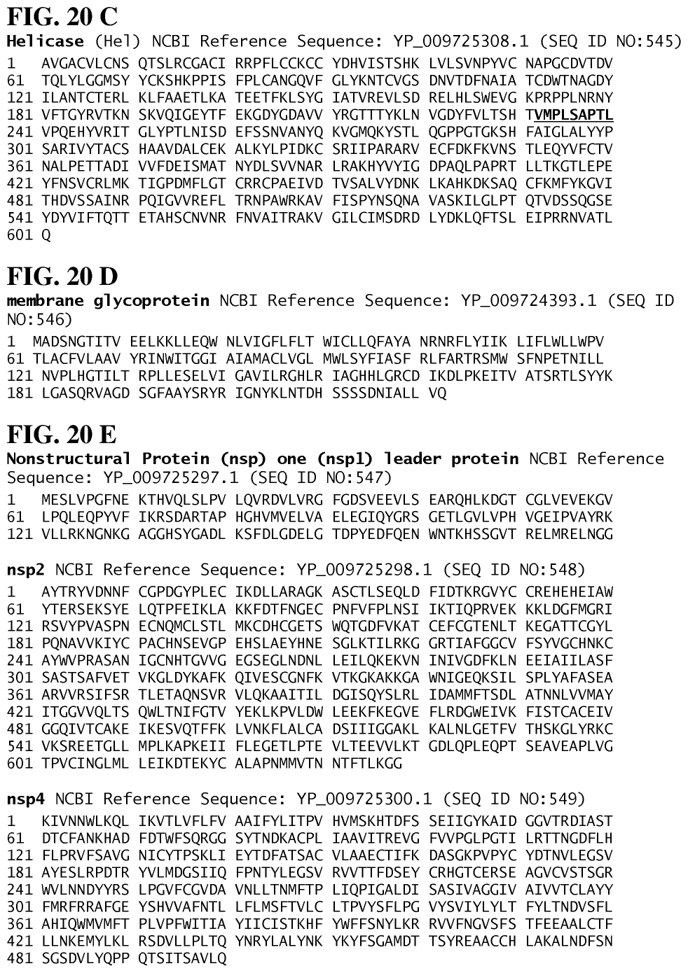

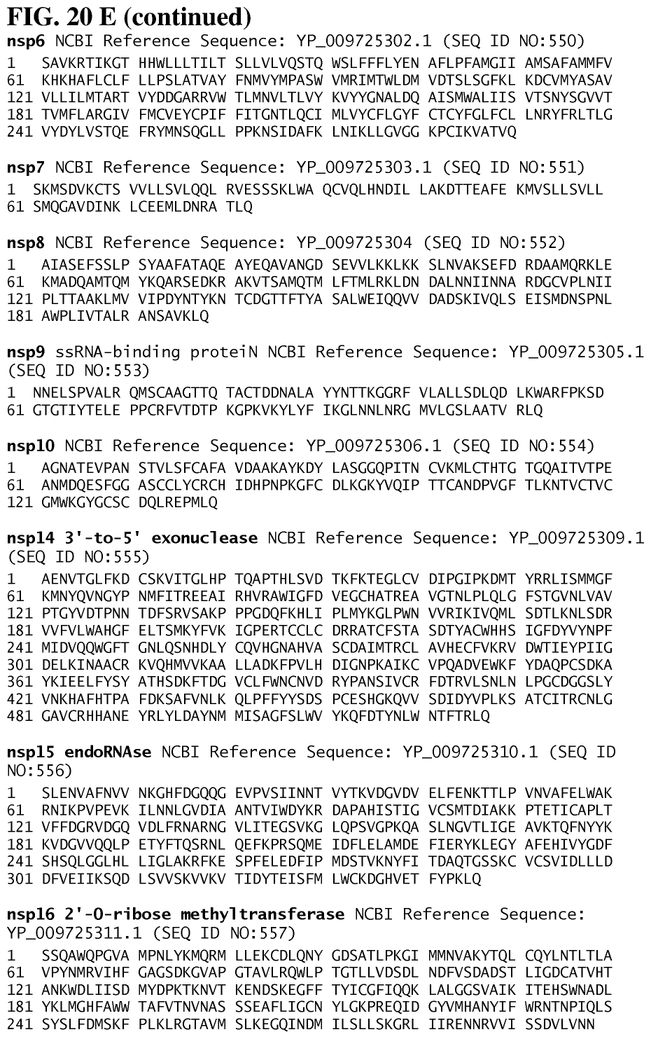

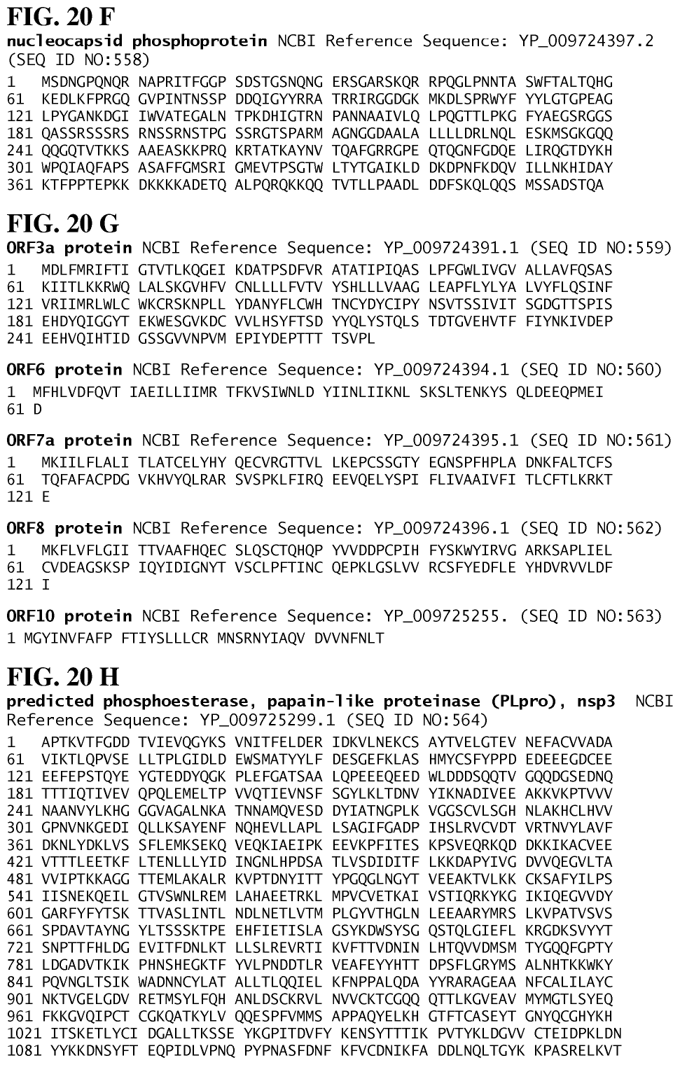

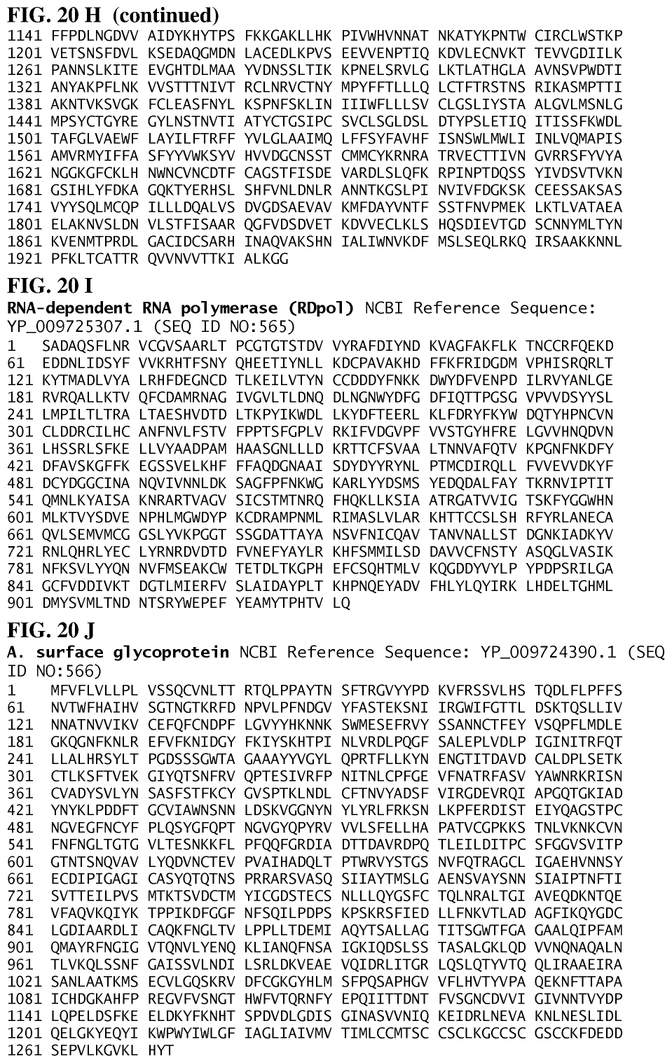

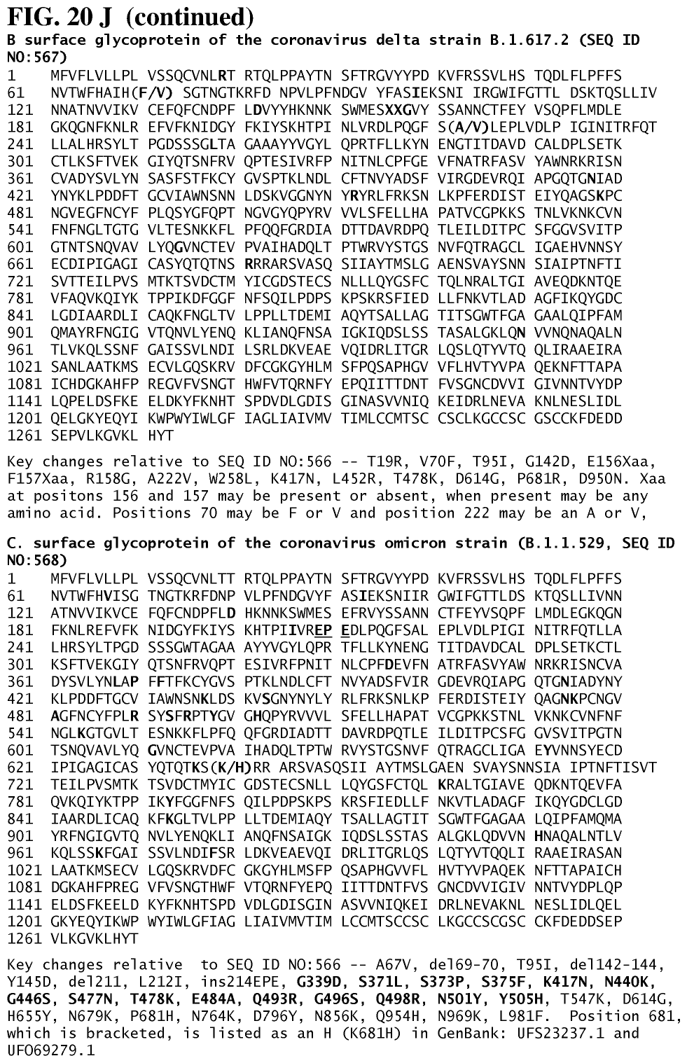

A peptide presenting one or more epitopes (a peptide epitope) presented by a T-Cell-MP-epitope conjugate may be a peptide that presents an epitope derived from a coronavirus protein (e.g., Core and iCore peptides that differ from coronavirus peptides, see FIGS. 20A-20J, and FIG. 21). A portion of a coronavirus protein that presents one or more epitopes is referred to herein as a coronavirus peptide epitope. A coronavirus peptide epitope present in a T-Cell-MP-coronavirus epitope conjugate can comprise a peptide having from 4 to 25 contiguous aas (e.g., 4 aa, 5 aa, 6 aa, 7 aa, 8 aa, 9 aa, 10 aa, 11 aa, 12 aa, 6-15 aa, 7-15 aa, 10⁻¹⁵ aa, 15-20 aa, or 20-25 aa) of an aa sequence having at least 80%, at least 90%, at least 95%, or 100% aa sequence identity to any of the aa sequences depicted in Table 2, FIGS. 20A to 20J, or FIG. 21. A peptide presenting a coronavirus epitope present in a T-Cell-MP-coronavirus epitope conjugate can be a peptide having from 4 to 25 contiguous aas (e.g., 4aa, 5aa, 6 aa, 7 aa, 8 aa, 9 aa, 6-12 aa, 8-15 aa, 15-20 aa, or 20-25 aa) of an aa sequence having at least 80%, at least 90%, at least 95%, or 100% aa sequence identity to any one of the coronavirus aa sequences depicted in FIG. 21, FIG. 22, Table 2, Table 3, or Table 3a.

Coronavirus human T cell epitopes present in a T-Cell-MP-coronavirus epitope conjugate may comprise the aa sequence of any of the peptides (aa sequences) presented in FIG. 21. The peptide presenting a coronavirus epitope present in a T-Cell-MP-coronavirus epitope conjugate may comprise the sequence of a peptide presented under the heading “Peptide” in FIG. 21. The peptide presenting a coronavirus epitope present in a T-Cell-MP-coronavirus epitope conjugate may comprise the sequence of a peptide presented under the heading “Core” in FIG. 21. The peptide presenting a coronavirus epitope present in a T-Cell-MP-coronavirus epitope conjugate may comprise the sequence of a peptide presented under the heading “iCore” in FIG. 21. The peptide presenting a coronavirus epitope present in a T-Cell-MP-coronavirus epitope conjugate may comprise the aa sequence of a peptide of: surface glycoprotein, membrane protein, or nucleocapsid phosphoprotein in FIG. 21, or a sequence having one or two aa substitutions or deletions of any of those sequences. The peptide presenting a coronavirus epitope present in a T-Cell-MP-coronavirus epitope conjugate may comprise the aa sequence of a peptide of: open reading frame (ORF) 1b protein, ORF3a protein, ORF6 protein, ORF7a protein, ORF8 protein, or ORF10 protein in FIG. 21, or a sequence having one or two aa substitutions or deletions of any of those sequences. The peptide presenting a coronavirus epitope present in a T-Cell-MP-coronavirus epitope conjugate may comprise the aa sequence of a peptide of an envelope protein, membrane glycoprotein, nonstructural protein (nsp) one (nsp1), nsp2, nsp4, nsp6, nsp7, nsp8, nsp9, nsp10, nsp14, nsp15, or nsp16 in FIG. 21, or a sequence having one or two aa substitutions or deletions of any of those sequences. The peptide presenting a coronavirus epitope present in a T-Cell-MP-coronavirus epitope conjugate may comprise the aa sequence of a peptide of: nucleocapsid phosphoprotein, papain-like cysteine protease (PLpro), 3C-like protease (3CL), RNA-dependent RNA polymerase (RDpol), or helicase (Hel) in FIG. 21, or a sequence having one or two aa substitutions or deletions of any of those sequences.

Some suitable peptides presenting coronavirus epitopes for T-Cell-MP-epitope conjugate formation include those common to SARS-CoV and SARS-CoV-2set forth in Table 2 (see Grifoni et al., Cell Host & Microbe 27 (4): 671-680 (2020); Cell Host & Microbe (2020), https://doi.org/10.1016/j.chom.2020.03.002).

TABLE 2¹

| | HLA | SARS-CoV-2 | | Start and | |
|---|---|---|---|---|---|
| | Restric- | (Covid-19) | Coronavirus | End | Identity |
| SARS-CoV² Sequence | tion | Sequence³ | Protein⁴ | Position | Percent |
| GLMWLSYFV (381) | A*02:01 | GLMWLSYFI (382) | M | 89-97 | 89 |
| TLACFVLAAV (383) | A*02:01 | TLACFVLAAV (383) | M | 61-70 | 100 |
| ALNTPKDHI (384) | A*02:01 | ALNTPKDHI (384) | N | 138-146 | 100 |
| GMSRIGMEV (385) | A*02:01 | GMSRIGMEV (385) | N | 316-324 | 100 |
| LALLLLDRL (386) | A*02:01 | LALLLLDRL (386) | N | 219-227 | 100 |
| LLLDRLNQL (387) | A*02:01 | LLLDRLNQL (387) | N | 222-230 | 100 |
| LQLPQGTTL (388) | A*02:01 | LQLPQGTTL (388) | N | 159-167 | 100 |
| RLNQLESKV (389) | A*02:01 | RLNQLESKM (390) | N | 226-234 | 89 |
| ALSGVFCGV (391) | A*02:01 | SLPGVFCGV (392) | ORF 1ab | 2942-2950 | 78 |
| CLDAGINYV (393) | A*02:01 | CLEASFNYL (394) | ORF 1ab | 2139-2147 | 56 |
| ILLLDQVLV (395) | A*02:01 | ILLLDQALV (396) | ORF 1ab | 2498-2506 | 89 |
| LLCVLAALV (397) | A*02:01 | SACVLAAEC (398) | ORF 1ab | 2840-2848 | 56 |
| SMWALVISV (399) | A*02:01 | SMWALIISV (400) | ORF 1ab | 3661-3669 | 89 |
| TLMNVITLV (401) | A*02:01 | TLMNVLTLV (402) | ORF 1ab | 3639-3647 | 89 |
| WLMWFIISI (403) | A*02:01 | WLMWLIINL (404) | ORF 1ab | 2292-2300 | 67 |
| ALNTLVKQL (405) | A*02:01 | ALNTLVKQL (405) | S | 958-966 | 100 |
| FIAGLIAIV (406) | A*02:01 | FIAGLIAIV (406) | S | 1220-1228 | 100 |
| KLPDDFMGCV (407) | A*02:01 | KLPDDFTGCV (408) | S | 424-433 | 90 |
| LITGRLQSL (409) | A*02:01 | LITGRLQSL (409) | S | 996-1004 | 100 |
| NLNESLIDL (410) | A*02:01 | NLNESLIDL (410) | S | 1192-1200 | 100 |
| RLNEVAKNL (411) | A*02:01 | RLNEVAKNL (411) | S | 1185-1193 | 100 |
| SIVAYTMSL (412) | A*02:01 | SIIAYTMSL (413) | S | 691-699 | 89 |
| VLNDILSRL (414) | A*02:01 | VLNDILSRL (414) | S | 976-984 | 100 |
| QFKDNVILL (415) | A*24:02 | NFKDQVILL (416) | N | 345-353 | 78 |
| GETALALLLL (417) | B*40:01 | GDAALALLLL (418) | N | 215-224 | 80 |
| MEVTPSGTWL (419) | B*40:01 | MEVTPSGTWL (419) | N | 322-331 | 100 |
| RFFTLGSITAQPVKI (420) | B*58:01 | RIFTIGTVTLKQGEI (421) | ORF 3a | 6-20 | 40 |
| SITAQPVKI (422) | B*58:01 | TVTLKQGEI (423) | ORF 3a | 12-20 | 22 |
| HLRMAGHSL (424) | Class I | HLRIAGHHL (425) | M | 148-156 | 78 |
| TKQYNVTQAF (426) | Class I | TKAYNVTQAF (427) | N | 265-274 | 90 |

¹SEQ ID NOs. are given in parenthesis following each sequence. Restrictions defined only in HLA-transgenic mice are indicated by the italicized font.
²SARS-CoV TOR-2 strain (see Grifoni https://doi.org/10.1016/j.chom.2020.03.002 at FIG. 1) and Dwyer, O. BMJ, 326(7397): 999 (2003).
³SARS-CoV-2 Wuhan-Hu-1 see Grifoni https://doi.org/10.1016/j.chom.2020.03.002 at FIG. 1.
⁴S, surface glycoprotein; M, membrane protein; N, nucleocapsid phosphoprotein, ORF 1ab polyprotein (see NCBI Reference Sequence: YP_009724389.1).

A peptide presenting a coronavirus epitope present in a T-Cell-MP-coronavirus epitope conjugate may comprise a peptide having from 6 to 25 contiguous aas (e.g., 6 aa, 7 aa, 8 aa, 9 aa, 8-15 aa, or 15-20 aa) of an aa sequence having at least 80%, at least 90%, at least 95%, or 100% aa sequence identity to any one of the coronavirus aa sequences depicted in Table 2.

The peptide presenting a coronavirus epitope present in a T-Cell-MP-coronavirus epitope conjugate may comprise a peptide set forth in Table 2 or an aa sequence having 1 or 2 aa substitutions, deletions or insertions in any of those peptide sequences. In some cases, the epitope may comprise a surface glycoprotein peptide. In some cases, the epitope may comprise a membrane protein peptide. In some cases the epitope may comprise a nucleocapsid phosphoprotein peptide. In some cases, the epitope may comprise an ORF 1ab polyprotein peptide from Table 2.

The SARS-CoV-2 human T cell epitopes present in a T-Cell-MP-coronavirus epitope conjugate may comprise a peptide of a protein set forth in FIG. 22, or an aa sequence having 0, 1, or 2 aa substitutions, deletions or insertions in any of the proteins or peptide sequences in FIG. 22. See Grifoni et al., Cell Host & Microbe 27:1-10 (2020); Cell Host & Microbe (2020), https://doi.org/10.1016/j.chom.2021.05.010 for a discussion of the epitopes along with the HLA alleles capable of presenting them. The peptide presenting a coronavirus epitope present in a T-Cell-MP-coronavirus epitope conjugate may comprise the aa sequence of a peptide of an envelope protein (E), membrane glycoprotein (M), or nucleocapsid phosphoprotein (nucleocapsid protein or “N”) set forth in FIG. 22, or a sequence having one or two aa substitutions or deletions of any of those sequences. The peptide presenting a coronavirus epitope present in a T-Cell-MP-coronavirus epitope conjugate may comprise the aa sequence of a peptide of: nonstructural protein (nsp) NSP1, NSP2, NSP3, NSP6, NSP7, NSP8, NSP9, NSP10, NSP12, NSP13, NSP14, NSP15, or NSP16 set forth in FIG. 22, or a sequence having one or two aa substitutions or deletions of any of those sequences. The peptide presenting a coronavirus epitope present in a T-Cell-MP-coronavirus epitope conjugate may comprise the aa sequence of a peptide of: open reading frame (ORF) ORF3a, ORF6, ORF7a, ORF7b, ORF8, or ORF10 set forth in FIG. 22, or a sequence having one or two aa substitutions or deletions of any of those sequences.

The peptide presenting a coronavirus epitope present in a T-Cell-MP-coronavirus epitope conjugate may comprise the aa sequence of a peptide of a: surface glycoprotein (spike protein or S protein) set forth in FIG. 20J, a peptide of a coronavirus surface glycoprotein (spike protein) set forth in FIG. 22, or a sequence having 1, or 2 aa substitutions, insertions, or deletions in any of those sequences.

Some suitable SARS-CoV-2 spike protein epitopes for T-Cell-MP-epitope conjugate formation and the HLA alleles presenting them are set forth in Table 3 (derived from Grifoni et al., Cell Host and Microbe 29 (7): 1076-1092 (2021). Those spike protein epitopes along are provided in Table 3.

TABLE 3^{†,‡}

| SARS-CoV | HLA- | Coronavirus | SARS-CoV | HLA- | Coronavirus |
|---|---|---|---|---|---|
| Sequence¹ | Restriction | Protein² | Sequence | Restriction | Protein |
| STQDLFLPFF (428) | A*01:01 | S | YYVGYLQPRTF (429) | A*24:02 | S |
| FCNDPFLGVYY (430) | A*01:01 | S | NYNYLYRLF (431) | A*24:02 | S |
| SSANNCTFEY (432) | A*01:01 | S | YFPLQSYGF (433) | A*24:02 | S |
| STECSNLLLQY (434) | A*01:01 | S | VYDPLQPELDSF (435) | A*24:02 | S |
| YTNSFTRGVY (436) | A*01:01, | S | YEQYIKWPWYI (437) | A*24:02 | S |
| | A*26:01 | | | | |
| CVADYSVLY (438) | A*01:01, | S | KWPWYIWLGF (439) | A*24:02 | S |
| | A*26:01, | | | | |
| | A*29:02, | | | | |
| | B*15:01 | | | | |
| LTDEMIAQY (440) | A*01:01, | S | GTITSGWTF (441) | A*24:02, B*57:01 | S |
| | A*29:02, | | | | |
| | B*35:01, | | | | |
| | C*07:02 | | | | |
| KIADYNYKL (442) | A*02:01 | S | QYIKWPWYI (443) | A*24:02, C*06:02 | S |
| KLPDDFTGCV (408) | A*02:01 | S | RFDNPVLPF (444) | A*24:02, C*07:02 | S |
| SFELLHAPATV (445) | A*02:01 | S | EILDITPCSF (446) | A*26:01 | S |
| LLFNKVTLA (447) | A*02:01 | S | FVFKNIDGY (448) | A*26:01, A*29:02 | S |
| VLNDILSRL (414) | A*02:01 | S | SVASQSIIAY (449) | A*26:01, A*29:02 | S |
| RLDKVEAEV (450) | A*02:01 | S | WTAGAAAYY (451) | A*26:01, | S |
| | | | | A*29:02, B*35:01 | |
| RLQSLQTYV (452) | A*02:01 | S | VASQSIIAY (453) | A*29:02, | S |
| | | | | B*15:01, B*35:01 | |
| HLMSFPQSA (454) | A*02:01 | S | TPINLVRDL (455) | B*07:02 | S |
| RLNEVAKNL (411) | A*02:01 | S | SPRRARSVA (456) | B*07:02 | S |
| NLNESLIDL (410) | A*02:01 | S | APHGVVFLHV (457) | B*07:02 | S |
| FIAGLIAIV (406) | A*02:01 | S | LPQGFSAL (458) | B*07:02, B*08:01 | S |
| VVFLHVTYV (459) | A*02:01, | S | QPYRVVVL (460) | B*07:02, B*08:01 | S |
| | A*02:06 | | | | |
| YLQPRTFLL (461) | A*02:01, | S | MIAQYTSAL (462) | B*07:02, B*08:01 | S |
| | A*24:02, | | | | |
| | B*08:01 | | | | |
| SIIAYTMSL (413) | A*02:01, | S | FPQSAPHGVVF (463) | B*08:01, B*35:01 | S |
| | B*08:01 | | | | |
| TLDSKTQSL (464) | A*02:01, | S | SEPVLKGVKL (465) | B*40:01 | S |
| | B*08:01, | | | | |
| | C*07:02 | | | | |
| KCYGVSPTK (466) | A*03:01 | S | TEKSNIIRGW (467) | B*44:02, B*44:03 | S |
| TVYDPLQPELDSFK | A*03:01 | S | TECSNLLLQY (469) | B*44:02, B*44:03 | S |
| (468) | | | | | |
| GVYFASTEK (470) | A*03:01, | S | REGVFVSNGTHW (471) | B*44:03 | S |
| | A*11:01, | | | | |
| | A*68:01 | | | | |
| GTHWFVTQR (472) | A*11:01, | S | | | |
| | A*31:01 | | | | |

^{^{†}}SEQ ID NO is given in parenthesis following each sequence.
^{^{‡}}S, surface glycoprotein (see NCBI Reference Sequence: YP_009724389.1).

Because coronaviruses like SARS-CoV-2 continuously evolve as genetic mutations occur during replication, they produce of a variety of related variants. A number of emerging coronavirus variants (e.g., SARS-CoV-2 variants) are listed as being Variants of Concern (VOC), Variants of Interest (VOI), Variants Being Monitored (VBM) or Variant of High Consequence (VOHC). At present include VBMs include the: Alpha (B.1.1.7 and Q lineages, Beta (B.1.351 and descendent lineages), Gamma (P.1 and descendent lineages), Epsilon (B.1.427 and B.1.429), Eta (B.1.525), lota (B.1.526), Kappa (B.1.617.1), 1.617.3, Mu (B.1.621, B.1.621.1) and Zeta (P.2). Currently the delta variant Delta (B. 1.617.2 and AY lineages) and Omicron (B.1.1.529 and BA lineages) are VOC. See, e.g., www.cdc.gov/coronavirus/2019-ncov/variants/variant-info.html#Concern. Those variants have been sequenced and mutations identified relative to the SARs-CoV-2 sequences NCBI Accession NC_045512.2 (see, e.g., https://www.ncbi.nlm.nih.gov/sars-cov-2/), designated as the wild-type. For example, variants in the spike protein may be designated relevant to NCBI Ref. Seq. YP_009724390.1. The variant mutations include numerous amino acid changes, including substitutions and deletions in, for example, the surface glycoprotein or spike(S) protein. The mutations may make the variant less susceptible to circulating antibodies in vaccinated individuals and may alter (e.g., increase) the transmission, give rise to more severe disease, and even increase mortality.

Going forward it is expected that additional VOCs, VOIs, VBMs and potentially even VOHCs will arise. The modular nature of the T-Cell-MP system described herein permits the rapid preparation of coronavirus epitope-conjugates capable of presenting epitopes presented by coronavirus variants, or the use of redirected T-Cell-MP-epitope conjugates as discussed below to address variants. For example, one skilled in the art can sequence coronavirus variants and compare them to the wild-type sequence to determine altered or new epitopes for the variant(s). This is within the skill and capability of one skilled in the art including by direct sequence comparison. For instance, surface glycoprotein sequences for each of the alpha (UK B1.1.7), beta (B1.351; QRN78347.1), gamma (P.1) delta (B1.617.2; QWK65230.1), and omicron (B.1.1.529) Sars-Cov2 variants were compared with wild-type Sars-Cov2 surface protein sequence. In particular, the surface glycoprotein epitope sequences epitopes provided in FIG. 21 were assessed and compared in each of the variants to identify mutations or amino acid changes in the epitope sequence. These provide epitopes and antigens specific for the variant(s), and can be utilized to include variants, or in the alternative to specifically target or treat one or more variant(s), using T-Cell-MPs. Some variant sequences for surface glycoprotein epitopes (variations bolded and italicized for emphasis) appearing in FIG. 21 include: KVGGNYNYR (SEQ ID NO:473), delta variant of KVGGNYNYL (SEQ ID NO:474); KGFNCYFPL (SEQ ID NO: 475), delta and beta variant of EGFNCYFPL (SEQ ID NO:476); DVYYHKNNK (SEQ ID NO:477), delta variant and GVY-HKNNK (SEQ ID NO:478) alpha variant of GVYYHKNNK (479); NSANLAAIK (SEQ ID NO:480), gamma variant of ASANLAATK (SEQ ID NO:481); RFANPVLPF (SEQ ID NO:482), beta variant of YYHKNNKSW (SEQ ID NO: 483); AYSNNSIAIPINF (SEQ ID NO:484), alpha variant of AYSNNSIAIPTNF (SEQ ID NO:485); PINLVRGL (SEQ ID NO: 486), beta variant of TPINLVRDL (SEQ ID NO:455); SRRRARSVA (SEQ ID NO:487), delta variant and SHRRARSVA (SEQ ID NO:488) alpha variant of SPRRARSVA (SEQ ID NO:456); IPINFTISV (SEQ ID NO:489), alpha variant of IPTNFTISV (SEQ ID NO:490); NLRTRTQL (SEQ ID NO:491), delta variant and NFTNRTQL (SEQ ID NO: 492) gamma variant of NLTTRTQL (SEQ ID NO:493); LXXXHRSYL (where each X is absent or any amino acid, SEQ ID NO:494), beta variant of LLALHRSYL (SEQ ID NO:495); LPSAYTNSF (SEQ ID NO:496), gamma variant of LPPAYTNSF SEQ ID NO:497); and AEYVNNSY (SEQ ID NO:498), gamma variant of AEHVNNSY (SEQ ID NO: 499).

Other suitable coronavirus peptides that can serve as peptides presenting coronavirus epitopes include spike protein sequences of the Wuhan-Hu1 strain (FIG. 20J at A, SEQ ID NO:566) having one or more insertions, deletions or substitutions found in the delta and/or omicron strains selected from the group consisting of T19R, A67V, del69-70, (V70F*), T95I, G142D, del142-144, Y145D, R158G, del211, L212I, ins214EPE, (A222V*), (W258L*), G339D, S371L, S373P, S375F, K417N, N440K, G446S, L452R, S477N, T478K, E484A, Q493R, G496S, Q498R, N501Y, Y505H, T547K, D614G, H655Y, N679K, P681H, P681R, N764K, D796Y, N856K, D950N, Q954H, N969K, and L981F. The substitutions may also be selected from T95I, K417N*T478K, and D614G, which are common to both the delta and omicron strain. A suitable SARS-CoV-2 spike protein epitope may comprise a peptide from about 6 to about 25 contiguous aas (e.g., from about 6 to about 15 aas) of the coronavirus delta variant spike protein sequence provided in FIG. 20J at B, optionally having 1 or 2 aa substitutions. A suitable SARS-CoV-2 spike protein epitope may comprise a peptide from about 6 to about 25 contiguous aas (e.g., from about 6 to about 15 aas) of the coronavirus omicron variant spike protein sequence provided in FIG. 20J at C, optionally having 1 or 2 aa substitutions.

Advantageously, peptides presenting coronavirus epitopes associated with memory T cells (see, e.g., Table 3a) may be employed as epitopes in the T-Cell-MPs described herein.

TABLE 3a
Coronavirus Epitopes Associated with Memory T Cells

| | | | Reported | % of | |
|---|---|---|---|---|---|
| HLA | | Parent | Affinity | Patients | Variant |
| Allele | Peptide Epitope | protein | (nM) | (Screen) | of Concern |
| A*02:01 | KLWAQCVQL (699) | ORF1ab | 17.7 | 88.9 | |
| A*02:01 | YLQPRTFLL (461) | S | 5.4 | 77.8 | |
| A*02:01 | LLYDANYFL (706) | ORF3a | 3.1 | 88.9 | |
| A*02:01 | LFYDANYFL (380) | ORF3a | | | B.1.617.2 BA.2 |
| A*02:01 | ALWEIQQVV (700) | ORF1ab | 7.8 | 88.9 | |
| A*02:01 | LLLDRLNQL (387) | N | 14.8 | 33.3 | |
| A*02:01 | YLFDESGEFKL (709) | ORF1ab | 22.2 | 44.4 | |
| A*02:01 | YLFDESGDFKL (709) | ORF1ab | | | B.1.1.7 |
| A*02:01 | GLMWLSYFI (382) | M | | | |
| A*02:01 | HLVDFQVTI (707) | ORF6 | | | |
| A*02:01 | HLVGFQVTI (707) | ORF6 | | | BA.5 |
| A*02:01 | FLLNKEMYL (695) | ORF1ab | | | |
| A*02:01 | RLQSLQTYV (452) | S | | | |
| A*01:01 | FTSDYYQLY (1545) | ORF3a | 3.2 | 100 | |
| A*01:01 | TTDPSFLGRY (612) | ORF1ab | 7.2 | 100 | |
| A*01:01 | TTDLSFLGRY (612) | ORF1ab | | | B.1.617.2 |
| A*01:01 | PTDNYITTY (1816) | ORF1ab | 6.1 | 80 | |
| A*01:01 | ATSRTLSYY (1329) | M | 16.7 | 60 | |
| A*01:01 | CTDDNALAYY (602) | ORF1ab | 5.3 | 100 | |
| A*01:01 | NTCDGTTFTY (1494) | ORF1ab | 121.8 | 60 | |
| A*01:01 | DTDFVNEFY (1543) | ORF1ab | 2.8 | 40 | |
| A*01:01 | GTDLEGNFY (1430) | ORF1ab | 6 | 40 | |
| A*01:01 | LTDEMIAQY (440) | S | | | |
| A*01:01 | ALSKGVHFV (703) | ORF3a | | | |
| A*01:01 | SLSKGVHFV (2043) | ORF3a | | | B.1.351 |
| A*01:01 | ALSKGVYFV (2044) | ORF3a | | | B1.1.529 BA.2 |
| A*03:01 | KTFPPTEPK (1447) | N | 20.8 | 100 | |
| A*03:01 | KIFPPTEPK (2045) | N | | | B.1.351 |
| A*03:01 | KCYGVSPTK (466) | S | 152.6 | 100 | |
| A*03:01 | KCYGVSLTK (2046) | S | | | B.1.351 |
| A*03:01 | VTNNTFTLK (751) | ORF1ab | 19.8 | 40 | |
| A*03:01 | KTIQPRVEK (1796) | ORF1ab | 113.2 | 40 | |
| A*11:01 | KTFPPTEPK (1447) | N | 6.3 | 100 | |
| A*11:01 | KIFPPTEPK (2045) | N | | | B.1.351 |
| A*11:01 | VTDTPKGPK (1520) | ORF1ab | 160.6 | 60 | |
| A*11:01 | ATEGALNTPK (1448) | N | 55.5 | 80 | |
| A*11:01 | ATDGALNTPK (2047 | | | | BA.5 |
| A*11:01 | ASAFFGMSR (825) | N | 14.4 | 40 | |
| A*11:01 | ATSRTLSYYK (811)) | M | 7.9 | 60 | |
| A*11:01 | KSTNLVKNK (2048) | S | | | |
| A*11:01 | STFNVPMEK (1888) | ORF1ab | | | |
| A*11:01 | ASMPTTIAK (1890) | ORF1ab | | | |
| A*11:01 | SAFAMMFVK (821) | ORF1ab | | | |
| A*24:02 | QYIKWPWYI (443) | S | 13.2 | 60 | |
| A*24:02 | VYFLQSINF (877) | ORF3a | 47.4 | 80 | |
| A*24:02 | VYIGDPAQL (848) | ORF1ab | 206 | 40 | |
| B*07:02 | SPRWYFYYL (986) | N | 6.3 | 80 | |
| B*07:02 | RPDTRYVL (1383) | ORF1ab | 56.9 | 80 | |
| B*07:02 | IPRRNVATL (944) | ORF1ab | 5.1 | 20 | |
| B*07:02 | KPRQKRTAT (1480) | N | | | |
| B*07:02 | FPRGQGVPI (1478) | N | | | |
| B*07:02 | RAAEIRASA (2049) | S | | | |
| B*07:02 | SPRRARSVA (456) | S | | | |
| B*07:02 | SRRRARSVA (487) | S | | | B.1.617.2 |
| B*07:02 | SHRRARSVA (488) | S | | | B.1.1.7, P.1, |
| | | | | | B.1.1.529, BA.2, BA.5 |
| B*07:02 | SPRRARSVV (2050) | S | | | P1 |
| B*15:01 | AQFAPSASAF (1459) | N | | | |
| β27:05 | QRNAPRITF (1484) | N | | | |
| β27:05 | HRNAPRITF (2051) | N | | | B.1.351 |
| β27:05 | LRNAPRITF (2052) | N | | | B.1.617.2 |
| β27:05 | QRNALRITF (2053) | N | | | B.1.1.529, BA.2, BA.5 |
| β27:05 | QRNASRITF (2054) | N | | | B.1.351 |
| β27:05 | QRNAFRITF (2055) | N | | | BA.5 |
| B35:01 | TPSGTWLTY (1117) | N | | | |
| B35:01 | TPLGTWLTY (2056) | N | | | B.1.617.2 |
| B35:01 | LLNKHIDAY (1482) | N | | | |
| B35:01 | LPIGINITRF (1142) | S | | | |
| B40:01 | MEVTPSGTWL (419) | N | | | |
| B40:01 | MEVTPLGTWL (2057) | N | | | B.1.617.2 |
| B40:01 | IEYPIIGDEL (1163) | ORF1ab | | | |
| B40:01 | AEIRASANL (1234) | S | | | |

*SEQ ID NOs are provided in parenthesis after the sequence.

### (ii) Redirected T-Cell-MPs

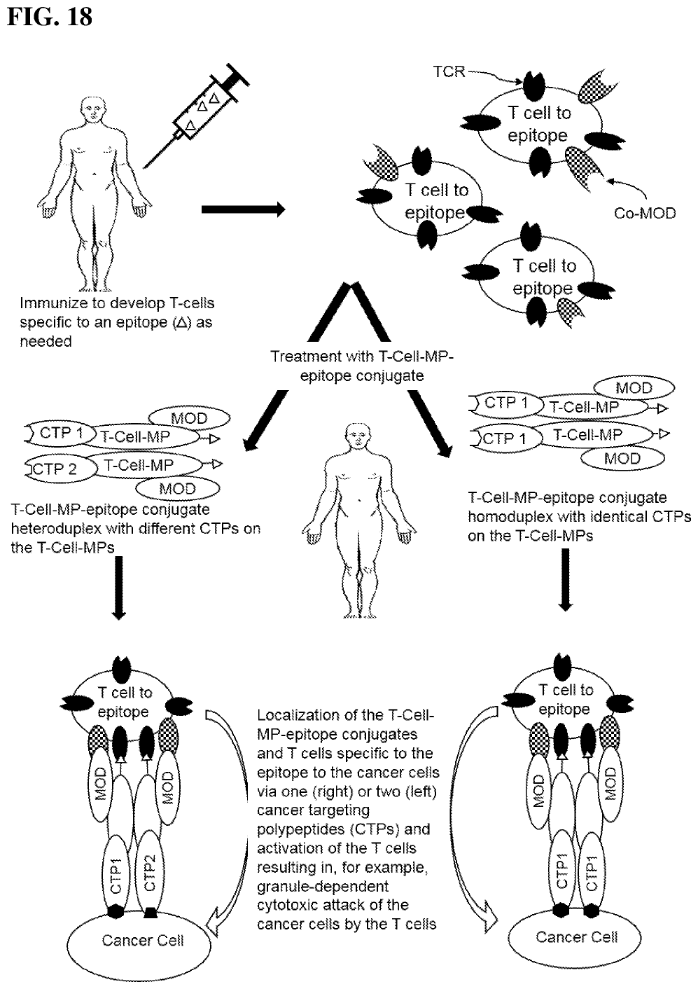

The present disclosure also includes and provides for methods of redirecting a T cell (e.g., a CD8+ effector T cell) having a TCR directed to a specified epitope (e.g., a specified epitope of a SARS-CoV-2 protein) toward tissues or cells (e.g., neoplastic cells that are benign (comprised of non-cancerous cells), precancerous, or malignant (comprised of cancerous cells) utilizing a “redirected T-Cell-MP-epitope conjugate”. Redirected T-Cell-MP-epitope conjugates are T-Cell-MP-epitope conjugates that bear at least one targeting sequences (e.g., CTPs) that bind to and directed the T-Cell-MP-epitope conjugate to one or more markers found on specific tissues or cells (e.g., found on neoplastic cells). For example, T cells (e.g., a CD8+ effector T cell) having a TCR directed to a specified epitope (e.g., an epitope of a SARS-CoV-2 protein) can be redirected toward cancerous cells through the use of a T-Cell-MP-epitope conjugate bearing one or more CTPs directed epitopes found on the cancerous cell as illustrated in FIG. 18. Targeting sequences are discussed in more detail above.

Where the T-Cell-MP-epitope conjugate includes targeting sequence(s) directed against one or more cancer antigens (e.g., CTPs) that direct the T-Cell-MP-epitope conjugate to a target cancer cell or tissue, the epitope of the T-cell-MP-epitope conjugate (the epitope covalently bound to a chemical conjugation site) is advantageously one that binds T cells that are already are present in the patient, e.g., resulting from exposure to a foreign agent such as a coronavirus (e.g. SARS-CoV-2) and/or from vaccination (e.g. with a SARS-CoV-2 vaccine, e.g., Pfizer, Moderna or J&J). For example, the epitope can be an epitope present in a viral antigen encoded by a virus that infects a substantial number of the human population such as SARS-CoV-2, and for which a majority of the human population has T cells directed against the virus through infection and/or vaccination. Thus, for example, in order to prime, or increase the number of, T cells that will specifically bind and be stimulated by the T-cell-MP-epitope conjugate, the patient can be vaccinated, e.g., with a SARS-CoV-2 vaccine, prior to treatment with the T-Cell-MP-epitope conjugate. The result is that T cells present in the patient directed to epitopes other than those found on the neoplastic cells are effectively redirected to the target the neoplastic cells (e.g., cancer cells) by the targeting sequence (e.g., CTP). Activation of the T cells by the T-Cell-MP-epitope conjugate can result in killing of the targeted cell, e.g., through granule-dependent cytotoxic attack. Moreover, the ability to use more than one targeting sequence (e.g., CTPs against different cancer antigens or different epitopes of a single antigen) can decrease the cancer's ability to mutate and thereby escape effective therapeutic intervention. See, e.g., FIG. 18.

A indicated above, the peptide conjugated to a redirected T-Cell-MP may present a coronavirus epitope. Where the T-Cell-MP comprises an HLA-E polypeptide sequence the peptide presenting a coronavirus epitope may be an HLA-E restricted epitope. Because of the essentially invariant nature of HLA-E molecules, such T-Cell-MP-epitope conjugates can be used to treat a large divers group of individuals having otherwise genetically diverse HLA genotypes using a single T-Cell-MP-epitope conjugate. An example of a SARs-Co-V epitope that may be employed is the Nsp 13 epitope VMPLSAPTL (SEQ ID NO:2058, aas 232-240 of the helicase provided in FIG. 20C), which can be conjugated to a redirected T-Cell-MP comprising an HLA-E heavy chain sequence to form an epitope conjugate that may engage T cells via the TCR and drive an adaptive immune response. VMPLSAPTL epitope does not, however, bind the inhibitory NKG2A receptors of NK cells, and accordingly, HLA-E presentation of VMPLSAPTL may also lead to NK cell cytotoxic activity (e.g., release of lytic granule contents (such as granzymes and perforin), and induction of apoptosis mediated by expression of Fas Ligand or TRAIL). Hammer et al., 2022, Cell Reports 38, 110503. Thus, T-Cell-MPs conjugated to that epitope directed to tumors or other target tissues may be utilized to locally stimulate NK cell action (e.g., by releasing the cells from CD94/NKG2A mediated suppression of those activities).

Peptides presenting epitopes other than coronavirus epitopes (e.g., other than SARS-CoV-2 epitopes) may also be employed with redirected T-Cell-MP to form epitope conjugates that target specific tissues or cells. Among the non-coronavirus epitopes that may be employed are those of other viral antigens. See, e.g., the list of viral antigens in Published PCT application WO 2020/243315 (Cue Biopharma, Inc.). The epitope may be chosen from a virus that infects a majority of the human population such as cytomegalovirus (CMV), Epstein-Barr virus (EBV), human papilloma virus, adenovirus, and the like, or which is included in a vaccine that can be administered to a patient prior to or concurrently with administration of the T-Cell-MP-epitope conjugate that includes a CTP.

Non-coronavirus epitopes that may be conjugated to T-Cell-MPs comprising an HLA-E aa polypeptide sequence (see e.g., FIG. 3 H) include those described by Huisman et al., (BioRxiv preprint posted Aug. 8, 2022, doi.org/10.1101/2022.08.03.502719). Because HLA-E-epitope complexes not only bind the TCR present on T cells, but may also engage both NK cell inhibitory CD94/NKG2A receptors and the NK cell stimulatory CD94/NKG2C receptors, different patterns of response can be obtained depending upon the epitope conjugated to the HLA-E containing T-Cell-MP. T-Cell-MP HLA-E restricted epitope conjugates that engage NK cell inhibitory NKG2A receptors conjugated to a T-Cell-MP would favor a T cell mediated response with less NK cell activation than would occur using an epitope that engages NK cell stimulatory NKG2C receptors conjugated to an otherwise identical T-Cell-MP. The extent and type of response obtained with a specific HLA-E restricted epitope conjugated to a T-Cell-MP in vitro or in vivo will depend upon, among other things, the level of CD94/NKG2A and CD94/NKG2C receptors present on the cells.

HLA-E restricted peptides that engage the CD94/NKG2A receptor that may be conjugated to a T-Cell-MP comprising an HLA-E polypeptide sequence include, but are not limited to: QMPSRSLLF (SEQ ID NO:2059), TLPKRGLFL (SEQ ID NO:2060), TGPWRSLWI (SEQ ID NO:2061), ILTDRSLWL (SEQ ID NO:2062), VNPGRSLFL (SEQ ID NO:2063), WNRLFPPLR (SEQ ID NO:2064), TLPERTLYL (SEQ ID NO:2065), FLPNRSLLF (SEQ ID NO: 2066), VMGDRSVLY (SEQ ID NO:2067), and VMADKSIFY (SEQ ID NO:2068).

HLA-E restricted peptides that engage the CD94/NKG2C receptor that may be conjugated to a T-Cell-MP comprising an HLA-E polypeptide sequence include, but are not limited to: VMPPRTLLL (SEQ ID NO.: 2069), VMAPRTLFL (SEQ ID NO:2070), VNPGRSLFL (SEQ ID NO:2063), QMPSRSLLF (SEQ ID NO.: 2071), TLPERTLYL (SEQ ID NO:2065), VMPGRTLCF (SEQ ID NO:2072), ILTDRSLWL (SEQ ID NO:262), RMPPRSVLL (SEQ ID NO: 2073), TAPARTMFL (SEQ ID NO.: 2074), and NMPARTVLF SEQ ID NO:2075. The peptide WNRILPNAY SEQ ID NO: 2076, may also be employed as an HLA-E restricted epitope. Other HLA-E restricted peptides that may be conjugated to an T-Cell-MP comprising an HLA-E polypeptide MHC-H sequence include those comprising the sequence X1-X2-X3-X4-R-X6-X7-X8-X9-X10, where: X1 is S, K, A, I, L, T, or absent; X2 is L, I, K or V; X3 is P, A or S; X4 is G, V, P, D or R; X6 is S or T; X7 is L, I, or V; X8 is F, W, or E; X9 is L. F, W or E; and X10 F, H, S, A or absent SEQ ID NO:2077. In two specific instances the HLA-E restricted peptide comprises the sequences LP (G or V) RSLFL SEQ ID NO:2078, or LP (G or V) RSLWL (SEQ ID NO:2079).

HLA-E restricted peptides from Peptides from CMV that may be conjugated to a T-Cell-MP comprising an HLA-E polypeptide sequence include, but are not limited to: VLPHRTQFL (SEQ ID NO:2080), TGAARSFFF (SEQ ID NO: 2081), and VMAPRTLIL (SEQ ID NO:2082). The CMV peptides SAPLKTRFL “(SEQ ID NO:2083), and SVPLKTRFL (SEQ ID NO:2084) may also be employed.

### 9 Payloads-Drug And Other Conjugates

A broad variety of payloads may be associated with unconjugated T-Cell-MPs and T-Cell-MP-coronavirus epitope conjugates, which may incorporate more than one type of payload conjugated (covalently) to the T-Cell-MPs at chemical conjugation sites. In addition, where the T-Cell-MP molecules or their epitope conjugates multimerize (e.g., form a heteroduplex using interspecific scaffolds), it may be possible to incorporate T-Cell-MPs with different payloads into a multimer. Accordingly, it is possible to introduce one or more payloads selected, for example, from the group consisting of anticancer or other therapeutic agents such as antiviral agents, antibiotics, diagnostic agents or labels, and the like.

T-Cell-MP polypeptides (e.g., a scaffold or Fc polypeptide) can be modified with crosslinking reagents to conjugate payloads and/or epitopes to the T-Cell-MP (e.g., at a chemical conjugation site such as an engineered cysteine or lysine). Exemplary crosslinking agents include, but are not limited to, succinimidyl 4-(N-maleimidomethyl)-cyclohexane-1-carboxylate (SMCC), sulfo-SMCC, maleimidobenzoyl-N-hydroxysuccinimide ester (MBS), sulfo-MBS or succinimidyl-iodoacetate. Introducing payloads using an excess of such crosslinking agents can result in multiple molecules of payload being incorporated into the T-Cell-MP. Some bifunctional linkers for introducing payloads into T-Cell-MPs and their epitope conjugates include cleavable linkers or non-cleavable linkers. In some cases, the payload linker is a protease-cleavable linker. Suitable payload linkers include, but are not limited to, molecules comprising peptides (e.g., from 2 to 10 amino acids in length; such as 2, 3, 4, 5, 6, 7, 8, 9, or 10 amino acids in length), alkyl chains, poly(ethylene glycol), disulfide groups, thioether groups, acid labile groups, photolabile groups, peptidase labile groups, or esterase labile groups. Non-limiting examples of suitable reagents for use as payload linkers include: N-succinimidyl-[(N-maleimidopropionamido)-tetraethyleneglycol]ester (NHS-PEG4-maleimide); N-succinimidyl 4-(2-pyridyldithio) butanoate (SPDB); N-succinimidyl 4-(2-pyridyldithio) 2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); κ-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); N-(α-maleimidoacetoxy)-succinimide ester (AMAS); succinimidyl-6-(β-maleimidopropionamide) hexanoate (SMPH); N-succinimidyl 4-(p-maleimidophenyl) butyrate (SMPB); N-(p-maleimidophenyl) isocyanate (PMPI); N-succinimidyl 4 (2-pyridylthio) pentanoate (SPP); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexanecarboxylate (SMCC); N-succinimidyl-4-(N-maleimidomethyl)-cyclohexane-1-carboxy-(6-amidocaproate), a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido) propionate (SBAP)

Control of the stoichiometry of the reaction between a T-Cell-MP and a payload or a crosslinker used to introduce a payload may result in some selective modification where engineered sites with chemistry orthogonal to all other groups in the molecule is not utilized. Reagents that display far more selectivity, such as the bis-thio linkers discussed above, tend to permit more precise control of the location and stoichiometry than reagents that react with single lysine, or cysteine residues.

Where a T-Cell-MP comprises a Fc polypeptide, the Fc polypeptide can comprise one or more covalently attached molecules of payload that are attached directly or indirectly through a linker. By way of example, where a T-Cell-MP comprises a Fc polypeptide, the polypeptide chain comprising the Fc polypeptide can be of the formula (A)-(L)-(C), where (A) is the polypeptide chain comprising the Fc polypeptide; where (L), if present, is a linker; and where (C) is a payload. (L), if present, links (A) to (C). In some cases, the polypeptide chain comprising the Fc polypeptide can be coupled to more than one molecule of payload (e.g., 2, 3, 4, 5, or more than 5 payload molecules).

Payloads may be selected from the group consisting of therapeutic agents, diagnostic agents (e.g., labels), nucleotide or nucleoside analogs, nucleic acids or synthetic nucleic acids (e.g., antisense nucleic acids, small interfering RNA, double stranded (ds) DNA, single stranded (ss) DNA, ssRNA, dsRNA), toxins, liposomes (e.g., incorporating a therapeutic agent), nanoparticles (e.g., gold or other metal bearing nucleic acids or other molecules, lipids, particle bearing nucleic acids or other molecules), and combinations thereof.

As discussed above, a polypeptide chain of a T-Cell-MP or its epitope conjugate can comprise a payload linked (e.g., covalently attached) to the T-Cell-MP polypeptide chain at one or more chemical conjugation sites. The linkage between a payload and a polypeptide chain of a T-Cell-MP may be a direct or an indirect linkage. Direct linkage can involve linkage directly to an amino acid side chain. Indirect linkage can be linkage via a linker. The payload (e.g., an anticancer agent) can be linked to a polypeptide chain (e.g., a Fc polypeptide) of an unconjugated T-Cell-MP or a T-Cell-MP-coronavirus epitope conjugate via a thioether bond, an amide bond, a carbamate bond, a disulfide bond, or an ether bond.

The polypeptide chain(s) of a T-Cell-MP may comprise as payload(s) one or more molecules of photo detectable labels (e.g., dyes, fluorescent labels, phosphorescent labels, luminescent labels), contrast agents (e.g., iodine or barium containing materials), radiolabels, imaging agents, spin labels, Forster Resonance Energy Transfer (FRET)-type labels, paramagnetic labels/imaging agents (e.g., gadolinium containing magnetic resonance imaging labels), ultrasound labels and combinations thereof.

In some embodiments, the payload comprises a label that is, or that includes, a radioisotope. Examples of radioisotopes or other labels include, but are not limited to, ³H, ¹¹C, ¹⁴C, ¹⁵N, ¹⁷O, ³⁵S, ¹⁸F, ³²P, ³³P, ⁶⁴Cu, ⁶⁸Ga, ⁸⁹Zr, ⁹⁰Y, ⁹⁹Tc, ¹²³I, ¹²⁴I, ¹²⁵I, ¹³¹I, ¹¹¹In, ¹³¹In, ¹⁵³Sm, ¹⁸⁶Re, ¹⁸⁸Re, ²¹¹At, ²¹²Bi, and ¹⁵³Pb.

### VII. NUCLEIC ACIDS

The present disclosure provides a nucleic acid comprising a nucleotide sequence encoding a T-Cell-MP or more than one T-Cell-MP (e.g., a pair of T-Cell-MPs that form an interspecific heteroduplex). The individual T-Cell-MPs of a heteromer (e.g., an interspecific pair forming a heteroduplex) may be encoded in separate nucleic acids. Alternatively, the T-Cell-MPs of a heteromeric T-Cell-MP (e.g., an interspecific pair) may also be encoded in a single nucleic acid. Such nucleic acids include those comprising a nucleotide sequence encoding a T-Cell-MP having at least one chemical conjugation site (e.g., cysteine residues) that are provided in the MHC-H, β2M or scaffold polypeptide sequences of the T-Cell-MP, or into any linker (e.g., an L3 linker) joining those polypeptide sequences.

### A. Nucleic Acids Encoding Unconjugated T-Cell-MPs

The present disclosure provides nucleic acids comprising nucleotide sequences encoding an unconjugated T-Cell-MP that may form higher order complexes (e.g., duplexes). The nucleotide sequences encoding an unconjugated T-Cell-MP may be operably linked to transcriptional control elements, e.g., promoters, such as promoters that are functional in a eukaryotic cell, where the promoter can be a constitutive promoter or an inducible promoter. As noted above, in some cases, the individual unconjugated T-Cell-MPs form heteromeric complexes (e.g., a heteroduplex T-Cell-MP comprising an interspecific scaffold pair). Heteromeric unconjugated T-Cell-MPs may be encoded in a single polycistronic nucleic acid sequence. Alternatively, heteromeric T cell-MPs may be encoded in separate monocistronic nucleic acid sequences with expression driven by separate transcriptional control elements. Where separate monocistronic sequences are utilized, they may be present in a single vector or in separate vectors.

The present disclosure includes and provides for a nucleic acid sequence encoding an unconjugated T-Cell-MP polypeptide that comprises (e.g., from N-terminus to C-terminus): (i) optionally one or more MOD polypeptide sequences (e.g., two or more MOD polypeptide sequences, such as in tandem, wherein when there are two or more MOD polypeptide sequences they are optionally joined to each other by independently selected L1 linkers); (ii) an optional L2 linker polypeptide sequence joining the one or more MOD polypeptide sequences to a β2M polypeptide sequence; (iii) the β2M polypeptide sequence; (iv) an optional L3 linker polypeptide sequence (e.g., from 10⁻⁵⁰ aa in length); (v) a class I MHC-H polypeptide sequence; (vi) an optional L4 linker polypeptide sequence; (vii) a scaffold polypeptide sequence (e.g., an Ig Fc sequence); (viii) an optional L5 linker polypeptide sequence; and (ix) optionally one or more MOD polypeptide sequence (e.g., two or more MOD polypeptide sequences, such as in tandem, wherein when there are two or more MOD polypeptide sequences they are optionally joined to each other by independently selected L6 linkers); wherein the unconjugated T cell modulatory polypeptide comprises at least one MOD polypeptide sequence (e.g., the MOD(s) of element (i) and/or (ix); and wherein at least one of the β2M polypeptide sequence, the L3 linker polypeptide sequence, and/or the MHC-H polypeptide sequence comprises a chemical conjugation site for epitope conjugation.

The present disclosure includes and provides for a nucleic acid sequence encoding an unconjugated T-Cell-MP polypeptide that comprises from N- to C-terminus: (i) optionally one or more MOD polypeptide sequences (e.g., two or more MOD polypeptide sequences, such as in tandem, wherein when there are two or more MOD polypeptide sequences they are optionally joined to each other by independently selected L1 linkers); (ii) an optional L2 linker polypeptide sequence; (iii) a β2M polypeptide sequence; (iv) an optional L3 linker polypeptide sequence (e.g., from 10-50 aa in length); (v) a class I MHC-H polypeptide sequence; (vi) an optional L4 linker polypeptide sequence; (vii) a scaffold polypeptide sequence (e.g., an Ig Fc sequence); (viii) an optional L5 linker polypeptide sequence; and (ix) optionally one or more MOD polypeptide sequence (e.g., two or more MOD polypeptide sequences, such as in tandem, wherein when there are two or more MOD polypeptide sequences they are optionally joined to each other by independently selected L6 linkers); wherein the unconjugated T cell modulatory polypeptide comprises at least one MOD polypeptide sequence (e.g., the MOD(s) of element (i) and/or (ix)); and wherein at least one of the β2M polypeptide sequence, the L3 linker polypeptide sequence, and/or the MHC-H polypeptide sequence comprises a chemical conjugation site for epitope conjugation.

The present disclosure includes and provides for a nucleic acid sequence encoding an unconjugated T-Cell-MP polypeptide that comprises from N- to C-terminus: (i) one or more MOD polypeptide sequences (e.g., two or more MOD polypeptide sequences, such as in tandem, wherein when there are two or more MOD polypeptide sequences they are optionally joined to each other by independently selected L1 linkers); (ii) an optional L2 linker polypeptide sequence; (iii) a β2M polypeptide sequence; (iv) an optional L3 linker polypeptide sequence (e.g., from 10-50 aa in length); (v) a class I MHC-H polypeptide sequence; (vi) an optional L4 linker polypeptide sequence; (vii) a scaffold polypeptide sequence (e.g., an Ig Fc sequence); (viii) an optional L5 linker polypeptide sequence; and (ix) optionally one or more MOD polypeptide sequence (e.g., two or more MOD polypeptide sequences, such as in tandem, wherein when there are two or more MOD polypeptide sequences they are optionally joined to each other by independently selected L6 linkers); wherein the unconjugated T cell modulatory polypeptide comprises at least one MOD polypeptide sequence (e.g., the MOD(s) of element (i) and/or (ix); and wherein at least one of the β2M polypeptide sequence, the L3 linker polypeptide sequence, and/or the MHC-H polypeptide sequence comprises a chemical conjugation site for epitope conjugation.

Suitable MHC-H, B2-microglobulin (β2M) polypeptide, and scaffold polypeptides are described above. The MHC-H polypeptide may be an HLA-A, HLA-B, HLA-C, HLA-E, HLA-F, or HLA-G heavy chain. In some cases, the MHC-H polypeptide comprises an amino acid sequence having at least 85% aa sequence identity to the amino acid sequence depicted in any one of FIGS. 3A-3H. In such an embodiment the MHC Class I heavy chain polypeptide may not include a transmembrane anchoring domain and intracellular domain (see, e.g., the MHC-H polypeptides in FIG. 3D). In some cases, the first MHC polypeptide comprises a 32-microglobulin (β2M) polypeptide; and the second MHC polypeptide comprises a MHC Class I heavy chain polypeptide. In some cases, the β2M polypeptide comprises an amino acid sequence having at least about 85% (e.g., at lease about 90%, 95%, 98%, 99%, or even 100%) aa sequence identity to a β2M amino acid sequence depicted in FIG. 4

### B. Recombinant Expression Vectors

The present disclosure provides recombinant expression vectors comprising a nucleic acid sequence encoding at least one T-Cell-MP (e.g., two or more T-Cell-MPs, as in, for example, an interspecific pair forming a duplex T-Cell-MP). In some cases, the recombinant expression vector is a non-viral vector. In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example for eukaryotic host cells: pXT1, pSG5 (Stratagene®), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc., may be used in the expression vector (see, e.g., Bitter et al. (1987), Methods in Enzymology, 153:516-544).

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### VIII. METHODS OF GENERATING AND SELECTING T-CELL-MPS

The present disclosure provides a method of obtaining T-Cell-MPs (both unconjugated T-Cell-MPs and/or T-Cell-MP-epitope conjugates) including in duplex and other higher order aggregates, which may include one or more wt. MOD polypeptide sequences and/or one or more variant MOD polypeptide sequences that exhibit lower affinity for a Co-MOD compared to the affinity of the corresponding wt. MOD polypeptide sequence for the Co-MOD, the method comprising:
A) generating a T-Cell-MP (or a higher order complex such as a duplex) by introducing into cells or cell-free systems one or more nucleic acids encoding an unconjugated T-Cell-MP or each of the unconjugated T-Cell-MPs that make up a heteromer (e.g., a heterodimeric duplex of unconjugated T-Cell-MPs);
wherein when the T-Cell-MP comprises one or more nascent chemical conjugation sites, the nascent chemical conjugation site may be activated to produce an unconjugated T-Cell-MP with a chemical conjugation site (e.g., reacting sulfatase motifs with an FGE to convert a Cys residue to a fGly residue if the cells translating the T-Cell-MP nucleic acids do not express a formylglycine generating enzyme).

The above-mentioned method of generating T-Cell-MPs may further comprise providing one or more nucleic acids encoding the unconjugated T-Cell-MP, including those specifically described in the present disclosure, which may be present in a recombinant expression vector and/or operably linked to a transcriptional control element such as those functional in a eukaryotic cell. The method may be stopped at this point and the unconjugated T-Cell-MP (e.g., unconjugated duplex T-Cell-MP) that is unpurified (including cell lysates and unpurified media) may be obtained. Alternatively, the unconjugated T-Cell-MP may be purified using, for example, one or more of salt precipitation (e.g., ammonium sulfate), affinity chromatography, and/or size exclusion chromatography, to produce crude (less than 60% by weight), initially refined (at least 60% by weight), partly refined (at least 80% by weight), substantially refined (at least 95% by weight), partially pure or partially purified (at least 98% by weight), substantially pure or substantially purified (at least 99% by weight), essentially pure or essentially purified (at least 99.5% by weight) or purified (at least 99.8%) or highly purified (at least 99.9% by weight) unconjugated T-Cell-MP based on the total weight of protein present in the sample may be obtained by purification. Purification of T-Cell-MPs comprising an Ig Fc sequence may be purified by a method comprising chromatography on immobilized protein A or protein G, after which size based chromatographic separation (e.g., size exclusion chromatography) may be employed to further purify the T-Cell-MP. Where a T-Cell-MP-epitope conjugate is desired, the method may be continued by reacting anywhere from a crude preparation to a highly purified preparation of T-Cell-MP with an epitope presenting molecule as in step B:
B) providing an epitope (e.g., an epitope-presenting peptide) suitable for conjugation with the chemical conjugation site present in the unconjugated T-Cell-MP of step A (e.g., a hydrazinyl or hydrazinyl indole modified peptide for reaction with a formyl glycine of a sulfatase motif or a maleimide containing peptide for reaction with a cysteine residue), and contacting the epitope with the T-Cell-MP (e.g., under suitable reaction conditions) to produce a T-Cell-MP-epitope conjugate.

The choice of how purified the unconjugated T-Cell-MP entering into the conjugation reaction needs to be depends on a number of factors including, but not limited to, the conjugation reaction and conditions, the potential for side reactions, and the degree to which the final epitope conjugate will need to be purified for its intended use.

The T-Cell-MP-epitope conjugate (e.g., as a duplex or a higher order complex) may be purified by, for example, salt precipitation, size based separation (e.g., chromatography or dialysis), isoelectric focusing, and/or affinity chromatography, so that it is at least partly refined (at least 80% by weight of protein present in the sample), substantially refined (at least 95% by weight), partially pure or partially purified (at least 98% by weight), substantially pure or substantially purified (at least 99% by weight), essentially pure or essentially purified (at least 99.5% by weight), purified (at least 99.8%), or highly purified (at least 99.9% by weight) of the T-Cell-MP-epitope conjugate based on the total weight of protein present in the sample.

Where it is desirable for a T-Cell-MP or higher order complexes to contain a payload, the payload may be reacted with the unconjugated T-Cell-MP or the T-Cell-MP-epitope conjugate. The selectivity of the epitope and the payload for different conjugation sites may be controlled through the use of orthogonal chemistries and/or control of stoichiometry in the conjugation reactions. In embodiments, linkers (e.g., polypeptides or other bifunctional chemical linkers) may be used to attach the epitope and/or payloads to their conjugation sites.

A variety of cells and cell-free systems may be used for the preparation of unconjugated T-Cell-MPs. As discussed in the section titled “Genetically Modified Host Cells,” the cells may be eukaryotic origin, and more specifically of mammalian, primate or even human origin.

The present disclosure provides a method of obtaining an unconjugated T-Cell-MP or T-Cell-MP-epitope conjugate (or their higher order complexes, such as duplexes) comprising one or more wt. MODs and/or variant MODs that exhibit reduced affinity for a Co-MOD compared to the affinity of the corresponding parental wt. MOD for the Co-MOD. Where a variant MOD having reduced affinity is desired, the method can comprise preparing a library of variant MOD polypeptides (e.g., that have at least one insertion, deletion or substitution) and selecting from the library of MOD polypeptides a plurality of members that exhibit reduced affinity for their Co-MOD (such as by BLI as described above). Once a variant MOD is selected a nucleic acid encoding the unconjugated T-Cell-MP including the variant MOD is prepared and expressed. After the unconjugated T-Cell-MP has been expressed it can be purified, and if desired conjugated to an epitope to produce the selected T-Cell-MP-epitope conjugate. The process may be repeated to prepare a library of unconjugated T-Cell-MPs or their epitope conjugates.

The present disclosure provides a method of obtaining a T-Cell-MP-epitope conjugate or its higher order complexes, such as a duplex) that exhibits selective binding to a T cell, the method comprising:
A) generating a library of T-Cell-MP-epitope conjugates (or their higher order complexes) comprising a plurality of members, wherein each member comprises a different variant MOD on the T-Cell-MP-epitope conjugate, wherein the variant MOD differs in amino acid sequence (e.g., by from 1 aa to 10 aas) from its parental wt. MOD, and wherein the T-Cell-MP-epitope conjugate library members further comprise an epitope tag or a fluorescent label), and
B) contacting a T-Cell-MP-epitope conjugate library member with a target T cell expressing on its surface: i) a Co-MOD that binds the parental wt. MOD; and ii) a TCR that binds to the epitope;
C) selecting a T-Cell-MP-epitope conjugate library member that selectively binds the target T cell relative to its binding under the same conditions to a control T cell that comprises: i) the Co-MOD that binds the parental wt. MOD; and ii) a TCR that binds to an epitope other than the epitope present in the T-Cell-MP library member (e.g., choosing the T-Cell-MP-epitope conjugate that has higher avidity or affinity for the target T cell than the control T cell such as by BLI as described above).

A T-Cell-MP-epitope conjugate library member that is identified as selectively binding to a target T cell may be isolated from the library.

When the T-Cell-MP-epitope conjugate comprises an epitope tag or label, identifying a T-Cell-MP-epitope conjugate selective for a target T cell may comprise detecting the epitope tag or label associated with target and/or control T cells by using, for example, flow cytometry. While labeled T-Cell-MPs (e.g., fluorescently labeled) do not require modification to be detected, epitope tagged molecules may require contacting with an agent that renders the epitope tag visible (e.g., a fluorescent agent that binds the epitope tag). The affinity/avidity of the T-Cell-MP-epitope conjugate can be determined by measuring the agent or label associated with target and control T cells (e.g., by measuring the mean fluorescence intensity using flow cytometry) over a range of concentrations. The T-Cell-MP-epitope conjugate that binds with the highest affinity or avidity to the target T cell relative to the control T cell is understood to selectively bind to the target T cell.

### IX. GENETICALLY MODIFIED HOST CELLS

The present disclosure provides a genetically modified host cell, where the host cell is genetically modified with a nucleic acid (e.g., a nucleic acid encoding an unconjugated T-Cell-MP that may be operably linked to a promoter). Where such cells express T-Cell-MPs they may be utilized in methods of generating and selecting T-Cell-MPs as discussed in the preceding section.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, Hela cells (e.g., American Type Culture Collection (ATCC) No. CCL-2™), CHO cells (e.g., ATCC Nos. CRL-9618™, CCL-61™, CRL9096), 293 cells (e.g., ATCC No. CRL-1573™), Vero cells, NIH 3T3 cells (e.g., ATCC No. CRL-1658), Huh-7 cells, BHK cells (e.g., ATCC No. CCL-10™), PC12 cells (ATCC No. CRL-1721™), COS cells, COS-7 cells (ATCC No. CRL 1651), RAT1 cells, mouse L cells (ATCC No. CCLI.3), human embryonic kidney (HEK) cells (ATCC No. CRL 1573), HLHepG2 cells, and the like.

In some cases, the host cell is a mammalian cell that has been genetically modified such that it does not synthesize endogenous β2M and/or such that it does not synthesize endogenous MHC Class I heavy chains (MHC-H). In addition to the foregoing, host cells expressing formylglycine generating enzyme (FGE) activity are discussed above for use with T-Cell-MPs comprising a sulfatase motif, and such cells may advantageously be modified such that they do not express at least one, if not both, of the endogenous MHC β2M and MHC-H proteins.

### X. COMPOSITIONS AND FORMULATIONS

The present disclosure provides compositions and formulations, including pharmaceutical compositions and formulations. Compositions may comprise: a) a T-Cell-MP or T-Cell-MP-epitope conjugate and one or more pharmaceutically acceptable additives, e.g., nonionic surfactants, stabilizers, buffering agents, amino acids such as arginine and proline, etc., a variety of which are known in the art and therefore not discussed in detail herein. Pharmaceutically acceptable additives have been amply described in a variety of publications including but certainly not limited to publications such as, for example, “Remington: The Science and Practice of Pharmacy”, 19^{th} Ed. (1995), or latest edition, Mack Publishing Co; A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc., and updated editions of the foregoing.

The present disclosure also provides compositions and formulations, including pharmaceutical compositions, comprising a nucleic acid or a recombinant expression vector, where the nucleic acid or expression vector encodes all or part of a T-Cell-MP or its higher order complexes (e.g., one T-Cell-MP of a heterodimeric T-Cell-MP duplex).

Compositions will generally be in the form of aqueous or other solutions, but also may be in the form of powders, granules, tablets, pills, suppositories, capsules, suspensions, sprays, and the like. The composition may be formulated according to the various routes of administration described below.

Where a T-Cell-MP-epitope conjugate is administered as an injectable (e.g., subcutaneously, intraperitoneally, intramuscularly, and/or intravenously) directly into a tissue, a formulation can be provided as a ready-to-use dosage form, a non-aqueous form (e.g., a reconstitutable storage-stable powder), or an aqueous form, such as liquid composed of pharmaceutically acceptable carriers and excipients. T-Cell-MP formulations may also be provided so as to enhance serum half-life of the subject protein following administration. For example, the T-Cell-MP may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

Other examples of formulations suitable for parenteral administration include those comprising sterile injection solutions, salts, anti-oxidants, bacteriostats, and/or solutes that render the formulation isotonic with the blood of the intended recipient. Such parenteral formulations may also include one or more independently selected suspending agents, solubilizers, thickening agents, stabilizers, and preservatives.

Formulations or pharmaceutical composition comprising a T-Cell-MP or T-Cell-MP-epitope conjugate can be present in a container, e.g., a sterile container, such as a syringe. The formulations can also be presented in unit-dose or multi-dose sealed containers, such as ampules and vials, any of which may be sterile. The formulation or pharmaceutical compositions may be stored in a sterile freeze-dried (lyophilized) condition requiring only the addition of the sterile liquid excipient, for example, water, for injections, immediately prior to use. Extemporaneous injection solutions and suspensions can be prepared from sterile solutions, powders, granules, and/or tablets that comprise the T-Cell-MP or T-Cell-MP-epitope conjugate. When a T-Cell-MP-epitope conjugate is administered intravenously, it may be administered neat or diluted with sterile saline prior to administration.

The concentration of a T-Cell-MP in a formulation can vary widely (e.g., from less than about 0.1%, usually at or at least about 2% to as much as 20% to 50% or more by weight) and will usually be selected primarily based on the particular components of the formulation. The concentration of a T-Cell-MP in an aqueous formulation can be, for example, from about 0.1 mg/ml to about 50 or more mg/mL, e.g., from about 1 mg/ml to about 20 mg/mL, from about 5 mg/mL to about 15 mg/mL, e.g., about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL.

In some cases, a T-Cell-MP is present in a liquid composition. Thus, the present disclosure provides compositions (e.g., liquid compositions, including pharmaceutical compositions) comprising a T-Cell-MP. The present disclosure also provides a composition comprising: a) a T-Cell-MP; and b) saline (e.g., 0.9% or about 0.9% NaCl). In some cases, the composition is buffered and/or sterile. The composition may be suitable for administration to a human subject, e.g., where the composition is of suitable pH (e.g., from about 6.5 to 7.8 such as pH 7.4+/−0.2 pH units), sterile and is substantially free of detectable pyrogens and/or other toxins, or any such detectable pyrogens and/or other toxins are below permissible limits. Thus, the present disclosure provides a composition comprising: a) a T-Cell-MP-epitope conjugate; and b) saline (e.g., 0.9% or about 0.9% NaCl), where the composition is sterile and is free of detectable pyrogens and/or other toxins, or any such detectable pyrogens and/or other toxins are below permissible limits, and is optionally buffered to a suitable pH (e.g., with a phosphate buffer).

### A. Compositions Comprising a Nucleic Acid or a Recombinant Expression Vector

The present disclosure provides compositions (e.g., pharmaceutical compositions) comprising a nucleic acid or a recombinant expression vector (see, e.g., supra) that comprise one or more nucleic acid sequences encoding any one or more T-Cell-MP polypeptide (or each of the polypeptides of a duplex T-Cell-MP multimer such as a heterodimer). As discussed above, pharmaceutically acceptable excipients are known in the art and have been amply described in a variety of publications.

### XI. METHODS OF MODULATING IMMUNE RESPONSES AND TREATING DISEASES AND DISORDERS

T-Cell-MP-epitope conjugates and higher order T-Cell-MP-epitope conjugate complexes (e.g., duplex T-Cell-MP-epitope conjugates) are useful for modulating an activity of a T cell, and directly or indirectly modulating the activity of other cells of the immune system. The present disclosure provides methods of modulating an activity of a T cell selective for a epitope (e.g., an “epitope-specific T cell” or an “epitope selective T cell”), the methods generally involving contacting a target T cell with a T-Cell-MP-epitope conjugate or a higher order complex of T-Cell-MP-epitope conjugates (e.g., duplex T-Cell-MP-epitope conjugates). A T-Cell-MP-coronavirus epitope conjugate or its higher order complexes may comprise one or more independently selected MODs that activate an epitope-specific T cell that recognizes a coronavirus-infected cell. In some cases, the activated T cells are cytotoxic T cells (e.g., CD8⁺ cells). Accordingly, the disclosure includes and provides for a method of preventing or inhibiting a coronavirus infection, prophylactically providing immune protection to lessen the effect and/or symptoms of a coronavirus infection, and/or treating an existing coronavirus infection, the method comprising administering to an individual in need thereof an effective amount of a T-Cell-MP-coronavirus epitope conjugate or a higher order complex thereof that comprises one or more independently selected MODs that activate an epitope-specific T cell that recognizes an epitope specific to a coronavirus antigen. An effective amount of such a T-Cell-MP-coronavirus epitope conjugate or its higher order complex may be an amount that primes and/or activates a CD8⁺ T cell specific to the conjugated epitope (e.g., generating and/or increasing the number of the CD8⁺ T cells and/or increasing proliferation related cell signaling, increasing release of their cytotoxic agents such as granzyme, and/or inducing or enhancing release of their cytokines such as interferon γ).

A T-Cell-MP-coronavirus epitope conjugate or its higher order complexes may also comprise one or more independently selected MODs that inhibit an epitope-specific T cell. Such T-Cell-MP-coronavirus epitope conjugates are useful for the treatment of individuals experiencing a reaction to a coronavirus that results in an excessive immune response to a coronavirus infection (e.g., excessive production of cytokines and/or cytotoxic responses by, for example, T cells) resulting in tissue damage. Accordingly, one or more T-Cell-MP-coronavirus epitope conjugates or their higher order complexes may be utilized to treat individuals experiencing a “cytokine storm” or respiratory distress in response to a coronavirus infection.

### A. Methods of Modulating T Cell Activity

The present disclosure provides a method of selectively modulating the activity of a T cell, the method comprising contacting or administering to a subject a T-Cell-MP-epitope conjugate or a higher order complex thereof, in some instances with a targeting sequence (e.g., CTP) or payload. Where the T-Cell-MP-epitope conjugate is a T-Cell-MP-coronavirus epitope conjugate the contacting can result in modulation of T cells specific for the conjugated epitope. Modulating the activity of a T cell can include, but is not limited to, one or more of: i) activating a cytotoxic (e.g., CD8⁺) T cell (e.g., to proliferate); ii) inducing cytotoxic activity of a cytotoxic (e.g., CD8⁺) T-cell; iii) inducing production and release of a cytotoxin (e.g., a perforin; a granzyme; a granulysin) by a cytotoxic (e.g., CD8⁺) T-cell; and iv) increasing the number of epitope-specific T cells. The contacting or administration may occur in vitro or in vivo where the molecule is administered to an animal, typically a human, but also to other animals that are susceptible to coronavirus infections (e.g., a mammal such as a rat, mouse, dog, cat, pig, horse, or primate). The contacting or administration may constitute all or part of a method of treating a disease or disorder as discussed further below. The T cells subject to modulation may be, for example, CD8+ T cells, a NK-T cells, and/or T reg cells. In some cases, the T cell is a CD8+ effector T cell. In some cases, the T cell is a CD8+ T-cell, CD4+CD8+ double positive T-cell, or a NK-T cell as described below under Treatment Methods. In some cases, the T cell is a CD8+ T cell as described below under Treatment Methods.

The present disclosure provides a method of selectively modulating the activity of an epitope-specific T cell. The method comprises contacting a T cell with a T-Cell-MP-coronavirus epitope conjugate (e.g., in duplex form) bearing a peptide presenting a coronavirus epitope recognized by the epitope-specific T-Cell. The contacting results in selectively modulating the activity of the epitope-specific T cell with the selectivity driven by the coronavirus epitope and the resultant activation driven, at least in part, by the MOD polypeptide sequence of the T-Cell-MP-epitope conjugate. Contacting T cells with T-Cell-MP-epitope conjugates can result in activation or suppression of T cells expressing a TCR specific for the conjugated epitope (an epitope-specific T cell) including induction or suppression of granule dependent and independent responses. Granule-independent responses include, but are not limited to, changes in the number or percentage of epitope-specific CD 8+ T cell (e.g., in a population of cells such as in blood, lymphatics, and/or in a target tissue), changes in the expression of Fas ligand (Fas-L, which can result in activation of caspases and target cell death through apoptosis), and cytokine/chemokine production (e.g., production and release of interferon gamma (IFN-v). Granule-dependent effector actions include the release of granzymes, perforin, and/or granulysin. Activation of epitope-specific CD8⁺ cytotoxic T cells (e.g., CD8⁺ cytotoxic effector T cells) can result in the targeted killing of, for example, infected cells by epitope-specific T cells that recognize the epitope presented by the T-Cell-MP-epitope conjugate (or higher order complex thereof (e.g., a duplex) through granule-dependent and/or independent responses.

Contacting a T-Cell-MP-epitope conjugate or higher order complex thereof (e.g., a duplex) bearing an activating MOD, where the T-Cell-MP is conjugated to an epitope recognize by the TCR of a target T cell (an epitope-specific T cell), may result in one or more of: i) proliferation of the epitope-specific T cell (e.g., CD8+ cytotoxic T cells); ii) epitope-specific induction cytotoxic activity; iii) release of one or more cytotoxic molecules (e.g., a perforin; a granzyme; a granulysin) by the epitope-specific cytotoxic (e.g., CD8+) T cell.

Where a T-Cell-MP-epitope conjugate includes a MOD that is an activating polypeptide, contacting the T cell with the T-Cell-MP-epitope conjugate activates the epitope-specific T-cell. In some instances, the epitope-specific T cell is a T cell that is specific for a coronavirus epitope (e.g., peptide, phosphopeptide, or glycopeptide epitopes such as those from a spike glycoprotein, nucleoprotein, membrane protein, replicase protein, non-structural protein (nsp) and the like), and contacting the epitope-specific T cell with the T-Cell-MP-epitope conjugate increases cytotoxic activity of the T cell toward a coronavirus-infected cell. In some instances, the epitope-specific T cell is a T cell that is specific for a coronavirus epitope, and contacting the epitope-specific T cell with the T-Cell-MP-epitope conjugate increases the number of the epitope-specific T-cells.

In some instances, the epitope-specific T cell is a T cell that is specific for an epitope present on a coronavirus-infected cell and contacting the epitope-specific T cell with the T-Cell-MP-epitope conjugate increases cytotoxic activity of the T cell toward the coronavirus-infected cell. In some instances, the epitope-specific T cell is a T cell that is specific for an epitope present on a coronavirus-infected cell and contacting the epitope-specific T cell with the T-Cell-MP-epitope conjugate increases the number of the epitope-specific T-cells.

In contrast, contacting a T-Cell-MP-coronavirus epitope conjugate or higher order complex thereof (e.g., a duplex) bearing an inhibitory MOD, with a T cell having a TCR that recognizes the conjugated peptide presenting a coronavirus epitope may result in one or more of: i) suppression of proliferation and/or reduction the number of the epitope-specific T cells (e.g., CD8+ cytotoxic T cells); ii) epitope-specific suppression of a cytotoxic activity; iii) suppression the production and/or release of one or more cytotoxic molecules (e.g., a perforin; a granzyme; a granulysin) by the epitope-specific cytotoxic (e.g., CD8+) T cell. Contacting a T-Cell-MP-epitope conjugate or higher order complex thereof (e.g., a duplex) conjugated to an epitope recognize by TCR of a T cell (an epitope-specific T cell) and bearing an inhibitory MOD may also result in one or more of: i) epitope-specific inhibition autoreactive T cell; or ii) induction of epitope-specific CD8+T regulatory cells; and the like.

The present disclosure provides a method of modulating an immune response in an individual, the method comprising administering to the individual one or more doses of an effective amount of a T-Cell-MP-coronavirus epitope conjugate. As noted above, administering the T-Cell-MP-coronavirus epitope conjugate, may induce a coronavirus epitope-specific T cell response resulting in modulating the proliferation activity of a first T cell that displays both: i) a TCR specific for the epitope present in the T-Cell-MP; and ii) a Co-MOD that binds to the MOD present in the T-Cell-MP-epitope conjugate; and also induces an epitope non-specific T cell response by modulating the proliferation activity of a second T cell that displays: i) a TCR specific for an epitope other than the epitope present in the T-Cell-MP; and ii) a Co-MOD that binds to the MOD present in the T-Cell-MP. The ratio of the epitope-specific T cell response to the epitope-non-specific T cell response following administration of the T-Cell-MP-coronavirus epitope conjugate (e.g., when measured as the ratio of the increase of the number of epitope-specific T cells to the number of epitope non-specific T cells in the blood) may be at least 2:1 to at least 100:1. For example, following administration, the ratio of the proliferation of epitope-specific T cell response to the proliferation of epitope-non-specific T cell may be at least 5:1, at least 10:1, at least 25:1, at least 50:1, or at least 100:1. The increase of the number of epitope-specific T cells to the increase in the number of epitope non-specific T cells, e.g. in the blood of a coronavirus-infected patient, can be readily determined by known methods.

Increasing cytotoxic T cell response to a coronavirus-infected cell by administering a T-Cell-MP-epitope conjugate may occur in vivo where the T-Cell-MP-epitope conjugate is administered to a subject (e.g., intravenously, subcutaneously, or intramuscularly). The in vivo modulation may occur in a human subject or patient.

The present disclosure also provides a method of detecting, in a mixed population of cells (e.g., a mixed population of T cells) obtained from an individual, the presence of a target T cell that binds a coronavirus epitope of interest, the method comprising: a) contacting in vitro the mixed population of cells (e.g., mixed population of T cells) with a T-Cell-MP-coronavirus epitope conjugate; and b) detecting activation and/or proliferation of T cells in response to said contacting, wherein activated and/or proliferated T cells indicates the presence of the target T cell.

The present disclosure provides a method of increasing the proliferation (e.g., proliferation rate) and/or the total number of CD 8+ effector T cells directed against a coronavirus epitope in an animal or tissue that are specific to an epitope presented by a T-Cell-MP-epitope conjugate or higher order complex thereof (e.g., a duplex) bearing an activating MOD such as IL-2 or an IL-2 variant as described herein. A method of increasing T cell proliferation or numbers comprises contacting (e.g., in vitro or in vivo) T cells with a T-Cell-MP-epitope conjugate. Contacting may occur, for example, by administering to a subject in one or more doses a T-Cell-MP-epitope conjugate. The contacting or administering may increase the number of CD8+ effector T cells having a TCR capable of binding the epitope present in the T-Cell-MP-coronavirus-epitope conjugate relative to the number (e.g., total number or percentage) of T cells present in a tissue (e.g., in a population of cells such as in blood, lymphatics, and/or in a target tissue such as a tumor). For example, the absolute or relative number of CD 8+ effector T cells specific to the coronavirus epitope presented by a T-Cell-MP-epitope conjugate or a higher order complex (e.g., duplex) can be increased by at least 5%, at least 10%, at least 20%, at least 30%, at least 40%, at least 50%, least 75%, at least 100%, at least 2-fold, at least 2.5-fold, at least 5-fold, at least 10-fold, or more than 10-fold following one or more contacts with doses or administrations of the T-Cell-MP-epitope conjugate or a higher order complex thereof. The increase may be calculated relative the CD8+ T cell numbers present in the tissue prior to the contacting or administrations, or relative to the population of T cells present in the tissue (e.g., a sample of blood or tissue) that has not been contacted with the T-Cell-MP-epitope conjugate or its higher order complex. The increase may also be calculated relative the relative increase in epitope-specific CD8+ T cell numbers present after an otherwise identical population of T cells have been contacted with an otherwise identical T-Cell-MP conjugated to a control epitope not recognized by the target T cells, or by contact with the unconjugated T-Cell-MP. For example, within a tumor the number of CD8+ T cells specific for the epitope presented by the T-Cell-MP-epitope conjugate may increase in the absolute number per weight or volume of tissue (e.g., the number of epitope-specific T cells in histological sections or in disrupted tumor samples determined by flow cytometry using tetramers specific for the T cells). The increase in CD8+ T cells specific for the epitope presented by the T-Cell-MP-epitope conjugate may also increase as the fraction (e.g., percentage) of total CD8+ T cells present in the histological sections or tumor samples (e.g., accessed by tetramer staining using flow cytometry).

The present disclosure provides a method of increasing granule-dependent and/or granule-independent responses of epitope-specific CD 8+ T cell comprising contacting or administering (e.g., in vitro or in vivo) T cells with a T-Cell-MP-epitope conjugate or a higher order complex thereof, (e.g., with a CD80, and/or CD86 MOD). The contacting or administering may result in, for example, an increased expression of Fas ligand expression, cytokines/chemokines (e.g., IL-2, IL-4, and/or IL-5), release of interferons (e.g., IFN-γ), release of granzymes, release of perforin, and/or release of granulysin. For example, contacting a CD 8+ effector cell with a T-Cell-MP-epitope conjugate or complex thereof (e.g., a duplex) that presents a coronavirus epitope may increase one or more of Fas ligand expression, interferon gamma (IFN-γ) release, granzyme release, perforin release, and/or granulysin release by a T cell bearing a TCR that is specific to the epitope. The increase may be at least 5%, at least 10%, at least 20%, at least 30%, at least 40%, at least 50%, least 75%, at least 100%, at least 2-fold, at least 2.5-fold, at least 5-fold, at least 10-fold, or more than 10-fold. The increase may be calculated relative the level of expression or release prior to the contacting or administrations, or relative to the population of T cells present in a sample (e.g., a sample of blood or tissue) that has not been contacted with the T-Cell-MP-epitope conjugate or a complex thereof. The increase may also be calculated relative the level of expression or release by an otherwise identical population of T cells that have been contacted with an otherwise identical T-Cell-MP conjugated to a control epitope not recognized by the target T cells or by contact with the unconjugated T-Cell-MP.

### B. Methods of Selectively Delivering a MOD (Costimulatory Polypeptide)

The present disclosure provides a method of delivering one or more independently selected MODs and/or a reduced-affinity variant of naturally occurring MODs (such as a variant disclosed herein) to a selected T cell or a selected T cell population, e.g., in a manner such that T cells bearing a TCR specific for a given coronavirus epitope are targeted. The present disclosure provides a method of delivering a MOD, or a variant (e.g., a reduced-affinity variant) of a naturally occurring MOD disclosed herein, selectively to a target T cell bearing a TCR specific for the coronavirus epitope present in a T-Cell-MP-epitope conjugate. The method comprises contacting a population of T-cells with a T-Cell-MP-coronavirus epitope conjugate. The population of T-cells can be a mixed population that comprises: i) the target T-cell; and ii) non-target T-cells that are not specific for the epitope (e.g., T-cells that are specific for an epitope other than the epitope to which the epitope-specific T cell binds). The epitope-specific T cell is specific for the coronavirus epitope-presented by the T-Cell-MP-coronavirus epitope conjugate and binds to the peptide-HLA complex (or peptide-MHC complex) provided by the T-Cell-MP-coronavirus epitope conjugate. Accordingly, contacting the population of T-cells with the T-Cell-MP-epitope conjugate delivers the costimulatory polypeptide (e.g., a wild-type MOD or a variant MOD (e.g., a reduced-affinity variant of the wild-type MOD described herein) selectively to the T-cell(s) that are specific for the epitope present in the T-Cell-MP-epitope conjugate. In some cases, the population of T cells is in vitro. In some cases, the population of T cells is in vivo in an individual. In some cases, the method comprises administering the T-Cell-MP-epitope conjugate to the individual. In some case, the T cell is a cytotoxic T cell. In some cases, the mixed population of T cells is an in vitro population of mixed T cells obtained from an individual, and the contacting step results in activation and/or proliferation of the target T cell(s), generating a population of activated and/or proliferated target T cells; in some of these instances, the method further comprises administering the population of activated and/or proliferated target T cells to the individual.

In some cases, the population of T cells to which the MOD(s) and/or variant MOD(s) is/are delivered is present in vitro, and a biological response (e.g., T cell activation, expansion, and/or phenotypic differentiation) of the target T cell population to the T-Cell-MP-epitope conjugate or a higher order complex thereof (e.g., a duplex) is elicited in the context of an in vitro setting. For example, a mixed population of T cells can be obtained from an individual and can be contacted with the T-Cell-MP-epitope conjugate or a higher order complex thereof (e.g., a duplex) in vitro. Such contacting can comprise single or multiple exposures of the population of T cells to one or more doses of the T-Cell-MP-epitope conjugate. In some cases, said contacting results in selectively binding/activating and/or expanding target T cells within the population of T cells, and results in generation of a population of activated and/or expanded target T cells. As an example, a mixed population of T cells can be peripheral blood mononuclear cells (PBMCs) obtained by phlebotomy and standard enrichment techniques before being exposed to 0.1-1000 nM (e.g., 0.1 to 10 nM or 10 nm to 1,000 nm) of a T-Cell-MP-epitope conjugate or a higher order complex thereof (e.g., a duplex) under conditions suitable for lymphocyte culture. At time points before, during, and after exposure of the mixed T cell population at a defined dose and schedule, the abundance of target T cells in the in vitro culture can be monitored by specific peptide-MHC multimers, phenotypic markers, and/or functional activity (e.g. cytokine ELISpot assays). In some cases, upon achieving an optimal abundance and/or phenotype of antigen specific cells in vitro, all or a portion of the population of activated and/or expanded target T cells is administered to an individual (e.g., the individual from whom the mixed population of T cells was obtained as a treatment for a disease of disorder).

By way of example, a mixed population of T cells is obtained from an individual and is contacted with a T-Cell-MP-epitope conjugate or a higher order complex thereof (e.g., a duplex) in vitro. Such contacting, which can comprise single or multiple exposures of the T cells to one or more doses and/or exposures in the context of in vitro cell culture, can be used to determine whether the mixed population of T cells includes T cells that are specific for the epitope presented by the T-Cell-MP-epitope conjugate or higher order complex. The presence of T cells that are specific for the epitope can be determined by assaying a sample comprising a mixed population of T cells, which population of T cells comprises T cells that are not specific for the epitope (non-target T cells) and may comprise T cells that are specific for the epitope (target T cells). Known assays can be used to detect activation and/or proliferation of the target T cells, thereby providing an in vitro assay that can determine whether a particular T-Cell-MP-epitope conjugate or a higher order complex thereof possesses an epitope that binds to T cells present in the individual, and thus whether the epitope conjugate has potential use as a therapeutic composition for that individual. Suitable known assays for detection of activation and/or proliferation of target T cells include, e.g., flow cytometric characterization of T cell phenotype and/or antigen specificity and/or proliferation. Such assays maybe used to detect the presence of epitope-specific T cells, e.g., as a companion diagnostic. Additional assays (e.g. effector cytokine ELISpot assays) and/or appropriate controls (e.g. antigen-specific and antigen-nonspecific multimeric peptide-HLA staining reagents) to determine whether the T-Cell-MP-epitope conjugate or a higher order complex thereof (e.g., a duplex) is selectively binding, modulating (activating or inhibiting), and/or expanding the target T cells may also be employed. Thus, for example, the present disclosure provides a method of detecting, in a mixed population of T cells obtained from an individual, the presence of a target T cell that binds a coronavirus epitope of interest, the method comprising: a) contacting in vitro the mixed population of T cells with a T-Cell-MP-coronavirus epitope conjugate or a higher order complex thereof (e.g., a duplex); and b) detecting modulation (activation or inhibition) and/or proliferation of T cells in response to said contacting, wherein modulation of and/or proliferation of T cells indicates the presence of the target T cell. Alternatively, or in addition, if activation and/or expansion (proliferation) of the desired T cell population is obtained using a T-Cell-MP-coronavirus epitope conjugate or a higher order complex thereof (e.g., a duplex), then all or a portion of the population of T cells comprising the activated/expanded T cells can be administered back to the individual as a therapy for the prophylaxis/treatment of a coronavirus infection.

In some instances, the population of T cells is in vivo in an individual. In such instances, a method for selectively delivering one or more MOD polypeptides (e.g., IL-2 or PD-L1 or a reduced-affinity IL-2 or PD-L1) to an epitope-specific T cell comprises administering the T-Cell-MP-epitope conjugate or a higher order complex thereof (e.g., as a duplex) to the individual. The T-Cell-MP may comprise one or more (e.g., two or more) targeting sequences that redirect the T-Cell-MP to a specified cell or tissue type. In some instances, at least one of the targeting sequences is specific for coronavirus antigen (e.g., expressed on an infected cell surface) and the epitope present in the T-Cell-MP-epitope conjugate is not an epitope of the same (identical) coronavirus antigen. Such molecules may find use in, for example, redirecting CD8+ T cells of an individual immunized against a coronavirus infection (e.g., using an antigen that presents the covalently conjugated epitope in the T-cell-MP-epitope conjugate) to express effector T cell functions against cells of the immunized individual that are infected with a different strain of the coronavirus.

In some instances, the epitope-specific T cell to which one or more MOD polypeptide sequences (e.g., a wild-type or variant MODs such as reduced-affinity variants of IL-2 or PD-L1) is/are being selectively delivered is a target T cell that recognizes the epitope presented by the T-Cell-MP-epitope conjugate (e.g., T-Cell-MP-coronavirus epitope conjugate).

### C. Methods of Treatment

The present disclosure provides a method of treating or preventing a coronavirus infection in an individual comprising selectively modulating the activity of a coronavirus epitope-specific T cell in an individual. The method comprises administering to the individual an amount of a T-Cell-MP-epitope conjugate (e.g., a T-Cell-MP-coronavirus epitope conjugate). The coronavirus infection may be a primary infection, secondary infection, or may be associated with Long COVID. Such methods of treatment may function to increase the number of T cells specific to the epitope presented by the T-Cell-MP-coronavirus epitope conjugate or its higher order complexes (e.g., duplexes) when MODs that stimulate T cell proliferation are present in the T-Cell-MP-epitope conjugate. When the proliferated T cells are, for example, CD8+ effector T cells or NK T cells the method results in the killing of cells displaying the epitope conjugated to the T-Cell-MP. Also provided are T-Cell-MP-coronavirus epitope conjugates and redirected T-Cell-MP-epitope conjugates for use in a method of treatment of a human or non-human mammal.

A treatment method, which may be done prophylactically, may comprise administering to an individual in need thereof an effective amount of one or more T-Cell-MP-coronavirus epitope conjugates or higher order complexes of such proteins (e.g., duplexes). Where treatment is conducted prophylactically, it may prevent in the treated subject a coronavirus infection or at least prevent one or more symptoms or complications of an infection if it occurs such as those symptoms requiring hospitalization, relative to the rate of infection observed in a control population. Conditions that can be treated (or prevented in the case of prophylactic administration) include alpha-, beta-, gamma-, delta- and omicron-coronavirus infections. Coronavirus infections that can be treated include infections by Bat-SL-CoV, SARS-CoV, SARS-CoV-2 (Covid-19), or MERS-CoV. Coronavirus infections that can be treated also include infections by a SARS-CoV, SARS-CoV-2, or MERS-CoV. A coronavirus infection that can be treated may be a SARS-CoV-2 (Covid-19) infection. Treatment with one or more T-Cell-MP-coronavirus epitope conjugates may be used to boost the immunity of a previously immunized or previously infected individual.

The symptoms of SARS-CoV-2 vary, but individuals generally experience one or more of: fever or chills, cough, shortness of breath or difficulty breathing, fatigue, muscle or body aches, headache, new loss of taste or smell, sore throat, congestion or runny nose, nausea or vomiting, or diarrhea. See, e.g., Management of Patients with Confirmed Coronavirus Disease (COVID-19)|CDC at: www.cdc.gov/coronavirus/2019-ncov/hcp/clinical-guidance-management-patients.html.

The most common complications of severe SARS-CoV-2 include pneumonia, hypoxemic respiratory failure/ARDS, sepsis and septic shock, cardiomyopathy and arrhythmia, and acute kidney injury. Id. “Some patients with COVID-19 may have signs of a hypercoagulable state and be at increased risk for venous and arterial thrombosis of large and small vessels.” Id. Symptoms commonly observed among patients hospitalized with SARS-CoV-2 coagulopathies include: mild thrombocytopenia, increased D-dimer levels, increased fibrin degradation products, and prolonged prothrombin time; with elevated D-dimer levels strongly associated with greater risk of mortality. Id. Hospitalized patients may also experience thrombotic complications, including deep venous thrombosis, pulmonary embolism, microvascular thrombosis of the toes (“COVID toes”), clotting of intra-vascular catheters, myocardial injury with ST-segment elevation, and large vessel strokes. Id.

The present disclosure provides a method of treating a patient infected by a coronavirus comprising administering to the patient diagnosed to have a coronavirus infection an effective amount of one or more T-Cell-MP-coronavirus epitope conjugates or higher order complexes. Treatment may reduce one or more of: symptoms of the infection, the amount of time until the subject is free of detectable virus, morbidity, disease severity (severity of a symptom or complication), and/or mortality due to coronavirus infection relative to the average for a control population (e.g., an untreated patient group infected by a coronavirus matched for age and sex). The coronavirus may be a SARS-CoV2 coronavirus.

A control population is a population of patients or subjects that did not receive a treatment with any T-Cell-MP-coronavirus epitope conjugate or higher order complex of such protein or other treatments for a coronavirus infection. The control population may be matched by i) age, sex, immune disease, and country of residence. A control population may also be matched by (ii) age, weight, sex, immune disease, country of residence, and smoking status. A control population may also be matched by (iii) age, weight, sex, immune disease status including HIV infection status, country of residence, cancer, cerebrovascular disease, kidney disease, chronic obstructive pulmonary disease, (COPD), diabetes, coronary disease (heart failure, coronary artery disease, or cardiomyopathies), smoking, pregnancy, and/or asthma. See, e.g., www.cdc.gov/coronavirus/2019-ncov/need-extra-precautions/people-with-medical-conditions.html. Additional factors deemed relevant to coronavirus disease, its symptoms, progression, and/or mortality may be added to those factors used to select a control population. Where necessary for valid comparison, such as for the amount of viral shedding, the control population may be a coronavirus-infected control population, which may be further selected for the specific virus and/or length of time since the individuals in the population were initially infected.

Because the T-Cell-MP-coronavirus epitope conjugates and higher order complexes thereof induce T cell responses that can result in killing virus infected cells, they can result in a lower viral load in treated patients or subjects, including those treated prophylactically. The lower viral load results in lower amounts of virus that can be shed and or transmitted by infected individuals. Accordingly, the present disclosure provides a method of reducing viral load, shedding, and/or transmission of coronavirus by infected individuals relative to the viral load, shedding, and/or transmission of coronavirus by a control population, the method comprising administering to an individual in need thereof an effective amount of one or more T-Cell-MP-coronavirus epitope conjugates or higher order complex of such protein. The present disclosure provides a method of reducing the transmission of coronavirus comprising administering to an individual diagnosed to have coronavirus infection or suspected of or at risk of exposure to coronavirus in need thereof an effective amount of one or more T-Cell-MP-coronavirus epitope conjugates or higher order complex of such proteins. The coronavirus may be SARS-CoV2 coronavirus.

The present disclosure thus provides a method of reducing the viral load of coronavirus in an individual comprising administering to an individual positive for coronavirus infection an effective amount of a T-Cell-MP-epitope conjugate. In particular embodiments, the coronavirus is a SARS-CoV2 coronavirus.

The present disclosure provides a method of killing coronavirus-infected cells in an individual comprising administering to an individual positive for coronavirus infection an effective amount of a T-Cell-MP-coronavirus epitope conjugate. The coronavirus may be a SARS-CoV2 coronavirus. In some cases, the T-Cell-MP-coronavirus epitope conjugate comprises a MOD (e.g., IL-2 or an IL-2 variant) that activates an epitope-specific T cell that recognizes the coronavirus epitope. In some cases, the T cells are cytotoxic T-cells (CD8+ cells) and the T-Cell-MP-coronavirus epitope conjugate increases the activity of the T cell specific for a coronavirus-infected cell. Activation of CD8+ T cells may include increasing: proliferation of the T cells; the production of cytokines, chemokines, and/or cytotoxic materials (e.g., granulysin); the release of cytokines such as interferon γ; and/or the release of cytotoxic materials (e.g., perforin, granzyme, or granulysin).

In some cases, a T-Cell-MP-coronavirus epitope conjugate reduces proliferation and/or activity of an epitope restricted regulatory T cell or Treg (e.g., CD8⁺ Tregs which are FoxP3⁺, CD8⁺ T cells). In some cases, e.g., where a T-Cell-MP-epitope conjugate comprises an inhibitory MOD (e.g., PD-L1, FasL, and the like), the T-Cell-MP-epitope conjugate reduces the proliferation and/or activity of a Treg and may result in apoptosis of the T reg.

T-Cell-MP-coronavirus epitope conjugates may comprise a viral lipopeptide, glycopeptide or phosphopeptide epitope, and may be administered to an individual in need thereof to treat a coronavirus infection in the individual, where the infectious agent expresses the epitope present in the T-Cell-MP-coronavirus epitope conjugate. Accordingly, the present disclosure provides a method of treating an infection in an individual, the method comprising administering to the individual an effective amount of a coronavirus lipopeptide, glycopeptide or phosphopeptide epitope containing T-Cell-MP-coronavirus epitope conjugate. Such treatments may be conducted prophylactically, to treat an active infection, or to boost the immunity of a previously infected or immunized individual.

In some instances, the epitope-specific T cell is a T cell that is specific for an epitope present on a coronavirus-infected cell, and contacting the epitope-specific T cell with the T-Cell-MP-coronavirus epitope conjugate (e.g., in vitro or in vivo by administration to a patient or subject) increases cytotoxic activity of the T cell toward the coronavirus-infected cell. In some instances, the epitope-specific T cell is a T cell that is specific for an epitope present on a coronavirus-infected cell, and contacting the epitope-specific T cell (e.g., in vitro or in vivo by administration to a patient or subject) with the T-Cell-MP-coronavirus epitope conjugate increases the number of epitope-specific T-cells. Accordingly, the present disclosure provides a method of treating a coronavirus infection in an individual, the method comprising administering to the individual an effective amount of a T-Cell-MP-coronavirus epitope conjugate, where the T-Cell-MP-coronavirus epitope conjugate comprises a stimulatory MOD.

The present disclosure provides a method of selectively modulating the activity of one or more coronavirus epitope-specific T-cell(s) in an individual (e.g., patient or subject), the method comprising administering to the individual an effective amount of one or more T-Cell-MP-coronavirus epitope conjugates that selectively modulate the activity of those one or more coronavirus epitope-specific T-cell(s) in the individual. As selectively modulating the activity of one or more coronavirus epitope-specific T-cell(s) can treat a disease or disorder in the individual, the present disclosure provides a treatment method comprising administering to an individual (e.g., an individual in need thereof) an effective amount of a T-Cell-MP-coronavirus epitope conjugate is sufficient to modulate the activity of one or more epitope-specific T cell(s), e.g., cause activation of such T-cell(s). The one or more T cell(s) may be specific for an epitope of a Bat-SL-CoV, SARS-CoV, SARS-CoV-2 (Covid-19), or MERS-CoV, and accordingly the method may treat a coronavirus infection by any one or more of those viruses. The one or more T cell(s) may be specific for SARS-CoV, SARS-CoV-2, or MERS-CoV, and accordingly, the treatment specific for infections by any one or more of those viruses. The one or more T cell(s) maybe specific for SARS-CoV or SARS-CoV-2, or MERS-CoV, and accordingly, the treatment specific for infections by either one or the other of those viruses. The one or more T cell(s) may be specific for SARS-CoV-2, and accordingly, the treatment specific for infections by that virus.

Administering an effective amount of T-Cell-MP-coronavirus epitope conjugates induces a coronavirus epitope-specific T cell response, and may also induce an epitope-non-specific T cell response. The ratio of the coronavirus epitope-specific T cell response to the epitope-non-specific T cell response may be at least 2:1. In some cases, the ratio of the coronavirus epitope-specific T cell response to the epitope-non-specific T cell response is at least 5:1. In some cases, the ratio of the coronavirus epitope-specific T cell response to the epitope-non-specific T cell response is at least 10:1. In some cases, the ratio of the coronavirus epitope-specific T cell response to the epitope-non-specific T cell response is at least 25:1. In some cases, the ratio of the coronavirus epitope-specific T cell response to the epitope-non-specific T cell response is at least 50:1. In some cases, the ratio of the coronavirus epitope-specific T cell response to the epitope-non-specific T cell response is at least 100:1. In some cases, the individual is a human.

An effective amount of a T-Cell-MP-coronavirus epitope conjugate or higher order complex of such protein may be an amount that, when administered in one or more doses to an individual in need thereof, reduces the number of coronavirus-infected cells in the individual. For example, in some cases, an “effective amount” of a T-Cell-MP-coronavirus epitope conjugate or a higher order complex of such protein, is an amount that, when administered in one or more doses to an individual in need thereof, reduces the number of coronavirus-infected cells in the individual by at least 10%, at least 15%, at least 20%, at least 25%, at least 30%, at least 40%, at least 50%, at least 60%, at least 70%, at least 80%, at least 90%, or at least 95%, compared to the number of coronavirus-infected cells in the individual prior to administration. In some cases, an effective amount of any of those proteins or their higher order complexes is an amount that, when administered in one or more doses to an individual in need thereof, reduces the number of coronavirus-infected cells in the individual to undetectable levels.

An “effective amount” of any one or more T-Cell-MP-coronavirus epitope conjugates or higher order complex of such proteins, including such proteins that also comprise a coronavirus targeting sequence, may be an amount that, when administered in one or more doses to an individual in need thereof, decreases one or more effects or symptoms of the coronavirus infection, and/or increases survival time of the individual.

An “effective amount” of any one or more redirected T-Cell-MP-coronavirus epitope conjugates, or higher order complexes of such proteins that comprise a CTP, may be an amount that, when administered in one or more doses to an individual in need thereof, decreases the number of cancerous cells (e.g., reduces the size or volume of the cancer), decreases the rate of growth of the cancer, decreases one or more effects or symptoms of the targeted cancer, and/or increases survival time of the individual.

In some cases, because T-Cell-MP-epitope conjugates also are able to prime naïve T cells, the pharmaceutical compositions of this disclosure comprising a T-Cell-MP-coronavirus epitope conjugate or a higher order complex thereof (e.g., a duplex) also may be used to prophylactically treat persons who are not infected but at risk of infection. For example, the pharmaceutical compositions can be administered to cause a human or non-human subject to prime and activate coronavirus epitope-specific T cells and/or develop memory T cells (see e.g., the peptide epitopes set forth in Table 3a) that will be therapeutically useful in the event of a coronavirus (e.g., SARS-CoV-2) infection. Accordingly, in some cases, an effective amount of a pharmaceutical composition comprising a T-Cell-MP-epitope conjugate or a higher order complex thereof (e.g., a duplex) is an amount that, when administered in one or more doses to individuals in a population who do not have an infection and are at risk of infection, and/or to individuals who are at greater risk of severe illness from infection by a coronavirus (e.g., SARs-CoV-2) than the general population, causes a human or non-human to prime and activate epitope-specific T cells and/or develop memory T cells that will be therapeutically useful (directed against coronavirus-infected cells or tissues) in the event of a coronavirus (e.g., a SARS-CoV-2) infection. Individuals who are at greater risk of severe illness from coronavirus (e.g., SARs-CoV-2) include, but are not limited to those having one or more of the following conditions: cancer, chronic kidney disease, chronic lung diseases (e.g., COPD, asthma, interstitial lung disease, cystic fibrosis, and pulmonary hypertension), dementia or neurological conditions, diabetes (types 1 or 2), Down syndrome, heart conditions, (e.g., heart failure, coronary artery disease, cardiomyopathies or hypertension), HIV infection, compromised immune system(s), liver disease, overweight, pregnancy, sickle cell disease or thalassemia, smoking (former or current), solid organ or blood stem cell transplant, stroke or cerebrovascular disease affecting blood flow to the brain, and/or substance use disorders. See e.g., CDC guidelines at:
www.cdc.gov/coronavirus/2019-ncov/need-extra-precautions/people-with-medical-conditions.html.

As noted above, in carrying out a subject treatment method, one or more T-Cell-MP-coronavirus epitope conjugates, redirected T-Cell-MP-epitope conjugates, or higher order complexes of either of those proteins may be administered to a patient or subject (e.g., individual in need thereof) either unformulated or formulated as a pharmaceutical composition.

The present disclosure also includes and provides for methods of redirecting a T cell (e.g., a CD8⁺ effector T cell) directed to a specified epitope (e.g., a specified epitope of a coronavirus such as SARS-CoV-2) toward a cancerous cell or tissue. If the patient does not have (or might not have) sufficient T cells directed to the specified epitope the method may comprise an initial step of immunizing the patient to be treated with a vaccine (e.g., a SARS-CoV-2 vaccine such as the Pfizer, Moderna or J&J vaccine) that induces or expands the number of T cells specific to the specified epitope (see FIG. 18 top section). A patient, having sufficient T cells specific to the epitope, is treated with a redirected T-Cell-MP conjugated to the specified epitope that further comprises one or more (e.g., two or more) targeting sequences directed to proteins associated with a cancer (e.g., CTPs) and one or more MODs. See FIG. 18 middle portion “Treatment with T-Cell-MP-epitope conjugate”. By way of example, the T-Cell-MP conjugated to the specified epitope may comprise cancer targeting sequences as discussed above. The T-Cell-MP-epitope conjugate bearing such a targeting sequence will be localized on a cancerous cell or tissue. Localization of the redirected T-Cell-MP-conjugated to the specified epitope causes the localization of T-cells specific to the specified epitope, while the signal from the MOD in combination with the signal from the TCR engaged by the specified epitope presented by the T-Cell-MP-epitope conjugate, directs the T cells response (e.g., cytotoxic attack of the target cell as shown in the lower section of FIG. 18). The overall process permits the actions of a T cell to be redirected from cells or tissues expressing the specified epitope to cells or tissues that express a molecule the targeting sequence(s) recognize. It may be advantageous to use heteromeric T-Cell-MPs (e.g., duplexes that are heterodimers) having more than one targeting sequence directed against different molecules presented by cancerous cells or tissues (e.g., CTP1 and CTP2 as in FIG. 18) to prevent malignant cells from escaping from the treatment by loss or mutation of a single target. Benign tumors, precancerous cells or tissues, tissues, and coronavirus infected cells may also be targeted with T-Cell-MPs comprising two or more targeting sequences.

Redirected T-Cell-MP-coronavirus epitope conjugates in which the peptide presenting a coronavirus epitope is a SARS-CoV-2 epitope offer several therapeutic advantages. For example, because a large portion of the world's population has been infected and/or vaccinated, many persons already will have T cells directed to SARS-CoV-2 proteins. Further, HLA loss is one of the mechanisms by which cancers can evade the immune system. Redirected T-Cell-MP-epitope conjugates, which do not rely on HLA, thus can attack such cancers. Another mechanism by which cancers evade the immune system is by mutation that eliminates presentation of an antigen. Redirected T-Cell-MP-epitope conjugates that present more than one targeting sequence (see, e.g., FIG. 18) can avoid the cancer from escaping immune surveillance and treatment because the cancer will have to obtain at least two different mutations that affect binding by the different targeting sequences. Alternatively, the patient can be administered two different Redirected T-Cell-MP-epitope conjugates, each of which has a targeting sequence directed to a different cancer antigen. Redirected T-Cell-MP-epitope conjugate therapy can be tailored to such individuals by prescreening them to confirm they have T cells that recognize the epitope conjugated to and presented by the T-Cell-MP MHC (HLA) portion of the molecule and/or by confirming that the targeting portion recognizes cancer cells in the individual to be treated.

An effective amount of a redirected T-Cell-MP-coronavirus-epitope conjugate or higher order complex of such protein may be an amount that, when administered in one or more doses to an individual in need thereof, reduces the number of cancerous cells expressing the antigen targeted by at least one CTP of the redirected T-Cell-MP-coronavirus-epitope conjugate or higher order complex thereof, as measured by the size or volume of the tumor in the case of a solid tumor, in the individual. For example, in some cases, an “effective amount” of a redirected T-Cell-MP-coronavirus-epitope conjugate or a higher order complex of such protein, is an amount that, when administered in one or more doses to an individual in need thereof, reduces the number of those cancerous (i.e., cancer cells expressing the target of the CTP) in the individual by at least 10%, at least 15%, at least 20%, at least 25%, at least 30%, at least 40%, at least 50%, at least 60%, at least 70%, at least 80%, at least 90%, or at least 95%, compared to the number of cancerous cells expressing the target of the CTP in the individual prior to the administration. The reduction may occur, for example, in two days, three days, four days, five days, six days, one week, two weeks, three weeks, one month, two months or more). In some cases, an effective amount of any of those proteins or their higher order complexes is an amount that, when administered in one or more doses to an individual in need thereof, reduces the number of cancer cells expressing the target of the CTP in the individual to undetectable levels.

An effective amount of a redirected T-Cell-MP-coronavirus-epitope conjugate or higher order complex of such protein may be an amount that, when administered in one or more doses to an individual in need thereof, reduces the amount of circulating tumor DNA (ctDNA) in the blood of a patient, For example, an “effective amount” of a redirected T-Cell-MP-coronavirus-epitope conjugate or a higher order complex of such protein, is an amount that, when administered in one or more doses to an individual in need thereof, reduces the level of ctDNA in the blood individual by at least 10%, at least 15%, at least 20%, at least 25%, at least 30%, at least 40%, at least 50%, at least 60%, at least 70%, at least 80%, at least 90%, or at least 95%, compared to the level of ctDNA prior to the administration. The reduction may occur, for example, in two days, three days, four days, five days, six days, one week, two weeks, three weeks, one month, two months or more).

### 1 Therapeutic Agents

Both T-Cell-MP-coronavirus-epitope conjugates and redirected T-Cell-MP-epitope conjugates or their higher order complexes (e.g., duplexes) may be employed in vitro or in vivo in conjunction with the administration of one or more therapeutic agents in method of contacting cells or tissue and/or methods of treating one or more patients or subjects. Therapeutic agents may be administered before, during (simultaneously or in combination) or after the administration of T-Cell-MP-coronavirus-epitope conjugates and redirected T-Cell-MP-epitope conjugates or their higher order complexes (e.g., duplexes). The therapeutic agents may supportive (e.g., oxygen therapy). Therapeutic agents may also be targeted at the coronavirus infection itself (e.g., modifying the cellular or humoral immune response to the infection). Alternatively, the therapeutic agents may be targeted at symptoms of the infection (e.g., the associated inflammatory response), which may result in substantial trauma to the cells or tissue of an infected individual.

Therapeutic agents targeted at the coronavirus infection itself or that modify the cellular or humoral immune response to the infection include antiviral agents and/or immunomodulatory agents such as cytokines. One group of therapeutic agents that may be employed with T-Cell-MP-coronavirus-epitope conjugates or their higher order complexes (e.g., duplexes) in the methods of treating and contacting described herein include nucleotide analogs, nucleoside analogs, chloroquine or hydroxychloroquine, anti-HIV agents, interferon beta (e.g., interferon beta 1a), protease inhibitors (e.g., cinanserin, flavonoids, diarylheptanoids), type I interferons (IFNs), recombinant IFN (e.g., rIFNβ or rIFNγ), inhibitors of IL-6 receptors (e.g., tocilizumab), inhibitors of IL-6 (e.g., anti-IL-6 antibodies such as lenzilumab), NSAIDs, and glucocorticoids. The nucleotide or nucleoside analogs may be selected from the group consisting of: remdesivir, molnupiravir, favipiravir, ribavirin and galidesivir. The therapeutic agents may be the anti-HIV agents are lopinavir or ritonavir. In any of the methods of treating a coronavirus (e.g., SARS-CoV-2) infections described herein, remdesivir may be administered in conjunction with T-Cell-MP-coronavirus-epitope conjugates or their higher order complexes (e.g., duplexes). In any of the methods of treating a coronavirus (e.g., SARS-CoV-2) infections described herein, molnupiravir may be administered in conjunction with T-Cell-MP-coronavirus-epitope conjugates or their higher order complexes (e.g., duplexes).

Antibodies directed against SARS-CoV-2, including antibodies that bind the spike protein may also be used as therapeutic agent administered in conjunction with T-Cell-MP-coronavirus-epitope conjugates or their higher order complexes (e.g., duplexes). Antibodies directed against spike protein that may be employed alone or in combination as therapeutic agents in coronavirus (e.g., SARS-CoV-2) infection include, but are not limited to, one or more of bamlanivimab, etesevimab, casirivimab, and/or imdevimab. By way of example, a combination of bamlanivimab and etesevimab or a combination of casirivimab and imdevimab may be employed. Those antibodies, or combinations of those antibodies, may function to block viral entry and may also neutralize the virus and/or subject viral particles to attack by immune cells.

In some instance compounds that inhibit coronavirus entry into cells may be administered in conjunction with T-Cell-MP-coronavirus-epitope conjugates or their higher order complexes (e.g., duplexes). Inhibitors of coronavirus entry into cells include, but are not limited to, tilorone, mitoxantrone, raloxifene, and piceatannol, sunitinib, and BNTX. More specifically, mitoxantrone, raloxifene, and piceatannol are reported to bind to heparin sulfate, while sunitinib (SUTENT®) and BNTX target the actin cytoskeleton and tilorone inhibits lysosomes. See, e.g., Zhang et al., bioRxiv preprint doi: https://doi.org/10.1101/2020.07.14.202549 and https://www.ott.nih.gov/bundle/tab-3428. Either or both of tilorone and raloxifene (e.g., in combination) can be administered orally.

Therapeutic agents that target symptoms of the infection (e.g., the associated inflammatory response) one or more NSAIDs and or corticosteroids. The NSAIDs may be Cox-1 and/or Cox-2 inhibitors. In particular, the NSAIDs may be selected from the group consisting of celecoxib, diclofenac, diflunisal, etodolac, ibuprofen, indomethacin, ketoprofen, and naproxen. Corticosteroids may be selected from, for example, cortisone, dexamethasone, hydrocortisone, ethamethasoneb, fludrocortisone, methylprednisolone, prednisone, prednisolone and triamcinolone.

Other therapeutic agents that may be employed in conjunction with T-Cell-MP-coronavirus-epitope conjugates or their higher order complexes (e.g., duplexes) include, but are not limited to pyronaridine, pyronaridine-artesunate, and/or quinacrine.

Where a patient is being treated for cancer, a method for treating cancer in an individual comprises: a) administering one or more redirected T-Cell-MP-epitope conjugates or their higher order complexes (e.g., duplexes); and b) administering at least one additional therapeutic agent or therapeutic treatment for treatment of the cancer. Suitable additional therapeutic agents include, but are not limited to, a small molecule cancer chemotherapeutic agent, and an immune checkpoint inhibitor. Suitable additional therapeutic treatments include, e.g., radiation, surgery (e.g., surgical resection of a tumor), and the like.

A treatment method of the present disclosure can comprise co-administration of one or more redirected T-Cell-MP-epitope conjugates or their higher order complexes (e.g., duplexes) and at least one additional therapeutic agent. By “co-administration” is meant that both a redirected T-Cell-MP-epitope conjugate or higher order complex and at least one additional therapeutic agent are administered to an individual, although not necessarily at the same time, in order to achieve a therapeutic effect that is the result of having administered both the redirected T-Cell-MP-epitope conjugate and the at least one additional therapeutic agent. The administration of the redirected T-Cell-MP-epitope conjugate and the at least one additional therapeutic agent can be substantially simultaneous, e.g., the redirected T-Cell-MP-epitope conjugate can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the at least one additional therapeutic agent. In some cases, a redirected T-Cell-MP-epitope conjugate of the present disclosure is administered to an individual who is undergoing treatment with, or who has undergone treatment with, the at least one additional therapeutic agent. The administration of the redirected T-Cell-MP-epitope conjugate can occur at different times and/or at different frequencies.

As an example, a treatment method can comprise co-administration of a redirected T-Cell-MP-epitope conjugate or higher order complex and an immune checkpoint inhibitor such as an antibody specific for an immune checkpoint. By “co-administration” is meant that both a redirected T-Cell-MP-epitope conjugate and an immune checkpoint inhibitor (e.g., an antibody specific for an immune checkpoint polypeptide) are administered to an individual, although not necessarily at the same time, in order to achieve a therapeutic effect that is the result of having administered both the redirected T-Cell-MP-epitope conjugate and the immune checkpoint inhibitor (e.g., an antibody specific for an immune checkpoint polypeptide). The administration of the redirected T-Cell-MP-epitope conjugate and the immune checkpoint inhibitor (e.g., an antibody specific for an immune checkpoint polypeptide) can be substantially simultaneous, e.g., the redirected T-Cell-MP-epitope conjugate can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, within about 24 hours, within 1 week, 3 weeks 3 weeks, four weeks or a month following administration of the immune checkpoint inhibitor (e.g., an antibody specific for an immune checkpoint polypeptide). In some cases, a redirected T-Cell-MP-epitope conjugate of the present disclosure is administered to an individual who is undergoing treatment with, or who has undergone treatment with, an immune checkpoint inhibitor (e.g., an antibody specific for an immune checkpoint polypeptide). The administration of the redirected T-Cell-MP-epitope conjugate and the immune checkpoint inhibitor (e.g., an antibody specific for an immune checkpoint polypeptide) can occur at different times and/or at different frequencies. Where there is an established dosing interval for the checkpoint inhibitor, depending on the interval, it may be possible to administer the redirected T-Cell-MP-epitope conjugate on the same day as the checkpoint inhibitor. For example, in some cases, where the dosing schedule for pembrolizumab is once every three weeks, the pharmaceutical composition comprising the redirected T-Cell-MP-epitope conjugate may be administered on the same day.

Exemplary immune checkpoint inhibitors include inhibitors that target an immune checkpoint polypeptide such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122 and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT and VISTA.

In some cases, the immune checkpoint inhibitor is an antibody specific for an immune checkpoint polypeptide. In some cases, the anti-immune checkpoint antibody is a monoclonal antibody. In some cases, the anti-immune checkpoint antibody is humanized, such that the antibody does not substantially elicit an immune response in a human. In some cases, the anti-immune checkpoint antibody is a humanized monoclonal antibody. In some cases, the anti-immune checkpoint antibody is a de-immunized monoclonal antibody. In some cases, the anti-immune checkpoint antibody is a fully human monoclonal antibody. In some cases, the anti-immune checkpoint antibody inhibits binding of the immune checkpoint polypeptide to a ligand for the immune checkpoint polypeptide. In some cases, the anti-immune checkpoint antibody inhibits binding of the immune checkpoint polypeptide to a receptor for the immune checkpoint polypeptide.

Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (MedImmune/AZ), CP-870,893 (Genentech), Mogamulizumab (Kyowa Hakko Kirin), Varlilumab (CellDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035; and combinations thereof. For example, in some cases, the immune checkpoint inhibitor is an anti-PD-1 antibody. Suitable anti-PD-1 antibodies include, e.g., nivolumab, pembrolizumab (also known as MK-3475), pidilizumab, SHR-1210, PDR001, and AMP-224. In some cases, the anti-PD-1 monoclonal antibody is nivolumab, pembrolizumab or PDR001. Suitable anti-PD1 antibodies are described in U.S. Patent Publication No. 2017/0044259. For pidilizumab, see, e.g., Rosenblatt et al. (2011) J. Immunother. 34:409-18. The immune checkpoint inhibitor may be an anti-CTLA-4 antibody. The anti-CTLA-4 antibody may be ipilimumab or tremelimumab. For tremelimumab, see, e.g., Ribas et al. (2013) J. Clin. Oncol. 31:616-22. The immune checkpoint inhibitor may be an anti-PD-L1 antibody. The anti-PD-L1 monoclonal antibody may be BMS-935559, MEDI4736, MPDL3280A (also known as RG7446), KN035, or MSB0010718C. The anti-PD-L1 monoclonal antibody may be MPDL3280A (atezolizumab) or MEDI4736 (durvalumab). For durvalumab, see, e.g., WO 2011/066389. For atezolizumab, see, e.g., U.S. Pat. No. 8,217,149. The immune checkpoint inhibitor may be an anti-TIGIT antibody that binds to T-cell immunoreceptor with Ig and ITIM domains (TIGIT). The anti-TIGIT antibody may be BMS-986207 (Bristol-Myers Squibb). The anti-TIGIT antibody may be tiragolumab. The anti-TIGIT antibody may be EOS88448 (EOS-448). See, e.g., U.S. Pat. Nos. 11,008,390 and 10,189,902; U.S. Patent Publication No. 2017/0088613; and WO 2019/137541. The anti-TIGIT antibody may be domvanalimab (iTeos/GSK), ociperlimab (Beigene/Novartis) or AGEN1777 (Agenus/BMS).

Among such checkpoint inhibitors, antibodies to PD-1, PD-L1, and CTLA-4 are the most common, with at least nivolumab, tremelimumab, pembrolizumab, ipilimumab, cemiplimab, atezolizumab, avelumab, tisleizumab and durvalumab having been approved by the FDA and/or regulatory agencies outside of the U.S. Use of anti-TIGIT checkpoint inhibitors also is becoming increasingly common. A redirected T-Cell-MP-epitope conjugate also may be co-administered with combinations of checkpoint inhibitors, e.g., a combination of (i) an antibody to PD-1 or PD-L1, (ii) an antibody to CTLA-4, and/or (iii) an anti-TIGIT antibody.

### XII. SUBJECTS SUITABLE FOR TREATMENT

Subjects suitable for treatment with a T-Cell-MP-coronavirus epitope conjugate or higher order complex include individuals who have coronavirus infection or who may be at risk of a coronavirus infection. In particular, subjects suitable for treatment with a T-Cell-MP-coronavirus epitope conjugate include individuals who have a betacoronavirus infection such as SARS or MERS, or who are at risk of incurring a SARS or MERS infection, e.g., a SARS-CoV-2 infection, and/or are at greater risk of severe illness from such infection (e.g., e.g., a SARS-CoV-2 infection) than the general population. Suitable subjects include individuals who have been diagnosed as having SARS-CoV-2 (Covid-19), individuals who have been treated for coronavirus infection (e.g., SARS, SARS-CoV-2, or MERS) but who failed to respond to the treatment, individuals suffering from Long COVID, and individuals who have been treated for coronavirus infection and who initially responded but subsequently became refractory to the treatment. Individuals who are at greater risk of severe illness from an infection such as a SARS-CoV-2 infection than the general population include, but are not limited to, individuals having one or more underlying medical conditions selected from the group consisting of chronic kidney disease, COPD (chronic obstructive pulmonary disease), Down Syndrome, heart conditions, such as heart failure, coronary artery disease, or cardiomyopathies, an immunocompromised state (weakened immune system) from solid organ transplant, obesity (body mass index [BMI] of 30 kg/m² or higher but <40 kg/m²), severe obesity (BMI ≥40 kg/m²), pregnancy, sickle cell disease, a history of smoking, Type 2 diabetes mellitus, asthma (moderate-to-severe), cerebrovascular disease (affects blood vessels and blood supply to the brain), cystic fibrosis, hypertension or high blood pressure, an immunocompromised state (weakened immune system) from blood or bone marrow transplant, immune deficiencies, HIV, use of corticosteroids, or use of other immune weakening medicines, neurologic conditions such as dementia, liver disease, overweight (BMI >25 kg/m², but <30 kg/m²), pulmonary fibrosis (having damaged or scarred lung tissues), thalassemia or other blood disorders, and Type 1 diabetes mellitus.

Subjects suitable for treatment, e.g., by selectively delivering an activating MOD to a T cell selective for a coronavirus epitope or by modulating the activity of at least one coronavirus specific T cell (e.g., inducing a coronavirus specific T cell to proliferate) include those with a confirmed coronavirus infection, those diagnosed as positive for an infection by a coronavirus, and those at risk of contracting a coronavirus infection.

Subjects suitable for treatment with one or more redirected T-Cell-MP-coronavirus epitope conjugates or higher order complexes thereof include individuals who have neoplasms in the form of benign growths (e.g., non-malignant tumors) or malignant growths (e.g., occurring as a solid metastatic cancer or non-solid cancer such as leukemia, lymphoma or myeloma) and who have had a coronavirus infection and/or been vaccinated such that the individuals have T cells that bind the coronavirus epitope presented by the redirected T-Cell-MP-coronavirus epitope conjugate. Such subjects include individuals who have been diagnosed as having cancer, individuals who have been treated for cancer but who failed to respond to the treatment, and individuals who have been treated for cancer and who initially responded but subsequently became refractory to the treatment and/or whose disease progressed while on the prior treatment.

In some cases, the subject is an individual who is undergoing treatment with an immune checkpoint inhibitor. In some cases, the subject is an individual who has undergone treatment with one or more immune checkpoint inhibitors, but whose disease has progressed despite having received such treatment. In some cases, the subject is an individual who is undergoing treatment with, or who has undergone treatment with, a cancer chemotherapeutic agent. In some cases, the subject is an individual who is preparing to undergo treatment with, is undergoing treatment with, or who has undergone treatment with, an immune checkpoint inhibitor. In some cases, the subject is an individual who is preparing to undergo treatment with, is undergoing treatment with, or who has undergone treatment with, a cancer chemotherapeutic agent, radiation treatment, surgery, and/or treatment with another therapeutic agent. In some cases, a pharmaceutical composition comprising the redirected T-Cell-MP-coronavirus epitope conjugate or higher order complex thereof is administered in the adjuvant or neoadjuvant setting.

### XIII. DOSAGES AND ROUTES OF ADMINISTRATION

### A. Dosages

A suitable dosage of a T-Cell-MP-epitope conjugate, redirected T-Cell-MP-coronavirus epitope conjugate, or their higher order complexes (e.g., duplexes) can be determined by an attending physician, or other qualified medical personnel, based on various clinical factors. As is well known in the medical arts, dosages for any one patient depend upon many factors, including the patient's size, body surface area, age, the particular T-Cell-MP-epitope conjugate (including the particular MOD(s) employed in the T-Cell-MP-epitope conjugate), redirected T-Cell-MP-coronavirus epitope conjugate (including the particular MOD(s) employed in the redirected T-Cell-MP-coronavirus epitope conjugate), or a higher order complex to be administered, sex of the patient, time, route of administration, general health, and other drugs being administered concurrently. The number and type of MODs per molecule also can play a significant factor. Depending on these factors, a T-Cell-MP-epitope conjugate or a higher order complex thereof, such as a duplex, may be administered in amounts between 1 ng/kg body weight and 20 mg/kg body weight per dose, e.g., from 0.1 mg/kg body weight to 10 mg/kg body weight, e.g., from 0.5 mg/kg body weight to 5 mg/kg body weight; from 1 mg/kg body weight to 5 mg/kg body weight, from 2 mg/kg body weight to 4 mg/kg body weight, however, doses below or above this exemplary range are envisioned, especially considering the aforementioned factors. For example, for duplex molecules comprising 4 reduced-affinity IL-2 MODs such as the H16 and F42 substitutions described above, (e.g., H61A and F42A) have been shown to provide a range of therapeutic activity when administer in dosages of 2 mg/kg body weight or higher, with dosages of 2 and 4 mg/kg providing therapeutic benefit and acceptable patient tolerability. If the regimen is a continuous infusion, it can also be in the range of 1 μg to 10 mg per kilogram of body weight per minute. A T-Cell-MP-epitope conjugate, redirected T-Cell-MP-coronavirus epitope conjugate, or a higher order complex can be administered in an amount of from 1 mg/kg body weight to 50 mg/kg body weight per dose. A T-Cell-MP-epitope conjugate, redirected T-Cell-MP-coronavirus epitope conjugate, or a higher order complex can be administered in an amount of from 1 mg/kg body weight to 5 mg/kg body weight per dose. A T-Cell-MP-epitope conjugate, redirected T-Cell-MP-coronavirus epitope conjugate, or a higher order complex, can be administered in an amount of from 5 mg/kg body weight to 10 mg/kg body weight per dose. A T-Cell-MP-epitope conjugate, redirected T-Cell-MP-coronavirus epitope conjugate, or a higher order complex can be administered in an amount of from 10 mg/kg body weight to 15 mg/kg body weight, or from 15 mg/kg body weight to 20 mg/kg body weight per dose. A T-Cell-MP-epitope conjugate, redirected T-Cell-MP-coronavirus epitope conjugate, or a higher order complex can be administered in an amount of from 20 mg/kg body weight to 25 mg/kg body weight, or from 25 mg/kg body weight to 30 mg/kg body weight per dose. A T-Cell-MP-epitope conjugate, redirected T-Cell-MP-coronavirus epitope conjugate, or a higher order complex can be administered in an amount of from 30 mg/kg body weight to 35 mg/kg body weight, or from 35 mg/kg body weight to 40 mg/kg body weight per dose. A T-Cell-MP-epitope conjugate, redirected T-Cell-MP-coronavirus epitope conjugate, or a higher order complex can be administered in an amount of from 40 mg/kg body weight to 50 mg/kg body weight per dose.

In some cases, a suitable dose of a T-Cell-MP-epitope conjugate, redirected T-Cell-MP-coronavirus epitope conjugate, or a higher order complex is from 0.01 μg to 100 mg per kg of body weight, e.g., from about 0.5 to about 1 mg/kg, from about 1 mg/kg to about 5 mg/kg, from about 5 mg/kg to about 10 mg/kg, or from about 10 mg/kg to about 20 mg/kg of body weight per dose. Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a T-Cell-MP-epitope conjugate, redirected T-Cell-MP-coronavirus epitope conjugate, or a higher order complex is administered in maintenance doses, in one of the above-recited ranges.

Those of skill will readily appreciate that dose levels can vary as a function of the specific T-Cell-MP-epitope conjugate, the severity of the symptoms and the susceptibility of the subject to side effects. Preferred dosages for a given compound are readily determinable by those of skill in the art by a variety of means.

In some cases, multiple doses of a T-Cell-MP-epitope conjugate are administered. The frequency of administration and dose of a T-Cell-MP-epitope conjugate can vary depending on any of a variety of factors, e.g., the level of immune protection generated, severity of the symptoms (in cases of infection), route of administration, etc. For example, in some cases, a T-Cell-MP-epitope conjugate is administered once every year, once every 2-6 months, or once per month. In other cases, a T-Cell-MP-epitope conjugate is administered once every three weeks, or more frequently. When administered to persons who are uninfected and at risk of infection in order to cause priming and/or expansion of epitope-specific T cells and/or induce epitope-specific T cell memory, the administration can comprise an initial dose followed by one or more subsequent doses that are administered within a month, within one to two months, within two to four months, within six months, within six to twelve months, or longer than twelve months after the prior dose. Where the T-Cell-MP-epitope conjugate is administered to an infected individual, it may be administered more often, e.g., once a week, twice a week or more often, or less frequently than once a week, e.g., once every two weeks or even less frequently.

The frequency of administration of one or more redirected T-Cell-MP-coronavirus epitopes, or a higher order complex such as a duplex, can vary depending on any of a variety of factors, but generally speaking will be administered once a week, once every two weeks, once every three weeks, once every four weeks, once per month, or less frequently than once per month, e.g., once every five weeks, once every six weeks, once every two months, once every three months, etc., but also can be administered more frequently than once per week, e.g., twice per week (biw), three times per week (tiw), four times per week, five times per week, six times per week, every other day (qod), or daily (qd). In some cases, the redirected T-Cell-MP-coronavirus epitope conjugate, or a higher order complex such as a duplex is administered once every three weeks. Administration generally should be stopped upon disease progression or unacceptable toxicity.

The duration of administration can vary, depending on any of a variety of factors, e.g., patient response, etc. For example, a redirected T-Cell-MP-coronavirus epitopes, or a higher order complex such as a duplex can be administered over a period of time ranging from one month to about two months, from about two months to about four months, from about four months to about six months, from about six months to about eight months, from about eight months to about 1 year, from about 1 year to about 2 years, or from about 2 years to about 4 years, or more. Typically, the TMMP will continue to be dosed for at least as long as the patient continues to receive a clinically determined benefit, which likely will be from at least many months to multiple years.

### B. Routes of Administration

An active agent (a T-Cell-MP-epitope conjugate) is administered to an individual using any available method and route suitable for drug delivery, including in vivo and in vitro methods, as well as systemic and localized routes of administration.

Conventional and pharmaceutically acceptable routes of administration include intramuscular, intralymphatically, intratracheal, intracranial, subcutaneous, intradermal, topical, intravenous, intra-arterial, rectal, nasal, oral, and other enteral and parenteral routes of administration. Routes of administration may be combined, if desired, or adjusted depending upon the T-Cell-MP-epitope conjugate and/or the desired effect. As noted above, a T-Cell-MP-epitope conjugate, can be administered in a single dose or in multiple doses.

In some embodiments, a T-Cell-MP-epitope conjugate or redirected T-Cell-MP-coronavirus epitopes, or a higher order complex thereof such as a duplex, is administered intravenously. In some embodiments, a T-Cell-MP-epitope conjugate is administered intramuscularly. In some embodiments, a T-Cell-MP-epitope conjugate is administered subcutaneously.

### XIV. CERTAIN ASPECTS

While the present invention has been described with reference to the specific embodiments thereof, it should be understood by those skilled in the art that various changes may be made, and equivalents may be substituted without departing from the true spirit and scope of the invention as set forth in the appended claims. In addition, many modifications may be made to adapt a particular situation, material, composition of matter, process, and/or process step or steps, to the objective, spirit and scope of the present invention. All such modifications are intended to be within the scope of the claims appended hereto.
1. A T cell modulatory polypeptide-epitope conjugate (T-Cell-MP-epitope conjugate), the polypeptide comprising (e.g., in the N-terminal to C-terminal direction):
   (i) optionally one or more independently selected MOD polypeptide sequences (e.g., two or more MOD polypeptide sequences, such as in tandem, wherein when there are two or more MOD polypeptide sequences they are optionally joined to each other by independently selected L1 linkers),
   (ii) an optional L2 linker polypeptide sequence joining the one or more MOD polypeptide sequences to a β2M polypeptide sequence,
   (iii) the β2M polypeptide sequence,
   (iv) an optional L3 linker polypeptide sequence (e.g., from 10-50 aas in length),
   (v) a class I MHC-H (e.g., HLA heavy chain or “HLA-H”) polypeptide sequence,
   (vi) an optional L4 linker polypeptide sequence,
   (vii) a scaffold polypeptide sequence (e.g., an Ig Fc sequence),
   (viii) an optional L5 linker polypeptide sequence, and
   (ix) optionally one or more independently selected MOD polypeptide sequences (e.g., two or more MOD polypeptide sequences, such as in tandem, wherein when there are two or more MOD polypeptide sequences they are optionally joined to each other by independently selected L6 linkers);
   wherein
   the T-Cell-MP-epitope conjugate comprises at least one MOD polypeptide sequence (e.g., at least one of the MODs of element (i) or (ix);
   at least one of the β2M polypeptide sequence, the L3 linker polypeptide sequence, and/or the MHC-H polypeptide sequence comprises one or more chemical conjugation sites to which a peptide presenting a coronavirus epitope (e.g., a peptide, phosphopeptide, glycopeptide, or lipopeptide presenting a coronavirus epitope) is covalently bound, directly or indirectly (e.g., through a linker) to at least one of the one or more chemical conjugation sites; and
   the peptide presenting a coronavirus epitope (e.g., a peptide, phosphopeptide, glycopeptide, lipopeptide or carbohydrate epitope) comprises five (5) or more (e.g., 6, 7, 8, 9, 10, 11, 12, 13, 14, 15 or 16, or 6 or more) contiguous amino acids of any of the coronavirus protein or a coronavirus peptide sequence set forth in FIGS. 20A to 20J, FIG. 2I, FIG. 22, Table 2, Table 3, or Table 3a.
   As noted in aspects 35 to 41, when the MHC-H polypeptide sequence is an HLA-E polypeptide sequence, the peptide presenting an HLA-E restricted non-coronavirus epitope may be present (conjugated to) in the T-Cell-MP in place of the peptide presenting a coronavirus epitope.
2. The T-Cell-MP-epitope conjugate of aspect 1, the polypeptide comprising in the N-terminal to C-terminal direction the elements:
   (i) optionally one or more independently selected MOD polypeptide sequences (e.g., two or more MOD polypeptide sequences, such as in tandem, wherein when there are two or more MOD polypeptide sequences they are optionally joined to each other by independently selected L1 linkers);
   (ii) an optional L2 linker polypeptide sequence;
   (iii) a β2M polypeptide sequence;
   (iv) an optional L3 linker polypeptide sequence (e.g., from 10-50 aas in length);
   (v) a class I MHC-H (e.g., HLA-H) polypeptide sequence;
   (vi) an optional L4 linker polypeptide sequence;
   (vii) a scaffold polypeptide sequence (e.g., an Ig Fc sequence);
   (viii) an optional L5 linker polypeptide sequence; and
   (ix) optionally one or more independently selected MOD polypeptide sequences (e.g., two or more MOD polypeptide sequences, such as in tandem, wherein when there are two or more MOD polypeptide sequences they are optionally joined to each other by independently selected L6 linkers).
The chemical conjugation site for epitope conjugation of aspects 1 and 2 permits the covalent attachment of an epitope presenting molecule (e.g., a peptide presenting an epitope) to the T-Cell-MP such that it can be bound by the MHC-H polypeptide and presented to a TCR. It is understood that the T-Cell-MP-epitope conjugates of aspects 1 and 2 do not comprise any peptide other than the conjugated epitope (either covalently attached to, or as a fusion with, the T-Cell-MP polypeptide) that can be located in the binding cleft of the MHC-H/β2M polypeptide sequences and presented to a TCR. Because the T-Cell-MP-epitope conjugates of aspects 1 and 2, and their dependent aspects, comprise a peptide presenting a coronavirus epitope, they may also be referred to as T-Cell-MP-coronavirus epitope conjugates.
3. The T-Cell-MP-epitope conjugate of aspect 1 or aspect 2, wherein the MHC-H polypeptide sequence comprises a human class I MHC-H chain polypeptide sequence selected from HLA-A, HLA-B, HLA-C, HLA-E, HLA-F, and HLA-G MHC-H polypeptide sequences having at least 90% (e.g., at least 95% or at least 98%) or 100% sequence identity to at least 200 (e.g., at least 225, at least 250, at least 260, or at least 275) contiguous aas of a MHC-H polypeptide provided in any of FIGS. 3A-3H, or an MHC-H consensus sequence polypeptide recited in FIG. 3I.
4. The T-Cell-MP-epitope conjugate of any preceding aspect, wherein the MHC-H sequence does not include the MHC-H transmembrane domain, or a portion thereof, that will anchor the T-Cell-MP in a cell membrane.
5. The T-Cell-MP-epitope conjugate of any preceding aspect, wherein the MHC-H polypeptide sequence has at least 90% (e.g., at least 95% or at least 98%) or 100% sequence identity to at least 200 (e.g., at least 225, at least 250, at least 260, or at least 275) contiguous aas of the α1, α2, and α3 domains of an HLA allele. For example, the MHC-H polypeptide sequence may have at least 95% or at least 98% sequence identity to at least 225 contiguous aas of an HLA polypeptide sequence (e.g., comprising the α1, α2, and α3 domains of an HLA allele) in any of FIGS. 3A-3H, or an HLA allele recited in FIG. 21 or FIG. 22.
6. The T-Cell-MP-epitope conjugate of any of aspects 1-5, wherein the coronavirus peptide epitope comprises 6 to 25, or 8 to 25, contiguous amino acids a SARS-CoV-2 variant spike protein epitope selected from the group consisting of: KVGGNYNYR (SEQ ID NO:473); KGFNCYFPL (SEQ ID NO:475); DVYYHKNNK (SEQ ID NO: 477); NSANLAAIK (SEQ ID NO:480); RFANPVLPF (SEQ ID NO:482); AYSNNSIAIPINF (SEQ ID NO:484); PINLVRGL (SEQ ID NO:486); SRRRARSVA (SEQ ID NO:487); IPINFTISV (SEQ ID NO:489); NLRTRTQL (SEQ ID NO:491); NFTNRTQL (SEQ ID NO:492); LXXXHRSYL (SEQ ID NO:494); LPSAYTNSF (SEQ ID NO: 496); and AENNSY (SEQ ID NO:498).
7. The T-Cell-MP-epitope conjugate of any of aspects 1-5, wherein the peptide presenting a coronavirus epitope comprises a portion of the spike protein of SEQ ID NO:566 bearing one or two sequence changes selected from the group consisting of T19R, A67V, del69-70, (V70F*), T95I, G142D, del142-144, Y145D, R158G, del211, L212I, ins214EPE, (A222V*), (W258L*), G339D, S371L, S373P, S375F, K417N, N440K, G446S, L452R, S477N, T478K, E484A, Q493R, G496S, Q498R, N501Y, Y505H, T547K, D614G, H655Y, N679K, P681H, P681R, N764K, D796Y, N856K, D950N, Q954H, N969K, and L981F.
8. The T-Cell-MP-epitope conjugate of any of aspects 1-5, wherein the peptide presenting a coronavirus epitope comprises a portion of the spike protein of SEQ ID NO:566 bearing one or two substitutions selected from the group consisting of T95I, K417N*T478K, and D614G.
9. The T-Cell-MP-epitope conjugate of any of aspects 1-5, wherein the MHC-H polypeptide sequence has at least 90% (e.g., at least 95%, or at least 98%) or 100% sequence identity to at least 200 (e.g., at least 225, at least 250, at least 260, or at least 275) contiguous aas of an HLA allele (e.g. as provided in FIG. 3E, SEQ ID NOs: 24, 27, and 32-38). For example, the MHC-H polypeptide sequence may have at least 95% or at least 98% sequence identity to at least 225 contiguous aas of HLA-A*0101, HLA-A*0201, HLA-A*0301, HLA-A*1101, HLA-A*2301, HLA-A*2402, HLA-A*2407, HLA-A*3303, or HLA-A*3401 polypeptide sequence provided in FIG. 3E (SEQ ID NOs: 24, 27, and 32-38), or an HLA-A allele recited in FIG. 21 or FIG. 22. The MHC-H polypeptide sequence may also have at least 95% or at least 98% sequence identity to at least 250 contiguous aas of an HLA-A allele such as those provided in FIG. 3E.
10. The T-Cell-MP-epitope conjugate of aspect 9, wherein the MHC-H polypeptide sequence has at least 90% (e.g., at least 95% or at least 98%) or 100% sequence identity to at least 200 (e.g., at least 225, at least 250, at least 260, or at least 275) contiguous aas of an HLA-A*0101, HLA-A*0201, HLA-A*1101, HLA-A*2402, HLA-A*3303, or HLA-A*3401 polypeptide sequence (e.g., as provided in FIG. 3E).
11. The T-Cell-MP-epitope conjugate of any of aspects 1-10, wherein the epitope is an HLA-A restricted epitope set forth in Table 2, Table 3, Table 3a, FIG. 21, or FIG. 22, or of a protein set forth in any of FIGS. 20A to 20J.
12. The T-Cell-MP-epitope conjugate of any of aspects 1-10, wherein the epitope is an HLA-A restricted epitope set forth in Table 2.
13. The T-Cell-MP-epitope conjugate of any of aspects 1-10, wherein the epitope is an HLA-A restricted epitope set forth in Table 3.
14. The T-Cell-MP-epitope conjugate of any of aspects 1-10, wherein the epitope is an HLA-A restricted epitope set forth in Table 3a.
15. The T-Cell-MP-epitope conjugate of any of aspects 1-4, wherein the MHC-H polypeptide sequence has at least 90% (e.g., at least 95% or at least 98%) or 100% sequence identity to at least 200 (e.g., at least 225, at least 250, at least 260, or at least 275) contiguous aas of the α1, α2, and α3 domains of an HLA-B allele (e.g., as provided in FIG. 3F, SEQ ID NOs: 40-46), or an HLA-B allele recited in FIG. 21 or FIG. 22. For example, the MHC-H polypeptide sequence may have at least 95% or at least 98% sequence identity to at least 225 contiguous aas of an HLA-B allele (e.g., as provided in FIG. 3F).
16. The T-Cell-MP-epitope conjugate of any of aspects 1-4 or 15, wherein the MHC-H polypeptide sequence has at least 90% (e.g., at least 95% or at least 98%) or 100% sequence identity to at least 200 (e.g., at least 225, at least 250, at least 260, or at least 275) contiguous aas of an HLA-B*0702, HLA-B*0801, HLA-B*1501, HLA-B*1502, B27 (subtypes HLA-B*2701-2759), HLA-B*2705, HLA-B*3501, HLA-B*3802, HLA-B*4001, HLA-B*4402, HLA-B*4403, HLA-B*4601, HLA-B*5301, or HLA-B*5801 polypeptide sequence (e.g., as provided in FIG. 3F, SEQ ID NOs: 40-46), or an HLA-B allele recited in FIG. 21 or FIG. 22. For example, the MHC-H polypeptide sequence may have at least 95% or at least 98% sequence identity to at least 250 contiguous aas of an HLA-B allele such as those provided in FIG. 3F, SEQ ID NOs: 40-46.
17. The T-Cell-MP-epitope conjugate of any of aspects 1-4, 15, or 16, wherein the MHC-H sequence has at least 90% (e.g., at least 95% or at least 98%) or 100% sequence identity to at least 200 (e.g., at least 225, at least 250, at least 260, or at least 275) contiguous aas of HLA-B*0702.
18. The T-Cell-MP-epitope conjugate of any of aspects 1-4 or 15, wherein the epitope is an HLA-B restricted epitope set forth in Table 2, Table 3, Table 3a, FIG. 21, or FIG. 22, or of a protein set forth in any of FIGS. 20A to 20J.
19. The T-Cell-MP-epitope conjugate of any of aspects 1-4 or 15, wherein the epitope is an HLA-B restricted epitope set forth in Table 2.
20. The T-Cell-MP-epitope conjugate of any of aspects 1-4 or 15, wherein the epitope is an HLA-B restricted epitope set forth in Table 3.
21. The T-Cell-MP-epitope conjugate of any of aspects 1-4 or 15, wherein the epitope is an HLA-B restricted epitope set forth in Table 3a.
22. The T-Cell-MP-epitope conjugate of any of aspects 1-4, wherein the MHC-H polypeptide sequence has at least 90% (e.g., at least 95% or at least 98%) or 100% sequence identity to at least 200 (e.g., at least 225, at least 250, at least 260, or at least 275) contiguous aas of the α1, α2, and α3 domains of an HLA-C allele (e.g., as provided in FIG. 3G). For example, the MHC-H polypeptide sequence may have at least 95% or at least 98% sequence identity to at least 225 contiguous aas of an HLA-C allele (e.g., as provided in FIG. 3G).
23. The T-Cell-MP-epitope conjugate of any of aspects 1-4 or 22, wherein the MHC-H sequence has at least 90% (e.g., at least 95% or at least 98%) or 100% sequence identity to at least 200 (e.g., at least 225, at least 250, at least 260, or at least 275) contiguous aas of an HLA-C*0102, HLA-C*0303, HLA-C*0304, HLA-C*0401, HLA-C*0602, HLA-C*0701, HLA-C*0702, HLA-C*0801, or HLA-C*1502 polypeptide sequence (e.g., as provided in FIG. 3G), or an HLA-C allele recited in FIG. 21 or FIG. 22. For example, the MHC-H polypeptide sequence may have at least 95% or at least 98% sequence identity to at least 250 contiguous aas of an HLA-C allele such as those provided in FIG. 3G.
24. The T-Cell-MP-epitope conjugate of any of aspects 1-4, 22 or 23, wherein the MHC-H polypeptide sequence has at least 90% (e.g., at least 95% or at least 98%) or 100% sequence identity to at least 200 (e.g., at least 225, at least 250, at least 260, or at least 275) contiguous aas of HLA-C*0701.
25. The T-Cell-MP-epitope conjugate of any of aspects 1-4, 22, or 23, wherein the epitope is an HLA-C restricted epitope set forth in Table 3 or FIG. 22, or of a protein set forth in any of FIGS. 20A to 20J.
26. The T-Cell-MP-epitope conjugate of any of aspects 1-4 or 15, wherein the epitope is an HLA-C restricted epitope set forth in Table 3.
27. The T-Cell-MP-epitope conjugate of any of aspects 1-4 or 15, wherein the epitope is an HLA-C restricted epitope set forth in FIG. 3.
28. The T-Cell-MP-epitope conjugate of any of aspects 1-4 or 15, wherein the epitope is an HLA-C restricted epitope of a protein set forth in any of FIGS. 20A to 20J.
29. The T-Cell-MP-epitope conjugate of any of aspects 1-4, wherein the MHC-H polypeptide sequence has at least 90% (e.g., at least 95% or at least 98%) or 100% sequence identity to at least 200 (e.g., at least 225, at least 250, at least 260, or at least 275) contiguous aas of the α1, α2, and α3 domains of an HLA-E allele (e.g., as provided in FIG. 3H). For example, the MHC-H polypeptide sequence may have at least 95% or at least 98% sequence identity to at least 225 contiguous aas of an HLA-E allele (e.g., as provided in FIG. 3H).
30. The T-Cell-MP-epitope conjugate of any of aspects 1-4 or 29, wherein the MHC-H polypeptide sequence has at least 90% (e.g., at least 95% or at least 98%) or 100% sequence identity to at least 200 (e.g., at least 225, at least 250, at least 260, or at least 275) contiguous aas of an HLA-E*0101, HLA-E*01:03, HLA-E*01:04, HLA-E*01:05, HLA-E*01:06, HLA-E*01:07, HLA-E*01:09, or HLA-E*01:10 polypeptide sequence (e.g., as provided in FIG. 3H). For example, the MHC-H polypeptide sequence may have at least 95% or at least 98% sequence identity to at least 250 contiguous aas of an HLA-E allele such as those provided in FIG. 3H.
31. The T-Cell-MP-epitope conjugate of any of aspects 1-4, 29 or 30, wherein the MHC-H polypeptide sequence has at least 90% (e.g., at least 95% or at least 98%) or 100% sequence identity to at least 200 (e.g., at least 225, at least 250, at least 260, or at least 275) contiguous aas of the HLA-E allele consensus sequence:

GSHSLKYFHT SVSRPGRGEP RFISVGYVDD
TQFVRFDNDA ASPRMVPRAP WMEQEGSEYW
DRETRSARDT AQIFRVNLRT LRGYYNQSX1A
GSHTLQWMHG CELGPDX2RFL RGYEQFAYDG
KDYLTLNEDL RSWTAVDTAA QISEQKSNDA
SEAEHQX3X4YL EDTCVEWLHK YLEKGKETLL
HLEPPKTHVT HHPISDHEAT LRCWALGFYP
AEITLTWQQD GEGHTQDTEL VETRPAGDGT
FQKWAAVVVP SGEEX5RYTCH VQHEGLX6EPV
TLRWKPASQP TIPI,

Wherein: X1=K or E, X2=R or G, X3=R or G, X4=A or V, X5=Q or P, and X6=P or S (SEQ ID NO:58).
32. The T-Cell-MP-epitope conjugate of aspect 31, wherein the MHC-H polypeptide sequence has at least at least 95% or at least 98% sequence identity to at least 250 contiguous aas of SEQ ID NO:58 when X1=K, X2=R, X3=R, X4=A, X5=Q, and X6=S.
33. The T-Cell-MP-epitope conjugate of any of aspects 1-4, 29, 30, 31, or 32, wherein the epitope is an HLA-E restricted epitope of a protein set forth in any of FIGS. 20A to 20J.
34. The T-Cell-MP-epitope conjugate of any of aspects 1-4, 29, 30, 31, or 32, wherein the epitope comprises the aa sequence VMPLSAPTL (SEQ ID NO:2058).

Although the aspects set forth above are directed to T-Cell-MP-epitope conjugates comprising a peptide presenting a coronavirus epitope, it will be understood that the T-Cell-MPs may be conjugated to peptides presenting epitopes other than coronavirus epitopes (i.e., non-coronavirus epitopes). Among those peptides presenting HLA-E restricted epitopes that associate with complexes of HLA-E and β2M including, but not limited to, the peptides set forth in aspects 35-41.
35. The T-Cell-MP-epitope conjugate of any of aspects 1-4, 29, 30, 31, or 32, wherein the peptide presenting an HLA-E restricted epitope comprises an aa sequence selected from the group consisting of: QMPSRSLLF (SEQ ID NO: 2059), TLPKRGLFL (SEQ ID NO:2060), TGPWRSLWI (SEQ ID NO:2061), ILTDRSLWL (SEQ ID NO: 2062), VNPGRSLFL (SEQ ID NO:2063), WNRLFPPLR (SEQ ID NO:2064), TLPERTLYL (SEQ ID NO: 2065), FLPNRSLLF (SEQ ID NO:2066), VMGDRSVLY (SEQ ID NO:2067), and VMADKSIFY (SEQ ID NO: 2068).
36. The T-Cell-MP-epitope conjugate of any of aspects 1-4, 29, 30, 31, or 32, wherein the peptide presenting an HLA-E restricted epitope comprises an aa sequence selected from the group consisting of: VMPPRTLLL (SEQ ID NO.: 2069), VMAPRTLFL (SEQ ID NO:2070), VNPGRSLFL (SEQ ID NO:2063), QMPSRSLLF (SEQ ID NO.: 2071), TLPERTLYL (SEQ ID NO:2065), VMPGRTLCF (SEQ ID NO:2072), ILTDRSLWL (SEQ ID NO:262), RMPPRSVLL (SEQ ID NO:2073), TAPARTMFL (SEQ ID NO.: 2074), and NMPARTVLF SEQ ID NO:2075.
37. The T-Cell-MP-epitope conjugate of any of aspects 1-4, 29, 30, 31, or 32, wherein the epitope comprises the aa sequence WNRILPNAY SEQ ID NO:2076.
38. The T-Cell-MP-epitope conjugate of any of aspects 1-4, 29, 30, 31, or 32, wherein the peptide presenting an HLA-E restricted epitope comprises the aa sequence X1-X2-X3-X4-R-X6-X7-X8-X9-X10, where: X1 is absent or S, K, A, I, L, or T; X2 is L, I, K or V; X3 is P, A or S; X4 is G, V, P, D or R; X6 is S or T; X7 is L, I, or V; X8 is F, W, or E; X9 is L. F, W or E; and X10 F, H, S, A or absent SEQ ID NO:2077.
39. The T-Cell-MP-epitope conjugate of any of aspects 1-4, 29, 30, 31, or 32, wherein the peptide presenting an HLA-E restricted epitope comprises the aa sequence LP (G or V) RSLFL SEQ ID NO:2078, or LP (G or V) RSLWL (SEQ ID NO:2079).
40. The T-Cell-MP-epitope conjugate of any of aspects 1-4, 29, 30, 31, or 32, wherein the peptide presenting an HLA-E restricted epitope comprises a CMV aa sequence selected from the group consisting of: VLPHRTQFL (SEQ ID NO:2080), TGAARSFFF (SEQ ID NO:2081), and VMAPRTLIL (SEQ ID NO:2082).
41. The T-Cell-MP-epitope conjugate of any of aspects 1-4, 29, 30, 31, or 32, wherein the peptide presenting an HLA-E restricted epitope comprises a CMV aa sequence selected from the group consisting of: SAPLKTRFL “(SEQ ID NO:2083), and SVPLKTRFL (SEQ ID NO:2084).
42. The T-Cell-MP-epitope conjugate of any of aspects 1-4, wherein the MHC-H polypeptide sequence has at least 90% (e.g., at least 95% or at least 98%) or 100% sequence identity to at least 200 (e.g., at least 225, at least 250, at least 260, or at least 275) contiguous aas of the α1, α2, and α3 domains of an HLA-F allele. For example, the MHC-H polypeptide sequence may have at least 95% or at least 98% sequence identity to at least 225 contiguous aas of an HLA-F allele.
43. The T-Cell-MP-epitope conjugate of any of aspects 1-4 or 42, wherein the MHC-H polypeptide sequence has at least 90% (e.g., at least 95% or at least 98%) or 100% sequence identity to at least 200 (e.g., at least 225, at least 250, at least 260, or at least 275) contiguous aas of an HLA-F*0101 (HLA-F*01:01:01:01), HLA-F*01:02, HLA-F*01:03 (HLA-F*01:03:01:01), HLA-F*01:04, HLA-F*01:05, or HLA-F*01:06 polypeptide sequence (e.g., as provided in FIG. 3H). For example, the MHC-H polypeptide sequence may have at least 95% or at least 98% sequence identity to at least 250 contiguous aas of an HLA-F allele such as those provided in FIG. 3H.
44. The T-Cell-MP-epitope conjugate of any of aspects 1-4, 42, or 43, wherein the MHC-H polypeptide sequence has at least 90% (e.g., at least 95% or at least 98%) or 100% sequence identity to at least 200 (e.g., at least 225, at least 250, at least 260, or at least 275) contiguous aas of the HLA-F allele consensus sequence: GSHSLRX1FST AVSRPGRGEP RYIAVEYVDD TQFLRFDSDA AIPRMEPREX2 WVEQEGPQYW EWTTGYAKAN AQTDRVALRN LLRRYNQSEA GSHTLQGMNG CDMGPDGRLL RGYHQHAYDG KDYISLNEDL RSWTAADTVA QITQRFYEAE EYAEEFRTYL EGECLELLRR YLENGKETLQ RADPPKAHVA HHPISDHEAT LRCWALGFYP AEITLTWORD GEEQTQDTEL VETRPAGDGT FQKWAAVVVP X3GEEQRYTCH VQHEGLPQPL ILRWEQSX4QP TIPI; wherein X1=Y or F; X2=P or Q; X3=S or P; and X4=P or L (SEQ ID NO:59).
45. The T-Cell-MP-epitope conjugate of aspect 44, wherein the MHC-H polypeptide sequence has at least at least 95% or at least 98% sequence identity to at least 250 contiguous aas of SEQ ID NO:59; when X1=Y, X2=P, X3=S, and X4=P.
46. The T-Cell-MP-epitope conjugate of any of aspects 1-4, 42, 43, 44, or 45, wherein the epitope is an HLA-F restricted epitope of a protein set forth in any of FIGS. 20A to 20J.
47. The T-Cell-MP-epitope conjugate of any of aspects 1-4, wherein the MHC-H polypeptide sequence has at least 90% (e.g., at least 95% or at least 98%) or 100% sequence identity to at least 200 (e.g., at least 225, at least 250, at least 260, or at least 275) contiguous aas of the α1, α2, and α3 domains of an HLA-G allele. For example, the MHC-H polypeptide sequence may have at least 95% or at least 98% sequence identity to at least 225 contiguous aas of an HLA-G allele.
48. The T-Cell-MP-epitope conjugate of any of aspects 1-4 or 47, wherein the MHC-H polypeptide sequence has at least 90% (e.g., at least 95% or at least 98%) or 100% sequence identity to at least 200 (e.g., at least 225, at least 250, at least 260, or at least 275) contiguous aas of an HLA-G*01:04 (HLA-G*01:04:01:01), HLA-G*01:06, HLA-G*01:07, HLA-G*01:08, HLA-G*01:09: HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, or HLA-G*01:22 polypeptide sequence (e.g., as provided in FIG. 3H). For example, the MHC-H polypeptide sequence may have at least 95% or at least 98% sequence identity to at least 250 contiguous aas of an HLA-G allele such as those provided in FIG. 3H.
49. The T-Cell-MP-epitope conjugate of any of aspects 1-4 or 47, wherein the MHC-H polypeptide sequence has at least 90% (e.g., at least 95% or at least 98%) or 100% sequence identity to at least 200 (e.g., at least 225, at least 250, at least 260, or at least 275) contiguous aas of the HLA-G allele consensus sequence:

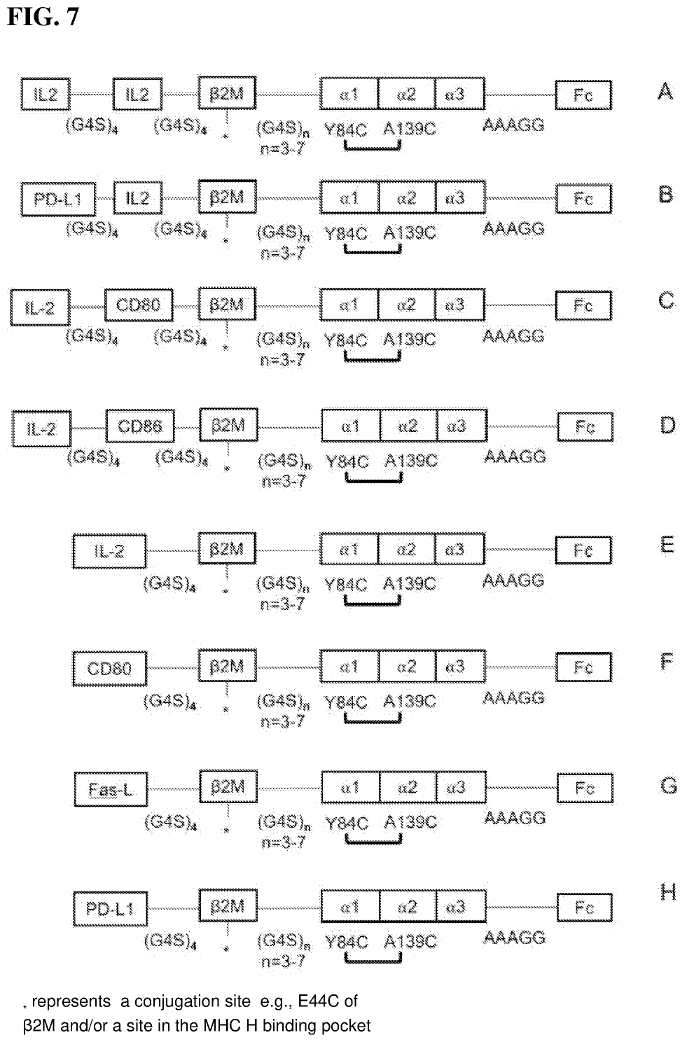

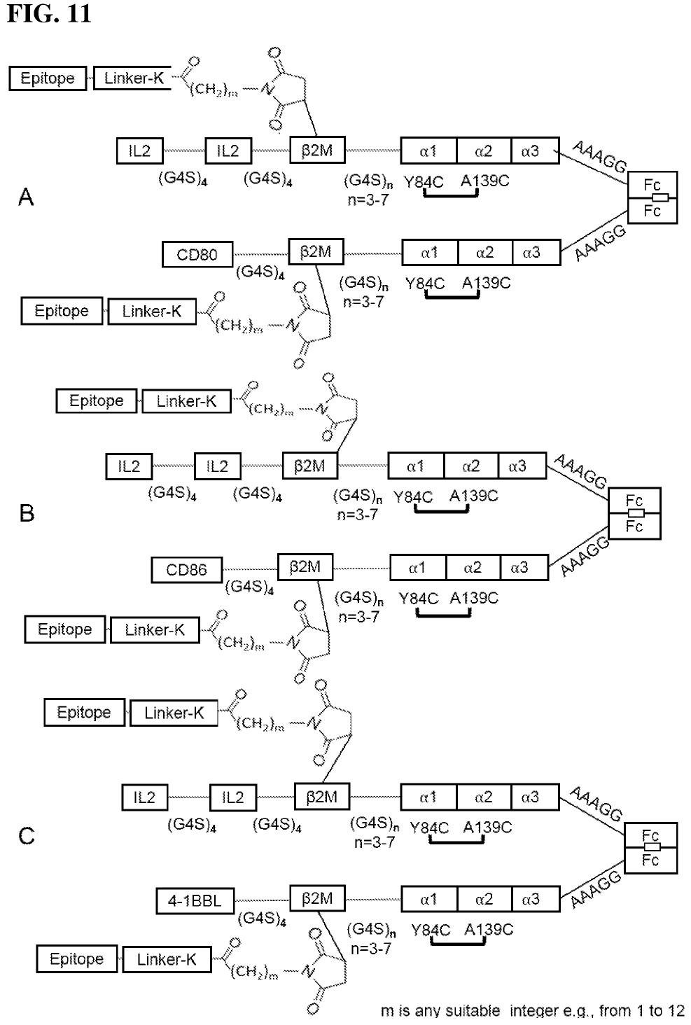

GSHSMRYFSA AVX1RPGRGEP RFIAMGX2VDD
X3QFX4RFDSDS ACPRMEPRAP WVEX5EGPEYW
EEETRNTKAH AQTDRMNLQT X6RGYYNQSEA
SSHTLQWMIX7 CDLX8X9DGRLX10 RGYEQYAYDG
KDYLALNEDL RSWTAADTAA QISKRKCEAA
NVAEQRRAX11L EGTCVEWLX12R X13LENGKEX14LQ
RADPX15KTHVT HHPVFDYEAT LRCWALGFYP
AEIILTWQX16D GEDQTQDVEL VETRPAGDGT
FQKWAAVWVP SGEEQRYX17CH VQHEGLPEPL
MLRWX18QSSLP TIPI,

wherein X1=S or F, X2=Y or H, X3=T, S, or M, X4=L or V; X5=Q or R, X6=P or L, X7=G or D, X8=G or V, X9=S or C, X10=L or I, X11=Y or H, X12=H or R, X13=Y or H, X14=M or T, X15=P or A, X16=R, W, or Q, X17=T or M, X18=K or E (SEQ ID NO:60).
50. The T-Cell-MP-epitope conjugate of aspect 49, wherein the MHC-H polypeptide sequence has at least at least 95% or at least 98% sequence identity to at least 250 contiguous aas of SEQ ID NO:60; X1=S, X2=Y, X3=T, X4=L, X5=, X6=P, X7=G, X8=G, X9=S, X10=L, X11=Y, X12=H, X13=Y, X14=M, X15=P, X16=R, X17=T, X18=K.
51. The T-Cell-MP-epitope conjugate of any of aspects 1-4, 47, 48, 49, or 50, wherein the epitope is an HLA-G restricted epitope of a protein set forth in any of FIGS. 20A to 20J.
52. The T-Cell-MP-epitope conjugate of any of aspects 1-51, wherein the MHC-H polypeptide sequence comprises at least one mutation (e.g., two, or three mutations) selected from the group consisting of: an alanine at position 84 (e.g., Y84A or R84A in the case of HLA-F), a cysteine at position 84 (e.g., Y84C or R84C in the case of HLA-F), a cysteine at position 139 (e.g., A139C or V139C in the case of HLA-F), and a cysteine at position 236 (e.g., A236C). See FIG. 3I for the location of those aa positions.
53. The T-Cell-MP-epitope conjugate of any of aspects 1-52, wherein the MHC-H polypeptide sequence comprises a combination of mutations selected from the group consisting of: Y84A and A139C; Y84A and A236C; Y84C and A139C; Y84C and A236C; and Y84C, A139C and A236C.
54. The T-Cell-MP-epitope conjugate of any of aspects 1-52, wherein the MHC-H polypeptide sequence comprises: a cysteine at position 84 (e.g., Y84C or R84C in the case of HLA-F), a cysteine at position 139 (e.g., A139C or V139C in the case of HLA-F), and optionally a cysteine at position 236 (e.g., A236C). See FIG. 3I for the location of those aa positions.
55. The T-Cell-MP-epitope conjugate of any preceding aspect, wherein the β2M sequence has at least 90% (e.g., at least 95% or at least 98%) or 100% sequence identity to at least 70 (e.g., at least 80, at least 90, at least 96, at; east 97, or at least 98) or all contiguous aas of a mature human β2M polypeptide (e.g., aas 21-119 of NCBI accession number NP_004039.1 SEQ ID NO:61 provided in FIG. 4), and optionally comprises a cysteine at position 12 (an R12C substitution).
56. The T-Cell-MP-epitope conjugate of any preceding aspect, wherein the β2M sequence has at least 95% or at least 98% sequence identity to at least 90 or all contiguous aas of a mature human β2M polypeptide (e.g., aas 21-119 of NCBI accession number NP_004039.1 SEQ ID NO:61 provided in FIG. 4), and optionally comprises a cysteine at position 12 (an R12C substitution).
57. The T-Cell-MP-epitope conjugate of any of aspects 1-56, wherein the β2M sequence has up to 6 (e.g., 1, 2, 3, 4, or 5) aa substitutions within an aa segment of at least 70 (e.g., at least 80, 90, 96, 97, or 98 or all) contiguous aas of a mature human β2M polypeptide (e.g., aas 21-119 of NCBI accession number NP_004039.1 provided in FIG. 4), and optionally comprises a cysteine at position 12 (an R12C substitution).
58. The T-Cell-MP-epitope conjugate of any of aspects 1-57, comprising at least one linker sequence comprising, consisting essentially of, consisting predominantly of (based on the number of aas), or consisting of: i) Gly and/or Ser; ii) Ala and Ser; iii) Gly, Ala, and Ser; iv) Gly, Ser, and Cys (e.g., a single Cys residue); v) Ala, Ser, and Cys (e.g., a single Cys residue); or vi) Gly, Ala, Ser, and Cys (e.g., a single Cys residue).
59. The T-Cell-MP-epitope conjugate any of aspects 1-57, comprising at least one linker (e.g., any of linkers L1-L6) that comprises one or more sequences selected independently from: polyG (e.g., comprising 1-10 Gly residues), GA, AG, AS, SA, GS, GSGGS (SEQ ID NO:121), GGGS (SEQ ID NO:122), GGSG (SEQ ID NO: 123), GGSGG (SEQ ID NO:124), GSGSG (SEQ ID NO:125), GSGGG (SEQ ID NO:126), GGGSG (SEQ ID NO: 127), GSSSG (SEQ ID NO:128), GGGGS (SEQ ID NO: 130), or AAAGG (SEQ ID NO: 132), any of which may be repeated 1, 2, 3, 4, 5, 6, 7, 8, 9, or 10 times.
60. The T-Cell-MP-epitope conjugate of any preceding aspect, wherein at least one, at least two, or at least three linker comprises a G4S sequence (SEQ ID NO:130) that may be repeated from 1-10 times (e.g., repeated 1, 2, 3, 4, 5, 6, 7, 8, 9, or 10 times), or an AAAGG sequence (SEQ ID NO:132) that may be repeated from 1-10 times.
61. The T-Cell-MP-epitope conjugate of any preceding aspect, wherein the scaffold polypeptide sequence comprises an independently selected non-interspecific sequence or interspecific sequence.
62. The T-Cell-MP-epitope conjugate of aspect 61, wherein the interspecific and non-interspecific sequences are selected from the group consisting of: Ig heavy chain constant regions (Ig Fc e.g., CH2-CH3); collectin polypeptides, coiled-coil domains, leucine-zipper domains; Fos polypeptides; Jun polypeptides; Ig CH1; Ig C_{L} κ; Ig C_{L} λ; knob-in-hole without disulfide (KiH); knob-in hole with a stabilizing disulfide bond (KiHs-s); HA-TF; ZW-1; 7.8.60; DD-KK; EW-RVT; EW-RVTs-s; and A107 sequences.
63. The T-Cell-MP-epitope conjugate of any preceding aspect complexed to form a duplex T-Cell-MP-epitope conjugate or other higher order T-Cell-MP-epitope conjugate comprising at least a first T-Cell-MP-epitope conjugate and a second T-Cell-MP-epitope conjugate of any of aspects 1-62, wherein:
   (i) the first T-Cell-MP-epitope conjugate comprises a first β2M polypeptide sequence; a first class I MHC-H polypeptide sequence; and a first scaffold polypeptide; and
   (ii) the second T-Cell-MP-epitope conjugate comprises a second β2M polypeptide sequence; a second class I MHC-H polypeptide sequence; and a second scaffold polypeptide; and wherein the first and second T-Cell-MP-epitope conjugates associate by binding interactions between the first and second scaffold polypeptides that optionally includes at least one or at least two interchain covalent bonds (e.g., one or two disulfide bonds). See e.g., the duplexes in FIGS. 9 to 11.
64. The T-Cell-MP-epitope conjugate or duplex T-Cell-MP-epitope conjugate of aspect 63, wherein the first and second scaffold polypeptides comprise a pair of non-Ig polypeptide sequences.
65. The duplex T-Cell-MP-epitope conjugate of aspect 64, wherein the pair of non-Ig polypeptide sequences are non-interspecific polypeptide sequences (e.g., non-interspecific coiled-coil or leucine zipper sequences).
66. The duplex T-Cell-MP-epitope conjugate of aspect 64, wherein the pair of non-Ig polypeptide sequences are a pair of interspecific polypeptide sequences selected from interspecific coiled-coil or leucine zipper sequences or a Fos protein polypeptide sequence that pairs with a Jun protein polypeptide sequence.
67. The duplex T-Cell-MP-epitope conjugate of aspect 63, wherein the first and second scaffold polypeptides comprise a pair of Ig polypeptide sequences.
68. The duplex T-Cell-MP-epitope conjugate of aspect 67, wherein the pair of Ig polypeptide sequences comprise one or more substitutions that reduce the binding with Ig Fc receptors and/or complement C1q protein relative to a T-Cell-MP where the Ig polypeptide sequence is unsubstituted.
69. The duplex T-Cell-MP-epitope conjugate of aspect 67 or 68, wherein the first and second scaffold polypeptides comprise a pair of non-interspecific Ig polypeptide aa sequences (e.g., an Ig CH2-CH3 containing aa sequence such as an Ig Fc polypeptide aa sequence).
70. The duplex T-Cell-MP-epitope conjugate of aspect 69, wherein the pair of non-interspecific Ig polypeptide sequence comprises a human IgA Fc, IgD Fc, or IgE Fc polypeptide sequence comprising an aa sequence having at least about 90% (e.g., at least about 95%, at least about 98%, or at least about 99%) or 100% aa sequence identity to at least 175 (e.g., at least 200, or at least 210) contiguous aas, or all aas of an aa sequence of an Ig Fc region depicted in FIGS. 2A-2C.
71. The duplex T-Cell-MP-epitope conjugate of aspect 69, wherein the pair of non-interspecific Ig polypeptide sequence comprises a human IgG1 Fc, IgG2 Fc IgG3 Fc or IgG4 Fc polypeptide sequence comprising an aa sequence having at least about 90% (e.g., at least about 95%, at least about 98%, or at least about 99%) or 100% aa sequence identity to at least 175 (e.g., at least 200, or at least 210) contiguous aas, or all aas of an aa sequence of an Ig Fc region depicted in FIGS. 2D-2G. For example, the pair of non-interspecific Ig polypeptide sequences may comprise a human IgG1 Fc, IgG2 Fc IgG3 Fc or IgG4 Fc aa sequence having at least about 95% or at least about 98% aa sequence identity to at least 200 contiguous aas of an Ig Fc region depicted in FIGS. 2D-2G.
72. The r duplex T-Cell-MP-epitope conjugate of any of aspects 69 or 70, wherein the pair of non-interspecific Ig polypeptide sequence comprises a human IgG1 Fc polypeptide sequence comprising an aa sequence having at least about 90% (e.g., at least about 95%, at least about 98%, or at least about 99%) or 100% aa sequence identity to at least 175 (e.g., at least 200, or at least 210) contiguous aas, or all aas of an aa sequence of the wild-type (wt.) Ig Fc sequence depicted in FIG. 2D. For example, the pair of non-interspecific Ig polypeptide sequences may comprise a human IgG1 Fc aa sequence having at least about 95% or at least about 98% aa sequence identity to at least 200 contiguous aas of the wild-type (wt.) Ig Fc sequence depicted in FIG. 2D (SEQ ID NO: 4).
73. The duplex T-Cell-MP-epitope conjugate of aspect 72, wherein the pair of non-interspecific Ig polypeptide sequences comprises polypeptide having at least one substitution at L234, L235, G236, G237, P238, S239, D270, N297, K322, P329, and/or P331 (respectively, aas L14, L15, G16, G17, P18, S19, D50, N77, K102, P109, and P111 of the wt. IgG1 aa sequence in FIG. 2D) or another substitution (e.g., a corresponding substitution) that reduces binding to the Fc A receptor and/or the C1q protein relative to the same sequence without the substitutions.
74. The duplex T-Cell-MP-epitope conjugate of aspect 72, wherein the pair of non-interspecific Ig polypeptide sequences comprises a substitution at P331 and: (i) a substitution of N297 (e.g., N297A); (ii) a substitution of any of aas 234 to 239; (iii) a substitution at L234; (iv) a substitution at L235; (v) a substitution at L234 and L235 (e.g., an L234A and L235A or “LALA” substitution); (vi) a substitution of P331; or (vii) substitutions of D270, K322, and/or P329; substitutions at L234 and/or L235 (e.g., L234F, L235E, and P331S substitutions).
75. The duplex or higher order T-Cell-MP-epitope conjugate of aspect 63, wherein the first and second scaffold polypeptide sequences comprise an IgM heavy chain constant region.
76. The duplex T-Cell-MP-epitope conjugate of aspect 63, wherein the first and second scaffold polypeptides comprise a pair of interspecific Ig polypeptide sequences.
77. The T-Cell-MP-epitope conjugate or duplex T-Cell-MP-epitope conjugate of aspect 76, wherein the pair of interspecific Ig sequences is selected from the group consisting of Ig heavy chain constant regions (Ig Fc CH2-CH3) comprising: knob-in-hole without disulfide (KiH), knob-in hole with a stabilizing disulfide bond (KiHs-s), HA-TF, ZW-1, 7.8.60, DD-KK, EW-RVT, EW-RVTs-s, or A107 sequence pairs; or an Ig CH1 paired with an Ig C_{L} κ or Ig C_{L} λ.
78. The duplex T-Cell-MP-epitope conjugate of aspect 76, wherein the pair of interspecific Ig sequences comprise KIH or a KIHs-s polypeptide sequences.
79. The duplex T-Cell-MP-epitope conjugate of aspect 76, wherein the pair of interspecific Ig sequences comprise EW-RVT or EW-RVTs-s polypeptide sequences.
80. The T-Cell-MP-epitope conjugate of aspect 76, wherein the pair of interspecific Ig sequences comprise HA-TF, ZW-1, 7.8.60, DD-KK, or A107 polypeptide sequences.
81. The duplex T-Cell-MP-epitope conjugate of any of aspects 76-80, further comprising one or more substitutions that reduce binding to the Fc A receptor and/or the C1q protein (e.g., substitutions at IgG1 aa L234 and/or L235, or K322) relative to the same sequence without the substitutions.
82. The T-Cell-MP-epitope conjugate of any of aspects 76-81, further comprising one or more substitutions that limit complement activation (e.g., reduce binding to the complement C1q protein such as by substitutions at IgG D270, N297, K322, P329, and/or P331) relative to the same sequence without the substitutions.
83. The duplex T-Cell-MP-epitope conjugate of any of aspects 76-82, wherein the pair of interspecific Ig polypeptide sequences comprise a human IgG1 Fc sequence comprising an aa sequence having at least about 90% (e.g., at least about 95%, at least about 98%, or at least about 99%) aa sequence identity to at least 175 (e.g., at least 200, or at least 210) contiguous aas of the wt. Ig Gg1 Fc sequence in FIG. 2D. For example, the pair of interspecific Ig polypeptide sequences may comprise a human IgG1 Fc sequence having at least about 90% or at least about 95% aa sequence identity to at least 200 contiguous aas of the wild-type (wt.) Ig Fc sequence depicted in FIG. 2D (SEQ ID NO:4).
84. The duplex T-Cell-MP-epitope conjugate of aspect 83, wherein the interspecific Ig polypeptide sequence comprises one or more Ig Fc regions, comprising at least one substitution at L234, L235, G236, G237, P238, S239, D270, N297, K322, P329, and/or P331 (respectively, aas L14, L15, G16, G17, P18, S19, D50, N77, K102, P109, and P111 of the wt. IgG1 aa sequence in FIG. 2D) or another substitution (e.g., a corresponding substitution) that reduces binding to the Fc A receptor and/or the C1q protein relative to the same sequence without the substitutions.
85. The duplex T-Cell-MP-epitope conjugate of aspect 83, comprising a substitution at P331 (e.g., L234F, L235E, and P331S substitutions) and: (i) a substitution of N297 (e.g., N297A); (ii) a substitution of any of aas 234 to 239; (iii) a substitution at L234; (iv) a substitution of L235; (v) a substitution of L234 and L235 (e.g., an L234A and L235A or “LALA” substitution); (vi) a substitution of P331; or (vii) substitutions of D270, K322, and/or P329; substitutions at L234 and/or L235.
86. The T-Cell-MP-epitope conjugate, or the duplex or higher order T-Cell-MP-epitope conjugate of any of aspects 1-85, comprising at least one (e.g., at least two, at least three, or at least four) wt. MOD and/or at least one (e.g., at least two, at least three, or at least four) variant MOD polypeptide sequences selected independently from the group consisting of: IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (Ig-like transcript 3), IL T4 (Ig-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, 4-1BBL and anti-CD28 polypeptide sequences.
87. The T-Cell-MP-epitope conjugate or duplex T-Cell-MP-epitope conjugate of any preceding aspect, comprising at least one (e.g., at least two, at least three, or at least four) wt. MOD and/or at least one (e.g., at least two, or at least three) variant MOD polypeptide sequence selected independently from the group consisting of: 4-1BBL, anti-CD28, PD-L1, IL-2, CD80, CD86, OX40L (CD252), Fas ligand (FasL), ICOS-L, ICAM, CD30L, CD40, CD83, HVEM (CD270), JAG1 (CD339), CD70, CD80, CD86, TGF-β1, TGF-β2, and TGF-β3 polypeptide sequences.
88. The T-Cell-MP-epitope conjugate or duplex T-Cell-MP-epitope conjugate of any preceding aspect, comprising at least one (e.g., at least two, at least three, or at least four) independently selected wt. MOD or at least one (e.g., at least two, at least three, or at least four) variant MOD polypeptide sequence selected independently from the group consisting of 4-1BBL, PD-L1, IL-2, CD80, CD86, FasL wt. MOD or variant MOD polypeptide sequences and anti-CD28. For example, the T-Cell-MP-epitope conjugate or duplex T-Cell-MP-epitope conjugate may comprise at least one wt. IL-2 MOD and/or variant IL-2 MOD polypeptide sequence, and at least one wt. CD80, wt. CD86, variant CD80 or variant CD86 polypeptide sequence.
89. The T-Cell-MP-epitope conjugate or duplex T-Cell-MP-epitope conjugate of any preceding aspect, comprising at least one (e.g., at least two) independently selected wt. IL-2 or at least one (e.g., at least two, at least three, or at least four) independently selected variant IL-2 MOD (e.g., comprising a H16A or T substitution and a F42A or T substitution, such as H16A and F42A substitutions) polypeptide sequence, or at least one pair of wt. IL-2 MOD polypeptide sequences in tandem or at least one pair of variant IL-2 MOD polypeptide sequences in tandem.
90. The T-Cell-MP-epitope conjugate or duplex T-Cell-MP-epitope conjugate of any preceding aspect comprising at least one (e.g., at least two, at least three, or at least four) independently selected wt. or variant: (i) CD80; (ii) CD86 MOD polypeptide sequence; (iii) PD-L1 MOD polypeptide sequence; and/or (iv) FasL MOD polypeptide sequence.
91. The T-Cell-MP-epitope conjugate or duplex T-Cell-MP-epitope conjugate of any preceding aspect, comprising an intrachain disulfide bond between a cysteine substituted into the carboxyl end portion of the α1 helix and a cysteine in the amino end portion of the α2-1 helix (e.g., substituted in the amino end portion of the α2-1 helix) of the MHC-H polypeptide sequence.
92. The T-Cell-MP-epitope conjugate or duplex T-Cell-MP-epitope conjugate of any preceding aspect, comprising an intrachain disulfide bond between a cysteine substituted into the carboxyl end portion of the α1 helix at position 84 and a cysteine in the amino end portion of the α2-1 helix at position 139 of the MHC-H polypeptide sequence;
   wherein when the MHC-H polypeptide has a sequence having at least 90% or at least 95% sequence identity to SEQ ID NO:84, the five residue clusters amino and carboxyl to position 84 (denoted aac1 and aac2, respectively) and, the five residue clusters amino and carboxyl to position 139 (denoted aac3, and aac4 respectively) may each be substituted with 1 to 5 independently selected naturally occurring aas, and the five residue clusters amino and carboxyl to position 236 (denoted aac5 and aac6, respectively) may each be substituted with 1 to 5 independently selected naturally occurring aas, and the sequence identity is calculated excluding the aas of the five residue clusters.
93. The T-Cell-MP-epitope conjugate or duplex T-Cell-MP-epitope conjugate of aspect 92, wherein each of the amino acids of aac1 to aac6 may each be substituted with independently selected naturally occurring aa other than proline.
94. The T-Cell-MP-epitope conjugate or duplex T-Cell-MP-epitope conjugate of aspect 92, wherein the carboxyl end portion of the α1 helix comprises a first sequence CYNQSE (see e.g., position 84 and aac2 in FIG. 3I) and the amino end portion of the α2-1 helix of the MHC-H polypeptide sequence comprises a second sequence D (M/T) CAQ (e.g., the sequence spanning aac3 and aac4 in FIG. 3I), and wherein the intrachain disulfide bond is formed between the cysteines in the first and second sequences.
95. The duplex T-Cell-MP-epitope conjugate of any of aspects 63-94, wherein the first T-Cell-MP-epitope conjugate and the second T-Cell-MP-epitope conjugate are not linked by disulfide bonds.
96. The duplex T-Cell-MP-epitope conjugate of any of aspects 63-94, wherein the first T-Cell-MP-epitope conjugate and the second T-Cell-MP-epitope conjugate are covalently linked by at least one (e.g., at least two) disulfide bond(s).
97. The duplex T-Cell-MP-epitope conjugate of aspect 96, wherein the first T-Cell-MP-epitope conjugate and the second T-Cell-MP-epitope conjugate are covalently linked by at least one (e.g., at least two) disulfide bond(s) between the scaffold polypeptide sequences of the first T-Cell-MP-epitope conjugate and the second T-Cell-MP-epitope conjugate.
98. The duplex T-Cell-MP-epitope conjugate of any of aspects 63-97, wherein the sequences of at least one of (e.g., both) the first T-Cell-MP-epitope conjugate and the second T-Cell-MP-epitope conjugate do not comprise an Ig CH1 domain polypeptide sequence.
99. The duplex T-Cell-MP-epitope conjugate of any of aspects 63-75 and 86-98, wherein the first T-Cell-MP-epitope conjugate and the second T-Cell-MP-epitope conjugate are identical, and the duplex T-Cell-MP-epitope conjugate is a homodimer. See, e.g., FIGS. 6, 7, and 9 structure C.
100. The duplex T-Cell-MP-epitope conjugate of aspect 99, wherein the first T-Cell-MP-epitope conjugate and the second T-Cell-MP-epitope conjugate comprise at least one (e.g., at least two, or at least three) wt. MOD or variant MOD polypeptide sequence selected independently from the group consisting of: IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (Ig-like transcript 3), ILT4 (Ig-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, 4-1BBL polypeptide sequences and anti-CD28.
101. The duplex T-Cell-MP-epitope conjugate of aspect 99, wherein the first T-Cell-MP-epitope conjugate and a second T-Cell-MP-epitope conjugate comprise at least one (e.g., at least two, or at least three) wt. MOD or variant MOD polypeptide sequence selected independently from the group consisting of: 4-1BBL, PD-L1, IL-2, CD80, CD86, FasL wt. MOD or variant MOD polypeptide sequences, and anti-CD28. For example, the T-Cell-MP-epitope conjugate or duplex T-Cell-MP-epitope conjugate may comprise at least one IL-2 wt. MOD or variant MOD polypeptide sequence, and at least one CD80, CD86, variant CD80 or variant CD86 polypeptide sequence.
102. The duplex T-Cell-MP-epitope conjugate of aspect 99, wherein the first T-Cell-MP-epitope conjugate and a second T-Cell-MP-epitope conjugate comprise at least one IL-2 wt. MOD or variant MOD (e.g., comprising a H16A or T substitution and a F42A substitution) polypeptide sequence, or at least one pair of IL-2 wt. MOD or variant MOD polypeptide sequences in tandem.
103. The duplex T-Cell-MP-epitope conjugate of any of aspects 99-102, wherein the first T-Cell-MP-epitope conjugate and the second T-Cell-MP-epitope conjugate further comprise at least one: (i) CD80 and/or CD86 wt. MOD or variant MOD polypeptide sequence; (ii) at least one PD-L1 wt. MOD or variant MOD polypeptide sequence; and/or (iii) at least one FasL wt. MOD or variant MOD polypeptide sequence.
104. The duplex T-Cell-MP-epitope conjugate of any of aspects 63-64 and 76-98, wherein the scaffold polypeptides of the first T-Cell-MP and the second T-Cell-MP-epitope conjugate are a pair of interspecific polypeptide sequences, and the duplex T-Cell-MP-epitope conjugate is a heterodimer. See, e.g., FIG. 10 structure C.
105. The duplex T-Cell-MP-epitope conjugate of aspect 104, wherein at least one (e.g., both) of the first T-Cell-MP-epitope conjugate and the second T-Cell-MP-epitope conjugate comprises at least one wt. MOD or variant MOD polypeptide sequence selected independently from the group consisting of: IL-1, IL-2, IL-4, IL-6, IL-7, IL-10, IL-12, IL-15, IL-17, IL-21, IL-23, CD7, CD30L, CD40, CD70, CD80 (B7-1), CD83, CD86 (B7-2), HVEM (CD270), ILT3 (Ig-like transcript 3), ILT4 (Ig-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, anti-CD28, and 4-1BBL polypeptide sequences.
106. The duplex T-Cell-MP-epitope conjugate of aspect 104, wherein at least one (e.g., both) of the first T-Cell-MP-epitope conjugate and the second T-Cell-MP-epitope conjugate comprises at least one wt. MOD or variant MOD polypeptide sequence selected independently from the group consisting of: 4-1BBL, anti-CD28, PD-L1, IL-2, CD80, CD86, and FasL wt. MOD or variant MOD polypeptide sequences. For example, the duplex T-Cell-MP-epitope conjugate may comprise at least one IL-2 wt. MOD or variant MOD polypeptide sequence, and at least one anti-CD28, CD80, CD86, variant CD80 or variant CD86 polypeptide sequence.
107. The duplex T-Cell-MP-epitope conjugate of aspect 104, wherein at least one (e.g., both) of the first T-Cell-MP-epitope conjugate and the second T-Cell-MP-epitope conjugate comprises at least one independently selected IL-2 wt. MOD or variant IL-2 MOD polypeptide sequence, or at least one pair of IL-2 wt. MOD polypeptide sequences in tandem or variant IL-2 MOD polypeptide sequences in tandem.
108. The duplex T-Cell-MP-epitope conjugate of aspect 104, wherein at least one (e.g., both) of the first T-Cell-MP-epitope conjugate and the second T-Cell-MP-epitope conjugate comprises at least one independently selected: (i) CD80 and/or CD86 wt. MOD or variant MOD polypeptide sequence; (ii) at least one PD-L1 wt. MOD or variant MOD polypeptide sequence; and/or (iii) at least one FasL wt. MOD or variant MOD polypeptide sequence.
109. The duplex T-Cell-MP-epitope conjugate of aspect 104, wherein at least one (e.g., both) of the first T-Cell-MP-epitope conjugate and the second T-Cell-MP-epitope conjugate comprises at least one CD80 and/or CD86 wt. MOD or variant MOD polypeptide sequence.
110. The duplex T-Cell-MP-epitope conjugate of aspect 104, wherein at least one (e.g., both) of the first T-Cell-MP-epitope conjugate and the second T-Cell-MP-epitope conjugate comprises at least one PD-L1 wt. MOD or variant MOD polypeptide sequence.
111. The duplex T-Cell-MP-epitope conjugate of any of aspects 104-110, wherein: (i) the first T-Cell-MP-epitope conjugate and the second T-Cell-MP-epitope conjugate do not comprise the same MODs; (ii) the first T-Cell-MP-epitope conjugate and the second T-Cell-MP-epitope conjugate do not comprise the same number of MODs; or (iii) the MODs are placed in different locations of the first T-Cell-MP-epitope conjugate and the second T-Cell-MP-epitope conjugate.
112. The T-Cell-MP-epitope conjugate of any of aspects 1-65 or 86-94, complexed to form triplex T-Cell-MP-epitope conjugates, quadraplex T-Cell-MP-epitope conjugates, pentaplex T-Cell-MP-epitope conjugates, or hexaplex T-Cell-MP-epitopes.
113. The T-Cell-MP-epitope conjugate or duplex T-Cell-MP-epitope conjugate of any of aspects 1-112, wherein each chemical conjugation site is jointly or independently selected from: a) amino acid chemical conjugation sites; b) non-natural amino acids and/or selenocysteines; c) peptide sequences that act as an enzymatic modification sequence (e.g., a sulfatase motif); d) carbohydrate or oligosaccharide moieties; and/or e) IgG nucleotide binding sites.
114. The T-Cell-MP-epitope conjugate or duplex T-Cell-MP-epitope conjugate of any of aspects 1-113, wherein at least one (e.g., two or more) chemical conjugation site comprises an enzymatic modification sequence.
115. The T-Cell-MP-epitope conjugate or duplex T-Cell-MP-epitope conjugate of any of aspects 1-114, wherein at least one (e.g., two or more) chemical conjugation site comprises a sulfatase motif.
116. The T-Cell-MP-epitope conjugate or duplex T-Cell-MP-epitope conjugate of aspect 115, wherein the sulfatase motif comprises the sequence X1Z1X2Z2X3Z3 (SEQ ID NO:66) wherein:
   Z1 is cysteine or serine; Z2 is either a proline or alanine residue; Z3 is a basic amino acid (arginine, lysine, or histidine, usually lysine), or an aliphatic amino acid (alanine, glycine, leucine, valine, isoleucine, or proline, usually A, G, L, V, or I);
   X1 is present or absent and, when present, can be any amino acid, though usually an aliphatic amino acid, a sulfur-containing amino acid, or a polar, uncharged amino acid (i.e., other than an aromatic amino acid or a charged amino acid), usually L, M, V, S or T, more usually L, M, S or V, with the proviso that, when the sulfatase motif is at the N-terminus of the target polypeptide, X1 is present; and
   X2 and X3 independently can be any amino acid, though usually an aliphatic amino acid, a polar, uncharged amino acid, or a sulfur containing amino acid (i.e., other than an aromatic amino acid or a charged amino acid), usually S, T, A, V, G or C, more usually S, T, A, V or G.
117. The T-Cell-MP-epitope conjugate or duplex T-Cell-MP-epitope conjugate of aspect 116, wherein at least one Z1 residue has been converted into an fGly amino acid residue.
118. The T-Cell-MP-epitope conjugate or duplex T-Cell-MP-epitope conjugate of any of aspects 1-114, wherein: at least one (e.g., or more, two) of the chemical conjugation sites comprises a Sortase A enzyme site (e.g., comprising the amino acid sequence LP (X5) TG/A (SEQ ID NO:69), or LPETGG (SEQ ID NO:74) positioned at the C-terminus of at least one (e.g., both) T-Cell-MP polypeptides; or at least one of the chemical conjugation sites is a Sortase A enzyme site comprising an oligoglycine (e.g., (G) 2, 3, 4, or 5) or an oligo alanine (e.g., (A) 2, 3, 4, or 5) at the amino terminus of at least one of or both the first or second T-Cell-MP polypeptides.
119. The T-Cell-MP-epitope conjugate or duplex T-Cell-MP-epitope conjugate of any of aspects 1-114, wherein at least one (e.g., two or more) chemical conjugation site comprises a transglutaminase site (e.g., selected from the group consisting of: LQG, LLQGG (SEQ ID NO:76), LLQG (SEQ ID NO:77), LSLSQG (SEQ ID NO:78), and LLQLQG (SEQ ID NO: 79).
120. The T-Cell-MP-epitope conjugate or duplex T-Cell-MP-epitope conjugate of any of aspects 1-114, wherein at least one (e.g., two or more) chemical conjugation site comprises a selenocysteine, or an amino acid sequence containing one or more independently selected non-natural amino acids.
121. The T-Cell-MP-epitope conjugate or duplex T-Cell-MP-epitope conjugate of aspect 120, wherein at least one of the one or more non-natural amino acids (e.g., two or more) is selected from the group consisting of para-acetylphenylalanine, para-azido phenylalanine and propynyl-tyrosine.
122. The T-Cell-MP-epitope conjugate or duplex T-Cell-MP-epitope conjugate of any of aspects 1-113, wherein at least one (e.g., two or more) chemical conjugation site comprises a carbohydrate, monosaccharide, disaccharide and/or oligosaccharide.
123. The T-Cell-MP-epitope conjugate or duplex T-Cell-MP-epitope conjugate of any of aspects 1-113, wherein at least one (e.g., two or more) chemical conjugation site comprises one or more IgG nucleotide binding sites.
124. The T-Cell-MP-epitope conjugate or duplex T-Cell-MP-epitope conjugate of any of aspects 1-113, wherein at least one (e.g., two or more) chemical conjugation site comprises an amino acid conjugation site (e.g., a cysteine provided in a T-Cell-MP-epitope conjugate by protein engineering of its sequence).
125. The T-Cell-MP-epitope conjugate or duplex T-Cell-MP-epitope conjugate of aspect 124, wherein the at least one chemical conjugation site comprises a sulfhydryl group of a cysteine provided in a T-Cell-MP-epitope conjugate polypeptide sequence, or in the polypeptide sequence of each of the first T-Cell-MP-epitope conjugate and second T-Cell-MP-epitope conjugate of a duplex T-Cell-MP-epitope conjugate (e.g., provided in a T-Cell-MP-epitope conjugate by protein engineering of the polypeptide sequence(s)).
126. The T-Cell-MP-epitope conjugate or duplex T-Cell-MP-epitope conjugate of aspect 124, wherein the at least one (e.g., two or more) chemical conjugation site comprises a selenol group of selenocysteine and/or a sulfhydryl group of a cysteine provided in a T-Cell-MP-epitope conjugate polypeptide sequence (e.g., provided in a T-Cell-MP-epitope conjugate by protein engineering of its polypeptide sequence).
127. The T-Cell-MP or duplex T-Cell-MP-epitope conjugate of aspect 124, wherein the at least one (e.g., two or more) chemical conjugation site comprises the epsilon amino group of a lysine provided in a T-Cell-MP-epitope conjugate polypeptide sequence (e.g., provided in a T-Cell-MP-epitope conjugate by protein engineering of its polypeptide sequence).
128. The T-Cell-MP-epitope conjugate or duplex T-Cell-MP-epitope conjugate of any of aspects 1-127, wherein each chemical conjugation site (e.g., for the conjugation of an epitope) present in the T-Cell-MP-epitope conjugate or duplexed T-Cell-MP-epitope conjugate is selected to be the same (e.g., the chemical conjugation sites of both the first and second T-Cell-MP in a duplex T-Cell-MP-epitope conjugate are the sulfhydryl of a cysteine provided by protein engineering of the polypeptide sequences and undergo a common conjugation reaction).
129. The T-Cell-MP-epitope conjugate or duplex T-Cell-MP-epitope conjugate of any of aspects 1-128, wherein a chemical conjugation site (e.g., for the conjugation of an epitope) is located at or within 25 aa of the N-terminus or C-terminus of a T-Cell-MP, or if present, at the N-terminus or C-terminus or within a linker located at the N-terminus or C-terminus of the T-Cell-MP.
130. The T-Cell-MP-epitope conjugate or duplex T-Cell-MP-epitope conjugate of any of aspects 1-128, wherein a chemical conjugation site is located in a linker of the T-Cell-MP (e.g., an L1-L6 linker).
131. The T-Cell-MP-epitope conjugate or duplex T-Cell-MP-epitope conjugate of any of aspects 1-128, wherein the one or more chemical conjugation site(s) (e.g., for the conjugation of an epitope) is/are located in the MHC-H polypeptide sequence, the β2M polypeptide sequence (e.g., a Q2C, E44C, E50C, E77C, V85C, S88C, K91C, or D98C substitution in a mature β2M polypeptide sequence), or a linker sequence joining the MHC-H and β2M polypeptide sequences (the L3 linker).
132. The T-Cell-MP-epitope conjugate or duplex T-Cell-MP-epitope conjugate of any of aspects 1-128, wherein the one or more chemical conjugation sites (e.g., for the conjugation of an epitope) is/are located in a linker sequence joining the MHC-H and β2M polypeptide sequences (the L3 linker).
133. The T-Cell-MP-epitope conjugate or duplex T-Cell-MP-epitope conjugate of aspect 132, where the one or more chemical conjugation site is/are sulfhydryl of a cysteine present in the linker sequence joining the MHC-H and β2M polypeptide sequences.
134. The T-Cell-MP-epitope conjugate or duplex T-Cell-MP-epitope conjugate of aspect 133, wherein the linker sequence joining the MHC-H and β2M polypeptide sequences further comprises a glycine, glycine and serine, alanine, alanine and serine, or alanine glycine and serine containing polypeptide sequence.
135. The T-Cell-MP-epitope conjugate or duplex T-Cell-MP-epitope conjugate of aspect 133, wherein the linker sequence joining the MHC-H and β2M polypeptide sequences comprises the polypeptide sequence GGGS (SEQ ID NO:122) or GGGGS (SEQ ID NO: 130).
136. The T-Cell-MP-epitope conjugate or duplex T-Cell-MP-epitope conjugate of aspect 133, wherein the linker sequence joining the MHC-H and β2M polypeptide sequences comprises a polypeptide sequence selected from the group consisting of: GCGGS (G4S) (SEQ ID NO: 156) where the G4S unit may be repeated from 1 to 10 times (e.g., repeated 1, 2, 3, 4, 5, 6, 7, 8, 9, or 10 times), GCGASGGGGSGGGGS (SEQ ID NO:157), GCGGSGGGGSGGGGSGGGGS (SEQ ID NO: 158) and GCGGSGGGGSGGGGS SEQ ID NO:159).
137. The T-Cell-MP-epitope conjugate or duplex T-Cell-MP-epitope conjugate of any of aspects 131-136, wherein the linker sequence joining the MHC-H and β2M polypeptide sequences (the L3 linker) comprises from 10 to 50 amino acids.
138. The T-Cell-MP-epitope conjugate or duplex T-Cell-MP-epitope conjugate of any of aspects 131-136, wherein the linker sequence joining the MHC-H and β2M polypeptide sequences (the L3 linker) comprises from 15 to 50 amino acids.
139. The T-Cell-MP-epitope conjugate or duplex T-Cell-MP-epitope conjugate of any of aspects 1-128, wherein the one or more chemical conjugation site (e.g., for the conjugation of an epitope) is/are located in the MHC-H polypeptide sequence, which has at least 95% (e.g., at least 97% or at least 98%) or 100%) aa sequence identity to at least 200, or 225 contiguous aas of a MHC-H sequence shown in FIGS. 3A-3I or an HLA allele recited in FIG. 3A to FIG. 3H.
140. The T-Cell-MP-epitope conjugate or duplex T-Cell-MP-epitope conjugate of aspect 139, wherein the one or more chemical conjugation sites comprise a cysteine or selenocysteine.
141. The T-Cell-MP-epitope conjugate or duplex T-Cell-MP-epitope conjugate of aspect 110, wherein at least one cysteine or selenocysteine chemical conjugation site is located at position 2, 5, 7, 59, 84, 116, 139, 167, 168, 170, or 171 of a MHC-H polypeptide with the numbering of the corresponding aas conducted by alignment (e.g. using Clustal Omega Version 1.2.2 using default parameters) as in FIGS. 3D-3I.
142. The T-Cell-MP-epitope conjugate or duplex T-Cell-MP-epitope conjugate of any of aspects 1-128, wherein a chemical conjugation site (e.g., for the conjugation of an epitope) is located in the β2M polypeptide sequence, which has at least 90% (e.g., at least 95%, at least 98%, or at least 99%) or 100% aa sequence identity to at least 80 (e.g., at least 90, at least 95, at least 97, or at least 98) or all contiguous aas of a mature β2M polypeptide sequence provided in FIG. 4 (e.g., the sequences shown in FIG. 4 starting at aa 21 and ending at their C-terminus, such as aas 21-119 of NCBI accession number NP_004039.1 SEQ ID NO:61).
143. The T-Cell-MP-epitope conjugate or duplex T-Cell-MP-epitope conjugate of aspect 142, wherein the one or more chemical conjugation sites is/are located between aas 35-55 (e.g., 40 to 50) of the β2M polypeptide sequence, and the β2M polypeptide sequence has 1 to 15 or 1 to 10 aa substitutions.
144. The T-Cell-MP-epitope conjugate or duplex T-Cell-MP-epitope conjugate of aspect 142, wherein at least one cysteine or selenocysteine chemical conjugation site is located at position 2, 44, 50, 77, 85, 88, 91, or 98 (aas 22, 64, 70, 97, 105, 108, 111, or 118 of the β2M sequences as shown in FIG. 4, e.g., the human β2M sequence of NP_004039.1, SEQ ID NO:61 or the corresponding positions in the other β2M sequences). For example, the T-Cell-MP-epitope conjugate or duplex T-Cell-MP-epitope conjugate may comprise a cysteine located at position 44 as an E44C substitution in NP_004039.1, SEQ ID NO:61.
145. The T-Cell-MP-epitope conjugate or duplex T-Cell-MP-epitope conjugate of any of aspects 1-128, wherein a chemical conjugation site (e.g., for the conjugation of an epitope) is located in the β2M polypeptide sequence, which has 1 to 15 or 1-10 (e.g., 1, 2, 3, 4, 5, 6, 7, 8, 9, 10, 11, 12, 13, 14, or 15) aa deletions, insertions and/or changes compared with a mature β2M polypeptide set forth in FIG. 4 (starting at aa 21 and ending at its C-terminus).
146. The T-Cell-MP-epitope conjugate or duplex T-Cell-MP-epitope conjugate of any of aspects 142-145, wherein the chemical conjugation site is a cysteine (e.g., the sulfhydryl of a cysteine).
147. The T-Cell-MP-epitope conjugate or duplex T-Cell-MP-epitope conjugate of any of aspects 142-145, wherein the β2M polypeptide sequence is a mature human β2M sequence of FIG. 4.
148. The duplex T-Cell-MP-epitope conjugate of any of aspects 63-147, wherein at least the first T-Cell-MP-epitope conjugate polypeptide sequence, and optionally the first and second T-Cell-MP-epitope conjugate polypeptide sequences comprise, from in the N-terminal to C-terminal direction:
   (i) one or more independently selected MOD polypeptide sequences optionally joined by L1 linkers;
   (ii) an L2 linker polypeptide sequence;
   (iii) a β2M polypeptide sequence;
   (iv) an L3 linker polypeptide sequence comprising from 10 to 50 or 10 to 30 (e.g., from 10 to 20, from 10 to 25, from 15 to 25, from 20 to 30, from 25 to 35, from 25 to 50, from 30 to 35, from 35 to 45, or from 40 to 50) amino acids;
   (v) a class I MHC-H polypeptide sequence comprising cysteines substituted at positions 84 and 139 (see FIGS. 3E-3H, e.g., Y84C and A139C substitutions) and forming a intrachain disulfide bond;
   (vi) an L4 linker polypeptide sequence;
   (vii) an interspecific or non-interspecific Ig Fc scaffold sequence;
   (viii) an optional L5 linker polypeptide sequence; and
   (ix) optionally one or more independently selected MOD polypeptide sequences (e.g., two or more MOD polypeptide sequences, such as in tandem, optionally joined by L6 linkers);
   wherein at least one of the β2M polypeptide sequence, the L3 linker polypeptide sequence, or the MHC-H polypeptide sequence comprises a chemical conjugation site (e.g., added by protein engineering) to which a peptide presenting an epitope (e.g., a peptide presenting a coronavirus epitope or an HLA-E restricted non-coronavirus epitope such as a peptide, phosphopeptide, glycopeptide, lipopeptide or carbohydrate epitope) is directly or indirectly (through a linker) covalently bound; and
   wherein the first and second T-Cell-MP-epitope conjugate are covalently linked through at least one disulfide bond between their Ig Fc scaffold sequences.
149. The duplex T-Cell-MP-epitope conjugate of any of aspects 63-147, wherein at least the first T-Cell-MP-epitope conjugate polypeptide sequence, and optionally the first and second T-Cell-MP-epitope conjugate polypeptide sequences comprise (e.g., in the N-terminal to C-terminal direction):
   (i) optionally one or more independently selected MOD polypeptide sequences optionally joined by L1 linkers;
   (ii) an optional L2 linker polypeptide sequence;
   (iii) a β2M polypeptide sequence;
   (iv) an L3 linker polypeptide sequence comprising from 10 to 50 amino acids;
   (v) a class I MHC-H polypeptide sequence comprising cysteines substituted at positions 84 and 139 (see FIGS. 3E-3H, e.g., Y84C and A139C substitutions) and forming a disulfide bond;
   (vi) an L4 linker polypeptide sequence;
   (vii) an interspecific or non-interspecific Ig Fc scaffold sequence;
   (viii) an L5 linker polypeptide sequence; and
   (ix) one or more independently selected MOD polypeptide sequences joined by L6 linker polypeptides;
   wherein at least one of the β2M polypeptide sequence, the L3 linker polypeptide sequence, or the MHC-H polypeptide sequence comprises a chemical conjugation site (e.g., added by protein engineering) to which a peptide presenting an epitope (e.g., a peptide presenting a coronavirus epitope or an HLA-E restricted non-coronavirus epitope such as a peptide, phosphopeptide, glycopeptide, lipopeptide or carbohydrate epitope) is directly or indirectly (e.g., through a linker) covalently bound; and
   wherein the first and second T-Cell-MPs are covalently linked through at least one disulfide bond between their Ig Fc scaffold sequences.
150. The duplex T-Cell-MP-epitope conjugate of aspects 148 or 149, wherein the chemical conjugation site(s) of the first and second T-Cell-MP-epitope conjugate polypeptides is within the L3 linker.
151. The duplex T-Cell-MP-epitope conjugate of aspect 150, wherein the chemical conjugation site(s) of the first and second T-Cell-MP-epitope conjugate polypeptides is the sulfhydryl of a cysteine present in the L3 linker comprises, consists essentially of consists predominantly of (based on the number of aas), or consists of a glycine, serine and/or alanine residues.
152. The duplex T-Cell-MP-epitope conjugate of aspects 148 or 149, wherein the chemical conjugation site(s) of the first and second T-Cell-MP-epitope conjugate polypeptides is within the β2M polypeptide sequence (e.g., a mature β2M polypeptide sequence or portion thereof having at least 90% (e.g., at least 95%, at least 98%, or at least 99%) or 100% aa sequence identity to at least 85 (e.g., at least 90, at least 95, at least 97, or at least 98) contiguous aas or all contiguous aas of a β2M polypeptide sequence provided in FIG. 4).
153. The duplex T-Cell-MP-epitope conjugate of aspect 152, wherein the chemical conjugation site(s) of the first and second T-Cell-MP-epitope conjugate polypeptides is the sulfhydryl of a cysteine provided at the β2M polypeptide sequence.
154. The duplex T-Cell-MP-epitope conjugate of aspect 153, wherein the chemical conjugation site(s) of the first and second T-Cell-MP-epitope conjugate polypeptides is the sulfhydryl of a cysteine provided at the β2M polypeptide at position 44 of a mature β2M polypeptide sequence provided in FIG. 4 (e.g., an E44C or K44C substitution in a mature β2M polypeptide sequence or portion thereof having at least 90% (e.g., at least 95%, at least 98%, or at least 99%) or 100% aa sequence identity to at least 70 (e.g., at least at least 80, at least 90, at least 95, at least 97, or at least 98) or all contiguous aas of a β2M polypeptide sequence provided in FIG. 4).
155. The duplex T-Cell-MP-epitope conjugate of any of aspects 148-154, wherein the one or more MOD polypeptide sequences comprise at least one (e.g., two or more) wt. IL-2 or variant IL-2 sequence (e.g., comprising a H16A or T substitution and a F42A substitution).
156. The duplex T-Cell-MP-epitope conjugate of any of aspects 148-155, wherein the one or more MOD polypeptide sequences comprise at least one wt. or variant CD80 or CD86 sequence.
157. The duplex T-Cell-MP-epitope conjugate of any of aspects 148-156, wherein the one or more MOD polypeptide sequences comprise at least one wt. or variant PD-L1 sequence.
158. The duplex T-Cell-MP-epitope conjugate of any of aspects 148-157, wherein the one or more MOD polypeptide sequences comprise at least one wt. or variant 4-1BBL or PD-L1 sequence.
159. The duplex T-Cell-MP-epitope conjugate of any of aspects 148-158, wherein:
   (i) the Ig Fc scaffold is non-interspecific scaffold polypeptide and the duplex is a homodimer comprising identical first and second T-Cell-MP-epitope conjugate polypeptides; or
   (ii) the first and second scaffold polypeptides are an interspecific pair of Ig Fc scaffold polypeptides (e.g., a KIH or KIHs-s pair), and the duplex is a heterodimer.
160. The duplex T-Cell-MP-epitope conjugate of aspect 159, wherein the first and second scaffold polypeptides are an interspecific pair of Ig Fc scaffold polypeptides, and the first T-Cell-MP-epitope conjugate polypeptide sequence comprises at least one independently selected MOD polypeptide sequence that is not present in the second T-Cell-MP-epitope conjugate polypeptide sequence.
161. The T-Cell-MP-epitope conjugate or duplex T-Cell-MP of any preceding aspect, wherein at least one T-Cell-MP is conjugated to a peptide, phosphopeptide, glycopeptide, lipopeptide epitope.
162. The T-Cell-MP-epitope conjugate or duplex T-Cell-MP-epitope conjugate of any preceding aspect, wherein the coronavirus epitope presented by the peptide is a SARS-CoV2 coronavirus epitope or an HLA-E restricted non-coronavirus epitope.
163. The T-Cell-MP-epitope conjugate or duplex T-Cell-MP-epitope conjugate of any of aspects 131-132, wherein the peptide presenting an epitope is a peptide, glycopeptide, phosphopeptide, or lipopeptide that comprises from 4 aas (aa) to 25 aa (e.g., the peptide can have a length of from 5 aa to 10 aa, from 6 aa to 12 aa, from 8 aa to 15 aa, from 15 aa to 20 aa, or from 20 aa to 25 aa).
164. The T-Cell-MP-epitope conjugate or duplex T-Cell-MP-epitope conjugate of aspect 161 to 164, wherein the peptide epitope is from 6 aas to 12 aas, from 8 aas to 15 aas or from 15 aas to 25 aas.
165. The T-Cell-MP-epitope conjugate or duplex T-Cell-MP-epitope conjugate of any of aspects 1-34 or 42-164, wherein the peptide presenting a coronavirus epitope comprises 6 to 25 contiguous amino acids of a coronavirus protein selected from the group consisting of: surface glycoprotein; membrane protein; nucleocapsid phosphoprotein; open reading frame (ORF) 1b protein; ORF3a protein; ORF6 protein; ORF7a protein; ORF8 protein; ORF10 protein; envelope protein; membrane glycoprotein; nonstructural protein (nsp) one (nsp1); nsp2; nsp4; nsp6; nsp7, nsp8, nsp9, nsp10; nsp14; nsp15; nsp16; nucleocapsid phosphoprotein; papain-like cysteine protease (Plpro); 3C-like protease (3C_{L}); RNA-dependent RNA polymerase (Rdpol) and helicase (Hel).
166. The T-Cell-MP-epitope conjugate or duplex T-Cell-MP-epitope conjugate of any of aspects 161-165, wherein the peptide presenting a coronavirus epitope comprises 8 to 15, or 15 to 20, contiguous amino acids of any one of the peptides set forth in FIG. 21 and/or Table 2.
167. The T-Cell-MP-epitope conjugate or duplex T-Cell-MP-epitope conjugate of any of aspects 161-165, wherein the peptide presenting a coronavirus epitope comprises 8 to 15, or 15 to 20, contiguous amino acids of any one of the peptides set forth in FIG. 22 and/or Table 3.
168. The T-Cell-MP-epitope conjugate or duplex T-Cell-MP-epitope conjugate of any of aspects 161-165, wherein the peptide presenting a coronavirus epitope comprises the aa sequence of a peptide set forth in Table 3a.
169. The T-Cell-MP-epitope conjugate or duplex T-Cell-MP-epitope conjugate of any of aspects 161-168, wherein the chemical conjugation site is the sulfhydryl of a cysteine and the coronavirus epitope is a SARS-CoV2 coronavirus epitope.
170. The T-Cell-MP-epitope conjugate or duplex T-Cell-MP-epitope conjugate of aspect 169, where the cysteine is located in the β2M polypeptide sequence or the L3 linker sequence joining the MHC-H and β2M polypeptide sequences.
171. The T-Cell-MP-epitope conjugate or duplex T-Cell-MP-epitope conjugate of aspect 170, where the cysteine is located in the β2M polypeptide sequence.
172. The T-Cell-MP-epitope conjugate or duplex T-Cell-MP-epitope conjugate of aspect 171, wherein in the cysteine is a cysteine substituted at position 2, 44, 50, 77, 85, 88, 91, or 98 of a mature β2M polypeptide sequence (e.g., a Q2C, E44C, E50C, E77C, V85C, S88C, K91C, or D98C substitution in GenBank accession NP_004039.1, SEQ ID NO:61 numbered lacking its signal sequence).
173. The T-Cell-MP-epitope conjugate or duplex T-Cell-MP-epitope conjugate of aspect 172, wherein in the cysteine is a cysteine substituted at position 44 of a mature β2M polypeptide sequence (e.g., an E44C substitution in GenBank accession NP_004039.1, SEQ ID NO:61 numbered lacking its signal sequence).
174. The T-Cell-MP-epitope conjugate or duplex T-Cell-MP-epitope conjugate of any of aspects 169-173, wherein the epitope is covalently linked directly or indirectly (through a linker) to the sulfhydryl of the cysteine, through a bond formed by reaction with a maleimide group (e.g., a bond to a pyrrolidone-2,5-dione group resulting from addition of the sulfhydryl to the maleimide group, see e.g., FIGS. 8-11).
175. The T-Cell-MP-epitope conjugate or duplex T-Cell-MP-epitope conjugate of any of aspects 161-174, wherein the MHC-H polypeptide sequence has at least 90% or 95% sequence identity to at least 200 contiguous aas of an MHC-H chain polypeptide selected from the group consisting of: HLA-A*0101 (SEQ ID NO:24), HLA-A*0201 (SEQ ID NO:27), HLA-A*0301 (SEQ ID NO:35), HLA-A*1101 (SEQ ID NO:32), HLA-A*2301 (SEQ ID NO: 36), HLA-A*2402 (SEQ ID NO:33), HLA-A*2407 (SEQ ID NO:37), HLA-A*3303 (SEQ ID NO:34), HLA-A*3401 (SEQ ID NO:38), HLA-B*0702 (SEQ ID NO:25), HLA-B*0801 (SEQ ID NO:41), HLA-B*1501 (SEQ ID NO: 2041), HLA-B*1502 (SEQ ID NO:42), HLA-B*2705 (SEQ ID NO:2042), HLA-B*3501 (SEQ ID NO:80), HLA-B*3802 (SEQ ID NO:43), HLA-B*4001 (SEQ ID NO:44), HLA-B*4402 (SEQ ID NO:81), HLA-B*4403 (SEQ ID NO:82), HLA-B*4601 (SEQ ID NO:45), HLA-B*5301 (SEQ ID NO:46), HLA-B*5801 (SEQ ID NO:83), HLA-C*0102, (SEQ ID NO:48), HLA-C*0303 (SEQ ID NO:49), HLA-C*0304 (SEQ ID NO:50), HLA-C*0401 (SEQ ID NO:51), HLA-C*0602 (SEQ ID NO:52), HLA-C*0701 (SEQ ID NO:53), HLA-C*0702 (SEQ ID NO:54), HLA-C*0801 (SEQ ID NO:55), and HLA-C*1502 (SEQ ID NO:56, an HLA-E polypeptide (SEQ ID NO: 58), an HLA-F polypeptide (SEQ ID NO: 59), and an HLA-G polypeptide (SEQ ID NO:60).
176. The T-Cell-MP-epitope conjugate or duplex T-Cell-MP-epitope conjugate of aspect 175, wherein the MHC-H polypeptide sequence has at least 90% or at least 95% sequence identity to at least 200 contiguous aas of an MHC-H chain polypeptide selected from the group consisting of: wherein the MHC-H polypeptide sequence has at least 90% sequence identity to at least 200 contiguous aas of an MHC-H chain polypeptide selected from the group consisting of: HLA-A*0201 (SEQ ID NO:27), HLA-A*2402 (SEQ ID NO:33), HLA-A*1101 (SEQ ID NO: 32), HLA-B*4001 (SEQ ID NO:44), and HLA-B*5801 (SEQ ID NO:83).
177. The T-Cell-MP-epitope conjugate or duplex T-Cell-MP-epitope conjugate of any of aspects 161-174, wherein the MHC-H polypeptide sequence has at least 90% sequence identity to at least 200 contiguous aas of an MHC-H chain polypeptide selected from the group consisting of: HLA-A allele consensus sequence SEQ ID NO: 39, HLA-B allele consensus sequence SEQ ID NO:47, and HLA-C allele consensus sequence SEQ ID NO: 57.
178. The T-Cell-MP-epitope conjugate, duplex T-Cell-MP-epitope conjugate, or higher order T-cell-MP-epitope conjugate of any preceding aspect further comprising: at least one (e.g., at least two) additional polypeptide, each of which is independently selected; and/or at least one (e.g., at least two) covalently attached payload, each of which is independently selected.
179. The T-Cell-MP-epitope conjugate or duplex T-Cell-MP-epitope conjugate of aspect 178, wherein the at least one additional peptide comprises an epitope tag or an affinity domain.
180. The T-Cell-MP-epitope conjugate or duplex T-Cell-MP-epitope conjugate of aspect 178 or 179, wherein the at least one payload comprises a therapeutic agent, antiviral agent, diagnostic agent, or label.
181. The T-Cell-MP-epitope conjugate or duplex T-Cell-MP-epitope conjugate of any of aspects 178-180, wherein the at least one payload comprises a therapeutic agent selected from a nonsteroidal anti-inflammatory drug (e.g., Cox-1 and/or Cox-2 inhibitors such as celecoxib, diclofenac, diflunisal, etodolac, ibuprofen, indomethacin, ketoprofen, or naproxen), or a steroidal anti-inflammatory drug (e.g., cortisone, dexamethasone, hydrocortisone, ethamethasoneb, fludrocortisone, methylprednisolone, prednisone, prednisolone, or triamcinolone).
182. The T-Cell-MP-epitope conjugate or duplex T-Cell-MP-epitope conjugate of any of aspects 178-180, wherein the at least one payload comprises an antiviral agent.
183. The T-Cell-MP-epitope conjugate or duplex T-Cell-MP-epitope conjugate of any of aspects 178-180, wherein the at least one payload comprises a therapeutic agent selected from the group consisting of pyronaridine (pyronaridine-artesunate), quinacrine, mitoxantrone, raloxifene, piceatannol, sunitinib, BNTX and Tilorone.
184. The T-Cell-MP-epitope conjugate or duplex T-Cell-MP-epitope conjugate of any of aspects 178-180, wherein the at least one payload comprises Remdesivir or molnupiravir.
185. The T-Cell-MP-epitope conjugate of any of aspects 178-184, wherein the at least one additional polypeptide comprises a targeting sequence.
186. The duplex T-Cell-MP-epitope conjugate of any of aspects 178-184, wherein (i) the first T-Cell-MP-epitope conjugate comprises at least one independently selected additional polypeptide comprising a targeting sequence, and (ii) the second T-Cell-MP-epitope conjugate comprise at least one independently selected additional polypeptide comprising a targeting sequence.
187. The duplex T-Cell-MP-epitope conjugate of aspect 186, wherein the at least one targeting sequence of the first T-Cell-MP-epitope conjugate and the at least one targeting sequence of the second T-Cell-MP-epitope conjugate are the same or different, and when different the targeting sequences may bind to the same or different targets (e.g., identical antigenic determinates or non-identical antigens or antigenic determinates). See, e.g., FIG. 18.
188. The T-Cell-MP-epitope conjugate or duplex T-Cell-MP-epitope conjugate of any of aspect 185-187, wherein at least one, or each, targeting sequence binds to an independently selected coronavirus protein (e.g., a protein of FIGS. 20A to 20J), or an independently selected coronavirus peptide set forth in Table 2, Table 3, Table 3a, FIG. 21, or FIG. 22).
189. The T-Cell-MP-epitope conjugate or duplex T-Cell-MP-epitope conjugate of aspect 188, wherein at least one, or each, targeting sequence binds to coronavirus spike (surface glycoprotein) protein (e.g., at an ACE receptor sequence or a sequence other than the ACE receptor sequence).
190. The T-Cell-MP-epitope conjugate or duplex T-Cell-MP-epitope conjugate of any of aspect 188-189 wherein the targeting sequences are (i) independently selected antibody sequence (e.g., scFV, nanobody, or diabodies), or (ii) TCR or single chain TCR aa sequences is directed against a coronavirus peptide epitope-MHC complex (coronavirus peptide epitope-HLA complex).
191. The T-Cell-MP-epitope conjugate or duplex T-Cell-MP-epitope conjugate of any of aspect 188-189 wherein the targeting sequence is a TCR or single chain TCR is directed against a coronavirus peptide epitope-MHC complex (coronavirus peptide epitope-HLA complex).
192. The duplex T-Cell-MP-epitope conjugate of any of aspects 186-191, wherein the first T-Cell-MP-epitope conjugate and the second T-Cell-MP-epitope conjugate comprise targeting sequences binding to different targets (e.g., two scFV sequences, two nanobodies, two diabodies, or a bispecific antibody, targeting two different coronavirus epitopes or coronavirus epitope-MHC complexes).
193. The duplex T-Cell-MP-epitope conjugate of aspect 192, wherein the different targets are located on different proteins or on the same protein.
194. The T-Cell-MP-epitope conjugate or duplex T-Cell-MP-epitope conjugate of any of aspects 185-187, wherein at least one (e.g., at least two) or each targeting sequence is an independently selected Cancer Targeting Polypeptide (CTP) (such molecules are forms of redirected T-Cell-MP-epitope conjugates). The CTPs do not bind to a coronavirus epitope, and specifically do not bind to the coronavirus epitope presented by the T-Cell-MP-epitope conjugate or duplex T-Cell-MP-epitope conjugate.
195. The T-Cell-MP-epitope conjugate or duplex T-Cell-MP-epitope conjugate of aspect 194, wherein the least one (e.g., at least two) or each CTP is a T cell receptor (TCR), or a TCR polypeptide sequence (e.g., a TCR beta chain or a TCR single-chain T-cell receptor (scTCR)).
196. The T-Cell-MP-epitope conjugate or duplex T-Cell-MP-epitope conjugate of aspect 195, wherein the least one (e.g., at least two) or each CTP comprises an antibody aa sequence (e.g., a sequence of one or more antibody complementarity determining regions).
197. The T-Cell-MP-epitope conjugate or duplex T-Cell-MP-epitope conjugate of aspect 196, wherein each antibody is a chimeric antibody, humanized antibody, diabody, bi-specific antibody, or multi-specific antibody.
198. The T-Cell-MP-epitope conjugate or duplex T-Cell-MP-epitope conjugate of aspect 196, wherein each antibody is an antibody fragment selected from an Fab, F(ab′)₂, Fv, scFv, or Fd fragment.
199. The T-Cell-MP-epitope conjugate or duplex T-Cell-MP-epitope conjugate of aspect 196, wherein each antibody is selected from single-chain antibodies, single chain camelid (e.g., llama) antibodies, single domain antibodies (dAb), single domain heavy chain antibodies, single domain light chain antibodies, nanobodies, and fusion proteins comprising an antigen-binding (also referred to herein as antigen binding) portion of an antibody and a non-antibody protein.
200. The T-Cell-MP-epitope conjugate or duplex T-Cell-MP-epitope conjugate of aspect 196, wherein each antibody is a nanobody or scFv.
201. The T-Cell-MP-epitope conjugate or duplex T-Cell-MP-epitope conjugate of any of aspects 194-200, wherein each CTP binds to an antigen present on the surface of a neoplasm (e.g., benign, precancerous, or malignant neoplasms).
202. The T-Cell-MP-epitope conjugate or duplex T-Cell-MP-epitope conjugate of any of aspects 194-201, where each CTP is directed against (e.g., binds to) an independently selected cancer associated antigen or antigenic determinant (e.g., binds to different antigenic determinates on the same or different antigens), and may be the same or different.
203. The T-Cell-MP-epitope conjugate or duplex T-Cell-MP-epitope conjugate of aspect 202, wherein each cancer associated antigen is selected independently from the group consisting of: 17-1A-antigen, alpha-fetoprotein (AFP), alpha-actinin-4, A3, antigen specific for A33 antibody, ART-4, B7, Ba 733, BAGE, bcl-2, bcl-6, BCMA, BrE3-antigen, CA125, CAMEL, CAP-1, carbonic anhydrase IX (CAIX), CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CEA, CEACAM5, CEACAM6, a claudin, a complement factor, colon-specific antigen-p (CSAp), c-Met, CTLA-4, CXCR4, CXCR7, CXCL12, DAM, Dickkopf-related protein (DKK), ED-B fibronectin, epidermal growth factor receptor (EGFR), EGFRvIII, EGP-1 (TROP-2), EGP-2, ELF2-M, Ep-CAM, EphA2, EphA3, fibroblast activation protein (FAP), fibroblast growth factor (FGF), Flt-1, Flt-3, folate binding protein, folate receptor, G250 antigen, gangliosides (such as GC2, GD3 and GM2), GAGE, GD2, gp100, GPC3, GRO-13, HLA-DR, HM1.24, human chorionic gonadotropin (HCG) and its subunits, HER2, HER3, HMGB-1, hypoxia inducible factor (HIF-1), HIF-1a, HSP70-2M, HST-2, la, IFN-gamma, IFN-alpha, IFN-beta, IFN-X, IL-4R, IL-6R, IL-13R, IL 13Ralpha2, IL-15R, IL-17R, IL-18R, IL-2, IL-6, IL-8, IL-12, IL-15, IL-17, IL-18, IL-23, IL-25, ILGF, ILGF-1R, insulin-like growth factor-1 (IGF-1), IGF-1R, integrin αVβ3, integrin α5β1, KC4-antigen, killer-cell immunoglobulin-like receptor (KIR), Kras, KS-1-antigen, KS1-4, LDR/FUT, Le^{gamma}, macrophage migration inhibitory factor (MIF), a MAGE (e.g., MAGE A1, MAGE A2, MAGE A2B, MAGE A3, MAGE A4, MAGE A6, MAGE A8, MAGE A9, MAGE A9B, MAGE A10, MAGE A11, MAGE A12, MAGE C1, MAGE C2 in Human Genome Nomenclature Committee or “HGNC” abbreviations), MART-1, MART-2, mCRP, MCP-1, melanoma glycoprotein, mesothelin, MIP-1A, MIP-1B, MIF, mucins (e.g., MUC1, MUC2, MUC3, MUC4, MUC5ac, MUC13, MUC16, MUM-1/2 and MUM-3), NCA66, NCA95, NCA90, Nectin-4, NY-ESO-1, NY-ESO-2 (also known as Cancer/testis antigen 2 or L antigen family member 1), PAM4 antigen, pancreatic cancer mucin, PD-1, PD-L1, PD-1 receptor, placental growth factor, p53, PLAGL2, prostatic acid phosphatase, PSA, PRAME, PSMA, P1GF, RSS, RANTES, SAGE, 5100, survivin, survivin-2B, T101, TAC, TAG-72, tenascin, Thomson-Friedenreich antigens, Tn antigen, TNF-alpha, tumor necrosis antigens, TRAG-3, TRAIL receptors, vascular endothelial growth factor (VEGF), VEGF receptor (VEGFR) and WT-1.
204. The T-Cell-MP-epitope conjugate or duplex T-Cell-MP-epitope conjugate of aspect 202, wherein each cancer associated antigen is selected independently from the group consisting of: NY-ESO (New York Esophageal Squamous Cell Carcinoma 1), melanoma antigen recognized by T cells or “MART”, HPV (human papilloma virus) E6, BCMA (B-cell maturation antigen), CD123, CD133, CD171, CD19, CD20, CD22, CD30, CD33, CD38, CD138, CEA (carcinoembryonic antigen), EGFR (epidermal growth factor receptor), EGFRvIII (epidermal growth factor receptor variant III), EpCAM (epithelial cell adhesion molecule), EphA2 (ephrin type-A receptor 2), disialoganglioside GD2, GPC3 (glypican-3), HER2, IL 13Ralpha2 (Interleukin 13 receptor subunit alpha-2), LeY (a difucosylated type 2 blood group-related antigen), a melanoma-associated antigen(s) or “MAGE” (e.g., MAGE A3 or MAGE A4), melanoma glycoprotein, mesothelin, a mucin (e.g., mucin 1 or “MUC1”), MUC16 (mucin-16), myelin, NKG2D (Natural Killer Group 2D) ligands, PSMA (prostate specific membrane antigen), and ROR1 (type I receptor tyrosine kinase-like orphan receptor)
205. The T-Cell-MP-epitope conjugate or duplex T-Cell-MP-epitope conjugate of aspect 202, wherein each cancer associated antigen is selected independently from the group consisting of: CAIX, cadherins, CEA, c-MET, CTLA-4, EGFR family members, EpCAM, EphA3, FAP, folate-binding protein, FR-alpha, gangliosides (such as GC2, GD3 and GM2), HER2, HER3, IGF-1R, integrin αVβ3, integrin α5β1, Le^{gamma}, Liv1, mesothelin, mucins, NaPi2b, PD-1, PD-L1, PD-1 receptor, pgA33, PSMA, RANKL, ROR1, TAG-72, tenascin, TRAILR1, TRAILR2, VEGF, VEGFR
206. The T-Cell-MP-epitope conjugate or duplex T-Cell-MP-epitope conjugate of aspect 202, wherein each cancer associated antigen is selected independently from the group consisting of: MAGE A1, MAGE A2, MAGE A2B, MAGE A3, MAGE A4, MAGE A6, MAGE A8, MAGE A9, MAGE A9B, MAGE A10, MAGE A11, MAGE A12, MAGE C1, MAGE C2.
207. The T-Cell-MP-epitope conjugate or duplex T-Cell-MP-epitope conjugate of aspect 202, wherein each cancer associated antigen is selected independently from MAGE A3 and MAGE A4.
208. The T-Cell-MP-epitope conjugate or duplex T-Cell-MP-epitope conjugate of aspect 202, wherein each cancer associated antigen is an NY-ESO-1 antigen.
209. The T-Cell-MP-epitope conjugate or duplex T-Cell-MP-epitope conjugate of aspect 195, wherein the T cell receptor (TCR), or a TCR polypeptide sequence binds to an epitope of a cancer associated antigen (e.g., complexed with an MHC).
210. The T-Cell-MP-epitope conjugate or duplex T-Cell-MP-epitope conjugate of aspect 209, wherein the cancer associated antigen is selected from the group consisting of: MUC1, LMP2, epidermal growth factor receptor (EGFR) vIII, HER-2/neu, a melanoma associated antigen (“MAGE”), MAGE A1, MAGE A3, MAGE A4, p53, a mutant p53, NY-ESO-1, folate hydrolase (prostate-specific membrane antigen; PSMA), carcinoembryonic antigen (CEA), a claudin (e.g., claudin-1, claudin-10, claudin-18 (e.g., claudin-18, isoform 2), Nectin-4, melanoma antigen recognized by T-cells (melanA/MART1), Ras, gp100, proteinase3 (PR1), bcr-abl, tyrosinase, survivin, prostate specific antigen (PSA), hTERT, a sarcoma translocation breakpoint polypeptide, a synovial sarcoma X (SSX) breakpoint polypeptide, EphA2, an acid phosphatase, prostate (PAP), melanoma inhibitor of apoptosis (ML-IAP), an epithelial cell adhesion molecule (EpCAM), an ERG (TMPRSS2 ETS fusion), NA17, a paired-box-3 (PAX3) polypeptide, an anaplastic lymphoma kinase (ALK), an androgen receptor, cyclin B1, an N-myc proto-oncogene (MYCN), a Ras homolog gene family member C (RhoC), a tyrosinase-related protein-2 (TRP-2), a mesothelin, a prostate stem cell antigen (PSCA), cytochrome P450 1B1 (CYP1B1), placenta-specific protein 1 (PLAC1), BORIS, an ETV6-AML, a breast cancer antigen, NY-BR-1, a regulator of G-protein signaling (RGS5), squamous cell carcinoma antigen recognized by T-cells (SART3), carbonic anhydrase IX, paired box-5 (PAX5), OY-TES1, sperm protein 17, lymphocyte cell-specific protein-tyrosine kinase (LCK), high molecular weight melanoma associated antigen (HMW-MAA), A-kinase anchoring protein-4 (AKAP-4), synovial sarcoma X breakpoint 2 (SSX2), aX antigen family member 1 (XAGE1), B7 homolog 3, a legumain (e.g., LGMN1), a tyrosine kinase with Ig and EGF homology domains-2 (Tie-2; also known as angiopoietin-1 receptor), a P antigen family member 4 (PAGE4), vascular endothelial growth factor receptor 2 (VEGF2), MAD-CT-1, fibroblast activation protein (FAP), PDGFB, a MAD-CT-2, Fos-related antigen-1; a human papilloma virus (“HPV”) antigen; alpha-feto protein (“AFP”) antigen; and Wilms tumor-1 (“WT1”) antigen.
211. The T-Cell-MP-epitope conjugate or duplex T-Cell-MP-epitope conjugate of any of aspects 194-210, wherein the targeting sequence (e.g., CTP) is fused to (part of) the T-Cell-MP-epitope conjugate, or in the case of a duplex T-Cell-MP-epitope conjugate is fused to (part of) at least one (e.g., both the first and second) T-Cell-MP-epitope conjugate polypeptide ((e.g., translated as part of the T-Cell-MP-epitope conjugate sequence(s)).
212. The T-Cell-MP-epitope conjugate or duplex T-Cell-MP-epitope conjugate of any of aspects 194-210, wherein the targeting sequence (e.g., CTP) is covalently attached (e.g., by a bifunctional crosslinker) or non-covalently attached to a T-Cell-MP-epitope conjugate or duplex T-Cell-MP-epitope conjugate.
213. A method of treatment or prophylaxis of a coronavirus infection or condition associated with coronavirus infection comprising:
   (i) administering to a patient or subject (e.g., a patient in need thereof) an effective amount of one or more T-Cell-MP-epitope conjugates or one or more duplex T-Cell-MP-epitope conjugates or other higher order complexes of T-Cell-MP-epitope conjugates of any of aspects 1-193; or
   (ii) contacting a cell or tissue in vitro or in vivo with one or more T-Cell-MP-epitope conjugates or one or more duplex T-Cell-MP-epitope conjugates or other higher order complexes of T-Cell-MP-epitope conjugates according to any of aspects 1-193 and administering the cell, tissue, or progeny thereof to a patient or subject (e.g., a patient in need thereof);
   wherein the coronavirus infection or condition associated with coronavirus infection includes, but not limited to, primary coronavirus infections, secondary coronavirus infections, and long COVID.
214. A method of controlling a coronavirus infection or of reducing transmission of coronavirus comprising (i) administering to a patient or subject (e.g., a patient in need thereof) an effective amount of one or more T-Cell-MP-epitope conjugates or one or more duplex T-Cell-MP-epitope conjugates or other higher order complexes of T-Cell-MP-epitope conjugates of T-Cell-MP-epitope conjugates of any of aspects 1-193; or (ii) contacting a cell or tissue in vitro or in vivo with one or more T-Cell-MP-epitope conjugates or one or more duplex T-Cell-MP-epitope conjugates or other higher order complexes of T-Cell-MP-epitope conjugates of any of aspects 1-193, and administering the cell, tissue, or progeny thereof to a patient or subject (e.g., a patient in need thereof).
215. The method of any of aspects 213-214, wherein the T-Cell-MP-epitope conjugate or duplex T-Cell-MP-epitope conjugate comprises at least one targeting sequence.
216. The method of aspect 215, wherein the at least one targeting sequence is directed to a cell or tissue antigen (e.g., a lung tissue antigen).
217. The method of aspect 215 wherein the at least one targeting sequence is an antibody (monoclonal antibody, scFv, or nanobody) specific for a coronavirus protein (e.g., a protein of any of FIGS. 20A to 20J) or single chain TCR directed to (specific for) a coronavirus peptide epitope-HLA complex.
218. The method of aspect 217 wherein the at least one targeting sequence comprises an antibody (e.g., a scFv) or single chain TCR sequenced that is fused to a T-Cell-MP-epitope conjugate, or is attached (covalently or non-covalently) a T-Cell-MP-epitope conjugate (e.g., by a crosslinking agent).
219. The method of any of aspects 215-218, wherein when the one or more T-Cell-MP-epitope conjugates, duplex T-Cell-MP-epitope conjugates, or other higher order complexes of T-Cell-MP-epitope conjugates comprises a MOD that stimulates CD8+ T cells, the administering may increase the number of CD8+ T cells and/or activity of CD8+ effector T cells specific for the coronavirus epitope presented by the one or more T-Cell-MP-epitope conjugates, duplex T-Cell-MP-epitope conjugates, or other higher order complexes of T-Cell-MP-epitope conjugates.
220. The method of any of aspects 215-218, wherein when the one or more T-Cell-MP-epitope conjugates, duplex T-Cell-MP-epitope conjugates or other higher order complexes of T-Cell-MP-epitope conjugates comprises a peptide presenting an HLA-E restricted coronavirus epitope (e.g., VMPLSAPTL, SEQ ID NO:2058) or a peptide presenting HLA-E restricted non-coronavirus epitope (e.g., epitopes that bind and activate signaling through CD94/NKG2C receptors of NK cells and may increase NK cell activity). Alternatively, HLA-E restricted epitopes that have affinity for CD94/NKG2A receptors of NK cells may suppress NK cell activity).
221. The method of any of aspects 213-220, wherein the one or more T-Cell-MP-epitope conjugates or duplex T-Cell-MP-epitope conjugates comprise an IL-2 or variant IL-2.
222. The method of aspect 221, wherein the one or more T-Cell-MP-epitope conjugates or duplex T-Cell-MP-epitope conjugates comprise a variant IL-2 having substitutions at H16 and/or F42 (e.g., H16A or H16T and either F42A or F42T).
223. The method of any of aspects 213-222, wherein administering to the patient or subject functions prophylactically (prevents infection or symptomatic infection e.g., requiring hospitalization) relative to a control population.
224. The method of any of aspects 213-223, wherein administering to the patient or subject: functions to control a coronavirus infection (e.g., reduce disease severity, morbidity, mortality, or the amount of time until the patient/subject is free of detectable virus) relative a control population; or functions to reduce one or more symptoms associated with long COVID relative to a population having but untreated for long COVID.
225. The method of any of aspects 213-223, wherein administering to the patient/subject functions to reduce viral load, shedding, and/or transmission of coronaviruses by infected individuals relative to a control population (e.g., of infected individuals).
226. The method of any of aspects 213-223, wherein administering to the patient/subject functions to increase the number of T cells (e.g., cytotoxic T cells) specific for the epitope conjugated to the T-cell-MP-epitope conjugate or duplex T-cell-MP-epitope conjugate thereof relative to the level prior to administration or relative a control population.
227. The method of any of aspects 223-226, wherein the control population is a population that did not receive the T-cell-MP-epitope conjugate, duplex T-Cell-MP-epitope conjugate or higher order complex of any thereof; and wherein the control population may be matched to the patient(s) or subject(s) by:
   (i) age, sex, immune disease, and country of residence;
   (ii) age, weight, sex, immune disease, country of residence, and smoking status; or
   (iii) age, weight, sex, immune disease, country of residence, cancer, cerebrovascular disease. Kidney disease, Chronic Obstructive Pulmonary Disease, (COPD), diabetes, coronary disease (heart failure, coronary artery disease, or cardiomyopathies), smoking, pregnancy, and/or asthma.
228. The method of any of aspects 213-227, further comprising administering one or more additional therapeutic agents (e.g., antiviral and/or immunomodulatory agents).
229. The method of aspect 228, wherein the one or more therapeutic agents are selected from the group consisting of: nucleotide analogs, nucleoside analogs, chloroquine or hydroxychloroquine, anti-HIV agents, interferon beta (e.g., interferon beta 1a), protease inhibitors (e.g., cinanserin, flavonoids, diarylheptanoids), type I interferons (IFNs), recombinant IFN (e.g., rIFNβ or rIFNγ), inhibitors of IL-6 receptors (e.g., tocilizumab), inhibitors of IL-6 (e.g., anti-IL-6 antibodies such as lenzilumab), NSAIDs, and glucocorticoids.
230. The method of aspect 229, wherein the nucleotide or nucleoside analogs are selected from the group consisting of: remdesivir, molnupiravir, favipiravir, ribavirin and galidesivir.
231. The method of aspect 229, wherein the anti-HIV agents are lopinavir or ritonavir.
232. The method of any of aspects 213-231, further comprising administering one or more NSAIDs.
233. The method of aspect 232, wherein the one or more NSAIDs are Cox-1 and/or Cox-2 inhibitors selected from the group consisting of celecoxib, diclofenac, diflunisal, etodolac, ibuprofen, indomethacin, ketoprofen, and naproxen.
234. The method of any of aspects 213-233, further comprising administering one or more corticosteroids.
235. The method of aspect 234, wherein the one or more corticosteroids are selected from the group consisting of cortisone, dexamethasone, hydrocortisone, ethamethasoneb, fludrocortisone, methylprednisolone, prednisone, prednisolone and triamcinolone.
236. The method of any of aspects 213-235, further comprising administering one or more inhibitors of viral entry.
237. The method of aspect 236, wherein the inhibitors of viral entry are selected from Tilorone, Mitoxantrone, Raloxifene, and Piceatannol, Sunitinib, and BNTX.
238. The method of any of aspects 213-237, further comprising administering one or more agents selected from the group consisting of: pyronaridine, pyronaridine-artesunate, quinacrine.
239. The method of any of aspects 213-238, further comprising administering one or more antibodies directed against SARS-CoV-2 spike protein selected from the group consisting of bamlanivimab, etesevimab, casirivimab, imdevimab.
240. The method of aspect 239, wherein the one or more antibodies comprise a combination of bamlanivimab and etesevimab, or a combination of casirivimab plus imdevimab.
241. A method of providing treatment or prophylaxis to a patient or subject (e.g., a patient or subject in need thereof) having, or suspected of having, or developing (e.g., in association with genetic propensity or family history) a neoplasm comprising:
   (i) administering to a patient or subject an effective amount of one or more T-Cell-MP-epitope conjugates, one or more duplex T-Cell-MP-epitope conjugates, or other higher order complexes of T-Cell-MP-epitope conjugates of any of aspects 185-187 or 194-212; or
   (ii) contacting a cell or tissue (e.g., a cell or tissue of the patient or subject) in vitro or in vivo with one or more T-Cell-MP-epitope conjugates or duplex T-Cell-MP-epitope conjugates of any of aspects 185-187 or 194-212 and administering the cell, tissue, or progeny thereof to a patient/subject (e.g., a patient in need thereof).
242. The method of aspect 241, wherein the neoplasm is a benign or precancerous neoplasm.
243. The method of aspect 241, wherein the neoplasm is a malignant neoplasm (cancerous neoplasm).
244. The method of any of aspects 241-243, optionally comprising administering (prior to or concurrent with the any of the one or more T-Cell-MP-epitope conjugates, one or more duplex T-Cell-MP-epitope conjugates, or other higher order complexes of T-Cell-MP-epitope conjugates) one or more doses of an antigen presenting the coronavirus epitope (e.g., a protein, polypeptide, peptide, or other antigen such as a phosphopeptide or glycopeptide presenting the coronavirus epitope that is covalent bound to a chemical conjugation site in the T-Cell-MP-epitope conjugate and presented to T cells).
245. The method of aspect 244, wherein administering the protein, polypeptide, peptide or other antigen increases the number of CD8+ T-Cells (e.g., the number of circulating CD8+ T-cells/ml of blood) specific of the coronavirus epitope covalently bound to the one or more T-Cell-MP-epitope conjugates, one or more duplex T-Cell-MP-epitope conjugates, or other higher order complexes of T-Cell-MP-epitope conjugates.
246. The method of any of aspects 241-245, wherein the coronavirus epitope is an epitope of a Bat-SL-CoV, SARS-CoV, SARS-CoV-2 (Covid-19), or MERS-CoV.
247. The method of any of aspects 241-245, wherein the coronavirus epitope is an epitope of a SARS-CoV or SARS-CoV-2 (Covid-19).
248. The method of any of aspects 241-245, wherein the coronavirus epitope is an epitope of a SARS-CoV-2 (Covid-19) (e.g., the delta or omicron strain).
249. The method of any of aspects 241-248, wherein the coronavirus epitope is an epitope of a surface glycoprotein (spike protein see FIG. 20J), a nucleocapsid protein (see FIG. 20F), or membrane glycoprotein (See FIG. 20D).
250. The method of any of aspects 241-249, wherein the coronavirus epitope is an epitope of a surface glycoprotein (spike protein see FIG. 20J).
251. The method of any of aspects 241-250, wherein the one or more T-Cell-MP-epitope conjugates or one or more duplex T-Cell-MP-epitope conjugates or other higher order complexes of T-Cell-MP-epitope conjugates comprise an independently selected targeting sequence (e.g., a CTP) as part of at least one (e.g., each) T-Cell-MP-epitope conjugate polypeptide (e.g., fused to at least one or both of the first T-Cell-MP-epitope conjugate or the second T-Cell-MP-epitope conjugate of a duplex T-Cell-MP-epitope conjugate).
252. The method of any of aspects 241-250, wherein the one or more T-Cell-MP-epitope conjugates or one or more duplex T-Cell-MP-epitope conjugates or other higher order complexes of T-Cell-MP-epitope conjugates comprise a targeting sequence (e.g., a CTP) covalently or non-covalently attached to at least one or each T-Cell-MP-epitope conjugate (e.g., attached by a bifunctional crosslinker to at least one T-Cell-MP-epitope conjugate of a duplex T-Cell-MP-epitope conjugate).
253. The method of any of aspects 251-252, wherein the MHC-H polypeptide sequence of the one or more T-Cell-MP-epitope conjugates, one or more duplex T-Cell-MP-epitope conjugates, or other higher order complexes of T-Cell-MP-epitope conjugates comprise is an HLA-E polypeptide sequence.
254. The method of aspects 253, wherein the a peptide presenting a coronavirus epitope comprises the sequence VMPLSAPTL (SEQ ID NO:2058.
255. The method of aspects 253, wherein the one or more T-Cell-MP-epitope conjugates or one or more duplex T-Cell-MP-epitope conjugates or other higher order complexes of T-Cell-MP-epitope conjugates are conjugate to a non-coronavirus HLA-E restricted epitope. See e.g., aspects 35-41.
256. The method of aspects 255, wherein the HLA-E restricted epitope comprises an aa sequence selected from the group consisting of: QMPSRSLLF (SEQ ID NO:2059), TLPKRGLFL (SEQ ID NO:2060), TGPWRSLWI (SEQ ID NO: 2061), ILTDRSLWL (SEQ ID NO:2062), VNPGRSLFL (SEQ ID NO:2063), WNRLFPPLR (SEQ ID NO: 2064), TLPERTLYL (SEQ ID NO:2065), FLPNRSLLF (SEQ ID NO:2066), VMGDRSVLY (SEQ ID NO: 2067), and VMADKSIFY (SEQ ID NO:2068).
257. The method of aspects 255, wherein the HLA-E restricted epitope comprises an aa sequence selected from the group consisting of: VMPPRTLLL (SEQ ID NO.: 2069), VMAPRTLFL (SEQ ID NO:2070), VNPGRSLFL (SEQ ID NO: 2063), QMPSRSLLF (SEQ ID NO.: 2071), TLPERTLYL (SEQ ID NO:2065), VMPGRTLCF (SEQ ID NO: 2072), ILTDRSLWL (SEQ ID NO:262), RMPPRSVLL (SEQ ID NO:2073), TAPARTMFL (SEQ ID NO.: 2074), and NMPARTVLF SEQ ID NO:2075.
258. The method of aspects 255, wherein the epitope comprises the aa sequence WNRILPNAY SEQ ID NO:2076.
259. The method of aspects 255, wherein the HLA-E restricted epitope comprises the aa sequence X1-X2-X3-X4-R-X6-X7-X8-X9-X10, where: X1 is absent or S, K, A, I, L, or T; X2 is L, I, K or V; X3 is P, A or S; X4 is G, V, P, D or R; X6 is S or T; X7 is L, I, or V; X8 is F, W, or E; X9 is L. F, W or E; and X10 F, H, S, A or absent SEQ ID NO: 2077.
260. The method of aspects 255, wherein the HLA-E restricted epitope comprises the aa sequence LP (G or V) RSLFL SEQ ID NO:2078, or LP (G or V) RSLWL (SEQ ID NO:2079).
261. The method of aspects 255, wherein the epitope comprises a CMV aa sequence selected from the group consisting of: VLPHRTQFL (SEQ ID NO:2080), TGAARSFFF (SEQ ID NO:2081), and VMAPRTLIL (SEQ ID NO: 2082).
262. The method of aspects 255, wherein the HLA-E restricted epitope comprises a CMV aa sequence selected from the group consisting of: SAPLKTRFL (SEQ ID NO:2083), and SVPLKTRFL (SEQ ID NO:2084).
263. The method of any of aspects 241-2262, wherein the one or more independently selected MOD polypeptide sequences comprise an IL-2 MOD polypeptide sequence and/or a variant IL-2 MOD polypeptide sequence.
264. The method of aspect 263, wherein the one or more independently selected MOD polypeptide sequences comprises a variant MOD polypeptide sequence.
265. The method of aspect 264, wherein the one or more independently selected MOD polypeptide sequences comprises an H16 substitution and/or an F42 substitution (e.g., H16A and F42A).
266. The method of any of aspects 213-265, further comprising administering one or more therapeutic agents that enhance CD8+ T cell functions (e.g., effector function such as granule-dependent and granule-independent cytotoxic response, differentiation, and/or proliferation), one or more immunomodulatory agents, and/or one or more additional therapeutic agents (e.g., chemotherapeutic agents or immune checkpoint inhibitors).
267. The method of aspect 266, wherein the one or more therapeutic agents are selected from the group consisting of: type I interferons (IFNs), recombinant IFN (e.g., rIFNβ or rIFNγ).
268. The use of one or more T-Cell-MP-epitope conjugates, one or more duplex T-Cell-MP-epitope conjugates, or one or more higher order complexes of a T-Cell-MP-epitope conjugate according to any of aspects 1-212, for the preparation of a medicament for the treatment of a coronavirus infection, a neoplasm, or ablation of a tissue or cell type.
269. The use according to aspect 268, for the treatment of a malignant neoplasm.
270. One or more T-Cell-MP-epitope conjugates, one or more duplex T-Cell-MP-epitope conjugates, or one or more higher order complexes of a T-Cell-MP-epitope conjugate according to any of aspects 1-212, for use in treating a coronavirus infection or long COVID.
271. One or more T-Cell-MP-epitope conjugates, one or more duplex T-Cell-MP-epitope conjugates, or one or more higher order complexes of a T-Cell-MP-epitope conjugate according to any of aspects 1-212, for use in treating a neoplasm or for the ablation of a tissue or cell type.
272. The one or more T-Cell-MP-epitope conjugates, one or more duplex T-Cell-MP-epitope conjugates, or one or more higher order complexes of a T-Cell-MP-epitope conjugates of aspect 271, wherein the neoplasm is benign or precancerous.
273. The one or more T-Cell-MP-epitope conjugates, one or more duplex T-Cell-MP-epitope conjugates, or one or more higher order complexes of a T-Cell-MP-epitope conjugates of aspect 271, wherein the neoplasm is malignant.
274. A method of selectively delivering one or more (e.g., two or more) wt. MOD polypeptides and/or one or more (e.g., two or more) variant MOD polypeptides to one or more coronavirus epitope-specific CD8+ T cells of a patient or subject, the method comprising contacting the one or more cells or tissues of the patient or subject, or administering to the patient or subject (e.g., a patient in need thereof) an effective amount of: (i) one or more T-Cell-MP-epitope conjugates or higher complexes thereof (e.g., duplexes) of any of aspects 1-212.
275. The method of aspect 274, wherein the one or more wt. MOD polypeptides and/or variant MOD polypeptides are selected independently from the group consisting of: 4-1BBL, PD-L1, IL-2, CD80, CD86, OX40L (CD252), Fas ligand (FasL), ICOS-L, ICAM, CD30L, CD40, CD83, HVEM (CD270), JAG1 (CD339), CD70, TGF-β1, TGF-β2, and TGF-β3 wt. MOD or variant MOD polypeptide sequences of any thereof.
276. The method of aspect 274 or 275, wherein the one or more wt. MOD polypeptides and/or variant MOD polypeptides are selected independently from the group consisting of: 4-1BBL, PD-L1 IL-2, CD80, CD86, and FasL wt. MOD and variant MOD polypeptide sequences of any thereof.
277. The method of any of aspects 274-276, wherein the T-Cell-MP or duplex T-Cell-MP comprises at least one IL-2 wt. MOD or at least one IL-2 variant MOD polypeptide sequence, and at least one CD80, CD86, variant CD80 or variant CD86 polypeptide sequence.
278. The method of any of aspects 274-277, wherein the T-Cell-MP or duplex T-Cell-MP comprises at least one IL-2 wt. MOD or variant IL-2 MOD polypeptide sequence, or at least one pair of IL-2 wt. MOD or at least one pair of IL-2 variant MOD polypeptide sequences in tandem.
279. The method or use of any of aspects 274-277, wherein the T-Cell-MP or duplex T-Cell-MP comprises at least one wt. MOD or variant CD80 and/or CD86 MOD polypeptide sequence.
280. The method of any of aspects 274-277, wherein the T-Cell-MP or duplex T-Cell-MP comprises at least one PD-L1 wt. MOD or variant MOD polypeptide sequence.
281. The method of any of aspects 274-277, wherein the T-Cell-MP or duplex T-Cell-MP comprises at least one FasL wt. MOD or variant MOD polypeptide sequence.
282. The method or use of any of aspects 213-281, wherein the patient or subject is human.
283. The method of any of aspects 213-281, wherein the patient or subject is non-human mammal (e.g., rodent, lagomorph, bovine, canine, feline, rodent, murine, caprine, simian, ovine, equine, lapine, porcine, etc.).
284. The method or use of any of aspects 213-283, wherein the administering is conducted intramuscularly, intralymphatically, intratracheally, intracranially, subcutaneously, intradermally, topically, intravenously, intraarterially, rectally, nasally, orally, or by other enteral or parenteral routes of administration.
285. The method or use of any of aspects 213-284, wherein effective amount of one or more T-Cell-MP-epitope conjugates or one or more duplex T-Cell-MP-epitope conjugates or other higher order complexes of T-Cell-MP-epitope conjugates administered to the patient or subject is from about 1 ng/kg body weight and about 20 mg/kg body weight per dose or from about 1 mg/kg body weight to about 50 mg/kg body weight per dose.
286. The method or use of aspect 285, wherein the effective amount is from about 0.1 mg/kg body weight to 25 mg/kg body weight per dose, or about 0.1 mg/kg body weight to about 10 mg/kg body weight dose.
287. The method or use of any of aspects 213-286, wherein multiple doses of one or more T-Cell-MP-epitope conjugates or one or more duplex T-Cell-MP-epitope conjugates or other higher order complexes of T-Cell-MP-epitope conjugates are administered to a patient or subject.
288. The method or use of aspect 287, wherein doses are administered once every 2-6 months, or once per month.
289. The method or use of aspect 287, wherein doses are administered once every three weeks, or more frequently than once every three weeks.
290. The method or use of aspect 287, wherein doses are administered once a week, once every two weeks, once every three weeks, once every four weeks, once per month.
291. The method or use of aspect 287, wherein doses are administered once per week, e.g., twice per week (biw), three times per week (tiw), four times per week, five times per week, six times per week, every other day (qod), or daily (qd).
292. The method or use of any of aspects 213-291, wherein the duration of administration is from about one month to about two months, from about two months to about four months, or from about four months to about six months.
293. The method or use of any of aspects 213-291, wherein the duration of administration is from about six months to about eight months, or from about eight months to about 1 year.
294. The method or use of any of aspects 213-291, wherein the duration of administration is from about 1 year to about 2 years, or from about 2 years to about 4 years.
295. A method of inducing or causing the proliferation of memory T cells specific to a coronavirus epitope, the method comprising: administering to a patient or subject an effective amount of: (i) one or more T-Cell-MP-epitope conjugates, duplex T-Cell-MP-epitope conjugates, or other higher complexes thereof of any of aspects 1-34 or 42-212, wherein the epitope is a coronavirus epitope set forth in Table 3a.

### XV. EXAMPLES

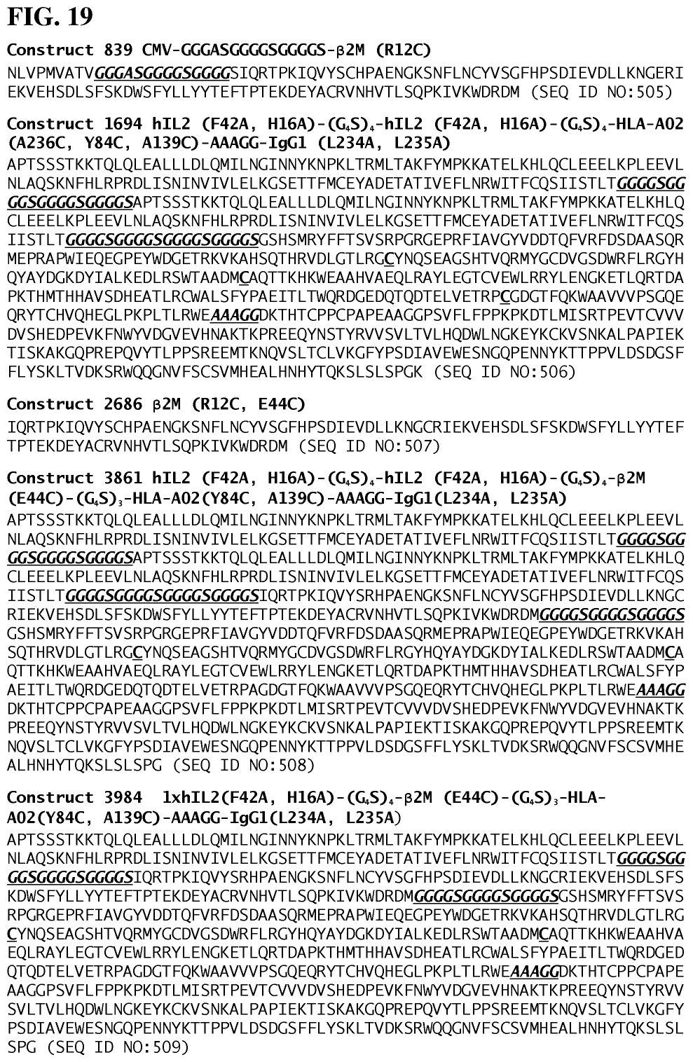

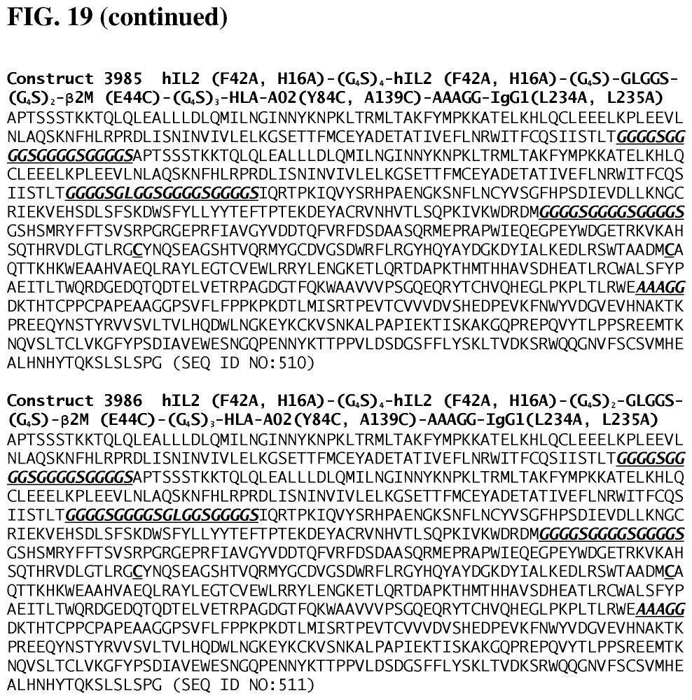

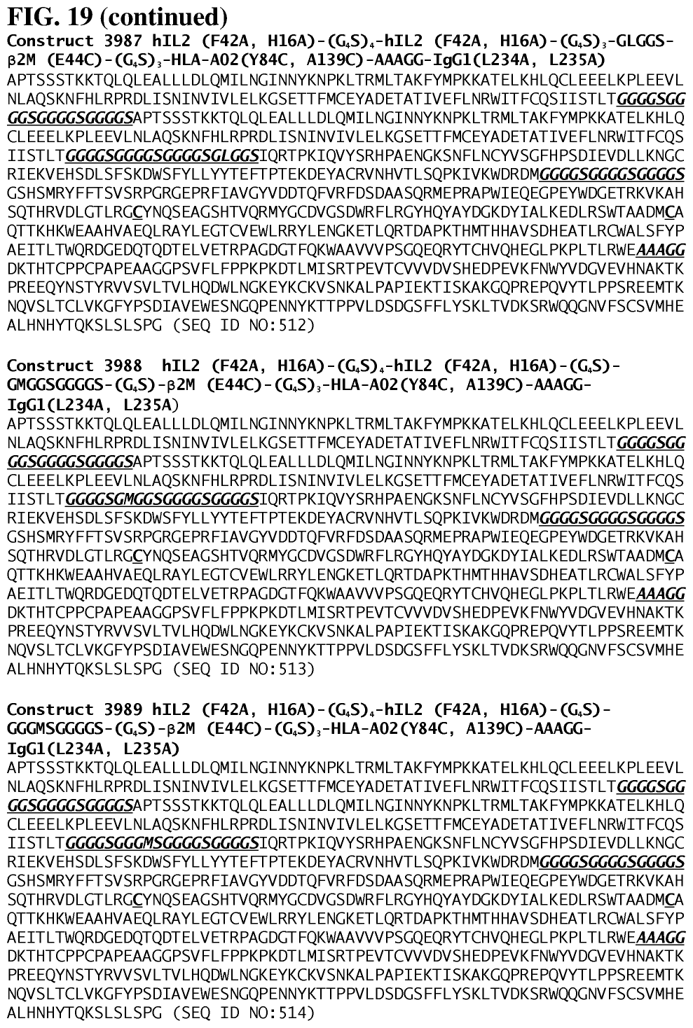

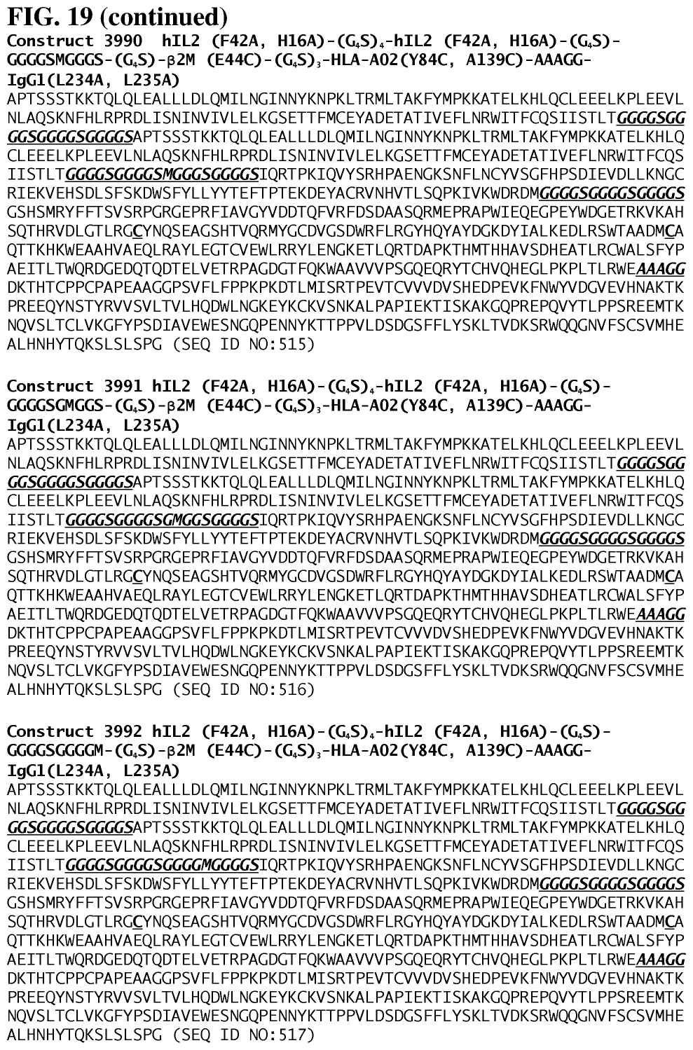

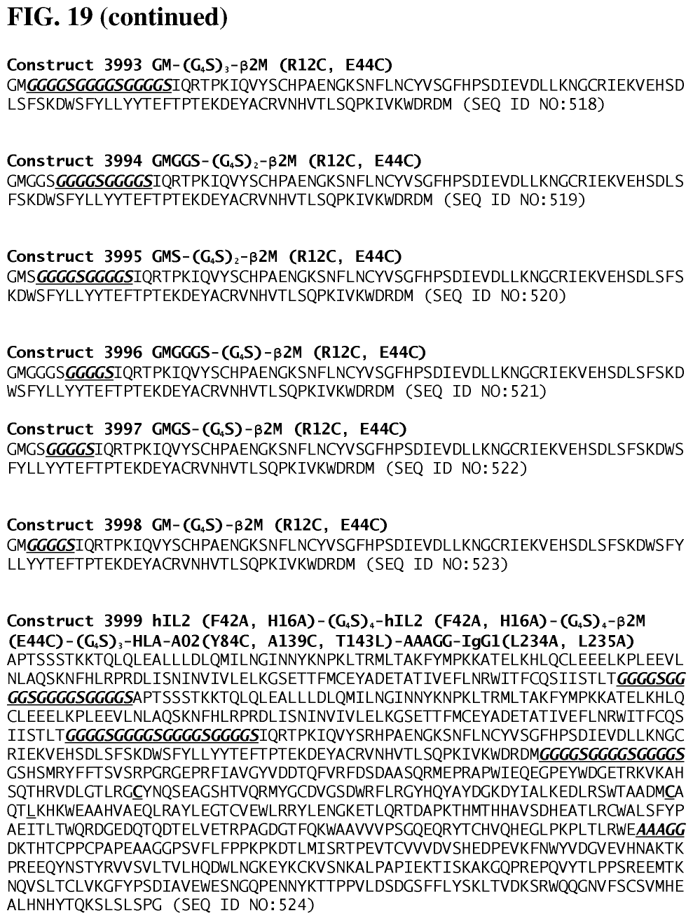

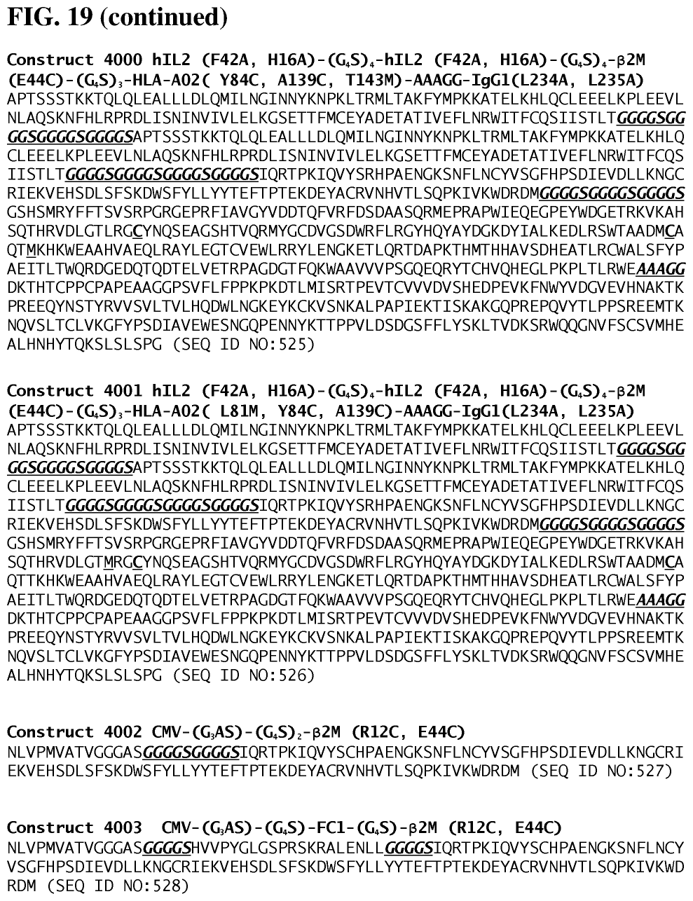

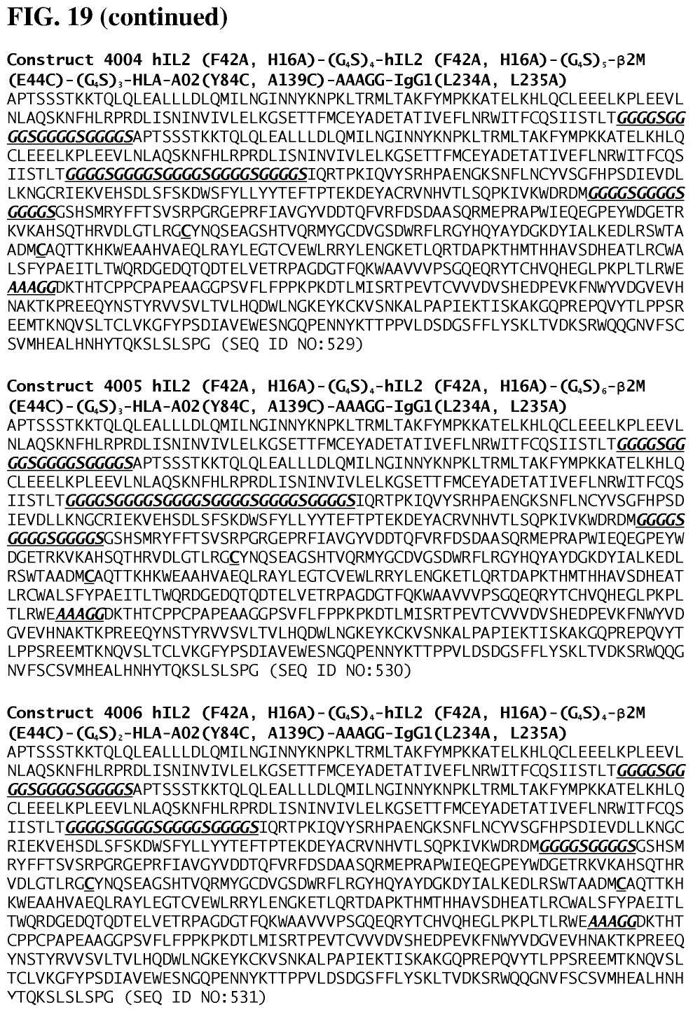

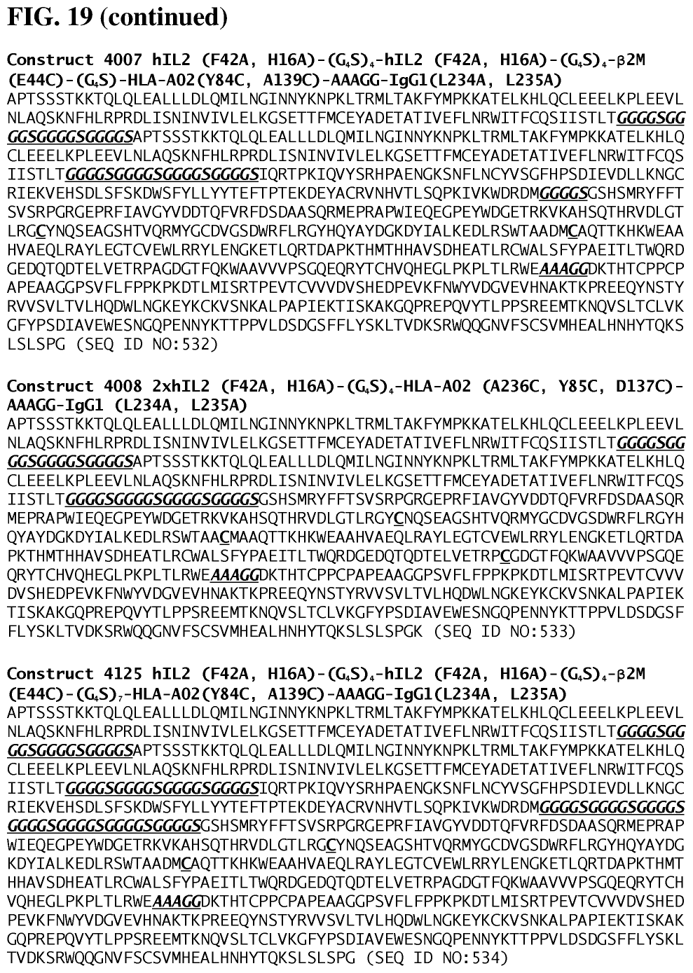

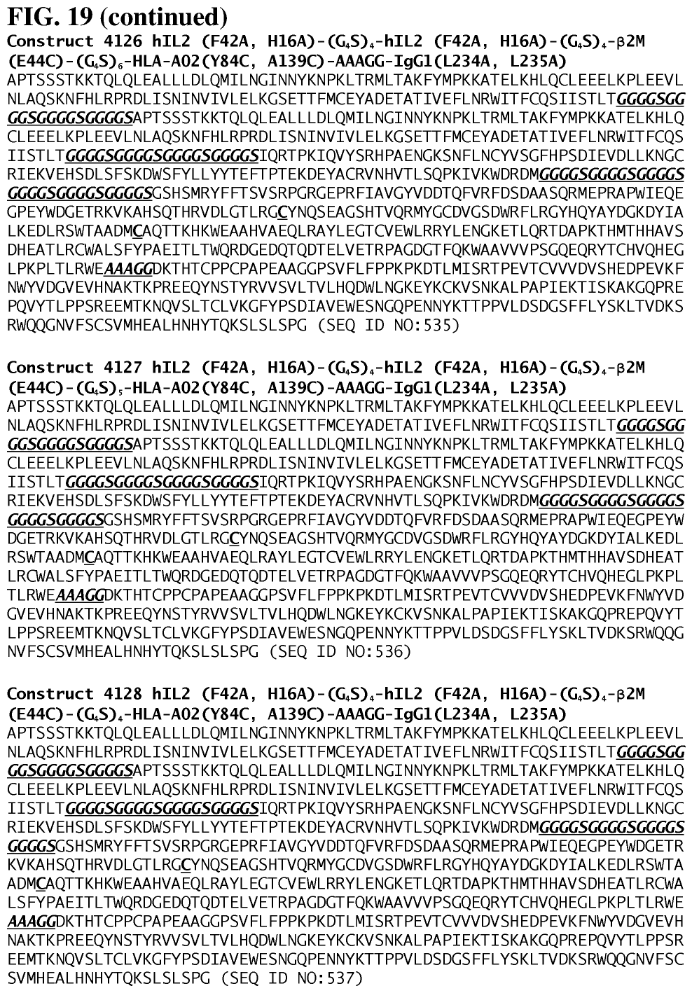

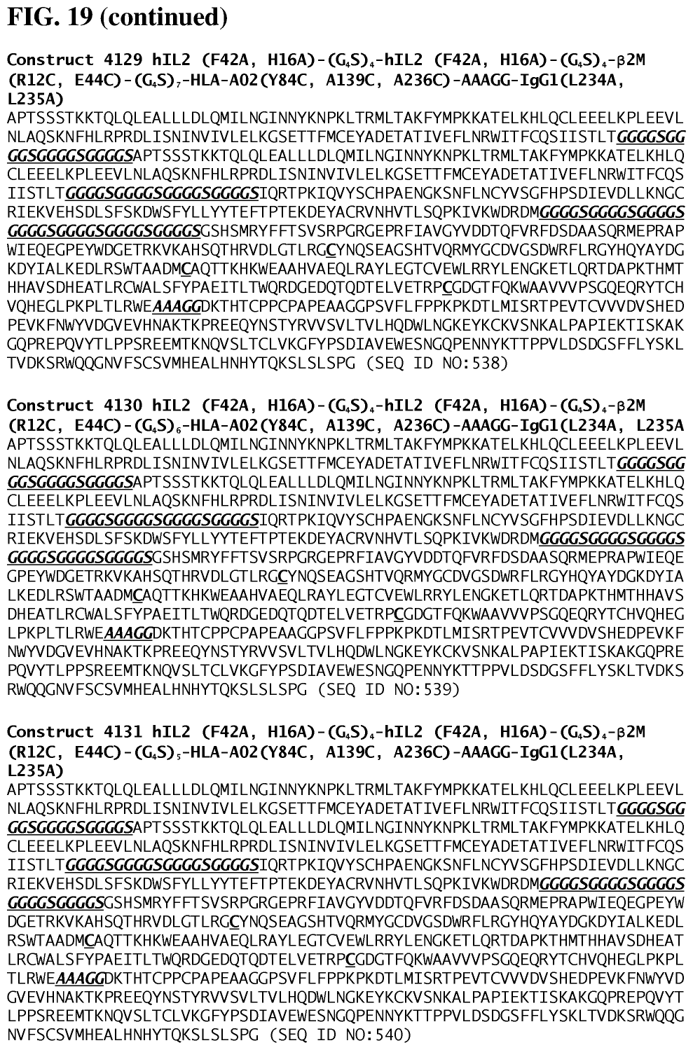

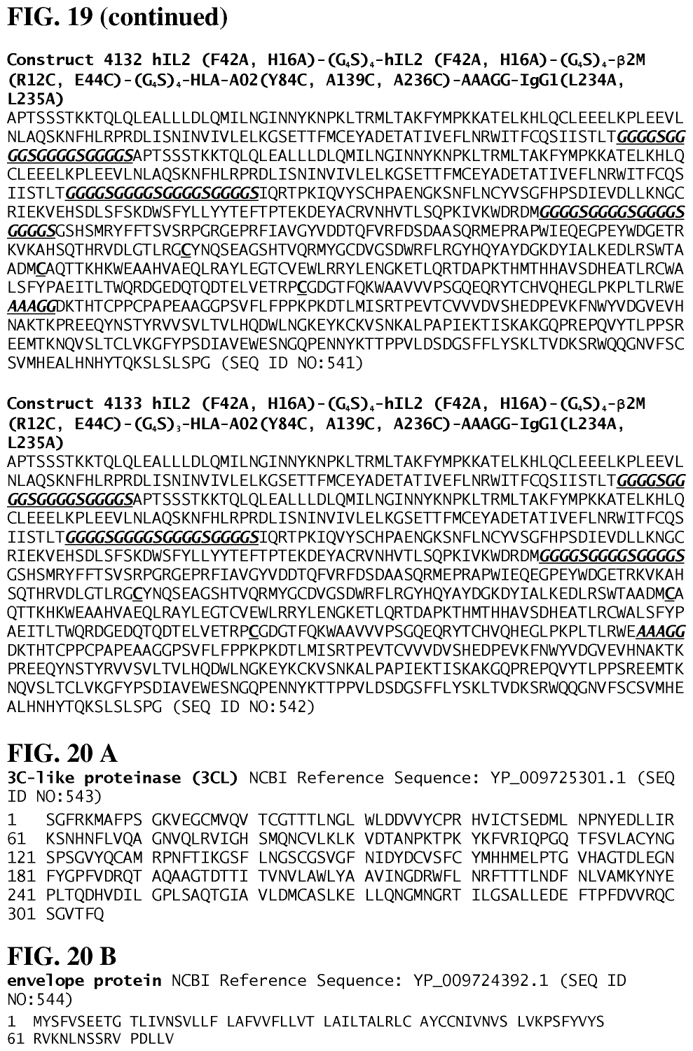

A series of examples are provided herein using T-Cell-MPs and their epitope conjugates. To the extent that the examples illustrate T-cell-MP-epitope conjugates comprising epitopes other than those described herein, they are provided to demonstrate a number of the properties of T-Cell-MPs and their epitope conjugates including, but not limited to, their manufacturability and the ability of T-Cell-MP-epitope conjugates induce responses in T cells specific to the conjugated epitope. The sequences of the constructs described in the examples are set forth in FIG. 19.

### Example 1

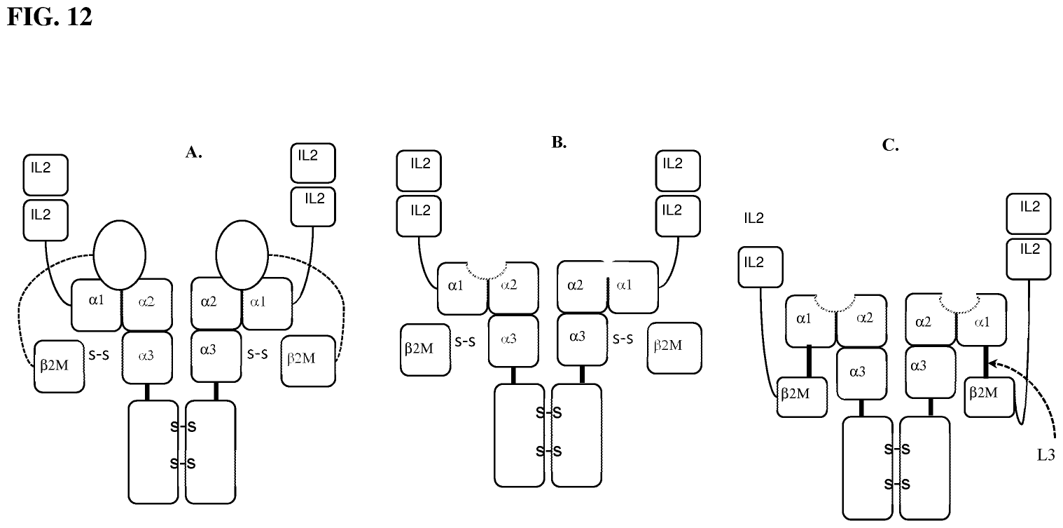

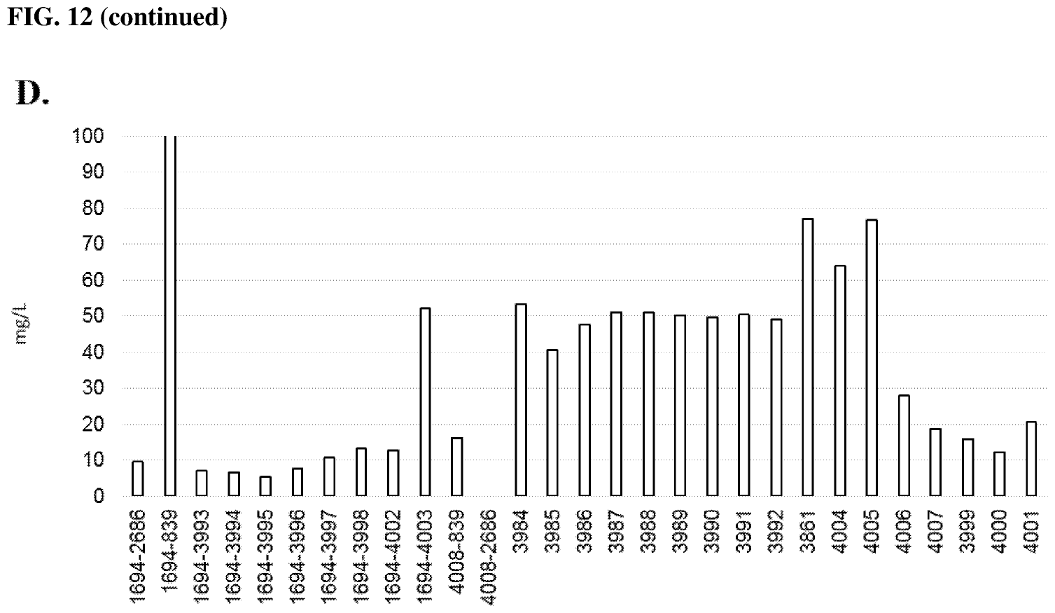

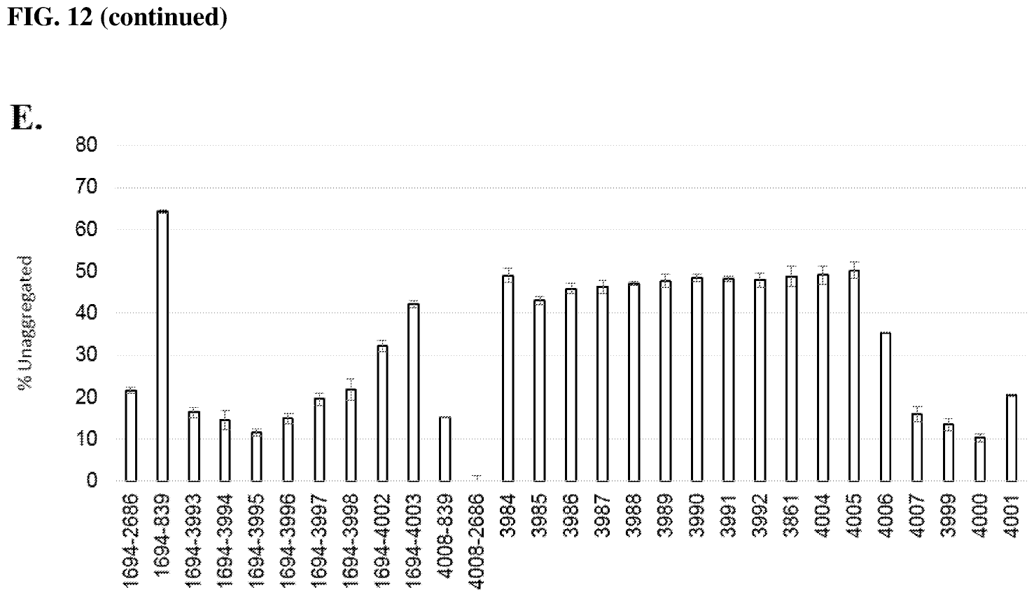

Nucleic acids were prepared encoding a series of constructs comprising an HLA-A*02:01 (HLA-A02) class I heavy chain polypeptide sequence, a human β2M polypeptide sequence, and an IgG scaffold sequence, as core elements of split chain or single chain constructs shown as duplexes in FIG. 12 at A, B and C.

Each of the split chain constructs (structures A or B) has a first polypeptide sequence that comprises from the N-terminus to the C-terminus tandem human IL-2 polypeptide sequences (2xhIL2) with F42A, H16A substitutions, HLA-A*02:01 (A02) α1, α2, and α3 domains, and a human IgG1 scaffold with L234A and L235A substitutions. The 1694 first polypeptide appearing in most of the split chain constructs comprises an A236C, Y84C and A139C substitutions 2xhIL2 (F42A, H16A)-(G₄S)₄-HLA-A02 (A236C, Y84C, A139C)-AAAGG-IgG1 (L234A, L235A): APTSSSTKKTQLQLEALLLDLQMILNGINNYKNPKLTRMLTAKFYMPKKATELKHLQCLEEELKPLEEVLNLA QSKNFHLRPRDLISNINVIVLELKGSETTFMCEYADETATIVEFLNRWITFCQSIISTLTGGGGGGGGSGGGGSGGG GSAPTSSSTKKTQLQLEALLLDLQMILNGINNYKNPKLTRMLTAKFYMPKKATELKHLQCLEEELKPLEEVLNLAQSKN FHLRPRDLISNINVIVLELKGSETTFMCEYADETATIVEFLNRWITFCQSIISTLTGGGGGGGGSGGGGSGGGGSGS HSMRYFFTSVSRPGRGEPRFIAVGYVDDTQFVRFDSDAASQRMEPRAPWIEQEGPEYWDGETRKVKAHSQTHRVD LGTLRGCYNQSEAGSHTVQRMYGCDVGSDWRFLRGYHQYAYDGKDYIALKEDLRSWTAADMCAQTTKHKWEAAH VAEQLRAYLEGTCVEWLRRYLENGKETLQRTDAPKTHMTHHAVSDHEATLRCWALSFYPAEITLTWQRDGEDQTQD TELVETRPCGDGTFQKWAAVVVPSGQEQRYTCHVQHEGLPKPLTLRWEAAAGGDKTHTCPPCPAPEAAGGPSVFL FPPKPKDTLMISRTPEVTCVVVDVSHEDPEVKFNWYVDGVEVHNAKTKPREEQYNSTYRVVSVLTVLHQDWLNGKE YKCKVSNKALPAPIEKTISKAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (linker sequences are indicated in bold and italics) (SEQ ID NO:506).

The 4008 polypeptide appearing in two split chain constructs parallels the 1694 construct, but comprises A236C, Y85C, and D137C substitutions in the HLA-A02 sequence-2xhIL2 (F42A, H16A)-(G₄S)₄-HLA-A02 (A236C, Y85C, D137C)-AAAGG-IgG1 (L234A, L235A): APTSSSTKKTQLQLEALLLDLQMILNGINNYKNPKLTRMLTAKFYMP KKATELKHLQCLEEELKPLEEVLNLAQSKNFHLRPRDLISNINVIVLELKGSETTFMCEYADETATIVEFLNRWITFCQSII STLTGGGGSGGGGGGGGSGGGGSAPTSSSTKKTQLQLEALLLDLQMILNGINNYKNPKLTRMLTAKFYMPKKATE LKHLQCLEEELKPLEEVLNLAQSKNFHLRPRDLISNINVIVLELKGSETTFMCEYADETATIVEFLNRWITFCQSIISTLTG GGGSGGGGSGGGGSGGGGSGSHSMRYFFTSVSRPGRGEPRFIAVGYVDDTQFVRFDSDAASQRMEPRAPWIEQ EGPEYWDGETRKVKAHSQTHRVDLGTLRGYCNQSEAGSHTVQRMYGCDVGSDWRFLRGYHQYAYDGKDYIALKE DLRSWTAACMAAQTTKHKWEAAHVAEQLRAYLEGTCVEWLRRYLENGKETLQRTDAPKTHMTHHAVSDHEATLRC WALSFYPAEITLTWQRDGEDQTQDTELVETRPCGDGTFQKWAAVVVPSGQEQRYTCHVQHEGLPKPLTLRWEAAA GGDKTHTCPPCPAPEAAGGPSVFLFPPKPKDTLMISRTPEVTCVVVDVSHEDPEVKFNWYVDGVEVHNAKTKPREE QYNSTYRVVSVLTVLHQDWLNGKEYKCKVSNKALPAPIEKTISKAKGQPREPQVYTLPPSREEMTKNQVSLTCLVKG FYPSDIAVEWESNGQPENNYKTTPPVLDSDGSFFLYSKLTVDKSRWQQGNVFSCSVMHEALHNHYTQKSLSLSPGK (SEQ ID NO:533).

Each of the split chain constructs (structures A and B) in FIG. 12 comprises a second polypeptide comprising a β2M polypeptide sequence having R12C and E44C substitutions: IQRTPKIQVYSCHPAENGKSNFLNC YVSGFHPSDIEVDLLKNGCRIEKVEHSDLSFSKDWSFYLLYYTEFTPTEKDEYACRVNHVTLSQPKIVKWDRDM (SEQ ID NO: 507); to which ether the indicated linker, or virus peptide (CMV peptide epitope NLVPMVATV (SEQ ID NO: 500) and linker, is added at their N-termini as indicated in the table provided below.

The unconjugated single chain T-Cell-MP conjugates listed in FIG. 12 each comprise as a single polypeptide chain from N-terminus to C-terminus IL-2, β2M, HLA-A*02:01 (A02) α1, α2, and α3 domains, and human IgG1 polypeptide sequences. The aa sequence of the 3861 construct is provided below, and the remainder of the single chain T-Cell-MP constructs may be considered variations of the 3861 construct, which has tandem 2xIL-2 sequences with F42A and H16A substitutions-a (G₄S)₄ linker-β2M (E44C)-a (G₄S)₃ linker-HLA-A02 with Y84C, A139C-a AAAGG linker- and an IgG1 with L234A and L235A substitutions: APTSSSTKKTQLQLEALLLDLQMILNGI NNYKNPKLTRMLTAKFYMPKKATELKHLQCLEEELKPLEEVLNLAQSKNFHLRPRDLISNINVIVLELKGSETTFMCEY ADETATIVEFLNRWITFCQSIISTLTGGGGSGGGGSGGGGSGGGGSAPTSSSTKKTQLQLEALLLDLQMILNGINNYK NPKLTRMLTAKFYMPKKATELKHLQCLEEELKPLEEVLNLAQSKNFHLRPRDLISNINVIVLELKGSETTFMCEYADET ATIVEFLNRWITFCQSIISTLTGGGGSGGGGSGGGGSGGGGSIQRTPKIQVYSRHPAENGKSNFLNCYVSGFHPSDIE VDLLKNGCRIEKVEHSDLSFSKDWSFYLLYYTEFTPTEKDEYACRVNHVTLSQPKIVKWDRDMGGGGSGGGGSGG GGSGSHSMRYFFTSVSRPGRGEPRFIAVGYVDDTQFVRFDSDAASQRMEPRAPWIEQEGPEYWDGETRKVKAHS QTHRVDLGTLRGCYNQSEAGSHTVQRMYGCDVGSDWRFLRGYHQYAYDGKDYIALKEDLRSWTAADMCAQTTKH KWEAAHVAEQLRAYLEGTCVEWLRRYLENGKETLQRTDAPKTHMTHHAVSDHEATLRCWALSFYPAEITLTWQRDG EDQTQDTELVETRPAGDGTFQKWAAVVVPSGQEQRYTCHVQHEGLPKPLTLRWEAAAGGDKTHTCPPCPAPEAA GGPSVFLFPPKPKDTLMISRTPEVTCVVVDVSHEDPEVKFNWYVDGVEVHNAKTKPREEQYNSTYRVVSVLTVLHQ DWLNGKEYKCKVSNKALPAPIEKTISKAKGQPREPQVYTLPPSREEMTKNQVSLTCLVKGFYPSDIAVEWESNGQPE NNYKTTPPVLDSDGSFFLYSKLTVDKSRWQQGNVFSCSVMHEALHNHYTQKSLSLSPG (SEQ ID NO:508).

Where tandem IL-2 sequences are present in the constructs of this example, they are separated by a (G₄S)₄ linker (SEQ ID NO:501). Each of the sequences other than 3861 has variations in the linkers present between the IL-2 and β2M, and/or β2M and HLA-A02 sequences (the L3 linker) as indicated. Additionally, construct 3984 has only a single IL-2 sequence, and each of 3999-4002 have an additional aa substitution in the HLA-A02 polypeptide sequence as indicated in the table that follows.

TABLE 4

| | | |
|---|---|---|
| | Form in | |
| Construct | FIG. 12 | Construct Content and Organization |

Split Chain Construct

| | | | |
|---|---|---|---|
| 1694 | 2686 | B | β2M (R12C, E44C) |
| | 839 | A | CMV-GGGASGGGGSGGGGS-β2M (R12C) |
| | 3993 | B | GM-(G₄S)₃-β2M (R12C, E44C) |
| | 3994 | B | GMGGS-(G₄S)₂-β2M (R12C, E44C) |
| | 3995 | B | GMS-(G₄S)₂-β2M (R12C, E44C) |
| | 3996 | B | GMGGGS-(G₄S)-β2M (R12C, E44C) |
| | 3997 | B | GMGS-(G₄S)-β2M (R12C, E44C) |
| | 3998 | B | GM-(G₄S)-β2M (R12C, E44C) |
| | 4002 | A | CMV-(G₃AS)-(G₄S)₂-β2M (R12C, E44C) |
| | 4003 | A | CMV-(G₃AS)-(GAS)-FC1-(G₄S)-β2M (R12C, E44C) |
| 4008 | 839 | A | CMV-GGGASGGGGSGGGGS-β2M (R12C) |
| | 2686 | B | β2M (R12C, E44C) |

Unconjugated T-Cell-MP (Single Chain Construct)

| | | |
|---|---|---|
| | C | 1xhIL2(F42A, H16A)-(G₄S)₄-β2M (E44C)-(G₄S)₃-HLA-A02(Y84C, A139C)- |
| | | AAAGG-IgG1(L234A, L235A) |
| 3985 | C | hIL2 (F42A, H16A)-(G₄S)₄-hIL2 (F42A, H16A)-(G₄S)-GLGGS-(G₄S)₂-β2M |
| | | (E44C)-(G₄S)₃-HLA-A02(Y84C, A139C)-AAAGG-IgG1(L234A, L235A) |
| 3986 | C | hIL2 (F42A, H16A)-(G₄S)₄-hIL2 (F42A, H16A)-(G₄S)₂-GLGGS-(G₄S)-β2M |
| | | (E44C)-(G₄S)₃-HLA-A02(Y84C, A139C)-AAAGG-IgG1(L234A, L235A) |
| 3987 | C | hIL2 (F42A, H16A)-(G₄S)₄-hIL2 (F42A, H16A)-(G₄S)₃-GLGGS-β2M (E44C)- |
| | | (G₄S)₃-HLA-A02(Y84C, A139C)-AAAGG-IgG1(L234A, L235A) |
| 3988 | C | hIL2 (F42A, H16A)-(G₄S)₄-hIL2 (F42A, H16A)-(G₄S)-GMGGSGGGGS-(G₄S)- |
| | | β2M (E44C)-(G₄S)₃-HLA-A02(Y84C, A139C)-AAAGG-IgG1(L234A, L235A) |
| 3989 | C | hIL2 (F42A, H16A)-(G₄S)₄-hIL2 (F42A, H16A)-(G₄S)-GGGMSGGGGS-(G₄S)- |
| | | β2M (E44C)-(G₄S)₃-HLA-A02(Y84C, A139C)-AAAGG-IgG1(L234A, L235A) |
| 3990 | C | hIL2 (F42A, H16A)-(G₄S)₄-hIL2 (F42A, H16A)-(G₄S)-GGGGSMGGGS-(G₄S)- |
| | | β2M (E44C)-(G₄S)₃-HLA-A02(Y84C, A139C)-AAAGG-IgG1(L234A, L235A) |
| 3991 | C | hIL2 (F42A, H16A)-(G₄S)₄-hIL2 (F42A, H16A)-(G₄S)-GGGGSGMGGS-(G₄S)- |
| | | β2M (E44C)-(G₄S)₃-HLA-A02(Y84C, A139C)-AAAGG-IgG1(L234A, L235A) |
| 3992 | C | hIL2 (F42A, H16A)-(G₄S)₄-hIL2 (F42A, H16A)-(G₄S)-GGGGSGGGGM-(G₄S)- |
| | | β2M (E44C)-(G₄S)₃-HLA-A02(Y84C, A139C)-AAAGG-IgG1(L234A, L235A) |
| 3861 | C | hIL2 (F42A, H16A)-(G₄S)₄-hIL2 (F42A, H16A)-(G₄S)₄-β2M (E44C)-(G₄S)₃- |
| | | HLA-A02(Y84C, A139C)-AAAGG-IgG1(L234A, L235A) |
| 4004 | C | hIL2 (F42A, H16A)-(G₄S)₄-hIL2 (F42A, H16A)-(G₄S)₅-β2M (E44C)-(G₄S)₃- |
| | | HLA-A02(Y84C, A139C)-AAAGG-IgG1(L234A, L235A) |
| 4005 | C | hIL2 (F42A, H16A)-(G₄S)₄-hIL2 (F42A, H16A)-(G₄S)₆-β2M (E44C)-(G₄S)₃- |
| | | HLA-A02(Y84C, A139C)-AAAGG-IgG1(L234A, L235A) |
| 4006 | C | hIL2 (F42A, H16A)-(G₄S)₄-hIL2 (F42A, H16A)-(G₄S)₄-β2M (E44C)-(G₄S)₂- |
| | | HLA-A02(Y84C, A139C)-AAAGG-IgG1(L234A, L235A) |
| 4007 | C | hIL2 (F42A, H16A)-(G₄S)₄-hIL2 (F42A, H16A)-(G₄S)₄-β2M (E44C)-(G₄S)- |
| | | HLA-A02(Y84C, A139C)-AAAGG-IgG1(L234A, L235A) |
| 3999 | C | hIL2 (F42A, H16A)-(G₄S)₄-hIL2 (F42A, H16A)-(G₄S)₄-β2M (E44C)-(G₄S)₃- |
| | | HLA-A02(Y84C, A139C, T143L)-AAAGG-IgG1(L234A, L235A) |
| 4000 | C | hIL2 (F42A, H16A)-(G₄S)₄-hIL2 (F42A, H16A)-(G₄S)₄-β2M (E44C)-(G₄S)₃- |
| | | HLA-A02(Y84C, A139C, T143M)-AAAGG-IgG1(L234A, L235A) |
| 4001 | C | hIL2 (F42A, H16A)-(G₄S)₄-hIL2 (F42A, H16A)-(G₄S)₄-β2M (E44C)-(G₄S)₃- |
| | | HLA-A02(L81M, Y84C, A139C)-AAAGG-IgG1(L234A, L235A) |

The nucleic acids encoding the protein constructs were transfected into and expressed by CHO cells as soluble protein in the culture media. The level of protein expressed in the culture media after 7 days was determined by BLI assay using protein A to capture the expressed protein (FIG. 12 at D). The fraction of protein appearing in unaggregated duplex form is assessed by isolating the protein from the culture media using magnetic protein A beads. After washing, the bound protein is eluted from the beads by reducing the pH, and then subject to analytical size exclusion chromatography using UV detection on an AGILENT® chromatography system. The fraction of unaggregated protein reported in FIG. 12 at E is based on the area of the peak corresponding to the molecular weight of the duplex relative to the total area of the chromatographed protein.

The results indicate that unconjugated single chain T-Cell-MP constructs appear to be expressed more uniformly at higher levels than their unconjugated split chain construct counterparts.

### Example 2

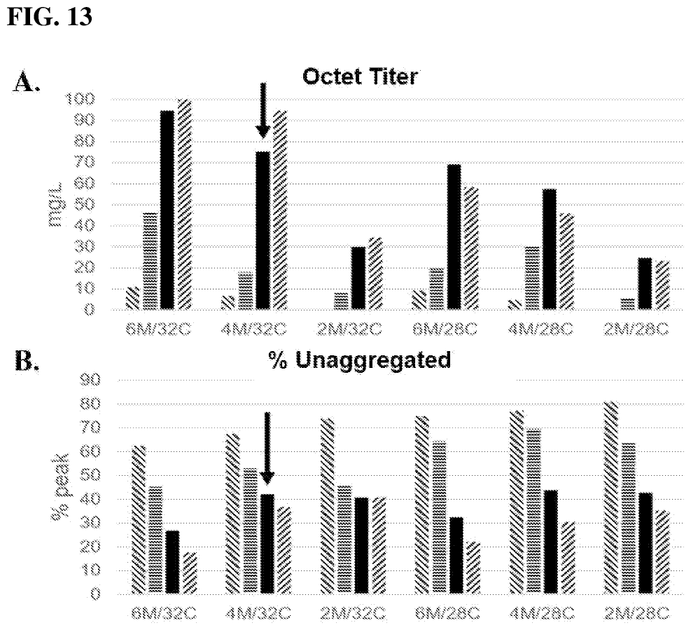

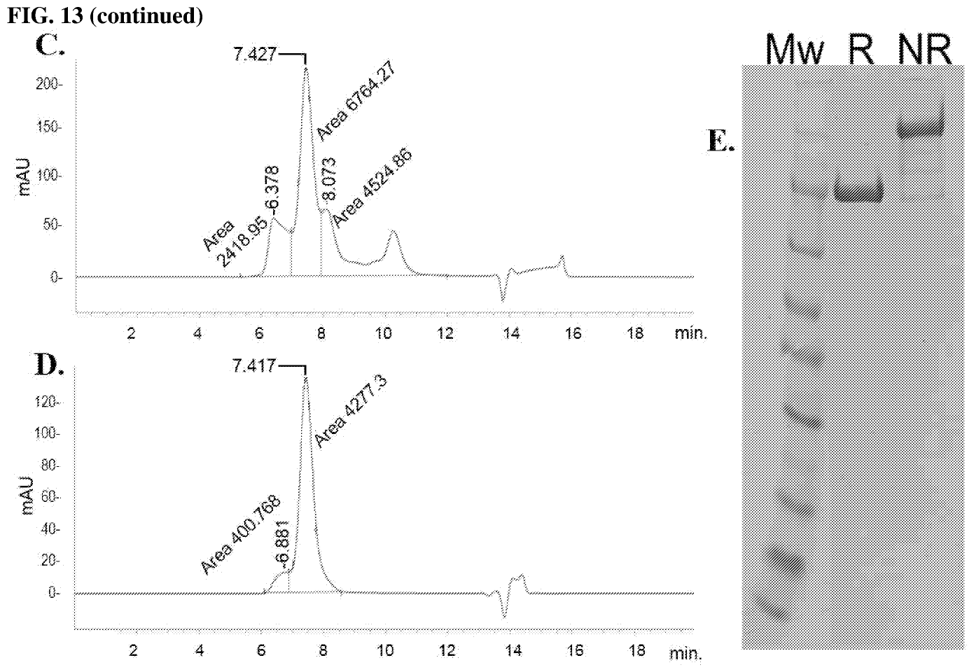

The effect of time in culture, cell culture density, and culture temperature on unconjugated T-Cell-MPs was examined by transiently expressing the construct 3861 (see Example 1) in CHO cells at 28 and 32° C. Transfection was accomplished with expiCHO® transfection kits (Gibco™/ThermoFisher Scientific, Skokie, IL) using a recombinant pTT5 vector into which the cassette encoding the polypeptide was cloned. The transfected cells were diluted to 2, 4 or 6 million cells per milliliter and T-Cell-MP 3861 expression levels and the fraction of unaggregated protein in duplex form determined at days 2, 4, 7, and/or 9 as indicated by removing a portion of the culture. Analyses were conducted as in Example 1 and are shown in FIG. 13 (at A and B) with the number of cells and culture temperature shown below each histogram set (e.g., six million cells at 32° C. denoted as 6M/32C). Also shown in FIG. 13 are size exclusion chromatograms (C and D) of the unconjugated 3861 T-Cell-MP harvested from a culture using protein A and after further purification by size exclusion chromatography (upper and lower chromatograms respectively). Coomassie blue stained SDS PAGE analysis (at E) confirms the purity and homogeneity of the purified material, samples of which were applied to the gel in reduced (R) and non-reduced form (NR).

### Example 3

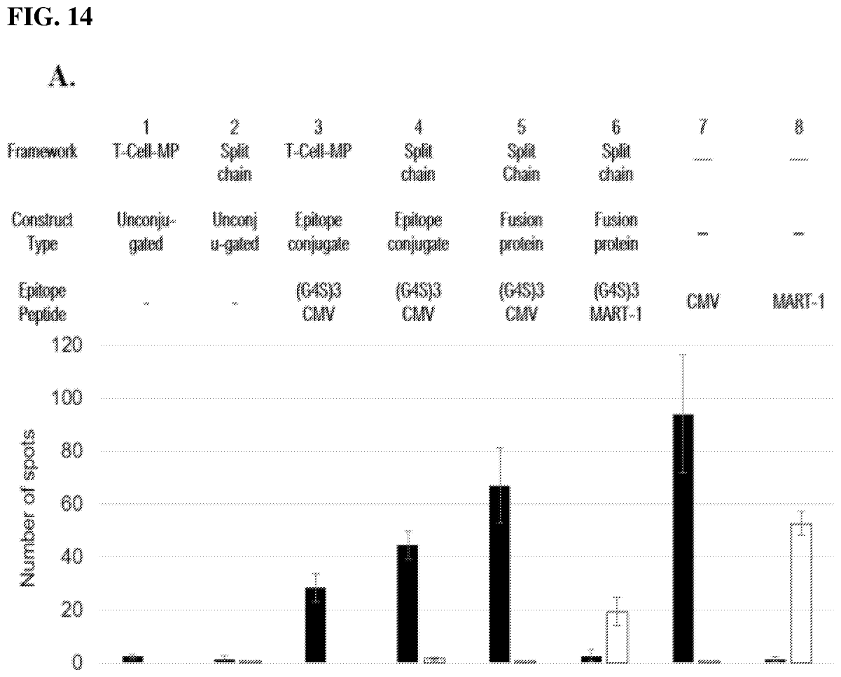

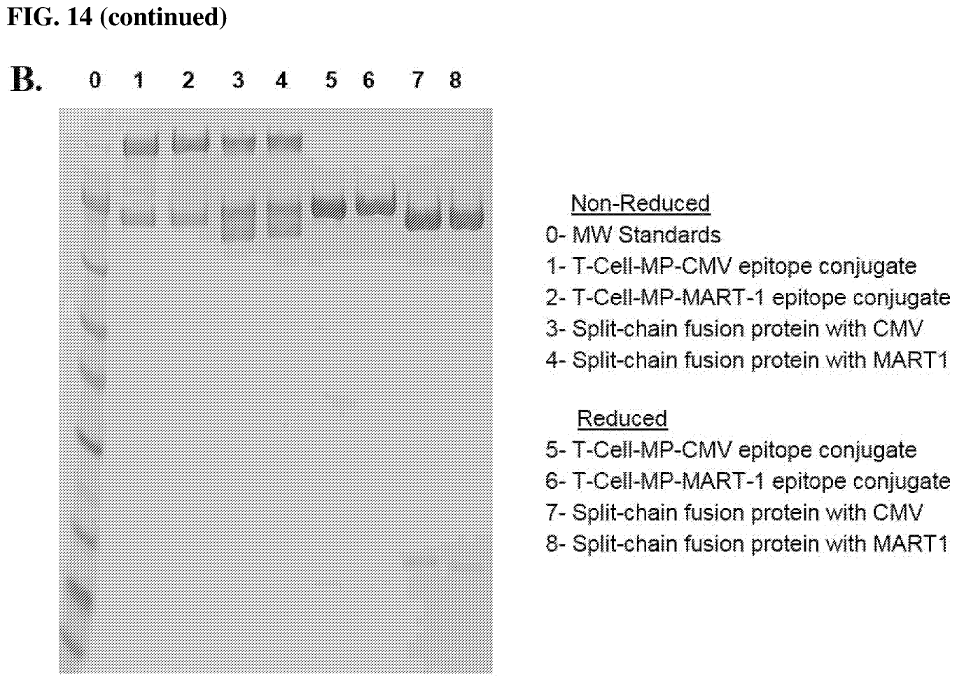

The specific interaction of T-Cell-MP-epitope conjugates and control constructs with epitope-specific T cells was assessed by incubating the molecules with T cells responsive to either the CMV peptide NLVPMVATV (black bars) (SEQ ID NO:500) or the Melan-A and Mucin Related Peptide (MART-1) ELAGIGIL TV (white bars) (SEQ ID NO: 502) in the histogram of Elispot data provided FIG. 14A. Control samples of the unconjugated 3861 T-Cell-MP duplex (see FIG. 12 at C for the general structure) group 1, and an unconjugated split chain construct comprising polypeptides 1694 and 2686 (duplexed as in FIG. 12 at B) group 2 were run in parallel with test samples. T-Cell-MP and split chain constructs conjugated to the E44C position of β2M through a (G₄S)₃ linker by a maleimide group are shown in groups 3 and 4. The effect of control construct split chain fusion proteins (FIG. 12, structure A) having a CMV or MART-1 polypeptide as part of the fusion protein are shown in groups 5 and 6 respectively. Control stimulation by CMV and MART-1 peptides is shown in groups 7 and 8 respectively. The histogram indicates the number of spots due to captured interferon gamma indicating activation of the T cells by the treatments.

The SDS-PAGE gel shown in FIG. 14 B provides an analysis of reducing and non-reducing samples of the epitope conjugates and fusion proteins, indicating their purity and homogeneity.

### Example 4

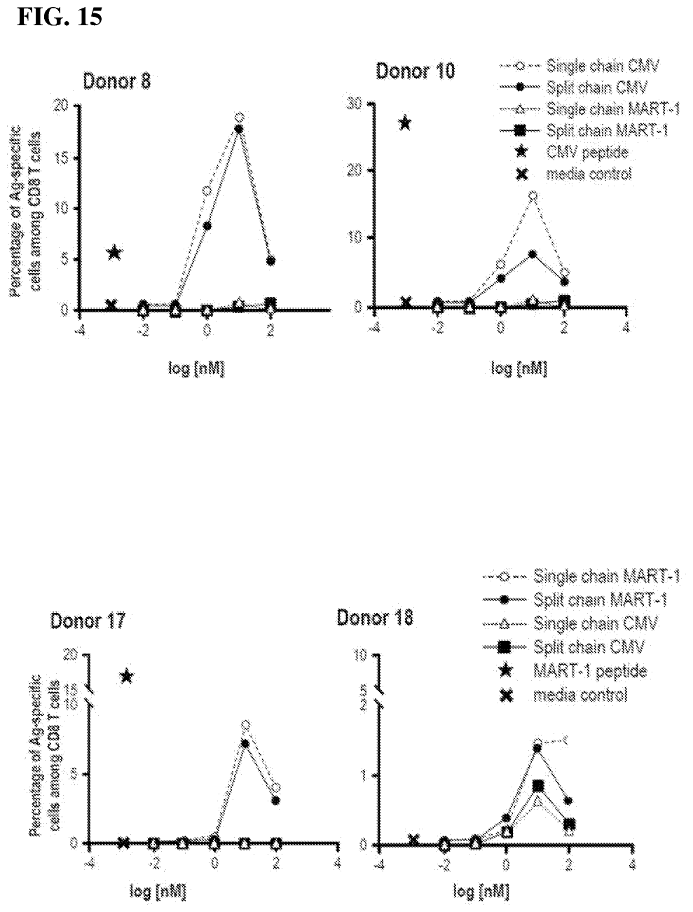

Ficoll-Paque® purified samples of leukocytes from CMV responsive donors (Donors 8, 10, 38, and 39) and MART-1 responsive donors (Donors 17 and 18) were prepared and used to demonstrate the ability of T-Cell-MP-epitope conjugates to expand T cells specific to CMV or MART-1 specific epitopes. MART-1 responsive Donor 18 also displays some responsiveness to the CMV peptide. Positive and negative control treatments included: treatment with split chain constructs conjugated to CMV and MART-1 peptides; treatment with the CMV or MART-1 peptides in culture media; and media only control treatment. For the experiments, leukocytes were suspended at 2.5×10⁶ cells per ml in ImmunoCult™ media (Stemcell Technologies, Vancouver, British Columbia) containing the indicated amounts of the control or T-Cell-MP-epitope conjugate or control treatments. After 10 days in culture the number of cells responsive to CMV or MART-1 were assessed by Flow cytometry using CMV or MART-1 tetramers purchased from MBL International Corp. The results indicate that both the T-Cell-MP and split chain constructs conjugated to the CMV peptide, and to a lesser degree cMV peptide, stimulate expansion of CMV specific T cells from CMV responsive donors in a concentration dependent manner. T-Cell-MP and split chain constructs conjugated to the MART-1 peptide, and to a lesser degree the MART-1 peptide stimulate expansion of MART-1 specific T cells from MART-1 responsive donors in a concentration dependent manner. In each instance, CMV peptide conjugates selectively stimulated T cells from CMV responsive donors but not MART-1 responsive donors and vice versa. Free peptide in the absence of IL-2 failed to produce an effect equal to the effect observed with the T-Cell-MP-epitope conjugates. Results are provided in FIG. 15.

The T-Cell-MP-epitope conjugate employed for the assays was a duplex of the 3186 polypeptide (see Example 1 and FIG. 12 structure C for the general form of the unconjugated duplex) conjugated at a cysteine (E44C) in the β2M polypeptide sequence to either a CMV (NLVPMVATV, SEQ ID NO:500) or MART-1 (ELAGIGILTV) (SEQ ID NO: 502) peptide via a (G₄S)₃ linker bearing a maleimide group (e.g., for the CMV peptide NLVPMVATV-(G₄S)₃-lysine-epsilon amino-maleimide). The split chain epitope conjugate was a duplex of two split chain constructs each comprising α1694 and 2686 polypeptide (see Example 1 and FIG. 12 structure B for the general form of the unconjugated duplex), which was conjugated at a cysteine (E44C) in the β2M polypeptide sequence to a either a CMV (NLVPMVATV, SEQ ID NO:500) or MART-1 (ELAGIGILTV, SEQ ID NO:502) peptide via a (G₄S)₃ (SEQ ID NO: 285) linker bearing a maleimide group. After reduction to remove any capping from the cysteine conjugation sites, the conjugation was conducted as described in for maleimide coupling reactions using at least two additions of the peptide bearing a maleimide group.

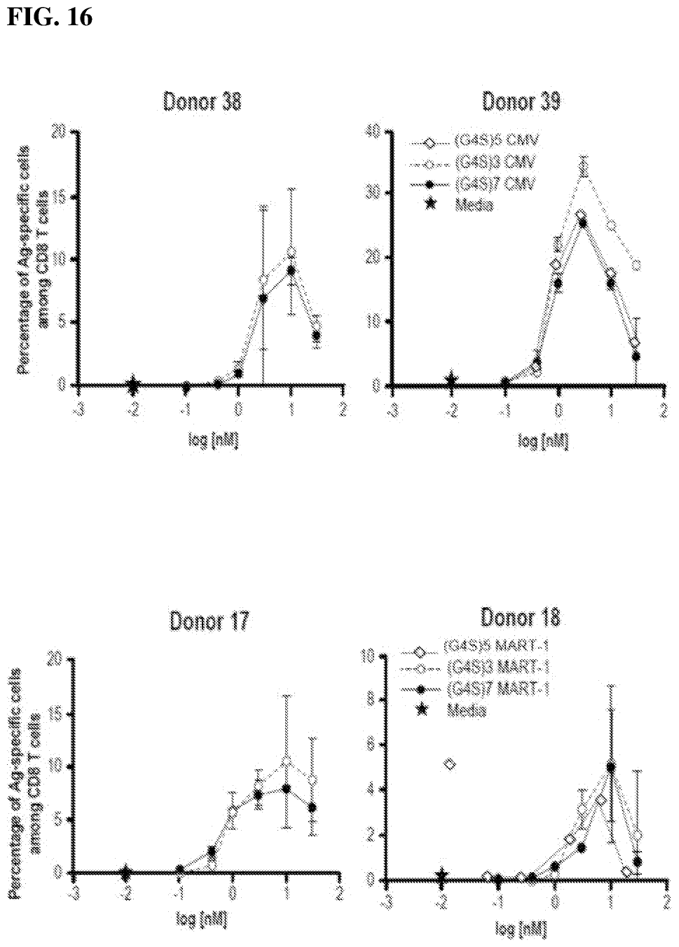

In an additional test, the effect of a construct bearing a (G₄S)₇ (SEQ ID NO:503) L3 linker (the linker between the β2M and HLA-A02 sequences), but otherwise identical to 3861, was compared with the 3861 polypeptide duplex (i.e., construct 4125 2xIL2 (F42A, H16A)-(G₄S)₇-β2M (E44C)-(G₄S)₃-HLA-A02 (Y84C, A139C)-AAAGG-IgG1 (L234A, L235A)). Duplexes of both the 3861 and 4125 constructs were conjugated to a CMV or MART-1 peptide by a maleimide terminated (G₄S)₃ linker and tested side-by-side for the ability to expand T cells in an epitope-specific manner. The assays were conducted as described above for the 3861 epitope conjugates, except only a media alone control was conducted. The results, shown in FIG. 16, indicate that extending the linker length did not substantively alter the expansion of T cells seen with the 3861 epitope conjugates.

### Example 5

In order to examine the effect of L3 linker length on the level of cell expression and the quality (fraction unaggregated) of T-Cell-MP proteins a series of nucleic acids encoding constructs 4125 through 4128 that are related to construct 3861 but with L3 linkers of increasing length were prepared and inserted into an expression vector (pTT5). A second set of constructs (4129-4133) bearing an additional R12C substitution in the β2M polypeptide (R12C, E44C) and an A236C substitution in the HLA-A02 peptide that can form an interchain disulfide bond was also prepared. The vectors were transfected into CHO cells with expiCHO® transfection kits and both the amount of protein expressed in the culture media and the fraction of unaggregated protein after purification using magnetic beads was assessed at days 4, 6, 8, and/or 11 as indicated. The specific constructs included those recited in the following table.

| | Form in | L3 linker | |
|---|---|---|---|
| Construct | FIG. 12 | (G₄S)n | Construct Content and Organization |
| 3861 | C | n = 3 | hIL2 (F42A, H16A)-(G₄S)₄-hIL2 (F42A, H16A)-(G₄S)₄-β2M (E44C)-(G₄S)₃- |
| | | | HLA-A02(Y84C, A139C)-AAAGG-IgG1(L234A, L235A) |
| 4128 | C | n = 4 | hIL2 (F42A, H16A)-(G₄S)₄-hIL2 (F42A, H16A)-(G₄S)₄-β2M (E44C)-(G₄S)₄- |
| | | | HLA-A02(Y84C, A139C)-AAAGG-IgG1(L234A, L235A) |
| 4127 | C | n = 5 | hIL2 (F42A, H16A)-(G₄S)₄-hIL2 (F42A, H16A)-(G₄S)₄-β2M (E44C)-(G₄S)₅- |
| | | | HLA-A02(Y84C, A139C)-AAAGG-IgG1(L234A, L235A) |
| 4126 | C | n = 6 | hIL2 (F42A, H16A)-(G₄S)₄-hIL2 (F42A, H16A)-(G₄S)₄-β2M (E44C)-(G₄S)₆- |
| | | | HLA-A02(Y84C, A139C)-AAAGG-IgG1(L234A, L235A) |
| 4125 | C | n = 7 | hIL2 (F42A, H16A)-(G₄S)₄-hIL2 (F42A, H16A)-(G₄S)₄-β2M (E44C)-(G₄S)₇- |
| | | | HLA-A02(Y84C, A139C)-AAAGG-IgG1(L234A, L235A) |
| 4129 | C | n = 7 | hIL2 (F42A, H16A)-(G₄S)₄-hIL2 (F42A, H16A)-(G₄S)₄-β2M (R12C, E44C)- |
| | | | (G₄S)₇-HLA-A02(Y84C, A139C, A236C)-AAAGG-IgG1(L234A, L235A) |
| 4130 | C | n = 6 | hIL2 (F42A, H16A)-(G₄S)₄-hIL2 (F42A, H16A)-(G₄S)₄-β2M (R12C, E44C)- |
| | | | (G₄S)₆-HLA-A02(Y84C, A139C, A236C)-AAAGG-IgG1(L234A, L235A) |
| 4131 | C | n = 5 | hIL2 (F42A, H16A)-(G₄S)₄-hIL2 (F42A, H16A)-(G₄S)₄-β2M (R12C, E44C)- |
| | | | (G₄S)₅-HLA-A02(Y84C, A139C, A236C)-AAAGG-IgG1(L234A, L235A) |
| 4132 | C | n = 4 | hIL2 (F42A, H16A)-(G₄S)₄-hIL2 (F42A, H16A)-(G₄S)₄-β2M (R12C, E44C)- |
| | | | (G₄S)₄-HLA-A02(Y84C, A139C, A236C)-AAAGG-IgG1(L234A, L235A) |
| 4133 | C | n = 3 | hIL2 (F42A, H16A)-(G₄S)₄-hIL2 (F42A, H16A)-(G₄S)₄-β2M (R12C, E44C)- |
| | | | (G₄S)₃-HLA-A02(Y84C, A139C, A236C)-AAAGG-IgG1(L234A, L235A) |

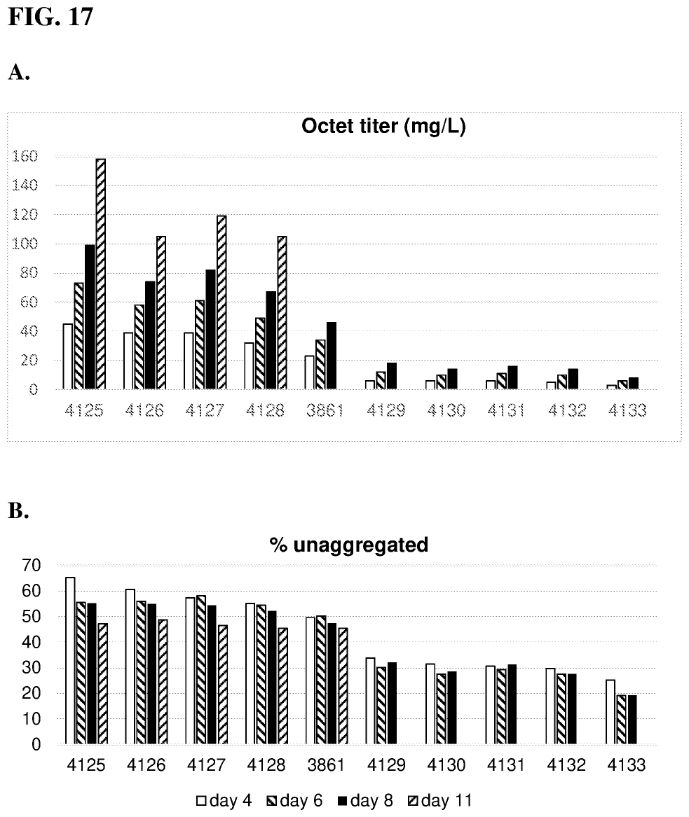

The amount of the expressed unconjugated T-Cell-MP constructs were determined by BLI assay using protein A for capture on a BioForte instrument using the methods described in Example 1. Results are provided in FIG. 17 histogram A.

The fraction of unconjugated T-Cell-MP that is unaggregated (present in duplex form) after purification on magnetic protein A beads was determined by size exclusion chromatography. The fraction was determined using the area of the chromatographic peak corresponding to the molecular weight of the duplex relative to the area under the chromatogram as described in Example 1. Results are shown in FIG. 17 histogram B.

Additional optimization indicates that higher yields are possible. Construct 4125 has been observed to reach 200 mg/ml and construct 4127 has been observed to reach 170 mg/ml in CHO culture cell media prior to isolation.

### Example 6 Expansion of T Cells Recognizing a SARs-CoV-2 Epitope

A T-Cell-MP-Epitope Conjugate designated NST-0032 comprising a conjugated coronavirus peptide epitope (a T-Cell-MP-coronavirus epitope conjugate) was prepared and tested for its ability to expand T-cells specific for the epitope. For the preparation of NST-0032 the unconjugated T-cell-MP 4125, which has tandem IL-2 MODs, was prepared by as outlined in Example 5. The aa sequence of the unconjugated T-Cell-MP 4125 is provided in FIG. 19 and is generally of the form of the depicted in FIG. 9 structure A, self-assembling into a duplex as in structure B. The unconjugated duplex of construct 4125, which bears solvent accessible cysteine chemical conjugation site due to a substitution of Lys 44 with a Cys (E44C), was conjugated at that cysteine to the spike protein peptide epitope YLQPRTELL (SEQ ID NO:461). The conjugation, which was indirect through a 3× G₄S (SEQ ID NO:285) peptide linker, employed a maleimide reactive group to form the covalent bond. The peptide construct used for the conjugation was of the form YLQPRTFLLGGGGSGGGGSGGGGSX (SEQ ID NO:504), where X is a lysine modified on its sidechain amino group with the reactive maleimide (see, e.g., FIG. 8 structure C). The resulting T-Cell-MP-epitope conjugate designated NST-0032, which is forms a duplex (see FIG. 9 structure C) was separated from unreacted peptide epitope maleimide reagent and placed in PBS-saline.

Samples of peripheral blood mononuclear cells (PBMCs) from prepared from the blood of four donors vaccinated against SARS-CoV-2 that were pre-screened for reactivity to YLQPRTELL (SEQ ID NO:461) were obtained along with control PBMCs from an unvaccinated individual. Vaccination with Moderna vaccine is indicated by MRNA, Johnson & Johnson vaccine by JNJ, and Pfizer vaccine by PFE; the date and number of vaccinations is unknown.

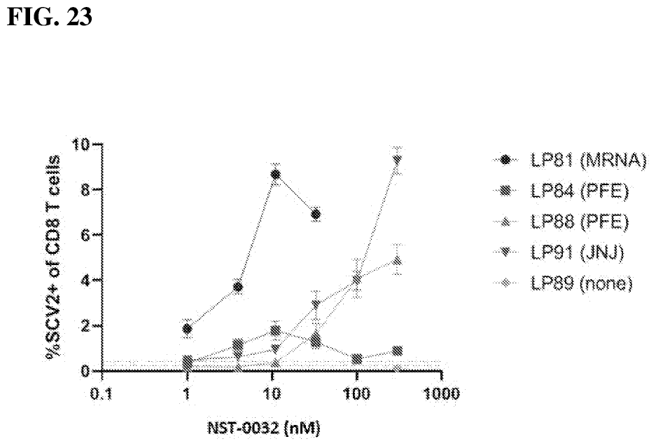

The PMCs from the vaccinated donors and the control unvaccinated donor were exposed to a dose-response of NST-0032 (from 1 nM to 500 nm) for nine days, with a feeding of fresh culture media on day 5. At day 9 the percentage of CD8+ T cells specific to the epitope (% SCV2+ of CD8 T Cells) was determined by FACS for each concentration of NST-0032 tested. The results, shown in FIG. 23 show a clear dose response for the proliferation of CD8+ T cells specific to the epitope in three of the four vaccinated individuals.

### Example 7

Coronavirus peptide epitopes are conjugated to a T-Cell-MP as described and detailed above, thereby forming T-Cell-MP-epitope conjugates. Non-limiting examples of peptides presenting coronavirus epitopes that can be used to form T-Cell-MP-epitope conjugates include those recited in Table 2 and FIG. 21.

The above Examples illustrate the ability to produce T-Cell-MPs and conjugate them to a peptide resulting in a T-Cell-MP-epitope conjugate protein that is not aggregated, displays suitable stability for use at 37° C., and can be purified. In particular, in Examples 3 and 4 above T-Cell-MP-epitope conjugates were generated and tested wherein the T-Cell-MP is conjugated to a CMV polypeptide (“CMV+ T-Cell-MP”) or a melanoma antigen MART-1 (“MART+ T-Cell-MP”) via a maleimide reactive linker attached to the peptide. The specific interaction of T-Cell-MP-epitope conjugates with epitope-specific T cells was confirmed assessed by incubating the molecules with T cells responsive to either the CMV peptide NLVPMVATV (SEQ ID NO:500) or the Melan-A and Mucin Related Peptide (MART-1) ELAGIGILTV (SEQ ID NO:502) (FIG. 15 and FIG. 16). While the above Examples, including Examples 3 and 4, are conducted with a CMV peptide epitope and/or other peptide epitopes; coronavirus peptide epitopes can equally be utilized to form epitope conjugates providing similar results.

The antigen specificity in the responses are evidenced by the fact that CMV Control Construct and IL-2 T-Cell-MP molecules did not substantially stimulate expansion of MART-1 responsive CD8+ T-cells. Likewise, MART-1 Control Construct and IL-2 T-Cell-MP molecules did not stimulate expansion of CMV responsive CD8+ T-cells. Accordingly, the presence of IL-2 polypeptide sequences present in each of the molecules were not responsible for substantial nonspecific expansion of the leukocytes.

1. A T cell modulatory polypeptide-epitope conjugate (T-Cell-MP-epitope conjugate), the polypeptide comprising:
(i) optionally one or more independently selected MOD polypeptide sequences, or two or more MOD polypeptide sequences, wherein when there are two or more MOD polypeptide sequences they are optionally joined to each other by independently selected L1 linkers,
(ii) an optional L2 linker polypeptide sequence joining the one or more MOD polypeptide sequences to a β2M polypeptide sequence,
(iii) the β2M polypeptide sequence,
(iv) an optional L3 linker polypeptide sequence comprising from 15 to 50 amino acids,
(v) a class I MHC-H polypeptide sequence,
(vi) an optional L4 linker polypeptide sequence,
(vii) a scaffold polypeptide sequence,
(viii) an optional L5 linker polypeptide sequence, and
(ix) optionally one or more independently selected MOD polypeptide sequences or two or more MOD polypeptide sequences optionally joined to each other by independently selected L6 linkers;
wherein
the T-Cell-MP-epitope conjugate comprises at least one MOD polypeptide sequence,
the MHC-H polypeptide sequence comprises a cysteine at position 84 and position 139 that may form a disulfide bond,
at least one of the β2M polypeptide sequence, the L3 linker polypeptide sequence, and/or the MHC-H polypeptide sequence comprises one or more chemical conjugation sites for epitope conjugation to which a peptide presenting a coronavirus epitope is covalently bound, directly or indirectly,
the β2M polypeptide sequence has at least 90% or at least 90% sequence identity to at least 90 or 95, contiguous aas of a mature human β2M polypeptide of SEQ ID NO:61, and
the peptide presenting a coronavirus epitope comprises seven or more contiguous amino acids of any of the coronavirus protein or peptide sequences set forth in FIGS. 20A to 20J, FIG. 21, FIG. 22, Table 2, Table 3, or Table 3a.

2. (canceled)

3. The T-Cell-MP-epitope conjugate of claim 1, wherein the MHC-H polypeptide sequence comprises a human class I MHC-H chain polypeptide sequence selected from HLA-A, HLA-B, HLA-C, HLA-E, HLA-F, and HLA-G MHC-H polypeptide sequences having:
(i) at least 90%, or at least 95%, sequence identity to at least 200 or at least 225 contiguous aas of a MHC-H polypeptide sequence provided in any of FIGS. 3A-3H or an HLA consensus polypeptide sequence recited in FIG. 3I; or
(ii) at least 95% or at least 98% sequence identity to at least 250 contiguous aas of a MHC-H polypeptide provided in any of FIGS. 3A-3H.

4. The T-Cell-MP-epitope conjugate of claim 3, wherein:
the peptide presenting a coronavirus epitope comprises 6-25 or 8-25 contiguous amino acids of a SARS-CoV-2 variant spike protein epitope that comprises a sequence selected from the group consisting of: KVGGNYNYR (SEQ ID NO:473); KGFNCYFPL (SEQ ID NO: 475); DVYYHKNNK (SEQ ID NO:477); NSANLAAIK (SEQ ID NO:480); RFANPVLPF (SEQ ID NO:482); AYSNNSIAIPINF (SEQ ID NO:484); PINLVRGL (SEQ ID NO:486); SRRRARSVA (SEQ ID NO:487); IPINFTISV (SEQ ID NO:489); NLRTRTQL (SEQ ID NO:491); NFTNRTQL (SEQ ID NO:492); LXXXHRSYL (SEQ ID NO: 494); LPSAYTNSF (SEQ ID NO:496); and AEYVNNSY (SEQ ID NO:498); or
wherein the peptide presenting a coronavirus epitope comprises a peptide of the spike protein of SEQ ID NO:566 bearing one or two sequence changes selected from the group consisting of T19R, A67V, del69-70, (V70F*), T95I, G142D, del142-144, Y145D, R158G, del211, L212I, ins214EPE, (A222V*), (W258L*), G339D, S371L, S373P, S375F, K417N, N440K, G446S, L452R, S477N, T478K, E484A, Q493R, G496S, Q498R, N501Y, Y505H, T547K, D614G, H655Y, N679K, P681H, P681R, N764K, D796Y, N856K, D950N, Q954H, N969K, and L981F.

5-19. (canceled)

20. A T cell modulatory polypeptide-epitope conjugate (T-Cell-MP-epitope conjugate), the polypeptide comprising:
(i) optionally one or more independently selected MOD polypeptide sequences, or two or more MOD polypeptide sequences, wherein when there are two or more MOD polypeptide sequences they are optionally joined to each other by independently selected L1 linkers,
(ii) an optional L2 linker polypeptide sequence joining the one or more MOD polypeptide sequences to a β2M polypeptide sequence,
(iii) the β2M polypeptide sequence,
(iv) an optional L3 linker polypeptide sequence comprising from 15 to 50 amino acids,
(v) an HLA-E polypeptide sequence,
(vi) an optional L4 linker polypeptide sequence,
(vii) a scaffold polypeptide sequence,
(viii) an optional L5 linker polypeptide sequence, and
(ix) optionally one or more independently selected MOD polypeptide sequences or two or more MOD polypeptide sequences optionally joined to each other by independently selected L6 linkers;
wherein
the T-Cell-MP-epitope conjugate comprises at least one MOD polypeptide sequence,
the MHC-H polypeptide sequence comprises a cysteine at position 84 and position 139 that may form a disulfide bond,
at least one of the β2M polypeptide sequence, the L3 linker polypeptide sequence, and/or the HLA-E polypeptide sequence comprises one or more chemical conjugation sites to which a peptide that presents an HLA-E restricted non-coronavirus epitope is covalently bound, directly or indirectly, and
the β2M polypeptide sequence has at least 90% sequence identity at least 90 or 95 contiguous aas of a mature human β2M polypeptide of SEQ ID NO:61; and
wherein the peptide presenting an HLA-E restricted non-coronavirus epitope comprises
(a) an aa sequence selected from the group consisting of: QMPSRSLLF (SEQ ID NO: 2059), TLPKRGLFL (SEQ ID NO:2060), TGPWRSLWI (SEQ ID NO:2061), ILTDRSLWL (SEQ ID NO:2062), VNPGRSLFL (SEQ ID NO:2063), WNRLFPPLR (SEQ ID NO:2064), TLPERTLYL (SEQ ID NO:2065), FLPNRSLLF (SEQ ID NO: 2066), VMGDRSVLY (SEQ ID NO:2067), and VMADKSIFY (SEQ ID NO: 2068),
(b) an aa sequence selected from the group consisting of: VMPPRTLLL (SEQ ID NO.: 2069), VMAPRTLFL (SEQ ID NO:2070), VNPGRSLFL (SEQ ID NO:2063), QMPSRSLLF (SEQ ID NO.: 2071), TLPERTLYL (SEQ ID NO:2065), VMPGRTLCF (SEQ ID NO:2072), ILTDRSLWL (SEQ ID NO:262), RMPPRSVLL (SEQ ID NO:2073), TAPARTMFL (SEQ ID NO.: 2074), and NMPARTVLF (SEQ ID NO: 2075),
(c) aa sequence WNRILPNAY (SEQ ID NO:2076),
(d) the aa sequence X1-X2-X3-X4-R-X6-X7-X8-X9-X10, where: X1 is absent or S, K, A, I, L, or T; X2 is L, I, K or V; X3 is P, A or S; X4 is G, V, P, D or R; X6 is S or T; X7 is L, I, or V; X8 is F, W, or E; X9 is L. F, W or E; and X10 F, H, S, A or absent (SEQ ID NO:2077),
(e) the aa sequence LP (G or V) RSLFL (SEQ ID NO:2078), or LP (G or V) RSLWL (SEQ ID NO: 2079),
(f) a CMV aa sequence selected from the group consisting of: VLPHRTQFL (SEQ ID NO: 2080), TGAARSFFF (SEQ ID NO:2081), and VMAPRTLIL (SEQ ID NO: 2082), or
(g) a CMV aa sequence selected from the group consisting of: SAPLKTRFL (SEQ ID NO: 2083) and SVPLKTRFL (SEQ ID NO:2084).

21. (canceled)

22. The T-Cell-MP-epitope conjugate of claim 20, wherein the MHC-H polypeptide sequence has:
(i) at least 90% or at least 95% sequence identity to at least 225 contiguous aas of an HLA-E allele; or
(ii) at least 90% or at least 95% sequence identity to at least 225 or at least 250 contiguous aas of an HLA-E*0101, HLA-E*01:03, HLA-E*01:04, HLA-E*01:05, HLA-E*01:06, HLA-E*01:07, HLA-E*01:09, or HLA-E*01:10 polypeptide sequence, or the HLA-E consus sequence of FIG. 3H (SEQ ID NO:58).

23. The T-Cell-MP-epitope conjugate of claim 3, wherein the scaffold polypeptide sequence comprises a non-interspecific sequence or interspecific sequence.

24. The T-Cell-MP-epitope conjugate of claim 3, complexed to form a duplex T-Cell-MP-epitope conjugate or other higher order T-Cell-MP-epitope conjugate comprising at least a first T-Cell-MP-epitope conjugate and a second T-Cell-MP-epitope conjugate of claim 3,
wherein
(i) the first T-Cell-MP-epitope conjugate comprises a first β2M polypeptide sequence; a first class I MHC-H polypeptide sequence; and a first scaffold polypeptide; and
(ii) the second T-Cell-MP-epitope conjugate comprises a second β2M polypeptide sequence; a second class I MHC-H polypeptide sequence; and a second scaffold polypeptide; and
wherein the first and second T-Cell-MP-epitope conjugates associate by binding interactions between the first and second scaffold polypeptides that optionally include at least one, or at least two, interchain covalent bonds.

25-27. (canceled)

28. The T-Cell-MP-epitope conjugate or the duplex or higher order T-Cell-MP-epitope conjugate of claim 24, comprising:
(i) at least one or at least two wt. MOD and/or variant MOD polypeptide sequences; or
(ii) at least three or at least four wt. MOD and/or variant MOD polypeptide sequences.

29. The T-Cell-MP-epitope conjugate or duplex T-Cell-MP-epitope conjugate of claim 28, wherein the MOD polypeptide sequences are selected independently from the group consisting of: IL-1, IL-2, IL-4, IL-6, IL-7, IL-10, IL-12, IL-15, IL-17, IL-21, IL-23, CD7, CD30L, CD40, CD70, CD80, (B7-1), CD83, CD86 (B7-2), HVEM (CD270), ILT3 (Ig-like transcript 3), ILT4 (Ig-like transcript 4), Fas ligand (FasL), ICAM (intercellular adhesion molecule), ICOS-L (inducible costimulatory ligand), JAG1 (CD339), lymphotoxin beta receptor, 3/TR6, OX40L (CD252), PD-L1, PD-L2, TGF-β1, TGF-β2, TGF-β3, 4-1BBL and anti-CD28 polypeptide sequences.

30. (canceled)

31. The T-Cell-MP-epitope conjugate or duplex T-Cell-MP-epitope conjugate of claim 28, wherein the MOD polypeptide sequences comprise at least one independently selected wt. IL-2 and/or variant IL-2 MOD polypeptide sequence comprising aa substitution at H16 and/or F42.

32. The T-Cell-MP-epitope conjugate or duplex T-Cell-MP-epitope conjugate of claim 29, wherein each chemical conjugation site is jointly or independently selected from: a) amino acid chemical conjugation sites; b) non-natural amino acids and/or selenocysteines; c) peptide sequences that act as an enzymatic modification sequence (e.g., a sulfatase motif); d) carbohydrate or oligosaccharide moieties; and/or e) IgG nucleotide binding sites.

33. (canceled)

34. The T-Cell-MP-epitope conjugate, duplex T-Cell-MP-epitope conjugate, or higher order T-cell-MP-epitope conjugate of claim 32, further comprising at least one, or at least two, additional polypeptides each of which is an independently selected Cancer Targeting Polypeptide (CTP) that binds to an independently selected antigenic determinates on the same or different antigens, and may be the same or different.

35-37. (canceled)

38. A method of treatment or prophylaxis of a coronavirus infection or condition associated with coronavirus infection comprising:
(i) administering to a patient or subject (e.g., a patient in need thereof) an effective amount of one or more T-Cell-MP-epitope conjugates or one or more duplex T-Cell-MP-epitope conjugates or other higher order complexes of T-Cell-MP-epitope conjugates of claim 32; or
(ii) contacting a cell or tissue in vitro or in vivo with one or more T-Cell-MP-epitope conjugates or one or more duplex T-Cell-MP-epitope conjugates or other higher order complexes of T-Cell-MP-epitope conjugates according to claim 32, and administering the cell, tissue, or progeny thereof to a patient or subject (e.g., a patient in need thereof);
wherein the coronavirus infection or condition associated with coronavirus infection includes, but not limited to, primary coronavirus infections, secondary coronavirus infections, and long COVID.

39. A method of controlling a coronavirus infection or of reducing transmission of coronavirus comprising
(i) administering to a patient or subject (e.g., a patient in need thereof) an effective amount of one or more T-Cell-MP-epitope conjugates or one or more duplex T-Cell-MP-epitope conjugates or other higher order complexes of T-Cell-MP-epitope conjugates of T-Cell-MP-epitope conjugates of claim 32; or
(ii) contacting a cell or tissue in vitro or in vivo with one or more T-Cell-MP-epitope conjugates or one or more duplex T-Cell-MP-epitope conjugates or other higher order complexes of T-Cell-MP-epitope conjugates of claim 32, and administering the cell, tissue, or progeny thereof to a patient or subject (e.g., a patient in need thereof).

40. The method of claim 38, wherein when the one or more T-Cell-MP-epitope conjugates, duplex T-Cell-MP-epitope conjugates or other higher order complexes of T-Cell-MP-epitope conjugates comprise a peptide presenting an HLA-E restricted coronavirus epitope.

41. The method of claim 39, wherein when the one or more T-Cell-MP-epitope conjugates, duplex T-Cell-MP-epitope conjugates or other higher order complexes of T-Cell-MP-epitope conjugates comprise a peptide presenting an HLA-E restricted coronavirus epitope.

42. A method of providing treatment or prophylaxis to a patient or subject having, or suspected of having, or developing a neoplasm comprising:
(i) administering to a patient or subject an effective amount of one or more T-Cell-MP-epitope conjugates, one or more duplex T-Cell-MP-epitope conjugates, or other higher order complexes of T-Cell-MP-epitope conjugates of claim 34; or
(ii) contacting a cell or tissue (e.g., a cell or tissue of the patient or subject) in vitro or in vivo with one or more T-Cell-MP-epitope conjugates or duplex T-Cell-MP-epitope conjugates of claim 34, and administering the cell, tissue, or progeny thereof to a patient/subject (e.g., a patient in need thereof).

43-44. (canceled)

45. The method of claim 42, wherein the MHC-H polypeptide sequence of the one or more T-Cell-MP-epitope conjugates, one or more duplex T-Cell-MP-epitope conjugates, or other higher order complexes of T-Cell-MP-epitope conjugates comprises an HLA-E polypeptide sequence.

46. (canceled)

47. The method of 45, wherein the one or more T-Cell-MP-epitope conjugates or one or more duplex T-Cell-MP-epitope conjugates or other higher order complexes of T-Cell-MP-epitope conjugates are conjugate to a non-coronavirus HLA-E restricted epitope.

48-55. (canceled)
